(19) United States
(12) Patent Application Publication
(10) Pub. No.: US 2023/0220109 A1
(43) Pub. Date: Jul. 13, 2023

(54) COMPOSITIONS AND METHODS RELATING TO TUMOR ACTIVATED T CELL ENGAGERS

(71) Applicant: Janux Therapeutics, Inc., La Jolla, CA (US)

(72) Inventors: David CAMPBELL, La Jolla, CA (US); Ramesh BHATT, La Jolla, CA (US); Thomas R. DIRAIMONDO, La Jolla, CA (US)

(21) Appl. No.: 17/616,281

(22) PCT Filed: Jun. 5, 2020
(86) PCT No.: PCT/US2020/036493

§ 371 Date: Dec. 3, 2021

Related U.S. Application Data
(60) Provisional application No. 62/978,662, filed on Feb. 19, 2020.
(60) Provisional application No. 62/858,254, filed on Jun. 6, 2019.

(51) Int. Cl. C07K 16/32; C07K 16/28

(52) U.S. Cl. CPC C07K 16/32; C07K 16/2863; C07K 2317/622; C07K 2317/55; C07K 2319/50; C07K 2317/569; C07K 2317/92; C07K 2317/94; A61K 2039/505

(57) ABSTRACT

Provided herein are modified T cell engagers, pharmaceutical compositions thereof, as well as nucleic acids, and methods for making and discovering the same. The modified T cell engagers described herein are modified with a peptide and a half-life extending molecule.

### CROSS-REFERENCE

This application claims the benefit of U.S. Provisional Application No. 62/858,254, filed Jun. 6, 2019, and U.S. Provisional Application No. 62/978,662, filed Feb. 19, 2020, which applications are incorporated herein by reference.

### SEQUENCE LISTING

The instant application contains a Sequence Listing which has been submitted electronically in ASCII format and is hereby incorporated by reference in its entirety. Said ASCII copy, created on Jun. 4, 2020, is named 52426-716_601_SL.txt and is 121,292 bytes in size.

### BACKGROUND

Protein-based therapies comprising T cell engagers have proven effective as treatments for a variety of diseases. As with any therapeutic class, there is a need to improve toxicity and side effects of such treatments, along with improving the half-life of the therapeutic molecules.

### SUMMARY

Modified T cell engagers can be used for selective destruction of an individual cell or cell type such as cancer cells of a tumor. Such modified T cell engagers induce an immune response against the tumor to clear the tumor. However, current therapies using modified T cell engagers can be toxic and inefficacious. Further, such modified T-cell engagers can have poor pharmacokinetic properties (PK). Provided herein are modified T-cell engagers that reduce toxicity in healthy tissue and thus improving safety while having improved PK properties and efficacy in eliminating the tumor. In some embodiments, the modified T-cell engagers described herein are linked to a peptide that blocks interactions of the T-cell engager with its target in healthy tissue thereby reducing target mediated drug disposition (TMDD). The modified T-cell engagers as described herein are also linked to half-life extending molecule, such as single-domain antibody, which improves the PK profile of the modified T-cell engager as compared to an unmodified T-cell engager.

Disclosed herein, in certain embodiments, are polypeptide complexes comprising a structural arrangement according to the configuration shown in FIG. 50X, wherein the polypeptide complex comprises a single chain variable fragment (scFv) comprising a light chain variable domain and a heavy chain variable domain, wherein the scFv is linked to a peptide (P₁) at an N-terminus of the scFv with a linking moiety (L₁) that is a substrate for a tumor specific protease, wherein P₁ impairs binding of the scFv to an effector cell antigen, and P₁ is further linked to a half-life extending molecule; and an antigen recognizing molecule that binds to a tumor cell antigen, wherein the antigen recognizing molecule comprises a Fab light chain polypeptide and a Fab heavy chain polypeptide, wherein the antigen recognizing molecule is linked to the scFv, and the antigen recognizing molecule is further linked to P₂ and L₂, wherein P₂ comprises a peptide that impairs binding of the antigen recognizing molecule to the tumor cell antigen; and L₂ comprises a linking moiety that connects the antigen recognizing molecule to P₂ and is a substrate for a tumor specific protease. In some instances, the antigen recognizing molecule is a Fab or a Fab′. In some instances, the heavy chain variable domain is linked to an N-terminus of the Fab heavy chain polypeptide and L₂ is connected to an N-terminus of the Fab light chain polypeptide. In some instances, the heavy chain variable domain is linked to an N-terminus of the Fab light chain polypeptide and L₂ is connected to an N-terminus of the Fab heavy chain polypeptide. In some instances, the light chain variable domain is linked to an N-terminus of the Fab heavy chain polypeptide and L₂ is connected to an N-terminus of the Fab light chain polypeptide. In some instances, the light chain variable domain is linked to an N-terminus of the Fab light chain polypeptide and L₂ is connected to an N-terminus of the Fab heavy chain polypeptide. In some instances, the polypeptide complex has a molecular weight of less than about 110 kDa. In some instances, the heavy chain variable domain, light chain variable domain, Fab heavy chain polypeptide, Fab light chain polypeptide, and half-life extending molecule have a combined molecular weight of less than about 100 kDa. In some instances, the tumor cell antigen comprises epidermal growth factor receptor (EGFR), human epidermal growth factor receptor 2 (HER2), or mesothelin. In some instances, the effector cell antigen comprises cluster of differentiation 3 (CD3). In some instances, the scFv comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some instances, the scFv comprises complementary determining regions (CDR)s of SP34. In some instances, the scFv comprises an amino acid sequence that has at least 85% sequence identity to SEQ ID NO: 66, SEQ ID NO: 67, or SEQ ID NO: 68. In some instances, P₁ impairs binding of the scFv to the effector cell antigen by binding to the scFv through ionic interactions, electrostatic interactions, hydrophobic interactions, Pi-stacking interactions, and H-bonding interactions. In some instances, P₁ impairs binding of the scFv to the effector cell antigen by binding to the scFv at or near an antigen binding site. In some instances, P₁ comprises an amino acid sequence of at least 10 amino acids in length and no more than 20 amino acids in length. In some instances, P₁ has less than 70% sequence identity to an amino acid sequence of the effector cell antigen. In some instances, P₁ has less than 70% sequence identity to an amino acid sequence of CD3. In some instances, P₁ comprises an amino acid sequence according to SEQ ID NOs: 18, 19, 20, 21, 22, 23, 24, 25, 26, 27, or 28. In some instances, L₁ comprises a urokinase cleavable amino acid sequence, a matriptase cleavable amino acid sequence, matrix metalloprotease cleavable amino acid sequence, or a legumain cleavable amino acid sequence. In some instances, L₁ has a formula comprising (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), or (GSSGGS)ₙ (SEQ ID NO: 33), wherein n is an integer of at least 1. In some instances, L₁ comprises an amino acid sequence according to SEQ ID NOs: 34, 35, 36, 37, 38, 39, 40, 41, 42, 43, 44, 45, 46, 47, 48, 49, 50, or 55. In some instances, L₁ comprises an amino acid sequence according to SEQ ID NO: 42. In some instances, the half-life extending molecule comprises a linking moiety (L₃) that connects the half-life extending molecule to P₁. In some instances, L₃ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1. In some instances, L₃ comprises an amino acid sequence according to SEQ ID NO: 51. In some instances, the half-life extending molecule comprises an antibody. In some instances, the antibody comprises a single domain antibody, a single chain variable fragment, or a Fab. In some instances, the single domain antibody binds to albumin In some instances, the single domain antibody comprises 10G or 10GE. In some instances, the single domain antibody comprises 10G, and the single domain antibody comprises an amino acid sequence according to SEQ ID NO: 52. In some instances, P₂ impairs binding of the antigen recognizing molecule to the tumor cell antigen by binding to the antigen recognizing molecule through ionic interactions, electrostatic interactions, hydrophobic interactions, Pi-stacking interactions, and H-bonding interactions. In some instances, P₂ impairs binding of the antigen recognizing molecule to the tumor cell antigen by binding to the antigen recognizing molecule at or near an antigen binding site. In some instances, P₂ comprises an amino acid sequence of at least 10 amino acids in length and no more than 20 amino acids in length. In some instances, P₂ has less than 70% sequence identity to an amino acid sequence of the tumor cell antigen. In some instances, the tumor cell antigen comprises epidermal growth factor receptor (EGFR). In some instances, P₂ comprises an amino acid sequence according to SEQ ID NOs: 1, 2, 3, 4, 5, 6, or 7. In some instances, the Fab light chain polypeptide comprises an amino acid sequence that has at least 85% sequence identity to SEQ ID NO: 56 or SEQ ID NO: 57. In some instances, the Fab heavy chain polypeptide comprises an amino acid sequence that has at least 85% sequence identity to SEQ ID NO: 58, SEQ ID NO: 59, or SEQ ID NO: 60. In some instances, the tumor cell antigen comprises human epidermal growth factor receptor 2 (HER2). In some instances, P₂ comprises an amino acid sequence according to SEQ ID NOs: 8, 9, 10, 11, 12, 13, 14, 15, 16, or 17. In some instances, the Fab light chain polypeptide comprises an amino acid sequence that has at least 85% sequence identity to SEQ ID NO: 61. In some instances, the Fab heavy chain polypeptide comprises an amino acid sequence that has at least 85% sequence identity to SEQ ID NO: 62 or SEQ ID NO: 63. In some instances, L₂ comprises a urokinase cleavable amino acid sequence, a matriptase cleavable amino acid sequence, matrix metalloprotease cleavable amino acid sequence, or a legumain cleavable amino acid sequence. In some instances, L₂ has a formula comprising (G₂S)ₙ, (GS)ₙ, (GSGGS)ₙ (SEQ ID NO: 30), (GGGS)ₙ (SEQ ID NO: 31), (GGGGS))ₙ (SEQ ID NO: 32), or (GSSGGS)ₙ (SEQ ID NO: 33), wherein n is an integer of at least 1. In some instances, L₂ comprises an amino acid sequence according to SEQ ID NOs: 34, 35, 36, 37, 38, 39, 40, 41, 42, 43, 44, 45, 46, 47, 48, 49, 50, or 55. In some instances, L₂ comprises the amino acid sequence according to SEQ ID NO: 42.

Disclosed herein, in certain embodiments, are pharmaceutical compositions comprising: (i) the polypeptide complex as described herein; and (ii) a pharmaceutically acceptable excipient.

Disclosed herein, in certain embodiments, are isolated recombinant nucleic acid molecules encoding the polypeptide or polypeptide complex as described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1G illustrates preparation and biotinylation of cetuximab and trastuzumab. Antibodies were biotinylated prior to phage panning using he EZ-link Sulfo-NHS-LC-LC-Biotin reagent and evaluated for their ability to bind cognate antigen. FIG. 1A illustrates naked Ab-1, Ab-3 binding to EGFR-biotin using 3 nM, 1 nM, 0.3 nM, 0.1 nM, 0.03 nM, and 0 nM Ab-1, Ab-3 in solution. FIG. 1B illustrates EGFR binding to biotinylated Ab-1, Ab-3 using 25 nM, 12.5 nM, 6.25 nM, 3.125 nM, 1.5625 nM, and 0 nM EGFR in solution. FIG. 1C illustrates naked Ab-6, Ab-7 binding to HER2-biotin using 25 nM, 12.5 nM, 6.25 nM, 3.125 nM, 1.56 nM, 0.78 nM, 0.39 nM and 0 nM Ab-6, Ab-7 in solution. FIG. 1D illustrates HER2 binding to biotinylated Ab-6, Ab-7 using 70 nM HER2 in solution FIG. 1E illustrates HER2 binding to biotinylated Ab-6, Ab-7 using 0 nM HER2 in solution FIG. 1F illustrates naked Ab-9, Ab-10 binding to CD3-biotin using 3 nM, 1 nM, 0.3 nM, 0.1 nM, 0.03 nM, and 0 nM Ab-9, Ab-10 in solution. FIG. 1G illustrates CD3 binding to biotinylated Ab-9, Ab-10 using 200 nM and 0 nM CD3 in solution.

FIGS. 2A-2C illustrate peptide panning using phage display enables discovery of antibody inhibitory peptides. Peptides were displayed via p3 or p8 phage protein fusion and biopanned against Trastuzumab (Ab-6, Ab-7). FIG. 2A depicts a panning process involving standard bind, wash, elute, and amplify cycles. The eluted phage after multiple rounds of panning were used to infect bacteria, plated on agar, individual colonies picked and amplified, followed by binding assessments and sequencing. Figure discloses SEQ ID NOS 112-113, respectively, in order of appearance. FIG. 2B illustrates binding of clonal phagemid to plate captured Ab-6, Ab-7 characterized by ELISA. Biotinylated antibody was captured on neutravidin coated plates followed by incubation with phage. Bound phage was detected using an anti-m13 HRP antibody conjugate. Phage binding to neutravidin captured biotinylated antibody was compared to phage binding to neutravidin alone. FIG. 2C illustrates clonal phage binders of Ab-6, Ab-7 that did not bind neutravidin evaluated for their ability to bind in the presence and absence of the cognate antigen. Inhibition of phage binding using pre-incubation of soluble HER2 at 6 nM or 20 nM was used as an indicator that clonal phage bound within or near the antibody binding sites responsible for HER2 recognition.

FIGS. 3A-3K illustrate kinetic binding of Trastuzumab (Ab-6, Ab-7) to example peptides or HER2 via BLI. FIG. 3A illustrates kinetic binding of Trastuzumab (Ab-6, Ab-7) to Peptide-8. FIG. 3B illustrates kinetic binding of Trastuzumab (Ab-6, Ab-7) to Peptide-9. FIG. 3C illustrates kinetic binding of Trastuzumab (Ab-6, Ab-7) to Peptide-10. FIG. 3D illustrates kinetic binding of Trastuzumab (Ab-6, Ab-7) to Peptide-11. FIG. 3E illustrates kinetic binding of Trastuzumab (Ab-6, Ab-7) to Peptide-12. FIG. 3F illustrates kinetic binding of Trastuzumab (Ab-6, Ab-7) to Peptide-13. FIG. 3G illustrates kinetic binding of Trastuzumab (Ab-6, Ab-7) to Peptide-14. FIG. 3H illustrates kinetic binding of Trastuzumab (Ab-6, Ab-7) to Peptide-15. FIG. 3I illustrates kinetic binding of Trastuzumab (Ab-6, Ab-7) to Peptide-16. FIG. 3J illustrates kinetic binding of Trastuzumab (Ab-6, Ab-7) to Peptide-17. FIG. 3K illustrates kinetic binding of Trastuzumab (Ab-6, Ab-7) to Her2.

FIGS. 4A-4H illustrate kinetic binding of Cetuximab (Ab-1, Ab-3) to example peptides or EGFR via BLI. FIG. 4A illustrates kinetic binding of Cetuximab (Ab-1, Ab-3) to Peptide-1. FIG. 4B illustrates kinetic binding of Cetuximab (Ab-1, Ab-3) to Peptide-2. FIG. 4C illustrates kinetic binding of Cetuximab (Ab-1, Ab-3) to Peptide-3. FIG. 4D illustrates kinetic binding of Cetuximab (Ab-1, Ab-3) to Peptide-4. FIG. 4E illustrates kinetic binding of Cetuximab (Ab-1, Ab-3) to Peptide-5. FIG. 4F illustrates kinetic binding of Cetuximab (Ab-1, Ab-3) to Peptide-6. FIG. 4G illustrates kinetic binding of Cetuximab (Ab-1, Ab-3) to Peptide-7. FIG. 4H illustrates a blank.

FIGS. 5A-5L illustrates kinetic binding of SP34 (Ab-9, Ab-10) to example peptides or CD3 via BLI. FIG. 5A illustrates kinetic binding of SP34 (Ab-9, Ab-10) to Peptide-19. FIG. 5B illustrates kinetic binding of SP34 (Ab-9, Ab-10) to Peptide-20. FIG. 5C illustrates kinetic binding of SP34 (Ab-9, Ab-10) to Peptide-21. FIG. 5D illustrates kinetic binding of SP34 (Ab-9, Ab-10) to Peptide-22. FIG. 5E illustrates kinetic binding of SP34 (Ab-9, Ab-10) to Peptide-23. FIG. 5F illustrates kinetic binding of SP34 (Ab-9, Ab-10) to Peptide-24. FIG. 5G illustrates kinetic binding of SP34 (Ab-9, Ab-10) to Peptide-26. FIG. 5H illustrates kinetic binding of SP34 (Ab-9, Ab-10) to Peptide-26. FIG. 5I illustrates kinetic binding of SP34 (Ab-9, Ab-10) to Peptide-27. FIG. 5J illustrates kinetic binding of SP34 (Ab-9, Ab-10) to Peptide-28. FIG. 5K illustrates kinetic binding of SP34 (Ab-9, Ab-10) to Peptide-18. FIG. 5L illustrates a blank.

FIGS. 6A-6C illustrate equilibrium binding via ELISA. FIG. 6A illustrates equilibrium binding of Trastuzumab (Ab-6, Ab-7) to example peptides via ELISA. FIG. 6B illustrates equilibrium binding of Cetuximab (Ab-1, Ab-3) to example peptides via ELISA. FIG. 6C illustrates equilibrium binding of SP34 (Ab-9, Ab-10) to example peptides via ELISA.

FIGS. 7A-7F illustrate that 100 uM peptides inhibit kinetic binding of 2 nM Ab-6, Ab-7 to HER2 via BLI. FIG. 7A illustrates kinetic binding of 2 nM Ab-6, Ab-7 to HER2 via BLI in the absence of a peptide. FIG. 7B illustrates that 100 uM Peptide-8 inhibit kinetic binding of 2 nM Ab-6, Ab-7 to HER2 via BLI. FIG. 7C illustrates that 100 uM Peptide-9 inhibit kinetic binding of 2 nM Ab-6, Ab-7 to HER2 via BLI. FIG. 7D illustrates that 100 uM Peptide-10 inhibit kinetic binding of 2 nM Ab-6, Ab-7 to HER2 via BLI. FIG. 7E illustrates that 100 uM Peptide-11 inhibit kinetic binding of 2 nM Ab-6, Ab-7 to HER2 via BLI. FIG. 7F illustrates that 100 uM Peptide-12 inhibit kinetic binding of 2 nM Ab-6, Ab-7 to HER2 via BLI.

FIGS. 8A-8I illustrate that 100 uM peptides inhibit kinetic binding of 2 nM Ab-1, Ab-3 to EGFR via BLI. FIG. 8A illustrates kinetic binding of 2 nM Ab-1, Ab-3 to EGFR via BLI in the absence of a peptide. FIG. 8B illustrates that 100 uM Peptide-1 inhibit kinetic binding of 2 nM Ab-1, Ab-3 to EGFR via BLI. FIG. 8C illustrates that 100 uM Peptide-2 inhibit kinetic binding of 2 nM Ab-1, Ab-3 to EGFR via BLI. FIG. 8D illustrates that 100 uM Peptide-3 inhibit kinetic binding of 2 nM Ab-1, Ab-3 to EGFR via BLI. FIG. 8E illustrates that 100 uM Peptide-4 inhibit kinetic binding of 2 nM Ab-1, Ab-3 to EGFR via BLI. FIG. 8F illustrates that 100 uM Peptide-5 inhibit kinetic binding of 2 nM Ab-1, Ab-3 to EGFR via BLI. FIG. 8G illustrates that 100 uM Peptide-6 inhibit kinetic binding of 2 nM Ab-1, Ab-3 to EGFR via BLI. FIG. 8H illustrates that 100 uM Peptide-7 inhibit kinetic binding of 2 nM Ab-1, Ab-3 to EGFR via BLI. FIG. 8I illustrates a blank.

FIGS. 9A-9L illustrate that 100 uM peptides inhibit kinetic binding of 2 nM Ab-9, Ab-10 to CD3 via BLI. FIG. 9A illustrates kinetic binding of 2 nM Ab-9, Ab-10 to CD3 via BLI in the absence of a peptide. FIG. 9B illustrates that 100 uM Peptide-18 inhibit kinetic binding of 2 nM Ab-9, Ab-10 to CD3 via BLI. FIG. 9C illustrates that 100 uM Peptide-19 inhibit kinetic binding of 2 nM Ab-9, Ab-10 to CD3 via BLI. FIG. 9D illustrates that 100 uM Peptide-20 inhibit kinetic binding of 2 nM Ab-9, Ab-10 to CD3 via BLI. FIG. 9E illustrates that 100 uM Peptide-21 inhibit kinetic binding of 2 nM Ab-9, Ab-10 to CD3 via BLI. FIG. 9F illustrates that 100 uM Peptide-22 inhibit kinetic binding of 2 nM Ab-9, Ab-10 to CD3 via BLI. FIG. 9G illustrates that 100 uM Peptide-23 inhibit kinetic binding of 2 nM Ab-9, Ab-10 to CD3 via BLI. FIG. 9H illustrates that 100 uM Peptide-24 inhibit kinetic binding of 2 nM Ab-9, Ab-10 to CD3 via BLI. FIG. 9I illustrates that 100 uM Peptide-25 inhibit kinetic binding of 2 nM Ab-9, Ab-10 to CD3 via BLI. FIG. 9J illustrates that 100 uM Peptide-26 inhibit kinetic binding of 2 nM Ab-9, Ab-10 to CD3 via BLI. FIG. 9K illustrates that 100 uM Peptide-26 inhibit kinetic binding of 2 nM Ab-9, Ab-10 to CD3 via BLI. FIG. 9L illustrates that 100 uM Peptide-28 inhibit kinetic binding of 2 nM Ab-9, Ab-10 to CD3 via BLI.

FIGS. 10A-10C illustrate dose dependent inhibition. FIG. 10A illustrates that peptides inhibit 0.1 nM Ab-6, Ab-7 binding HER2 in a dose dependent manner by ELISA. FIG. 10B illustrates that peptides inhibit 0.2 nM Ab-1, Ab-3 binding EGFR in a dose dependent manner by ELISA. FIG. 10C illustrates that peptides inhibit 1 nM Ab-9, Ab-10 binding CD3 in a dose dependent manner by ELISA.

FIG. 11 depicts the tumor specific activity and cross over PK concepts within tumor activated T cell engager molecules or polypeptide complexes.

FIG. 12 depicts general dual mask and single mask polypeptide complex designs.

FIGS. 13A-13C illustrate analysis of non-masked polypeptide complex, PC-1. FIG. 13A illustrates SDS-PAGE of non-masked polypeptide complex, PC-1. FIG. 13B illustrates SEC-FPLC of non-masked polypeptide complex, PC-1. FIG. 13C illustrates liquid chromatography-mass spectrometry (LC-MS) analysis of non-masked polypeptide complex, PC-1.

FIGS. 14A-14C illustrate analysis of masked polypeptide complex, PC-2. FIG. 14A illustrates SDS-PAGE of masked polypeptide complex, PC-2. FIG. 14B illustrates SEC-FPLC of masked polypeptide complex, PC-2. FIG. 14C illustrates liquid chromatography-mass spectrometry (LC-MS) analysis of masked polypeptide complex, PC-2.

FIGS. 15A-15C illustrate analysis of masked polypeptide complex, PC-3. FIG. 15A illustrates SDS-PAGE of masked polypeptide complex, PC-3. FIG. 15B illustrates SEC-FPLC of masked polypeptide complex, PC-3. FIG. 15C illustrates liquid chromatography-mass spectrometry (LC-MS) analysis of masked polypeptide complex, PC-3.

FIGS. 16A-16C illustrate analysis of masked polypeptide complex, PC-4. FIG. 16A illustrates SDS-PAGE of masked polypeptide complex, PC-4. FIG. 16B illustrates SEC-FPLC of masked polypeptide complex, PC-4. FIG. 16C illustrates liquid chromatography-mass spectrometry (LC-MS) analysis of masked polypeptide complex, PC-4.

FIGS. 17A-17B illustrate analysis of masked polypeptide complex, PC-5. FIG. 17A illustrates SDS-PAGE of masked polypeptide complex, PC-5. FIG. 17B illustrates SEC-FPLC of masked polypeptide complex, PC-5.

FIGS. 18A-18C illustrate analysis of masked polypeptide complex, PC-6. FIG. 18A illustrates SDS-PAGE of masked polypeptide complex, PC-6. FIG. 18B illustrates SEC-FPLC of masked polypeptide complex, PC-6. FIG. 18C illustrates liquid chromatography-mass spectrometry (LC-MS) analysis of masked polypeptide complex, PC-6.

FIGS. 19A-19B illustrate analysis of masked polypeptide complex, PC-7. FIG. 19A illustrates SDS-PAGE of masked polypeptide complex, PC-7. FIG. 19B illustrates SEC-FPLC of masked polypeptide complex, PC-7.

FIGS. 20A-20B illustrate analysis of non-masked polypeptide complex, PC-8. FIG. 20A illustrates SDS-PAGE of non-masked polypeptide complex, PC-8. FIG. 20B illustrates SEC-FPLC of non-masked polypeptide complex, PC-8.

FIGS. 21A-21B illustrate analysis of masked polypeptide complex, PC-9. FIG. 21A illustrates SDS-PAGE of masked polypeptide complex, PC-9. FIG. 21B illustrates SEC-FPLC of masked polypeptide complex, PC-9.

FIGS. 22A-22B illustrate analysis of masked polypeptide complex, PC-10. FIG. 22A illustrates SDS-PAGE of masked polypeptide complex, PC-10. FIG. 22B illustrates SEC-FPLC of masked polypeptide complex, PC-10.

FIGS. 23A-23B illustrate analysis of non-masked polypeptide complex, PC-11. FIG. 23A illustrates SDS-PAGE of non-masked polypeptide complex, PC-11. FIG. 23B illustrates SEC-FPLC of non-masked polypeptide complex, PC-11.

FIGS. 24A-24B illustrate analysis of masked polypeptide complex, PC-12. FIG. 24A illustrates SDS-PAGE of masked polypeptide complex, PC-12. FIG. 24B illustrates SEC-FPLC of masked polypeptide complex, PC-12.

FIG. 25 illustrates polypeptide complex binding albumin from different species by ELISA. HSA=human serum albumin; CSA=cynomolgus serum albumin; MSA=mouse serum albumin; BSA=bovine serum albumin

FIGS. 26A-26D illustrate polypeptide complex kinetic binding to HER2 by BLI. FIG. 26A illustrates binding of non-masked polypeptide complex PC-11 to HER2. FIG. 26B illustrates binding of masked polypeptide complex PC-12 to HER2. FIG. 26C illustrate binding of masked polypeptide complex PC-12 treated with MTSP1 to HER2 FIG. 26D illustrates a blank

FIGS. 27A-27H illustrate polypeptide complex kinetic binding to EGFR in the presence of bovine serum albumin (BSA) buffer by BLI. FIG. 27A illustrates kinetic binding of PC-1 to EGFR in the presence of BSA buffer. FIG. 27B illustrates kinetic binding of PC-1 treated with uPa to EGFR in the presence of BSA buffer. FIG. 27C illustrates kinetic binding of PC-2 to EGFR in the presence of BSA buffer. FIG. 27D illustrates kinetic binding of PC-2 treated with uPa to EGFR in the presence of BSA buffer. FIG. 27E illustrates kinetic binding of PC-3 to EGFR in the presence of BSA buffer. FIG. 27F illustrates kinetic binding of PC-3 treated with uPa to EGFR in the presence of BSA buffer. FIG. 27G illustrates kinetic binding of PC-10 to EGFR in the presence of BSA buffer. FIG. 27H illustrates kinetic binding of PC-6 treated with uPa to EGFR in the presence of BSA buffer.

FIGS. 28A-28R illustrate polypeptide complex kinetic binding to EGFR in the presence of human serum albumin (HSA) buffer by BLI. FIG. 28A illustrates kinetic binding of PC-1 to EGFR in the presence of HSA buffer. FIG. 28B illustrates kinetic binding of PC-1 treated with uPa to EGFR in the presence of HSA buffer. FIG. 28C illustrates kinetic binding of PC-2 to EGFR in the presence of HSA buffer. FIG. 28D illustrates kinetic binding of PC-2 treated with uPa to EGFR in the presence of HSA buffer. FIG. 28E illustrates kinetic binding of PC-3 to EGFR in the presence of HSA buffer. FIG. 28F illustrates kinetic binding of PC-3 treated with uPa to EGFR in the presence of HSA buffer. FIG. 28G illustrates kinetic binding of PC-4 to EGFR in the presence of HSA buffer. FIG. 28H illustrates kinetic binding of PC-4 treated with MTSP1 to EGFR in the presence of HSA buffer. FIG. 28I illustrates kinetic binding of PC-5 to EGFR in the presence of HSA buffer. FIG. 28J illustrates kinetic binding of PC-5 treated with MTSP1 to EGFR in the presence of HSA buffer. FIG. 28K illustrates kinetic binding of PC-7 to EGFR in the presence of HSA buffer. FIG. 28L illustrates kinetic binding of PC-7 treated with MTSP1 to EGFR in the presence of HSA buffer. FIG. 28M illustrates kinetic binding of PC-8 to EGFR in the presence of HSA buffer. FIG. 28N illustrates kinetic binding of PC-8 treated with MTSP1 to EGFR in the presence of HSA buffer. FIG. 28O illustrates kinetic binding of PC-9 to EGFR in the presence of HSA buffer. FIG. 28P illustrates kinetic binding of PC-9 treated with MTSP1 to EGFR in the presence of HSA buffer. FIG. 28Q illustrates kinetic binding of PC-10 to EGFR in the presence of HSA buffer. FIG. 28R illustrates kinetic binding of PC-10treated with MTSP1 to EGFR in the presence of HSA buffer.

FIGS. 29A-29L illustrate polypeptide complex kinetic binding to CD3 in the presence of bovine serum albumin (BSA) buffer by BLI. FIG. 29A illustrates kinetic binding of PC-1 to CD3 in the presence of BSA buffer. FIG. 29B illustrates kinetic binding of PC-1 treated with uPa to CD3 in the presence of BSA buffer. FIG. 29C illustrates kinetic binding of PC-2 to CD3 in the presence of BSA buffer. FIG. 29D illustrates kinetic binding of PC-2 treated with uPa to CD3 in the presence of BSA buffer. FIG. 29E illustrates kinetic binding of PC-3 to CD3 in the presence of BSA buffer. FIG. 29F illustrates kinetic binding of PC-3 treated with uPa to CD3 in the presence of BSA buffer. FIG. 29G illustrates kinetic binding of PC-10 to CD3 in the presence of BSA buffer. FIG. 29H illustrates kinetic binding of PC-10 treated with MTSP1 to CD3 in the presence of BSA buffer. FIG. 29I illustrates kinetic binding of PC-12 to CD3 in the presence of BSA buffer. FIG. 29J illustrates kinetic binding of PC-12 treated with MTSP1 to CD3 in the presence of BSA buffer. FIG. 29K illustrates kinetic binding of PC-11 to CD3 in the presence of BSA buffer. FIG. 29L illustrates kinetic binding of PC-6 to CD3 in the presence of BSA buffer.

FIGS. 30A-30R illustrate polypeptide complex kinetic binding to CD3 in the presence of human serum albumin (HSA) buffer by BLI. FIG. 30A illustrates kinetic binding of PC-1 to CD3 in the presence of HSA buffer. FIG. 30B illustrates kinetic binding of PC-1 treated with uPa to CD3 in the presence of HSA buffer. FIG. 30C illustrates kinetic binding of PC-2 to CD3 in the presence of HSA buffer. FIG. 30D illustrates kinetic binding of PC-2 treated with uPa to CD3 in the presence of HSA buffer. FIG. 30E illustrates kinetic binding of PC-3 to CD3 in the presence of HSA buffer. FIG. 30F illustrates kinetic binding of PC-3 treated with uPa to CD3 in the presence of HSA buffer. FIG. 30G illustrates kinetic binding of PC-4 to CD3 in the presence of HSA buffer. FIG. 30H illustrates kinetic binding of PC-4 treated with MTSP1 to CD3 in the presence of HSA buffer. FIG. 30I illustrates kinetic binding of PC-5 to CD3 in the presence of HSA buffer. FIG. 30J illustrates kinetic binding of PC-5 treated with MTSP1 to CD3 in the presence of HSA buffer. FIG. 30K illustrates kinetic binding of PC-7 to CD3 in the presence of HSA buffer. FIG. 30L illustrates kinetic binding of PC-7 treated with MTSP1 to CD3 in the presence of HSA buffer. FIG. 30M illustrates kinetic binding of PC-8 to CD3 in the presence of HSA buffer. FIG. 30N illustrates kinetic binding of PC-8 treated with MTSP1 to CD3 in the presence of HSA buffer. FIG. 30O illustrates kinetic binding of PC-9 to CD3 in the presence of HSA buffer. FIG. 30P illustrates kinetic binding of PC-9 treated with MTSP1 to CD3 in the presence of HSA buffer. FIG. 30Q illustrates kinetic binding of PC-6 to CD3 in the presence of HSA buffer. FIG. 30R illustrates a blank

FIG. 31 illustrates polypeptide complex equilibrium binding HER2 in buffer containing bovine (BSA) or human albumin (HSA) by ELISA.

FIGS. 32A-32C illustrates polypeptide complex equilibrium binding EGFR in buffer containing BSA or HSA by ELISA, before or after a protease treatment. FIG. 32A illustrates polypeptide complex equilibrium binding EGFR in buffer containing HSA, before or after uPa treatment. FIG. 32B illustrates polypeptide complex equilibrium binding EGFR in buffer containing BSA, before or after uPa treatment. FIG. 32C illustrates polypeptide complex equilibrium binding EGFR in buffer containing BSA or HSA, before or after MTSP1 treatment.

FIGS. 33A-33D illustrates polypeptide complex equilibrium binding CD3 in buffer containing BSA or HSA by ELISA, before or after a protease treatment. FIG. 33A illustrate polypeptide complex equilibrium binding CD3 in buffer containing HSA by ELISA, before or after uPa treatment. FIG. 33B illustrate polypeptide complex equilibrium binding CD3 in buffer containing BSA by ELISA, before or after MTSP1 treatment. FIG. 33C illustrate polypeptide complex equilibrium binding CD3 in buffer containing HSA by ELISA, before or after MTSP1 treatment. FIG. 33C illustrate polypeptide complex equilibrium binding CD3 in buffer containing BSA by ELISA, before or after MTSP1 treatment.

FIG. 34 illustrates cellular CD3, polypeptide complex, and EGFR tetramer ternary complex formation on the surface of human T cells by flow cytometry.

FIG. 35 illustrate cellular EGFR, polypeptide complex, and CD3 tetramer ternary complex formation on the surface of HCT116 cells by flow cytometry.

FIG. 36 illustrates polypeptide complex mediated cytotoxicity against tumor target cells, HCC1569 by LDH-Glo assay.

FIG. 37 illustrates polypeptide complex mediated t cell activation against tumor target cells, HCC1569 by IFNγ ELISA.

FIG. 38 illustrates polypeptide complex mediated HCC1569 tumor cell killing using real time cell analyzer (RTCA).

FIGS. 39A-39C illustrate polypeptide complex mediated cytotoxicity against tumor target cells, HCT116, by LDH-Glo assay. FIG. 39A illustrates polypeptide complex (PC-1, PC-2, PC-3) mediated cytotoxicity against tumor target cells, HCT116, by LDH-Glo assay before or after uPa treatment. FIG. 39B illustrates polypeptide complex (PC-4) mediated cytotoxicity against tumor target cells, HCT116, by LDH-Glo assay before or after MTSP1 treatment. FIG. 39C illustrates polypeptide complex (PC-10) mediated cytotoxicity against tumor target cells, HCT116, by LDH-Glo assay before or after MTSP1 treatment.

FIGS. 40A-40C illustrate polypeptide complex mediated T cell activation against tumor target cells, HCT116 by IFNγ ELISA. FIG. 40A illustrates polypeptide complex (PC-1, PC-2, PC-3) mediated T cell activation against tumor target cells, HCT116 by IFNγ ELISA before or after uPa treatment. FIG. 40B illustrates polypeptide complex (PC-4) mediated T cell activation against tumor target cells, HCT116 by IFNγ ELISA before or after MTSP1 treatment. FIG. 40C illustrates polypeptide complex (PC-10) mediated T cell activation against tumor target cells, HCT116 by IFNγ ELISA before or after MTSP1 treatment.

FIG. 41 illustrate polypeptide complex (PC-8, PC-4) mediated HCT116 tumor cell killing using real time cell analyzer (RTCA).

FIG. 42 illustrates PC-8 and PC-4 mouse pharmacokinetics.

FIG. 43 illustrates HCT116 growth kinetics in tumor bearing NCG mice.

FIG. 44 illustrates mouse body weight over time.

FIG. 45 depicts polypeptide complexes used in Example 5.

FIG. 46 illustrates TRACT plasma concentrations (nM) in Cynomolgus monkey.

FIGS. 47A-47F illustrate pro-inflammatory cytokine release in Cynomolgus monkey plasma after dosing polypeptide complex molecules. FIG. 47A illustrates plasma IFNγ concentration (pg/mL). FIG. 47B illustrates plasma TNFα concentration (pg/mL). FIG. 47C illustrates plasma IL-6 concentration (pg/mL). FIG. 47D illustrates plasma IL-5 concentration (pg/mL). FIG. 47E illustrates plasma IL-4 concentration (pg/mL). FIG. 47F illustrates plasma IL-2 concentration (pg/mL0.

FIGS. 48A-48C illustrates PBMC populations from Cynomolgus monkeys after polypeptide complex dosing. FIG. 48A illustrates percent of PBMCs which were CD3+. FIG. 48B illustrates % of CD3+ cells which were CD69+. FIG. 48C illustrates % of CD3+ cells which were Ki-67+.

FIGS. 49A-49D illustrate clinical chemistry and hematology after polypeptide complex dosing in Cynomolgus monkeys. FIG. 49A illustrates lymphocyte (LYM) concentration over time after polypeptide complex dosing. FIG. 49B illustrates aspartate aminotransferase (AST) concentration over time after polypeptide complex dosing. FIG. 49C illustrates albumin (ALB) concentration over time after polypeptide complex dosing. FIG. 49D illustrates alamine aminotransferase (ALT) concentration over time after polypeptide complex dosing.

FIGS. 50A-50X illustrate exemplary schemas for polypeptide complexes described herein.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

“Fragment” as used herein refers to a peptide or a polypeptide that comprises less than the full length amino acid sequence.

“Antigen-binding site” as used herein refers to the region of a polypeptide that interacts with an antigen. The antigen binding site includes amino acid residues that interact directly with an antigen and those amino acid residues that are within proximity to the antigen but that may not interact directly with the antigen.

### Polypeptides or Polypeptide Complexes.

Disclosed herein, in some embodiments, are polypeptides or polypeptide complexes comprising a half-life extending molecule. In some embodiments, the polypeptides or polypeptide complexes comprise an antibody or an antibody fragment. In some embodiments, the polypeptides or polypeptide complexes bind to a tumor cell antigen. In some embodiments, the polypeptides or polypeptide complexes bind to an effector cell antigen.

In further embodiments, the polypeptide or polypeptide complexes described herein have an optimal molecular weight for enhanced tissue penetration and distribution. In some embodiments, the polypeptide or polypeptide complexes have a molecular weight of about 80 kDa. In some embodiments, the polypeptide or polypeptide complexes have a molecular weight of about 90 kDa. In some embodiments, the polypeptide or polypeptide complexes have a molecular weight of about 100 kDa. In some embodiments, the polypeptide or polypeptide complexes have a molecular weight of about 110 kDa. In some embodiments, the polypeptide or polypeptide complexes have a molecular weight of about 120 kDa. In some embodiments, the polypeptide or polypeptide complexes have a molecular weight of about 130 kDa. In some embodiments, the polypeptide or polypeptide complexes have a molecular weight of less than about 80 kDa. In some embodiments, the polypeptide or polypeptide complexes have a molecular weight of less than about 90 kDa. In some embodiments, the polypeptide or polypeptide complexes have a molecular weight of less than about 100 kDa. In some embodiments, the polypeptide or polypeptide complexes have a molecular weight of less than about 110 kDa. In some embodiments, the polypeptide or polypeptide complexes have a molecular weight of less than about 120 kDa. In some embodiments, the polypeptide or polypeptide complexes have a molecular weight of less than about 130 kDa.

Disclosed herein, in some embodiments, are polypeptides or polypeptide complexes according to Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Disclosed herein, in some embodiments, are polypeptides or polypeptide complexes according to Formula I:

A₂-A₁-L₁-P₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen.

Disclosed herein, in some embodiments, are polypeptides or polypeptide complexes comprising Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Disclosed herein, in some embodiments, are polypeptides or polypeptide complexes comprising Formula I:

A₂-A₁-L₁-P₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen. In some embodiments, the first target antigen comprises a tumor cell antigen and the second target antigen comprises an effector cell antigen. In some embodiments, the first target antigen comprises an effector cell antigen and the second target antigen comprises a tumor cell antigen. In some embodiments, the polypeptide or polypeptide complex of formula I binds to a target cell when L₁ is cleaved by the tumor specific protease. In some embodiments, the polypeptide of formula I binds to an effector cell when L₁ is cleaved by the tumor specific protease.

Disclosed herein, in some embodiments, are polypeptides or polypeptide complexes according to Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are polypeptides or polypeptide complexes according to Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease.

Disclosed herein, in some embodiments, are polypeptides or polypeptide complexes comprising Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are polypeptides or polypeptide complexes comprising Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease.

In some embodiments, the polypeptide or polypeptide complex comprises a modified amino acid, a non-natural amino acid, a modified non-natural amino acid, or a combination thereof. In some embodiments, the modified amino acid or modified non-natural amino acid comprises a post-translational modification.

Further disclosed herein, in some embodiments, are polypeptides or polypeptide complexes according to Formula II:

L₁ₐ-P₁ₐ-H₁ₐ (Formula II)

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule.

Further disclosed herein, in some embodiments, are polypeptides or polypeptide complexes according to Formula II:

L₁ₐ-P₁ₐ-H₁ₐ (Formula II)

wherein: L₁ₐ is a tumor specific protease-cleaved linking moiety that when uncleaved connects P₁ₐ to an antigen recognizing molecule that binds to a target antigen and; P₁ₐ is a peptide that binds to the antigen recognizing molecule when L₁ₐ is uncleaved; and H₁ₐ is a half-life extending molecule.

Further disclosed herein, in some embodiments, are polypeptides or polypeptide complexes comprising Formula II:

L₁ₐ-P₁ₐ-H₁ₐ (Formula II)

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule.

Further disclosed herein, in some embodiments, are polypeptides or polypeptide comprising Formula II:

L₁ₐ-P₁ₐ-H₁ₐ (Formula II)

wherein: L₁ₐ is a tumor specific protease-cleaved linking moiety that when uncleaved connects P₁ₐ to an antigen recognizing molecule that binds to a target antigen and; P₁ₐ is a peptide that binds to the antigen recognizing molecule when L₁ₐ is uncleaved; and H₁ₐ is a half-life extending molecule. In some embodiments, the antigen recognizing molecule comprises an antibody or antibody fragment. In some embodiments, the target antigen is an anti-CD3 effector cell antigen.

### Antigen Recognizing Molecule (A₁)

Disclosed herein, in some embodiments, are polypeptides or polypeptide complexes, wherein the first target antigen comprises an effector cell antigen and the second target antigen comprises a tumor cell antigen. In some embodiments, the effector cell antigen comprises CD3. In some embodiments, the tumor cell antigen comprises EGFR, HER2, mesothelin, or CEACAM5.

In some embodiments, A₁ comprises an antibody or antibody fragment. In some embodiments, A₁ comprises an antibody or antibody fragment that is human or humanized In some embodiments, L₁ is bound to N-terminus of the antibody or antibody fragment. In some embodiments, L₁ is bound to N-terminus of the antibody or antibody fragment and A₂ is bound to the other N-terminus of the antibody or antibody fragment. In some embodiments, A₂ is bound to C-terminus of the antibody or antibody fragment. In some embodiments, L₁ is bound to C-terminus of the antibody or antibody fragment. In some embodiments, A₂ is bound to N-terminus of the antibody or antibody fragment. In some embodiments, the antibody or antibody fragment comprises a single chain variable fragment, a single domain antibody, or a Fab fragment. In some embodiments, A₁ is the single chain variable fragment (scFv). In some embodiments, the scFv comprises a scFv heavy chain polypeptide and a scFv light chain polypeptide. In some embodiments, A₁ is the single domain antibody. In some embodiments, A₁ is a Fab fragment. In some embodiments, A₁ comprises an anti-CD3e single chain variable fragment. In some embodiments, A₁ comprises an anti-CD3e single chain variable fragment that has a KD binding of 1 μM or less to CD3 on CD3 expressing cells. In some embodiments, A₁ comprises a variable light chain and variable heavy chain each of which is capable of specifically binding to human CD3. In some embodiments, A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

In some embodiments, the polypeptide or polypeptide complex of formula I binds to an effector cell when L₁ is cleaved by the tumor specific protease. In some embodiments, the polypeptide or polypeptide complex of formula I binds to an effector cell when L₁ is cleaved by the tumor specific protease and A₁ binds to the effector cell. In some embodiments, the effector cell is a T cell. In some embodiments, A₁ binds to a polypeptide that is part of a TCR-CD3 complex on the effector cell. In some embodiments, the polypeptide that is part of the TCR-CD3 complex is human CD3_{ε}. In some embodiments, the effector cell antigen comprises CD3, and the scFv comprises an amino acid sequence according to SEQ ID NOs: 64, 65, or 66.

Disclosed herein, in some embodiments, are polypeptides or polypeptide complexes, wherein the first target antigen comprises a tumor cell antigen and the second target antigen comprises an effector cell antigen. In some embodiments, the tumor cell antigen comprises EGFR, HER2, mesothelin, or CEACAM5. In some embodiments, the effector cell antigen comprises CD3.

In some embodiments, A₁ comprises an antibody or antibody fragment. In some embodiments, A₁ comprises an antibody or antibody fragment that is human or humanized In some embodiments, L₁ is bound to N-terminus of the antibody or antibody fragment. In some embodiments, A₂ is bound to C-terminus of the antibody or antibody fragment. In some embodiments, L₁ is bound to C-terminus of the antibody or antibody fragment. In some embodiments, A₂ is bound to N-terminus of the antibody or antibody fragment. In some embodiments, the antibody or antibody fragment thereof comprises a single chain variable fragment, a single domain antibody, or a Fab. In some embodiments, the antibody or antibody fragment thereof comprises a single chain variable fragment (scFv), a heavy chain variable domain (VH domain), a light chain variable domain (VL domain), a variable domain (VHH) of a camelid derived single domain antibody. In some embodiments, the antibody or antibody fragment thereof is humanized or human.

In some embodiments, A₁ is the Fab. In some embodiments, the Fab comprises (a) a Fab light chain polypeptide and (b) a Fab heavy chain polypeptide. wherein the Fab light chain polypeptide of A₁ is bound to a C-terminus of the single chain variable fragment (scFv) of A₂. In some embodiments, the Fab heavy chain polypeptide of A₁ is bound to a C-terminus of the single chain variable fragment (scFv) A₂. In some embodiments, the Fab light chain polypeptide of A₁ is bound to a N-terminus of the single chain variable fragment (scFv) of A₂. In some embodiments, the Fab heavy chain polypeptide of A₁ is bound to a N-terminus of the single chain variable fragment (scFv) A₂. In some embodiments, the Fab heavy chain polypeptide of A₁ is bound to the scFv heavy chain polypeptide of A₂ and L₁ is bound to the Fab light chain polypeptide of A₁. In some embodiments, the Fab light chain polypeptide of A₁ is bound to the scFv heavy chain polypeptide of A₂ and L₁ is bound to the Fab heavy chain polypeptide of A₁. In some embodiments, the Fab heavy chain polypeptide of A₁ is bound to the scFv light chain polypeptide of A₂ and L₁ is bound to the Fab light chain polypeptide of A₁. In some embodiments, the Fab light chain polypeptide of A₁ is bound to the scFv light chain polypeptide of A₂ and L₁ is bound to the Fab heavy chain polypeptide of A₁. In some embodiments, A₂ further comprises P₂ and L₂, wherein P₂ comprises a peptide that binds to A₂; and L₂ comprises a linking moiety that connects A₂ to P₂ and is a substrate for a tumor specific protease. In some embodiments, the Fab heavy chain polypeptide of A₁ is bound to the scFv heavy chain polypeptide of A₂ and L₁ is bound to the Fab light chain polypeptide of A₁ and L₂ is bound to the scFv light chain polypeptide of A₂. In some embodiments, the Fab heavy chain polypeptide of A₁ is bound to the scFv heavy chain polypeptide of A₂ and L₁ is bound to the Fab light chain polypeptide of A₁ and L₂ is bound to the scFv light chain polypeptide of A₂, and the polypeptide complex comprises amino acid sequence according to SEQ ID NO: 72 and SEQ ID NO: 71. In some embodiments, the Fab light chain polypeptide of A₁ is bound to the scFv heavy chain polypeptide of A₂ and L₁ is bound to the Fab heavy chain polypeptide of A₁ and L₂ is bound to the scFv light chain polypeptide of A₂. In some embodiments, the Fab heavy chain polypeptide of A₁ is bound to the scFv light chain polypeptide of A₂ and L₁ is bound to the Fab light chain polypeptide of A₁ and L₂ is bound to the scFv heavy chain polypeptide of A₂. In some embodiments, the Fab light chain polypeptide of A₁ is bound to the scFv light chain polypeptide of A₂ and L₁ is bound to the Fab heavy chain polypeptide of A₁ and L₂ is bound to the scFv heavy chain polypeptide of A₂.

In some embodiments, the antibody or antibody fragment thereof comprises an epidermal growth factor receptor (EGFR) binding domain. In some embodiments, the antibody or antibody fragment thereof comprises a mesothelin binding domain. In some embodiments, the antibody or antibody fragment thereof comprises a carcinoembryonic antigen-related cell adhesion molecule CEACAM5 binding domain. In some embodiments, the antibody or antibody fragment thereof comprises a HER2 binding domain In some embodiments, the tumor cell antigen comprises EGFR, and the Fab light chain polypeptide comprises an amino acid sequence according to SEQ ID NOs: 56 or 57. In some embodiments, the tumor cell antigen comprises EGFR, and the Fab heavy chain polypeptide comprises an amino acid sequence according to SEQ ID NOs: 59 or 60. In some embodiments, the tumor cell antigen comprises HER2, and the Fab light chain polypeptide comprises an amino acid sequence according to SEQ ID NO: 61. In some embodiments, the tumor cell antigen comprises HER2 and the Fab heavy chain polypeptide comprises an amino acid sequence according to SEQ ID NOs: 62 or 63.

In some embodiments, the polypeptide or polypeptide complex has weaker binding affinity for the tumor cell antigen as compared to the binding affinity for the tumor cell antigen of a polypeptide or polypeptide complex that does not have P₁ or L₁. In some embodiments, the polypeptide or polypeptide complex has weaker binding affinity for the tumor cell antigen that is at least 5× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 8× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 10× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 15× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 20× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 25× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 30× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 35× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 40× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 45× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 50× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 55× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 60× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 65× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 70× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 75× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 80× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 85× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 90× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 95× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 100× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 120× higher than the binding affinity for the tumor cell antigen of a form of the polypeptide or polypeptide complex that does not have P₁ or L₁. In some embodiments, the polypeptide or polypeptide complex has weaker binding affinity for the tumor cell antigen that is at least 1000× higher than the binding affinity for the tumor cell antigen of a form of the polypeptide or polypeptide complex that does not have P₁ or L₁.

In some embodiments, the polypeptide or polypeptide complex has weaker binding affinity for the tumor cell antigen as compared to the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 5× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 8× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 10× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 15× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 20× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 25× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 30× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 35× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 40× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 45× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 50× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 55× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 60× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 65× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 70× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 75× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 80× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 85× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 90× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 95× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 100× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 120× higher than the binding affinity for the tumor cell antigen of the polypeptide or polypeptide complex in which L₁ has been cleaved by the tumor specific protease. In some embodiments, the polypeptide or polypeptide complex has weaker binding affinity for the tumor cell antigen that is at least 1000× higher than the binding affinity for the tumor cell antigen of the polypeptide or polypeptide complex in which L₁ has been cleaved by the tumor specific protease.

In some embodiments, the polypeptide or polypeptide complex has an increased EC₅₀ in an IFNγ release T-cell activation assay as compared to the EC₅₀ in an IFNγ release T-cell activation assay of a polypeptide or polypeptide complex that does not have P₁ or L₁. In some embodiments, the polypeptide or polypeptide complex has an increased EC₅₀ in an IFNγ release T-cell activation assay that is at least 10× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 20× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 30× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 40× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 50× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 60× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 70× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 80× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 90× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 100× higher than the EC₅₀ in an IFNγ release T-cell activation assay of a form of the polypeptide or polypeptide complex that does not have P₁ or L₁. In some embodiments, the polypeptide or polypeptide complex has an increased EC₅₀ in an IFNγ release T-cell activation assay that is at least 1000× higher than the EC₅₀ in an IFNγ release T-cell activation assay of a form of the polypeptide or polypeptide complex that does not have P₁ or L₁.

In some embodiments, the polypeptide or polypeptide complex has an increased EC₅₀ in an IFNγ release T-cell activation assay as compared to the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 10× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 20× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 30× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 40× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 50× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 60× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 70× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 80× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 90× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 100× higher than the EC₅₀ in an IFNγ release T-cell activation assay of the polypeptide or polypeptide complex in which L₁ has been cleaved by the tumor specific protease. In some embodiments, the polypeptide or polypeptide complex has an increased EC₅₀ in an IFNγ release T-cell activation assay that is at least 1000× higher than the EC₅₀ in an IFNγ release T-cell activation assay of the polypeptide or polypeptide complex in which L₁ has been cleaved by the tumor specific protease.

In some embodiments, the polypeptide or polypeptide complex has an increased EC₅₀ in a T-cell cytolysis assay as compared to the EC₅₀ in a T-cell cytolysis assay of a polypeptide or polypeptide complex that does not have P₁ or L₁. In some embodiments, the polypeptide or polypeptide complex has an increased EC₅₀ in a T-cell cytolysis assay that is at least 10× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 20× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 30× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 40× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 50× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 60× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 70× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 80× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 90× higher than the EC₅₀ in a T-cell cytolysis assay of a form of the polypeptide or polypeptide complex that does not have P₁ or L₁. In some embodiments, the polypeptide or polypeptide complex has an increased EC₅₀ in a T-cell cytolysis assay that is at least 100× higher than the EC₅₀ in a T-cell cytolysis assay of a polypeptide or polypeptide complex that does not have P₁ or L₁. In some embodiments, the polypeptide or polypeptide complex has an increased EC₅₀ in a T-cell cytolysis assay that is at least 1000× higher than the EC₅₀ in a T-cell cytolysis assay of a polypeptide or polypeptide complex that does not have P₁ or L₁.

In some embodiments, the polypeptide or polypeptide complex has an increased EC₅₀ in a T-cell cytolysis assay as compared to the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 10× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 20× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 30× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 40× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 50× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 60× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 70× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 80× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 90× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 100× higher than the EC₅₀ in a T-cell cytolysis assay of the polypeptide or polypeptide complex in which L₁ has been cleaved by the tumor specific protease. In some embodiments, the polypeptide or polypeptide complex has an increased EC₅₀ in a T-cell cytolysis assay that is at least 1,000× higher than the EC₅₀ in a T-cell cytolysis assay of the polypeptide or polypeptide complex in which L₁ has been cleaved by the tumor specific protease.

In some embodiments, the polypeptide or polypeptide complex P₂-L₂-A₂-A₁-L₁-P₁-H₁ (formula Ia) has weaker binding affinity for the tumor cell antigen as compared to the binding affinity for the tumor cell antigen of a polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 10× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 50× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 75× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 100× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 120× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 200× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 300× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 400× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 500× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 600× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 700× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 800× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 900× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 1000× higher than the binding affinity for the tumor cell antigen of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has weaker binding affinity for the tumor cell antigen that is at least 10,000× higher than the binding affinity for the tumor cell antigen of a form of the polypeptide or polypeptide complex of formula Ia that does not have P₁, L₁, P₂, or L₂.

In some embodiments, the polypeptide or polypeptide complex P₂-L₂-A₂-A₁-L₁-P₁-H₁ (formula Ia) has weaker binding affinity for the tumor cell antigen as compared to the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 10× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 50× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 75× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 100× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 120× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 200× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 300× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 400× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 500× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 600× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 700× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 800× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 900× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 1000× higher than the binding affinity for the tumor cell antigen of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has weaker binding affinity for the tumor cell antigen that is at least 10,000× higher than the binding affinity for the tumor cell antigen of the polypeptide or polypeptide complex of formula Ia in which L₁ and L₂ have been cleaved by the tumor specific proteases.

In some embodiments, the polypeptide or polypeptide complex P₂-L₂-A₂-A₁-L₁-P₁-H₁ (formula Ia) has an increased EC₅₀ in an IFNγ release T-cell activation assay as compared to the EC₅₀ in an IFNγ release T-cell activation assay of a polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 10× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 50× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₂-H₁ (formula Ia) has an increased EC₅₀ in an IFNγ release T-cell activation assay that is at least 75× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 100× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 200× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 300× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 400× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 500× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 600× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 700× higher than the EC₅₀ in an IFNγ release T-cell activation assay of a form of the polypeptide or polypeptide complex of formula Ia that does not P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 800× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 900× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 1000× higher than the EC₅₀ in an IFNγ release T-cell activation assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in an IFNγ release T-cell activation assay that is at least 10,000× higher than the EC₅₀ in an IFNγ release T-cell activation assay of a form of the polypeptide or polypeptide complex of formula Ia that does not have P₁, L₁, P₂, or L₂.

In some embodiments, the polypeptide or polypeptide complex P₂-L₂-A₂-A₁-L₁-P₁-H₁ (formula Ia) has an increased EC₅₀ in a T-cell cytolysis assay as compared to the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 10× higher than the EC₅₀ in a T-cell cytolysis assay of the polypeptide or polypeptide complex of formula Ia in which L₁ and L₂ have been cleaved by the tumor specific proteases. some embodiments, the polypeptide or polypeptide complex P₂-L₂-A₂-A₁-L₁-P₁-H₁ (formula Ia) has an increased EC₅₀ in a T-cell cytolysis assay that is at least 50× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 75× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 100× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 200× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 300× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 400× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 500× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 600× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 700× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 800× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 900× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 1000× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 10,000× higher than the EC₅₀ in a T-cell cytolysis assay of the polypeptide or polypeptide complex of formula Ia in which L₁ and L₂ have been cleaved by the tumor specific proteases.

In some embodiments, the polypeptide or polypeptide complex P₂-L₂-A₂-A₁-L₁-P₁-H₁ (formula Ia) has an increased EC₅₀ in a T-cell cytolysis assay as compared to the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 10× higher than the EC₅₀ in a T-cell cytolysis assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 50× higher than the EC₅₀ in a T-cell cytolysis assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 75× higher than the EC₅₀ in a T-cell cytolysis assay of a form of the polypeptide or polypeptide complex of formula Ia that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 100× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 200× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 300× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 400× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 500× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 600× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 700× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 800× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 900× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 1000× higher than the EC₅₀ in a T-cell cytolysis assay of a polypeptide or polypeptide complex that does not have P₁, L₁, P₂, or L₂. In some embodiments, the polypeptide or polypeptide complex P₂-L₂-A₂-A₁-L₁-P₁-H₁ (formula Ia) has an increased EC₅₀ in a T-cell cytolysis assay that is at least 10,000× higher than the EC₅₀ in a T-cell cytolysis assay of a polypeptide or polypeptide complex that does not have P₁, L₁, P₂, or L₂.

In some embodiments, the polypeptide or polypeptide complex P₂-L₂-A₂-A₁-L₁-P₁-H₁ (formula Ia) has an increased EC₅₀ in a T-cell cytolysis assay as compared to the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 10× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 50× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 75× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 100× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 200× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 300× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 400× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 500× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 600× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 700× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 800× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 900× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 1000× higher than the EC₅₀ in a T-cell cytolysis assay of the polypeptide or polypeptide complex of formula Ia in which L₁ and L₂ have been cleaved by the tumor specific proteases. In some embodiments, the polypeptide or polypeptide complex P₂-L₂-A₂-A₁-L₁-P₁-H₁ (formula Ia) has an increased EC₅₀ in a T-cell cytolysis assay that is at least 10,000× higher than the EC₅₀ in a T-cell cytolysis assay of the polypeptide or polypeptide complex of formula Ia in which L₁ and L₂ have been cleaved by the tumor specific proteases.

### Antigen Recognizing Molecule (A₂)

In some embodiments, A₂ comprises an antibody or antibody fragment. In some embodiments, the antibody or antibody fragment thereof comprises a single chain variable fragment, a single domain antibody, or a Fab. In some embodiments, the antibody or antibody fragment thereof comprises a single chain variable fragment (scFv), a heavy chain variable domain (VH domain), a light chain variable domain (VL domain), a variable domain (VHH) of a camelid derived single domain antibody. In some embodiments, the antibody or antibody fragment thereof is humanized or human In some embodiments, A₂ is the Fab. In some embodiments, the Fab comprises (a) a Fab light chain polypeptide and (b) a Fab heavy chain polypeptide. In some embodiments, the antibody or antibody fragment thereof comprises an epidermal growth factor receptor (EGFR) binding domain. In some embodiments, the antibody or antibody fragment thereof comprises a mesothelin binding domain. In some embodiments, the antibody or antibody fragment thereof comprises a carcinoembryonic antigen-related cell adhesion molecule CEACAM5 binding domain. In some embodiments, the antibody or antibody fragment thereof comprises a carcinoembryonic antigen-related cell adhesion molecule HER2 binding domain. In some embodiments, the tumor cell antigen comprises EGFR, and the Fab light chain polypeptide comprises an amino acid sequence according to SEQ ID NOs: 56 or 57. In some embodiments, the tumor cell antigen comprises EGFR, and the Fab heavy chain polypeptide comprises an amino acid sequence according to SEQ ID NOs: 59 or 60. In some embodiments, the tumor cell antigen comprises HER2, and the Fab light chain polypeptide comprises an amino acid sequence according to SEQ ID NO: 61. In some embodiments, the tumor cell antigen comprises HER2 and the Fab heavy chain polypeptide comprises an amino acid sequence according to SEQ ID NOs: 62 or 63.

In some embodiments, the Fab light chain polypeptide of A₂ is bound to a C-terminus of the single chain variable fragment (scFv) of A₁. In some embodiments, the Fab heavy chain polypeptide of A₂ is bound to a C-terminus of the single chain variable fragment (scFv) A₁. In some embodiments, the Fab light chain polypeptide of A₂ is bound to a N-terminus of the single chain variable fragment (scFv) of A₁. In some embodiments, the Fab heavy chain polypeptide of A₂ is bound to a N-terminus of the single chain variable fragment (scFv) A₁. In some embodiments, the Fab heavy chain polypeptide of A₂ is bound to the scFv heavy chain polypeptide of A₁. In some embodiments, the Fab heavy chain polypeptide of A₂ is bound to the scFv heavy chain polypeptide of A₁, and the polypeptide complex comprises amino acid sequences of SEQ ID NO: 57 and SEQ ID NO: 76. In some embodiments, the Fab heavy chain polypeptide of A₂ is bound to the scFv heavy chain polypeptide of A₁ and the polypeptide complex comprises amino acid sequences of SEQ ID NO: 57 and SEQ ID NO: 78. In some embodiments, the Fab heavy chain polypeptide of A₂ is bound to the scFv heavy chain polypeptide of A₁ and the polypeptide complex comprises amino acid sequences of SEQ ID NO: 57 and SEQ ID NO: 73. In some embodiments, the Fab light chain polypeptide of A₂ is bound to the scFv heavy chain polypeptide of A₁. In some embodiments, the Fab heavy chain polypeptide of A₂ is bound to the scFv light chain polypeptide of A₁. In some embodiments, the Fab heavy chain polypeptide of A₂ is bound to the scFv light chain polypeptide of A₁, and the polypeptide complex comprises amino acid sequences of SEQ ID NO: 57 and SEQ ID NO: 74. In some embodiments, the Fab light chain polypeptide of A₂ is bound to the scFv light chain polypeptide of A₁. In some embodiments, A₂ further comprises P₂ and L₂, wherein P₂ comprises a peptide that binds to A₂; and L₂ comprises a linking moiety that connects A₂to P₂ and is a substrate for a tumor specific protease. In some embodiments, the Fab heavy chain polypeptide of A₂ is bound to the scFv heavy chain polypeptide of A₁and L₂ is bound to the Fab light chain polypeptide of A₂. In some embodiments, the Fab heavy chain polypeptide of A₂ is bound to the scFv heavy chain polypeptide of A₁ and L₂ is bound to the Fab light chain polypeptide of A₂ and the polypeptide complex comprises amino acid sequences of SEQ ID NO: 70 and SEQ ID NO: 73. In some embodiments, the Fab heavy chain polypeptide of A₂ is bound to the scFv heavy chain polypeptide of A₁ and L₂ is bound to the Fab light chain polypeptide of A₂ and the polypeptide complex comprises amino acid sequences of SEQ ID NO: 80 and SEQ ID NO: 81. In some embodiments, the Fab light chain polypeptide of A₂ is bound to the scFv heavy chain polypeptide of A₁ and L₂ is bound to the Fab heavy chain polypeptide of A₂. In some embodiments, the Fab heavy chain polypeptide of A₂ is bound to the scFv light chain polypeptide of A₁ and L₂ is bound to the Fab light chain polypeptide of A₂. In some embodiments, the Fab light chain polypeptide of A₂ is bound to the scFv light chain polypeptide of A₁ and L₂ is bound to the Fab heavy chain polypeptide of A₂.

In some embodiments, A₂ comprises an anti-CD3e single chain variable fragment. In some embodiments, A₂ comprises an anti-CD3e single chain variable fragment that has a KD binding of 1 μM or less to CD3 on CD3 expressing cells. In some embodiments, A₂ comprises a variable light chain and variable heavy chain each of which is capable of specifically binding to human CD3. In some embodiments, A₂ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19. In some embodiments, the polypeptide or polypeptide complex of formula I binds to an effector cell. In some embodiments, the effector cell is a T cell. In some embodiments, A₂ binds to a polypeptide that is part of a TCR-CD3 complex on the effector cell. In some embodiments, the polypeptide that is part of the TCR-CD3 complex is human CD3_{ε}. In some embodiments, the effector cell antigen comprises CD3, and the scFv comprises an amino acid sequence according to SEQ ID NOs: 66, 67, or 68.

Peptide (P₁ and P₂ and P₁ₐ)

In some embodiments, P₁ impairs binding of A₁ to the first target antigen. In some embodiments, P₁ is bound to A₁ through ionic interactions, electrostatic interactions, hydrophobic interactions, Pi-stacking interactions, and H-bonding interactions, or a combination thereof. In some embodiments, P₁ is bound to A₁ at or near an antigen binding site. In some embodiments, P₁ becomes unbound from A₁ when L₁ is cleaved by the tumor specific protease thereby exposing A₁ to the first target antigen. In some embodiments, P₁ has less than 70% sequence identity to the first target antigen. In some embodiments, P₁ has less than 75% sequence identity to the first target antigen. In some embodiments, P₁ has less than 80% sequence identity to the first target antigen. In some embodiments, P₁ has less than 85% sequence identity to the first target antigen. In some embodiments, P₁ has less than 90% sequence identity to the first target antigen. In some embodiments, P₁ has less than 95% sequence identity to the first target antigen. In some embodiments, P₁ has less than 98% sequence identity to the first target antigen. In some embodiments, P₁ has less than 99% sequence identity to the first target antigen. In some embodiments, P₁ comprises a de novo amino acid sequence that shares less than 10% sequence identity to the first target antigen.

In some embodiments, P₂ impairs binding of A₂ to the second target antigen. In some embodiments, P₂ is bound to A₂ through ionic interactions, electrostatic interactions, hydrophobic interactions, Pi-stacking interactions, and H-bonding interactions, or a combination thereof. In some embodiments, P₂ is bound to A₂ at or near an antigen binding site. In some embodiments, P₂ becomes unbound from A₂ when L₂ is cleaved by the tumor specific protease thereby exposing A₂ to the second target antigen. In some embodiments, P₂ has less than 70% sequence identity to the second target antigen. In some embodiments, P₂ has less than 75% sequence identity to the second target antigen. In some embodiments, P₂ has less than 80% sequence identity to the second target antigen. In some embodiments, P₂ has less than 85% sequence identity to the second target antigen. In some embodiments, P₂ has less than 90% sequence identity to the second target antigen. In some embodiments, P₂ has less than 95% sequence identity to the second target antigen. In some embodiments, P₂ has less than 98% sequence identity to the second target antigen. In some embodiments, P₂ has less than 99% sequence identity to the second target antigen. In some embodiments, P₂ comprises a de novo amino acid sequence that shares less than 10% sequence identity to the second target antigen.

In some embodiments, P₁ₐ when L₁ₐ is uncleaved impairs binding of the antigen recognizing molecule to the target antigen. In some embodiments, the antigen recognizing molecule comprises an antibody or antibody fragment. In some embodiments, the target antigen is an anti-CD3 effector cell antigen. In some embodiments, the target antigen is a tumor cell antigen. In some embodiments, the tumor cell antigen is EGFR, HER2, mesothelin, or CEACAM5. In some embodiments, P₁ₐ has less than 70% sequence identity to the target antigen. In some embodiments, P₁ₐ has less than 75% sequence identity to the target antigen. In some embodiments, P₁ₐ has less than 80% sequence identity to the target antigen. In some embodiments, P₁ₐ has less than 85% sequence identity to the target antigen. In some embodiments, P₁ₐ has less than 90% sequence identity to the target antigen. In some embodiments, P₁ₐ has less than 95% sequence identity to the target antigen. In some embodiments, P₁ₐ has less than 98% sequence identity to the target antigen. In some embodiments, P₁ₐ has less than 99% sequence identity to the target antigen. In some embodiments, P₁ₐ comprises a de novo amino acid sequence that shares less than 10% sequence identity to the second target antigen.

In some embodiments, P₁, P₂, or P₁ₐ comprises a peptide sequence of at least 5 amino acids in length. In some embodiments, P₁, P₂, or P₁ₐ comprises a peptide sequence of at least 6 amino acids in length. In some embodiments, P₁, P₂, or P₁ₐ comprises a peptide sequence of at least 10 amino acids in length. In some embodiments, P₁, P₂, or P₁ₐ comprises a peptide sequence of at least 10 amino acids in length and no more than 20 amino acids in length. In some embodiments, P₁, P₂, or P₁ₐ comprises a peptide sequence of at least 16 amino acids in length. In some embodiments, P₁, P₂, or P₁ₐ comprises a peptide sequence of no more than 40 amino acids in length. In some embodiments, P₁, P₂, or P₁ₐ comprises at least two cysteine amino acid residues. In some embodiments, P₁, P₂, or P₁ₐ comprises a cyclic peptide or a linear peptide. In some embodiments, P₁, P₂, or P₁ₐ comprises a cyclic peptide. In some embodiments, P₁, P₂, or P₁ₐ comprises a linear peptide. In some embodiments, the tumor cell antigen comprises EGFR, and the P₁ or P₂ comprises Peptide-1, Peptide-2, Peptide-3, Peptide-4, Peptide-5, Peptide-6, or Peptide-7. In some embodiments, the tumor cell antigen comprises EGFR, and the P₁ or P₂ comprises an amino acid sequence selected from the group consisting of GGDWCRSLMSYTDLCP (SEQ ID NO: 1), GGTSCADAHLIAPSCS (SEQ ID NO: 2), GGNCQWDRVEHTYACS (SEQ ID NO: 3), GGWVSCHDGSHMTCFH (SEQ ID NO: 4), GGMNCLNRLWVEYCLV (SEQ ID NO: 5), GGYCGQDNTWVREGCF (SEQ ID NO: 6) and QGQSGQLSCEGWAMNREQCRA (SEQ ID NO: 7). In some embodiments, the tumor cell antigen comprises HER2, and the P₁ or P₂ comprises Peptide-8, Peptide-9, Peptide-10, Peptide-11, Peptide-12, Peptide-13, Peptide-14, Peptide-15, Peptide-16 or Peptide-17. In some embodiments, the tumor cell antigen comprises HER2, and the P₁ or P₂ comprises an amino acid sequence selected from the group consisting of GGPLCSDLDHITRLCD (SEQ ID NO: 8), GGIDCASLDHYTESCY (SEQ ID NO: 9), GGNPVCTLGDPYECSH (SEQ ID NO: 10), GGTFCQLNADPYECQS (SEQ ID NO: 11), GGGYCELIGDYVVCSP (SEQ ID NO: 12), GGLCDRWGWIDAPYCH (SEQ ID NO: 13), GGTGCTEGHWHWGTCS (SEQ ID NO: 14), GGNICMDYSWRSGCAV (SEQ ID NO: 15), GGHSCTFGDWSLGTCA (SEQ ID NO: 16), and GGFICTLGNWWDGSCE (SEQ ID NO: 17). In some embodiments, the effector cell antigen comprises CD3, and the P₁ or P₂ comprises Peptide-18, Peptide-19, Peptide-20, Peptide-21, Peptide-22, Peptide-23, Peptide-24, Peptide-25, Peptide-26, Peptide-27, Peptide-28, or Peptide-29. In some embodiments, the effector cell antigen comprises CD3, and the P₁ or P₂ comprises an amino acid sequence selected from the group consisting of QGQSGQGYLWGCEWNCGGITT (SEQ ID NO: 18), GGDSVCADPEVPICEI (SEQ ID NO: 19), GGMSDCGDPGVEICTH (SEQ ID NO: 20), GGIQCHDPDLPSPCYI (SEQ ID NO: 21), GGEWCLFDPDVPTCQD (SEQ ID NO: 22), GGLGCNDIDPGEQCIV (SEQ ID NO: 23), GGLECFDPEIPEAFCI (SEQ ID NO: 24), GGQGCGTIADPEPHCW (SEQ ID NO: 25), GGNCHDPDIPAYVLCS (SEQ ID NO: 26), GGLCPINDWEPQDICW (SEQ ID NO: 27), and GGLCMIGDWLPGDVCL (SEQ ID NO: 28).

In some embodiments, P₁, P₂, or P₁ₐ or P₁, P₂, and P₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments P₁, P₂, or P₁ₐ or P₁, P₂, and P₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to P₁, P₂, or P₁ₐ or P₁, P₂, and P₁ₐ including the peptide backbone, the amino acid side chains, and the terminus.

In some embodiments, P₁, P₂, or P₁ₐ does not comprise albumin or an albumin fragment. In some embodiments, P₁, P₂, or P₁ₐ does not comprise an albumin binding domain.

Linking Moiety (L₁, L₂, L₃, and L₃ₐ)

In some embodiments, L₁, L₂, L₃, or L₃ₐ is a peptide sequence having at least 5 to no more than 50 amino acids. In some embodiments, L₁, L₂, L₃, or L₃ₐ is a peptide sequence having at least 10 to no more than 30 amino acids. In some embodiments, L₁, L₂, L₃, or L₃ₐ is a peptide sequence having at least 10 amino acids. In some embodiments, L₁, L₂, L₃, or L₃ₐ is a peptide sequence having at least 18 amino acids. In some embodiments, L₁, L₂, L₃, or L₃ₐ is a peptide sequence having at least 26 amino acids. In some embodiments, L₁, L₂, L₃, or L₃a has a formula comprising (G₂S)ₙ, wherein n is an integer from 1 to 3 (SEQ ID NO: 29). In some embodiments, L₁, L₂, L₃, or L₃ₐhas a formula comprising (G₂S)ₙ, wherein n is an integer of at least 1. In some embodiments, L₁, L₂, L₃, or L₃ₐ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1. In some embodiments, the tumor specific protease is selected from the group consisting of metalloprotease, serine protease, cysteine protease, threonine protease, and aspartic protease. In some embodiments L₁, L₂, L₃, or L₃ₐ comprises a urokinase cleavable amino acid sequence, a matriptase cleavable amino acid sequence, a legumain cleavable amino acid sequence, or a matrix metalloprotease cleavable amino acid sequence.

In some embodiments, L₁, L₂, L₃, or L₃ₐ is Linker-1, Linker-2, Linker-3, Linker-4, Linker-5, Linker-6, Linker-7, Linker-8, Linker-9, Linker-10, Linker-11, Linker-12, Linker-13, Linker-14, Linker-15, Linker-16, Linker-17, Linker-18, Linker-19, or Linker 20. In some embodiments, L₁ or L₂ comprises an amino acid sequence selected from the group consisting of GGGGSLSGRSDNHGSSGT (SEQ ID NO: 34), GGGGSSGGSGGSGLSGRSDNHGSSGT (SEQ ID NO: 35), ASGRSDNH (SEQ ID NO: 36), LAGRSDNH (SEQ ID NO: 37), ISSGLASGRSDNH (SEQ ID NO: 38), ISSGLLAGRSDNH (SEQ ID NO: 39), LSGRSDNH (SEQ ID NO: 40), ISSGLLSGRSDNP (SEQ ID NO: 41), ISSGLLSGRSDNH (SEQ ID NO: 42), LSGRSDNHSPLGLAGS (SEQ ID NO: 43), SPLGLAGSLSGRSDNH (SEQ ID NO: 44), SPLGLSGRSDNH (SEQ ID NO: 45), LAGRSDNHSPLGLAGS (SEQ ID NO: 46), LSGRSDNHVPLSLKMG (SEQ ID NO: 47), LSGRSDNHVPLSLSMG (SEQ ID NO: 48), GSSGGSGGSGGSGISSGLLSGRSDNHGSSGT (SEQ ID NO: 49), and GSSGGSGGSGGISSGLLSGRSDNHGGGS (SEQ ID NO: 50). In some embodiments, L₁ or L₂ comprises an amino acid sequence ASGRSDNH (SEQ ID NO: 36), LAGRSDNH (SEQ ID NO: 37), ISSGLASGRSDNH (SEQ ID NO: 38), and ISSGLLAGRSDNH (SEQ ID NO: 39). In some embodiments, L₃ or L₃ₐ comprises an amino acid sequence GGGGSGGGS (SEQ ID NO: 51).

In some embodiments, L₁ is bound to N-terminus of A₁. In some embodiments, L₁ is bound to C-terminus of A₁. In some embodiments, L₂ is bound to N-terminus of A₂. In some embodiments, L₂ is bound to C-terminus of A₂. In some embodiments, P₁ becomes unbound from A₁ when L₁ is cleaved by the tumor specific protease thereby exposing A₁ to the first target antigen. In some embodiments, P₂ becomes unbound from A₂ when L₂ is cleaved by the tumor specific protease thereby exposing A₂to the second target antigen.

In some embodiments, L₁, L₂, L₃, or L₃ₐ or L₁, L₂, L₃, and L₃ₐ comprise a modified amino acid or non-natural amino acid, or a modified non-natural amino acid, or a combination thereof. In some embodiments, the modified amino acid or a modified non-natural amino acid comprises a post-translational modification. In some embodiments, L₁, L₂, L₃, or L₃ₐ or L₁, L₂, L₃, and L₃ₐ comprise a modification including, but not limited, to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to L₁, L₂, L₃, or L₃ₐ or L₁, L₂, L₃, and L₃ₐ including the peptide backbone, or the amino acid side chains.

Half-Life Extending Molecule (H₁ and H₁ₐ)

In some embodiments, H₁ does not block A₁ binding to the first target antigen. In some embodiments, H₁ comprises a linking moiety (L₃) that connects H₁ to P₁. In some embodiments, H₁ₐ does not block antigen recognizing molecule binding to the target antigen. In some embodiments, H₁ₐ comprises a linking moiety (L₃) that connects H₁ₐ to P₁ₐ. In some embodiments, half-life extending molecule (H₁ or H₁ₐ) does not have binding affinity to antigen recognizing molecule. In some embodiments, half-life extending molecule (H₁ or H₁ₐ) does not have binding affinity to the target antigen. In some embodiments, half-life extending molecule (H₁ or H₁ₐ) does not shield antigen recognizing molecule from the target antigen. In some embodiments, half-life extending molecule (H₁ or H₁ₐ) is not directly linked to antigen recognizing molecule.

In some embodiments, H₁ or H₁ₐ comprise an amino acid sequence that has repetitive sequence motifs. In some embodiments, H₁ or H₁ₐ comprises an amino acid sequence that has highly ordered secondary structure. “Highly ordered secondary structure,” as used in this context, means that at least about 50%, or about 70%, or about 80%, or about 90%, of amino acid residues of H₁ or H₁ₐ contribute to secondary structure, as measured or determined by means, including, but not limited to, spectrophotometry (e.g. by circular dichroism spectroscopy in the “far-UV” spectral region (190-250 nm), and computer programs or algorithms, such as the Chou-Fasman algorithm and the Garnier-Osguthorpe-Robson (“GOR”) algorithm.

In some embodiments, H₁ or H₁ₐ comprises a polymer. In some embodiments, the polymer is polyethylene glycol (PEG). In some embodiments, H₁ or H₁ₐ comprises albumin. In some embodiments, H₁ or H₁ₐ comprises an Fc domain. In some embodiments, the albumin is serum albumin. In some embodiments, the albumin is human serum albumin. In some embodiments, H₁ or H₁ₐ comprises a polypeptide, a ligand, or a small molecule. In some embodiments, the polypeptide, the ligand or the small molecule binds serum protein or a fragment thereof, a circulating immunoglobulin or a fragment thereof, or CD35/CR1. In some embodiments, the serum protein comprises a thyroxine-binding protein, a transthyretin, a 1-acid glycoprotein, a transferrin, transferrin receptor or a transferrin-binding portion thereof, a fibrinogen, or an albumin. In some embodiments, the circulating immunoglobulin molecule comprises IgG1, IgG2, IgG3, IgG4, slgA, IgM or IgD. In some embodiments, the serum protein is albumin. In some embodiments, the polypeptide is an antibody. In some embodiments, the antibody comprises a single domain antibody, a single chain variable fragment or a Fab. In some embodiments, the single domain antibody comprises a single domain antibody that binds to albumin. In some embodiments, the antibody is a human or humanized antibody. In some embodiments, the antibody is selected from the group consisting of 645gH1gL1, 645dsgH5gL4, 23-13-A01-sc02, A10m3 or a fragment thereof, DOM7r-31, DOM7h-11-15, Alb-1, Alb-8, Alb-23, 10G, 10GE, and SA21. In some embodiments, the single domain antibody is 10G, and the single domain antibody comprises an amino acid sequence

(SEQ ID NO: 52)
EVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTI
GGSLSVSSQGTLVTVSS.

In some embodiments, H₁ or H₁ₐ or H₁ and H₁ₐ comprise a modified amino acid or non-natural amino acid, or a modified non-natural amino acid, or a combination thereof. In some embodiments, the modified amino acid or a modified non-natural amino acid comprises a post-translational modification. In some embodiments H₁ or H₁ₐ or H1 and H₁ₐ comprise a modification including, but not limited to acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Modifications are made anywhere to H₁ or H₁ₐ or H₁ and H₁ₐ including the peptide backbone, the amino acid side chains, and the terminus.

In some embodiments, H₁comprises a linking moiety (L₃) that connects H1 to P₁. In some embodiments, L₃ is a peptide sequence having at least 5 to no more than 50 amino acids. In some embodiments, L₃ is a peptide sequence having at least 10 to no more than 30 amino acids. In some embodiments, L₃ is a peptide sequence having at least 10 amino acids. In some embodiments, L₃ is a peptide sequence having at least 18 amino acids. In some embodiments, L₃ is a peptide sequence having at least 26 amino acids. In some embodiments, L₃ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1. In some embodiments, L₃ comprises an amino acid sequence GGGGSGGGS (SEQ ID NO: 51).

In some embodiments, H₁ₐ comprises a linking moiety (L₃ₐ) that connects H₁ₐ to P₁ₐ. In some embodiments, L₃a is a peptide sequence having at least 5 to no more than 50 amino acids. In some embodiments, L₃ₐ is a peptide sequence having at least 10 to no more than 30 amino acids. In some embodiments, L₃ₐ is a peptide sequence having at least 10 amino acids. In some embodiments, L₃ₐ is a peptide sequence having at least 18 amino acids. In some embodiments, L₃ₐ is a peptide sequence having at least 26 amino acids. In some embodiments, L₃ₐ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1. In some embodiments, L₃ comprises an amino acid sequence GGGGSGGGS (SEQ ID NO: 51).

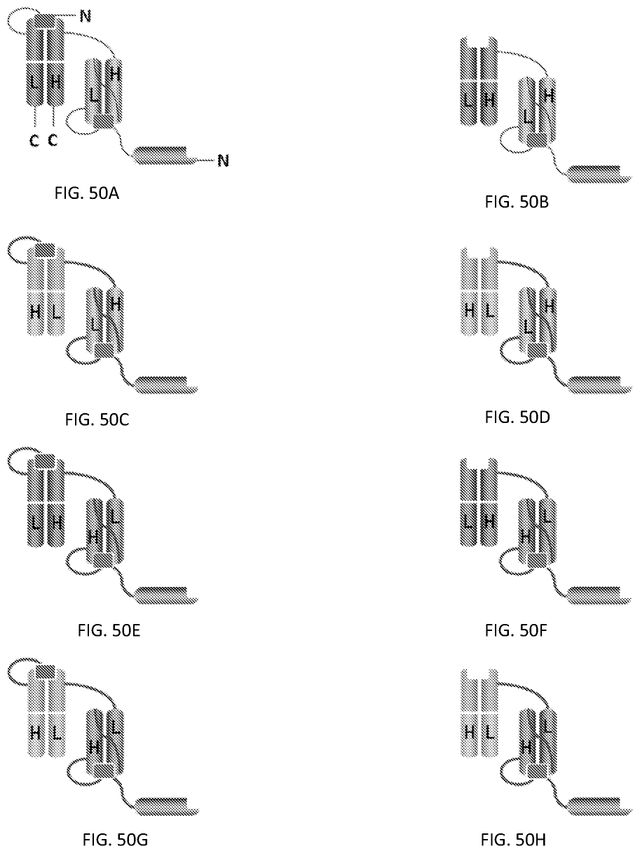

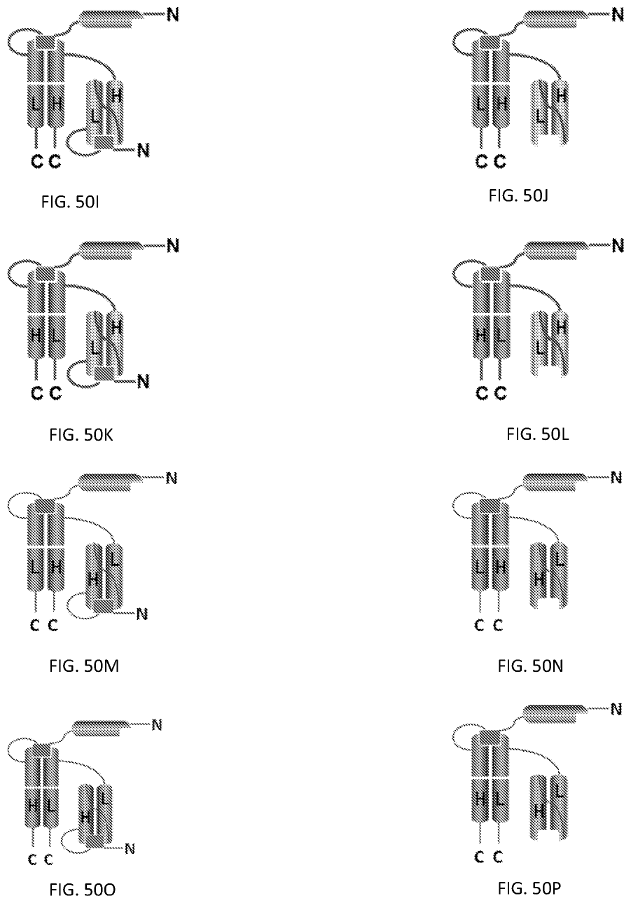

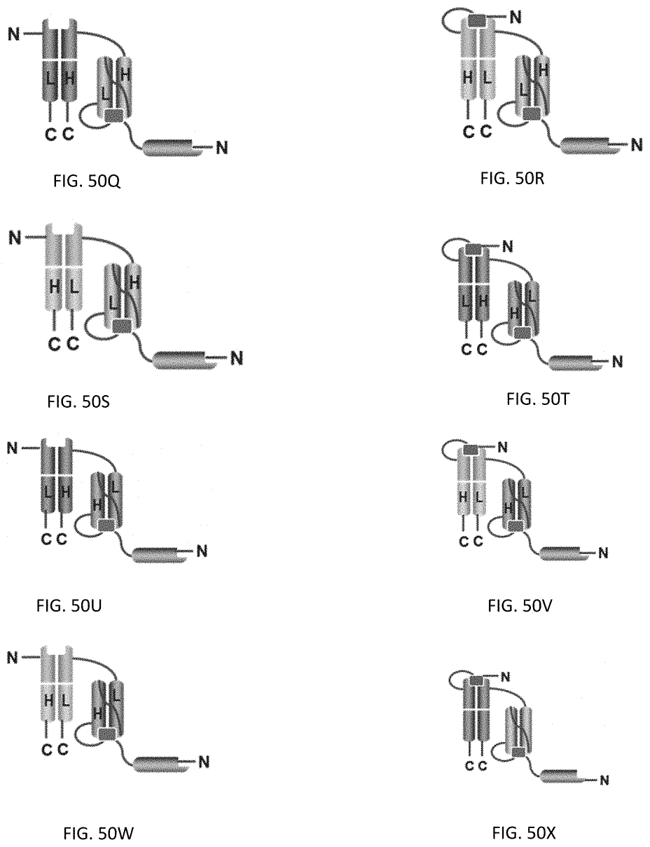

Disclosed herein, in some embodiments, are polypeptides or polypeptide complexes comprising a structural arrangement according to the configuration shown in FIG. 50A, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to the configuration shown in FIG. 50Q, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are polypeptides or polypeptide complexes comprising a structural arrangement according to the configuration shown in FIG. 50R, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to the configuration shown in FIG. 50S, wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are polypeptides or polypeptide complexes comprising a structural arrangement according to the configuration shown in FIG. 50T, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab light chain polypeptide to P₂ and is a substrate for a tumor specific protease.

Disclosed herein, in some embodiments, are polypeptides or polypeptide complexes comprising a structural arrangement according to the configuration shown in FIG. 50U, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are polypeptides or polypeptide complexes comprising a structural arrangement according to the configuration shown in FIG. 50V, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are polypeptides or polypeptide complexes comprising a structural arrangement according to the configuration shown in FIG. 50W, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv.

Disclosed herein, in some embodiments, are polypeptides or polypeptide complexes comprising a structural arrangement according to the configuration shown in FIG. 50I, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to the configuration shown in FIG. 50J, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Disclosed herein, in some embodiments, are polypeptides or polypeptide complexes comprising a structural arrangement according to the configuration shown in FIG. 50K, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are polypeptides or polypeptide complexes comprising a structural arrangement according to the configuration shown in FIG. 50L, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Disclosed herein, in some embodiments, are polypeptides or polypeptide complexes comprising a structural arrangement according to the configuration shown in FIG. 50M, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to the configuration shown in FIG. 50N, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Disclosed herein, in some embodiments, are polypeptides or polypeptide complexes comprising a structural arrangement according to the configuration shown in FIG. 50O, wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are polypeptides or polypeptide complexes comprising a structural arrangement according to the configuration shown in FIG. 50P, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### Polynucleotides Encoding Polypeptides or Polypeptide Complexes

Disclosed herein, in some embodiments, are isolated recombinant nucleic acid molecules encoding polypeptides or polypeptide complexes as disclosed herein. In some embodiments, the polypeptides or polypeptide complexes comprise an antibody or an antibody fragment. In some embodiments, the polypeptides or polypeptide complexes comprise a Fab and a single chain variable fragment (scFv).

Disclosed herein, in some embodiments, are isolated recombinant nucleic acid molecules encoding polypeptides or polypeptide complexes according to Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Disclosed herein, in some embodiments, are isolated recombinant nucleic acid molecules encoding polypeptides or polypeptide complexes according to Formula I:

A₂-A₁-L₁-P₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ comprises a half-life extending molecule; and A₂ is a second antigen recognizing molecule that binds to a second target antigen.

Disclosed herein, in some embodiments, are isolated recombinant nucleic acid molecules encoding polypeptides or polypeptide complexes comprising Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Disclosed herein, in some embodiments, are isolated recombinant nucleic acid molecules encoding polypeptides or polypeptide complexes comprising Formula I:

A₂-A₁-L₁-P₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is s a half-life extending molecule; and A₂ is a second antigen recognizing molecule that binds to a second target antigen.

Disclosed herein, in some embodiments, are isolated recombinant nucleic acid molecules encoding polypeptides or polypeptide complexes according to Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂to P₂ and is a substrate for a tumor specific protease.

Disclosed herein, in some embodiments, are isolated recombinant nucleic acid molecules encoding polypeptides or polypeptide complexes according to Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease.

Disclosed herein, in some embodiments, are isolated recombinant nucleic acid molecules encoding polypeptides or polypeptide complexes comprising Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease.

Disclosed herein, in some embodiments, are isolated recombinant nucleic acid molecules encoding polypeptides or polypeptide complexes comprising Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50A, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50Q, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Disclosed herein, in some embodiments, are isolated nucleic acid molecules encoding polypeptides or polypeptide complexes comprising a structural arrangement according to the configuration shown in FIG. 50R, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50S, wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv.

Disclosed herein, in some embodiments, are isolated nucleic acid molecules encoding polypeptides or polypeptide complexes comprising a structural arrangement according to the configuration shown in FIG. 50T, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab light chain polypeptide to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50U, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv.

Disclosed herein, in some embodiments, are isolated nucleic acid molecules encoding polypeptides or polypeptide complexes comprising a structural arrangement according to the configuration shown in FIG. 50V, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50W, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv.

Disclosed herein, in some embodiments, are isolated nucleic acid molecules encoding polypeptides or polypeptide complexes comprising a structural arrangement according to the configuration shown in FIG. 50I, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50J, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Disclosed herein, in some embodiments, are isolated nucleic acid molecules encoding polypeptides or polypeptide complexes comprising a structural arrangement according to the configuration shown in FIG. 50K, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50L, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Disclosed herein, in some embodiments, are isolated nucleic acid molecules encoding polypeptides or polypeptide complexes comprising a structural arrangement according to the configuration shown in FIG. 50M, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50N, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Disclosed herein, in some embodiments, are isolated nucleic acid molecules encoding polypeptides or polypeptide complexes comprising a structural arrangement according to the configuration shown in FIG. 50O, wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Disclosed herein, in some embodiments, are isolated nucleic acid molecules encoding polypeptides or polypeptide complexes comprising a structural arrangement according to the configuration shown in FIG. 50P, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### Pharmaceutical Compositions

Disclosed herein, in some embodiments, are pharmaceutical compositions comprising: (a) the polypeptides or polypeptide complexes as disclosed herein; and (b) a pharmaceutically acceptable excipient.

In some embodiments, the pharmaceutical composition comprises (a) polypeptides or polypeptide complexes according to Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen; and (b) a pharmaceutically acceptable excipient.

In some embodiments, the pharmaceutical composition comprises (a) polypeptides or polypeptide complexes according to Formula I:

A₂-A₁-L₁-F₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen; and (b) a pharmaceutically acceptable excipient.

In some embodiments, the pharmaceutical composition comprises (a) polypeptides or polypeptide complexes comprising Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen; and (b) a pharmaceutically acceptable excipient.

In some embodiments, the pharmaceutical composition comprises (a) polypeptides or polypeptide complexes comprising Formula I:

A₂-A₁-L₁-P₁-H₁ (Formula I)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; and A₂ is a second antigen recognizing molecule that binds to a second target antigen; and (b) a pharmaceutically acceptable excipient.

In some embodiments, the pharmaceutical composition comprises (a) polypeptides or polypeptide complexes according to Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes according to Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen; P₂ is a peptide that binds to A₂; and L₂ is a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes comprising Formula Ia:

P₂-L₂-A₂-A₁-L₁-P₁-H₁ (Formula Ia)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; A₂ comprises a second antigen recognizing molecule that binds to a second target antigen; P₂ comprises a peptide that binds to A₂; and L₂ comprises a linking moiety that connects A₂ to P₂ and is a substrate for a tumor specific protease; and (b) a pharmaceutically acceptable excipient.

In some embodiments, the pharmaceutical composition comprises (a) polypeptides or polypeptide complexes comprising Formula Ia:

P₂-L₂-A₂-A₁-L₁-P₁-H₁ (Formula Ia)

wherein: A₁ is a first antigen recognizing molecule that binds to a first target antigen; P₁ is a peptide that binds to A₁; L₁ is a linking moiety that connects A₁ to P₁ and is a substrate for a tumor specific protease; H₁ is a half-life extending molecule; A₂ is a second antigen recognizing molecule that binds to a second target antigen P₂ is a peptide that binds to A₂; and L₂ is a linking moiety that connects A₂ to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50A, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50Q, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50R, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50S, wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50T, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab light chain polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50U, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50V, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50W, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50I, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50J, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50K, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50L, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50M, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50N, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50O, wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease; and (b) a pharmaceutically acceptable excipient.

Disclosed herein, in some embodiments, the pharmaceutical composition comprises (a) polypeptides or polypeptide complexes comprising a structural arrangement according to the configuration shown in FIG. 50P, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide; and (b) a pharmaceutically acceptable excipient.

In some embodiments, the polypeptide or polypeptide complex further comprises a detectable label, a therapeutic agent, or a pharmacokinetic modifying moiety. In some embodiments, the detectable label comprises a fluorescent label, a radiolabel, an enzyme, a nucleic acid probe, or a contrast agent.

For administration to a subject, the polypeptide or polypeptide complex as disclosed herein, may be provided in a pharmaceutical composition together with one or more pharmaceutically acceptable carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, or intravenous) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

Table 1 provides the amino acid sequences of constructs described herein.

TABLE 1
Summary of Amino Acid Sequences

| | | | |
|---|---|---|---|
| Construct | | Amino Acid Sequence | SEQ ID |
| ID | Construct Description | (N to C) | NO: |

PEPTIDE MASK SEQUENCES

| | | | |
|---|---|---|---|
| Peptide-1 | anti-EGFR peptide | GGDWCRSLMSYTDLCP | 1 |
| | mask | | |
| Peptide-2 | anti-EGFR peptide | GGTSCADAHLIAPSCS | 2 |
| | mask | | |
| Peptide-3 | anti-EGFR peptide | GGNCQWDRVEHTYACS | 3 |
| | mask | | |
| Peptide-4 | anti-EGFR peptide | GGWVSCHDGSHMTCFH | 4 |
| | mask | | |
| Peptide-5 | anti-EGFR peptide | GGMNCLNRLWVEYCLV | 5 |
| | mask | | |
| Peptide-6 | anti-EGFR peptide | GGYCGQDNTWVREGCF | 6 |
| | mask | | |
| Peptide-7 | anti-EGFR peptide | QGQSGQLSCEGWAMNREQCRA | 7 |
| | mask | | |
| Peptide-8 | anti-HER2 peptide | GGPLCSDLDHITRLCD | 8 |
| | mask | | |
| Peptide-9 | anti-HER2 peptide | GGIDCASLDHYTESCY | 9 |
| | mask | | |
| Peptide-10 | anti-HER2 peptide | GGNPVCTLGDPYECSH | 10 |
| | mask | | |
| Peptide-11 | anti-HER2 peptide | GGTFCQLNADPYECQS | 11 |
| | mask | | |
| Peptide-12 | anti-HER2 peptide | GGGYCELIGDYVVCSP | 12 |
| | mask | | |
| Peptide-13 | anti-HER2 peptide | GGLCDRWGWIDAPYCH | 13 |
| | mask | | |
| Peptide-14 | anti-HER2 peptide | GGTGCTEGHWHWGTCS | 14 |
| | mask | | |
| Peptide-15 | anti-HER2 peptide | GGNICMDYSWRSGCAV | 15 |
| | mask | | |
| Peptide-16 | anti-HER2 peptide | GGHSCTFGDWSLGTCA | 16 |
| | mask | | |
| Peptide-17 | anti-HER2 peptide | GGFICTLGNWWDGSCE | 17 |
| | mask | | |
| Peptide-18 | anti-CD3 peptide mask | QGQSGQGYLWGCEWNCGGITT | 18 |
| Peptide-19 | anti-CD3 peptide mask | GGDSVCADPEVPICEI | 19 |
| Peptide-20 | anti-CD3 peptide mask | GGMSDCGDPGVEICTH | 20 |
| Peptide-21 | anti-CD3 peptide mask | GGIQCHDPDLPSPCYI | 21 |
| Peptide-22 | anti-CD3 peptide mask | GGEWCLFDPDVPTCQD | 22 |
| Peptide-23 | anti-CD3 peptide mask | GGLGCNDIDPGEQCIV | 23 |
| Peptide-24 | anti-CD3 peptide mask | GGLECFDPEIPEAFCI | 24 |
| Peptide-25 | anti-CD3 peptide mask | GGQGCGTIADPEPHCW | 25 |
| Peptide-26 | anti-CD3 peptide mask | GGNCHDPDIPAYVLCS | 26 |
| Peptide-27 | anti-CD3 peptide mask | GGLCPINDWEPQDICW | 27 |
| Peptide-28 | anti-CD3 peptide mask | GGLCMIGDWLPGDVCL | 28 |
| Peptide-29 | anti-CD3 peptide mask | QGQSGSGYLWGCEWNCGGITT | 53 |

LINKER SEQUENCES

| | | | |
|---|---|---|---|
| Linker-1 | linker | GGGGSLSGRSDNHGSSGT | 34 |
| Linker-2 | linker | GGGGSSGGSGGSGLSGRSDNHGSSGT | 35 |
| Linker-3 | linker | ASGRSDNH | 36 |
| Linker-4 | linker | LAGRSDNH | 37 |
| Linker-5 | linker | ISSGLASGRSDNH | 38 |
| Linker-6 | linker | ISSGLLAGRSDNH | 39 |
| Linker-7 | linker | LSGRSDNH | 40 |
| Linker-8 | linker | ISSGLLSGRSDNP | 41 |
| Linker-9 | linker | ISSGLLSGRSDNH | 42 |
| Linker-10 | linker | LSGRSDNHSPLGLAGS | 43 |
| Linker-11 | linker | SPLGLAGSLSGRSDNH | 44 |
| Linker-12 | linker | SPLGLSGRSDNH | 45 |
| Linker-13 | linker | LAGRSDNHSPLGLAGS | 46 |
| Linker-14 | linker | LSGRSDNHVPLSLKMG | 47 |
| Linker-15 | linker | LSGRSDNHVPLSLSMG | 48 |
| Linker-16 | linker | GSSGGSGGSGGSGISSGLLSGRSDNHGSSGT | 49 |
| Linker-17 | linker | GSSGGSGGSGGISSGLLSGRSDNHGGGS | 50 |
| Linker-18 | linker (noncleavable) | GSSGGSGGSGGASSGAGGSGGGSGGGGS | 54 |
| Linker-19 | linker | GGGGSGGGGSGGISSGLLSGRSDNHGSSGT | 55 |
| Linker-20 | linker | GGGGSGGGS | 51 |

ANTIBODY AND ANTIBODY FRAGMENT SEQUENCES

| | | | |
|---|---|---|---|
| Ab-1 | anti-EGFR light chain | DILLTQSPVILSVSPGERVSFSCRASQSIGTNI | 56 |
| | | HWYQQRTNGSPRLLIKYASESISGIPSRFSG | |
| | | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| Ab-2 | anti-EGFR light chain | QILLTQSPVILSVSPGERVSFSCRASQSIGTNI | 57 |
| | | HWYQQRTNGSPRLLIKYASESISGIPSRFSG | |
| | | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| Ab-3 | anti-EGFR heavy chain | QVQLKQSGPGLVQPSQSLSITCTVSGFSLTN | 58 |
| | | YGVHWVRQSPGKGLEWLGVIWSGGNTDY | |
| | | NTPFTSRLSINKDNSKSQVFFKMNSLQSND | |
| | | TAIYYCARALTYYDYEFAYWGQGTLVTVS | |
| | | AASTKGPSVFPLAPSSKSTSGGTAALGCLV | |
| | | KDYFPEPVTVSWNSGALTSGVHTFPAVLQS | |
| | | SGLYSLSSVVTVPSSSLGTQTYICNVNHKPS | |
| | | NTKVDKKVEPKSCDKTHTCPPCPAPELLGG | |
| | | PSVFLFPPKPKDTLMISRTPEVTCVVVDVSH | |
| | | EDPEVKFNWYVDGVEVHNAKTKPREEQY | |
| | | NSTYRVVSVLTVLHQDWLNGKEYKCKVS | |
| | | NKALPAPIEKTISKAKGQPREPQVYTLPPSR | |
| | | DELTKNQVSLTCLVKGFYPSDIAVEWESNG | |
| | | QPENNYKTTPPVLDSDGSFFLYSKLTVDKS | |
| | | RWQQGNVFSCSVMHEALHNHYTQKSLSLS | |
| | | PGK | |
| Ab-4 | anti-EGFR heavy chain | QVQLKQSGPGLVQPSQSLSITCTVSGFSLTN | 59 |
| | | YGVHWVRQSPGKGLEWLGVIWSGGNTDY | |
| | | NTPFTSRLSINKDNSKSQVFFKMNSLQSND | |
| | | TAIYYCARALTYYDYEFAYWGQGTLVTVS | |
| | | AASTKGPSVFPLAPSSKSTSGGTAALGCLV | |
| | | KDYFPEPVTVSWNSGALTSGVHTFPAVLQS | |
| | | SGLYSLSSVVTVPSSSLGTQTYICNVNHKPS | |
| | | NTKVDKKVEPKSC | |
| Ab-5 | anti-EGFR heavy chain | QVQLKQSGPGLVQPSQSLSITCTVSGFSLTN | 60 |
| | | YGVHWVRQSPGKGLEWLGVIWSGGNTDY | |
| | | NTPFTSRLSINKDNSKSQVFFKMNSLQSQD | |
| | | TAIYYCARALTYYDYEFAYWGQGTLVTVS | |
| | | AASTKGPSVFPLAPSSKSTSGGTAALGCLV | |
| | | KDYFPEPVTVSWNSGALTSGVHTFPAVLQS | |
| | | SGLYSLSSVVTVPSSSLGTQTYICNVNHKPS | |
| | | NTKVDKKVEPKSC | |
| Ab-6 | anti-HER2 light chain | DIQMTQSPSSLSASVGDRVTITCRASQDVN | 61 |
| | | TAVAWYQQKPGKAPKLLIYSASFLYSGVPS | |
| | | RFSGSRSGTDFTLTISSLQPEDFATYYCQQH | |
| | | YTTPPTFGQGTKVEIKRTVAAPSVFIFPPSDE | |
| | | QLKSGTASVVCLLNNFYPREAKVQWKVDN | |
| | | ALQSGNSQESVTEQDSKDSTYSLSSTLTLSK | |
| | | ADYEKHKVYACEVTHQGLSSPVTKSFNRG | |
| | | EC | |
| Ab-7 | anti-HER2 heavy chain | EVQLVESGGGLVQPGGSLRLSCAASGFNIK | 62 |
| | | DTYIHWVRQAPGKGLEWVARIYPTNGYTR | |
| | | YADSVKGRFTISADTSKNTAYLQMNSLRAE | |
| | | DTAVYYCSRWGGDGFYAMDYWGQGTLV | |
| | | TVSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNHK | |
| | | PSNTKVDKKVEPKSCDKTHTCPPCPAPELL | |
| | | GGPSVFLFPPKPKDTLMISRTPEVTCVVVD | |
| | | VSHEDPEVKFNWYVDGVEVHNAKTKPREE | |
| | | QYNSTYRVVSVLTVLHQDWLNGKEYKCK | |
| | | VSNKALPAPIEKTISKAKGQPREPQVYTLPP | |
| | | SREEMTKNQVSLTCLVKGFYPSDIAVEWES | |
| | | NGQPENNYKTTPPVLDSDGSFFLYSKLTVD | |
| | | KSRWQQGNVFSCSVMHEALHNHYTQKSLS | |
| | | LSPGK | |
| Ab-8 | anti-HER2 heavy chain | EVQLVESGGGLVQPGGSLRLSCAASGFNIK | 63 |
| | | DTYIHWVRQAPGKGLEWVARIYPTNGYTR | |
| | | YADSVKGRFTISADTSKNTAYLQMNSLRAE | |
| | | DTAVYYCSRWGGDGFYAMDYWGQGTLV | |
| | | TVSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNHK | |
| | | PSNTKVDKKVEPKSC | |
| Ab-9 | anti-CD3 light chain | QAVVTQESALTTSPGETVTLTCRSSTGAVT | 64 |
| | | TSNYANWVQEKPDHLFTGLIGGTNKRAPG | |
| | | VPARFSGSLIGDKAALTITGAQTEDEAIYFC | |
| | | ALWYSNLWVFGGGTKLTVLQPKSSPSVTL | |
| | | FPPSSEELETNKATLVCTITDFYPGVVTVD | |
| | | WKVDGTPVTQGMETTQPSKQSNNKYMAS | |
| | | SYLTLTARAWERHSSYSCQVTHEGHTVEK | |
| | | SLSRADCS | |
| Ab-10 | anti-CD3 heavy chain | EVQLVESGGGLVQPKGSLKLSCAASGFTFN | 65 |
| | | TYAMNWVRQAPGKGLEWVARIRSKYNNY | |
| | | ATYYADSVKDRFTISRDDSQSILYLQMNNL | |
| | | KTEDTAMYYCVRHGNFGNSYVSWFAYWG | |
| | | QGTLVTVSSAKTTPPSVYPLAPGSAAQTNS | |
| | | MVTLGCLVKGYFPEPVTVTWNSGSLSSGV | |
| | | HTFPAVLQSDLYTLSSSVTVPSSPRPSETVT | |
| | | CNVAHPASSTKVDKKIVPRDCGCKPCICTV | |
| | | PEVSSVFIFPPKPKDVLTITLTPKVTCVVVDI | |
| | | SKDDPEVQFSWFVDDVEVHTAQTQPREEQ | |
| | | FNSTFRSVSELPIMHQDWLNGKEFKCRVNS | |
| | | AAFPAPIEKTISKTKGRPKAPQVYTIPPPKEQ | |
| | | MAKDKVSLTCMITDFFPEDITVEWQWNGQ | |
| | | PAENYKNTQPIMNTNGSYFVYSKLNVQKS | |
| | | NWEAGNTFTCSVLHEGLHNHHTEKSLSHSP | |
| | | GK | |
| Ab-11 | anti-CD3 scFv (N- | EVQLVESGGGLVQPGGSLKLSCAASGFTFN | 66 |
| | heavy chain-light | KYAMNWVRQAPGKGLEWVARIRSKYNNY | |
| | chain-C) | ATYYADSVKDRFTISRDDSKNTAYLQMNN | |
| | SP34.185 (VH-VL) | LKTEDTAVYYCVRHGNFGNSYISYWAYW | |
| | | GQGTLVTVSSGGGGSGGGGSGGGGSQTVV | |
| | | TQEPSLTVSPGGTVTLTCGSSTGAVTSGNY | |
| | | PNWVQQKPGQAPRGLIGGTKFLAPGTPARF | |
| | | SGSLLGGKAALTLSGVQPEDEAEYYCVLW | |
| | | YSNRWVFGGGTKLTVL | |
| Ab-12 | anti-CD3 scFv | QTVVTQEPSFSVSPGGTVTLTCRSSTGAVTT | 67 |
| | (N-light chain-heavy | SNYANWVQQTPGQAPRGLIGGTNKRAPGV | |
| | chain-C) | PDRFSGSILGNKAALTITGAQADDESDYYC | |
| | SP34.V16 (VL-VH) | ALWYSNLWVFGGGTKLTVLGGGGSGGGG | |
| | | SGGGGSEVQLVESGGGLVQPGGSLKLSCA | |
| | | ASGFTFSTYAMNWVRQASGKGLEWVGRIR | |
| | | SKYNNYATYYADSVKDRFTISRDDSKNTA | |
| | | YLQMNSLKTEDTAVYYCTRHGNFGNSYVS | |
| | | WFAYWGQGTLVTVSS | |
| Ab-13 | anti-CD3 scFv (N- | QTVVTQEPSLTVSPGGTVTLTCRSSTGAVT | 68 |
| | heavy chain-light | TSNYANWVQQKPGQAPRGLIGGTNKRAPG | |
| | chain-C) | TPARFSGSLLGGKAALTLSGVQPEDEAEYY | |
| | SP34.194 (VL-VH) | CALWYSNLWVFGGGTKLTVLGGGGSGGG | |
| | | GSGGGGSEVQLVESGGGLVQPGGSLKLSC | |
| | | AASGFTFNTYAMNWVRQAPGKGLEWVAR | |
| | | IRSKYNNYATYYADSVKDRFTISRDDSKNT | |
| | | AYLQMNNLKTEDTAVYYCVRHGNFGNSY | |
| | | VSWFAYWGQGTLVTVSS | |

HALF LIFE EXTENDING MOIETIES

| | | | |
|---|---|---|---|
| HE-1 | 10G single domain | EVQLVESGGGLVQPGNSLRLSCAASGFTFS | 52 |
| | antibody | KFGMSWVRQAPGKGLEWVSSISGSGRDTL | |
| | | YADSVKGRFTISRDNAKTTLYLQMNSLRPE | |
| | | DTAVYYCTIGGSLSVSSQGTLVTVSS | |

POLYPEPTIDE COMPLEX FULL LENGTH SEQUENCES

| | | | |
|---|---|---|---|
| PC-1 | Light Chain Sequence: | DILLTQSPVILSVSPGERVSFSCRASQSIGTNI | 56 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPR | |
| | | EAKVQWKVDNALQSGNSQESVTEQDSKDS | |
| | | TYSLSSTLTLSKADYEKHKVYACEVTHQGL | |
| | | SSPVTKSFNRGEC | |
| PC-1 | Heavy Chain Sequence: | QTVVTQEPSLTVSPGGTVTLTCRSSTGAVT | 69 |
| | N-[anti-CD3 scFv | TSNYANWVQQKPGQAPRGLIGGTNKRAPG | |
| | (light chain-heavy | TPARFSGSLLGGKAALTLSGVQPEDEAEYY | |
| | chain)]-[anti-EGFR | CALWYSNLWVFGGGTKLTVLGGGGSGGG | |
| | Fab heavy chain]-C | GSGGGGSEVQLVESGGGLVQPGGSLKLSC | |
| | | AASGFTFNTYAMNWVRQAPGKGLEWVAR | |
| | | IRSKYNNYATYYADSVKDRFTISRDDSKNT | |
| | | AYLQMNNLKTEDTAVYYCVRHGNFGNSY | |
| | | VSWFAYWGQGTLVTVSSGGGGSQVQLKQ | |
| | | SGPGLVQPSQSLSITCTVSGFSLTNYGVHW | |
| | | VRQSPGKGLEWLGVIWSGGNTDYNTPFTS | |
| | | RLSINKDNSKSQVFFKMNSLQSNDTAIYYC | |
| | | ARALTYYDYEFAYWGQGTLVTVSAASTKG | |
| | | PSVFPLAPSSKSTSGGTAALGCLVKDYFPEP | |
| | | VTVSWNSGALTSGVHTFPAVLQSSGLYSLS | |
| | | SVVTVPSSSLGTQTYICNVNHKPSNTKVDK | |
| | | KVEPKSCGGHHHHHHHHGGGLNDIFEAQK | |
| | | IEWHE | |
| PC-2 | Light Chain Sequence: | QGQSGQLSCEGWAMNREQCRAGSSGGSG | 70 |
| | N-[Peptide-7]- | GSGGSGISSGLLSGRSDNHGSSGTDILLTQS | |
| | [linker]- | PVILSVSPGERVSFSCRASQSIGTNIHWYQQ | |
| | [anti-EGFR Fab light | RTNGSPRLLIKYASESISGIPSRFSGSGSGTD | |
| | chain]-C | FTLSINSVESEDIADYYCQQNNNWPTTFGA | |
| | | GTKLELKRTVAAPSVFIFPPSDEQLKSGTAS | |
| | | VVCLLNNFYPREAKVQWKVDNALQSGNS | |
| | | QESVTEQDSKDSTYSLSSTLTLSKADYEKH | |
| | | KVYACEVTHQGLSSPVTKSFNRGEC | |
| PC-2 | Heavy Chain Sequence: | QGQSGQGYLWGCEWNCGGITTGSSGGSGG | 71 |
| | N-[Peptide-18] | SGGISSGLLSGRSDNHGGGSQTVVTQEPSLT | |
| | [linker]-[anti-CD3 | VSPGGTVTLTCRSSTGAVTTSNYANWVQQ | |
| | scFv (light chain- | KPGQAPRGLIGGTNKRAPGTPARFSGSLLG | |
| | heavy chain)]- | GKAALTLSGVQPEDEAEYYCALWYSNLW | |
| | [anti-EGFR | VFGGGTKLTVLGGGGSGGGGSGGGGSEVQ | |
| | Fab heavy chain]-C | LVESGGGLVQPGGSLKLSCAASGFTFNTYA | |
| | | MNWVRQAPGKGLEWVARIRSKYNNYATY | |
| | | YADSVKDRFTISRDDSK | |
| | | NTAYLQMNNLKTEDTAVYYCVRHGNFGN | |
| | | SYVSWFAYWGQGTLVTVSSGGGGSQVQL | |
| | | KQSGPGLVQPSQSLSITCTVSGFSLTNYGVH | |
| | | WVRQSPGKGLEWLGVIWSGGNTDYNTPFT | |
| | | SRLSINKDNSKSQVFFKMNSLQSNDTAIYY | |
| | | CARALTYYDYEFAYWGQGTLVTVSAASTK | |
| | | GPSVFPLAPSSKSTSGGTAALGCLVKDYFPE | |
| | | PVTVSWNSGALTSGVHTFPAVLQSSGLYSL | |
| | | SSVVTVPSSSLGTQTYICNVNHKPSNTKVD | |
| | | KKVEPKSCGGHHHHHHHHGGGLNDIFEAQ | |
| | | KIEWHE | |
| PC-3 | Light Chain Sequence: | EVQLVESGGGLVQPGNSLRLSCAASGFTFS | 72 |
| | N-[10G SDA]- | KFGMSWVRQAPGKGLEWVSSISGSGRDTL | |
| | [Peptide-7]-[linker]- | YADSVKGRFTISRDNAKTTLYLQMNSLRPE | |
| | [anti-EGFR Fab light | DTAVYYCTIGGSLSVSSQGTLVTVSSGGGG | |
| | chain]-C | SGGGSQGQSGQLSCEGWAM | |
| | | NREQCRAGSSGGSGGSGGSGISSGLLSGRS | |
| | | DNHGSSGTDILLTQSPVILSVSPGERVSFSCR | |
| | | ASQSIGTNIHWYQQRTNGSPRLLIKYASESI | |
| | | SGIPSRFSGSGSGTDFTLSINSVESEDIADYY | |
| | | CQQNNNWPTTFGAGTKLELKRTVAAPSVFI | |
| | | FPPSDEQLKSGTASVVCLLNNFYPREAKVQ | |
| | | WKVDNALQSGNSQESVTEQDSKDSTYSLS | |
| | | STLTLSKADYEKHKVYACEVTHQGLSSPVT | |
| | | KSFNRGEC | |
| PC-3 | Heavy Chain Sequence: | QGQSGQGYLWGCEWNCGGITTGSSGGSGG | 71 |
| | N-[Peptide-18]- | SGGISSGLLSGRSDNHGGGSQTVVTQEPSLT | |
| | [linker]-[anti-CD3 | VSPGGTVTLTCRSSTGAVTTSNYANWVQQ | |
| | scFv (light chain- | KPGQAPRGLIGGTNKRAPGTPARFSGSLLG | |
| | heavy chain)]- | GKAALTLSGVQPEDEAEYYCALWYSNLW | |
| | [anti-EGFR | VFGGGTKLTVLGGGGSGGGGSGGGGSEVQ | |
| | Fab heavy chain]-C | LVESGGGLVQPGGSLKLSCAASGFTFNTYA | |
| | | MNWVRQAPGKGLEWVARIRSKYNNYATY | |
| | | YADSVKDRFTISRDDSKNTAYLQMNNLKT | |
| | | EDTAVYYCVRHGNFGNSYVSWFAYWGQG | |
| | | TLVTVSSGGGGSQVQLKQSGPGLVQPSQSL | |
| | | SITCTVSGFSLTNYGVHWVRQS | |
| | | PGKGLEWLGVIWSGGNTDYNTPFTSRLSIN | |
| | | KDNSKSQVFFKMNSLQSNDTAIYYCARAL | |
| | | TYYDYEFAYWGQGTLVTVSAASTKGPSVF | |
| | | PLAPSSKSTSGGTAALGCLVKDYFPEPVTV | |
| | | SWNSGALTSGVHTFPAVLQSSGLYSLSSVV | |
| | | TVPSSSLGTQTYICNVNHKPSNTKVDKKVE | |
| | | PKSCGGHHHHHHHHGGGLNDIFEAQKIEW | |
| | | HE | |
| PC-4 | Light Chain Sequence: | QGQSGQLSCEGWAMNREQCRAGSSGGSG | 70 |
| | N-[Peptide-7]- | GSGGSGISSGLLSGRSDNHGSSGTDILLTQS | |
| | [linker]- | PVILSVSPGERVSFSCRASQSIGTNIHWYQQ | |
| | [anti-EGFR Fab light | RTNGSPRLLIKYASESISGIPSRFSGSGSGTD | |
| | chain]-C | FTLSINSVESEDIADYYCQQNNNWPTTFGA | |
| | | GTKLELKRTVAAPSVFIFPPSDEQLKSGTAS | |
| | | VVCLLNNFYPREAKVQWKVDNALQSGNS | |
| | | QESVTEQDSKDSTYSLSSTLTLSKADYEKH | |
| | | KVYACEVTHQGLSSPVTKSFNRGEC | |
| PC-4 | Heavy Chain Sequence: | EVQLVESGGGLVQPGNSLRLSCAASGFTFS | 73 |
| | N-[10G SDA]- | KFGMSWVRQAPGKGLEWVSSISGSGRDTL | |
| | [Peptide-18]-[linker]- | YADSVKGRFTISRDNAKTTLYLQMNSLRPE | |
| | [anti-CD3 scFv (light | DTAVYYCTIGGSLSVSSQGTLVTVSSGGGG | |
| | chain-heavy chain)]- | SGGGSQGQSGQGYLWGCEW | |
| | [anti-EGFR Fab heavy | NCGGITTGSSGGSGGSGGISSGLLSGRSDNH | |
| | chain]-C | GGGSQTVVTQEPSLTVSPGGTVTLTCRSST | |
| | | GAVTTSNYANWVQQKPGQAPRGLIGGTNK | |
| | | RAPGTPARFSGSLLGGKAALTLSGVQPEDE | |
| | | AEYYCALWYSNLWVFGGGTKLTVLGGGG | |
| | | SGGGGSGGGGSEVQLVESGGGLVQPGGSL | |
| | | KLSCAASGFTFNTYAMNWVRQAPGKGLE | |
| | | WVARIRSKYNNYATYYADSVKDRFTISRD | |
| | | DSKNTAYLQMNNLKTEDTAVYYCVRHGN | |
| | | FGNSYVSWFAYWGQGTLVTVSSGGGGSQ | |
| | | VQLKQSGPGLVQPSQSLSITCTVSGFSLTNY | |
| | | GVHWVRQSPGKGLEWLGVIWSGGNTDYN | |
| | | TPFTSRLSINKDNSKSQVFFKMNSLQSNDT | |
| | | AIYYCARALTYYDYEFAYWGQGTLVTVSA | |
| | | ASTKGPSVFPLAPSSKSTSGGTAALGCLVK | |
| | | DYFPEPVTVSWNSGALTSGVHTFPAVLQSS | |
| | | GLYSLSSVVTVPSSSLGTQTYICNVNHKPSN | |
| | | TKVDKKVEPKSCGGHHHHHHHHGGGLNDI | |
| | | FEAQKIEWHE | |
| PC-5 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | c | |
| PC-5 | Heavy Chain Sequence: | EVQLVESGGGLVQPGNSLRLSCAASGFTFS | 74 |
| | N-[10G SDA]- | KFGMSWVRQAPGKGLEWVSSISGSGRDTL | |
| | [Peptide-25]-[linker]- | YADSVKGRFTISRDNAKTTLYLQMNSLRPE | |
| | [anti-CD3 scFv (heavy | DTAVYYCTIGGSLSVSSQGTLVTVSSGGGG | |
| | chain-light chain)] - | SGGGSGGQGCGTIADPEPHCWGSSGGSGGS | |
| | [anti-EGFR Fab heavy | GGISSGLLSGRSDNHGGGSEVQLVESGGGL | |
| | chain]-C | VQPGGSLKLSCAASGFTFNKYAMNWVRQ | |
| | | APGKGLEWVARIRSKYNNYATYYADSVKD | |
| | | RFTISRDDSKNTAYLQMNNLKTEDTAVYY | |
| | | CVRHGNFGNSYISYWAYWGQGTLVTVSSG | |
| | | GGGSGGGGSGGGGSQTVVTQEPSLTVSPG | |
| | | GTVTLTCGSSTGAVTSGNYPNWVQQKPGQ | |
| | | APRGLIGGTKFLAPGTPARFSGSLLGGKAA | |
| | | LTLSGVQPEDEAEYYCVLWYSNRWVFGGG | |
| | | TKLTVLGGGGSQVQLKQSGPGLVQPSQSLS | |
| | | ITCTVSGFSLTNYGVHWVRQSPGKGLEWL | |
| | | GVIWSGGNTDYNTPFTSRLSINKDNSKSQV | |
| | | FFKMNSLQSQDTAIYYCARALTYYDYEFA | |
| | | YWGQGTLVTVSAASTKGPSVFPLAPSSKST | |
| | | SGGTAALGCLVKDYFPEPVTVSWNSGALT | |
| | | SGVHTFPAVLQSSGLYSLSSVVTVPSSSLGT | |
| | | QTYICNVNHKPSNTKVDKKVEPKSCGGGH | |
| | | HHHHHHH | |
| PC-6 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| PC-6 | Heavy Chain Sequence: | EVQLVESGGGLVQPGNSLRLSCAASGFTFS | 75 |
| | N-[10G SDA]- | KFGMSWVRQAPGKGLEWVSSISGSGRDTL | |
| | [Peptide-25]- | YADSVKGRFTISRDNAKTTLYLQMNSLRPE | |
| | [noncleavable linker]- | DTAVYYCTIGGSLSVSSQGTLVTVSSGGGG | |
| | [anti-CD3 scFv (heavy | SGGGSGGQGCGTIADPEPHCWGSSGGSGGS | |
| | chain-light chain)]- | GGASSGAGGSGGGSGGGGSEVQLVESGGG | |
| | [anti-EGFR Fab heavy | LVQPGGSLKLSCAASGFTFNKYAMNWVRQ | |
| | chain]-C | APGKGLEWVARIRSKYNNYATYYADSVKD | |
| | | RFTISRDDSKNTAYLQMNNLKTEDTAVYY | |
| | | CVRHGNFGNSYISYWAYWGQGTLVTVSSG | |
| | | GGGSGGGGSGGGGSQTVVTQEPSLTVSPG | |
| | | GTVTLTCGSSTGAVTSGNYPNWVQQKPGQ | |
| | | APRGLIGGTKFLAPGTPARFSGSLLGGKAA | |
| | | LTLSGVQPEDEAEYYCVLWYSNRWVFGGG | |
| | | TKLTVLGGGGSQVQLKQSGPGLVQPSQSLS | |
| | | ITCTVSGFSLTNYGVHWVRQSPGKGLEWL | |
| | | GVIWSGGNTDYNTPFTSRLSINKDNSKSQV | |
| | | FFKMNSLQSQDTAIYYCARALTYYDYEFA | |
| | | YWGQGTLVTVSAASTKGPSVFPLAPSSKST | |
| | | SGGTAALGCLVKDYFPEPVTVSWNSGALT | |
| | | SGVHTFPAVLQSSGLYSLSSVVTVPSSSLGT | |
| | | QTYICNVNHKPSNTKVDKKVEPKSCGGHH | |
| | | HHHHHH | |
| PC-7 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| PC-7 | Heavy Chain Sequence: | EVQLVESGGGLVQPGNSLRLSCAASGFTFS | 76 |
| | N-[10G SDA]- | KFGMSWVRQAPGKGLEWVSSISGSGRDTL | |
| | [Peptide-25]-[linker]- | YADSVKGRFTISRDNAKTTLYLQMNSLRPE | |
| | [anti-CD3 scFv (light | DTAVYYCTIGGSLSVSSQGTLVTVSSGGGG | |
| | chain-heavy chain)]- | SGGGSGGQGCGTIADPEPHCWGSSGGSGGS | |
| | [anti-EGFR Fab heavy | GGISSGLLSGRSDNHGGGSQTVVTQEPSLT | |
| | chain]-C | VSPGGTVTLTCRSSTGAVTTSNYANWVQQ | |
| | | KPGQAPRGLIGGTNKRAPGTPARFSGSLLG | |
| | | GKAALTLSGVQPEDEAEYYCALWYSNLW | |
| | | VFGGGTKLTVLGGGGSGGGGSGGGGSEVQ | |
| | | LVESGGGLVQPGGSLKLSCAASGFTFNTYA | |
| | | MNWVRQAPGKGLEWVARIRSKYNNYATY | |
| | | YADSVKDRFTISRDDSKNTAYLQMNNLKT | |
| | | EDTAVYYCVRHGNFGNSYVSWFAYWGQG | |
| | | TLVTVSSGGGGSQVQLKQSGPGLVQPSQSL | |
| | | SITCTVSGFSLTNYGVHWVRQSPGKGLEWL | |
| | | GVIWSGGNTDYNTPFTSRLSINKDNSKSQV | |
| | | FFKMNSLQSQDTAIYYCARALTYYDYEFA | |
| | | YWGQGTLVTVSAASTKGPSVFPLAPSSKST | |
| | | SGGTAALGCLVKDYFPEPVTVSWNSGALT | |
| | | SGVHTFPAVLQSSGLYSLSSVVTVPSSSLGT | |
| | | QTYICNVNHKPSNTKVDKKVEPKSCGGGH | |
| | | HHHHHHH | |
| PC-8 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| PC-8 | Heavy Chain Sequence: | QTVVTQEPSLTVSPGGTVTLTCRSSTGAVT | 77 |
| | N-[anti-CD3 scFv | TSNYANWVQQKPGQAPRGLIGGTNKRAPG | |
| | (light chain-heavy | TPARFSGSLLGGKAALTLSGVQPEDEAEYY | |
| | chain)]-[anti-EGFR | CALWYSNLWVFGGGTKLTVLGGGGSGGG | |
| | Fab heavy chain]-C | GSGGGGSEVQLVESGGGLVQPGGSLKLSC | |
| | | AASGFTFNTYAMNWVRQAPGKGLEWVAR | |
| | | IRSKYNNYATYYADSVKDRFTISRDDSKNT | |
| | | AYLQMNNLKTEDTAVYYCVRHGNFGNSY | |
| | | VSWFAYWGQGTLVTVSSGGGGSQVQLKQ | |
| | | SGPGLVQPSQSLSITCTVSGFSLTNYGVHW | |
| | | VRQSPGKGLEWLGVIWSGGNTDYNTPFTS | |
| | | RLSINKDNSKSQVFFKMNSLQSQDTAIYYC | |
| | | ARALTYYDYEFAYWGQGTLVTVSAASTKG | |
| | | PSVFPLAPSSKSTSGGTAALGCLVKDYFPEP | |
| | | VTVSWNSGALTSGVHTFPAVLQSSGLYSLS | |
| | | SVVTVPSSSLGTQTYICNVNHKPSNTKVDK | |
| | | KVEPKSCGGHHHHHHHHGGGLNDIFEAQK | |
| | | IEWHE | |
| PC-9 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| PC-9 | Heavy Chain Sequence: | EVQLVESGGGLVQPGNSLRLSCAASGFTFS | 78 |
| | N-[10G SDA]-[Peptide | KFGMSWVRQAPGKGLEWVSSISGSGRDTL | |
| | 29]-[linker]-[anti-CD3 | YADSVKGRFTISRDNAKTTLYLQMNSLRPE | |
| | scFv (light chain- | DTAVYYCTIGGSLSVSSQGTLVTVSSGGGG | |
| | heavy chain)]- | SGGGSQGQSGSGYLWGCEWNCGGITTGSS | |
| | [anti-EGFR | GGSGGSGGISSGLLSGRSDNHGGGSQTVVT | |
| | Fab heavy chain]-C | QEPSFSVSPGGTVTLTCRSSTGAVTTSNYA | |
| | | NWVQQTPGQAPRGLIGGTNKRAPGVPDRF | |
| | | SGSILGNKAALTITGAQADDESDYYCALW | |
| | | YSNLWVFGGGTKLTVLGGGGSGGGGSGG | |
| | | GGSEVQLVESGGGLVQPGGSLKLSCAASGF | |
| | | TFSTYAMNWVRQASGKGLEWVGRIRSKY | |
| | | NNYATYYADSVKDRFTISRDDSKNTAYLQ | |
| | | MNSLKTEDTAVYYCTRHGNFGNSYVSWFA | |
| | | YWGQGTLVTVSSGGGGSQVQLKQSGPGLV | |
| | | QPSQSLSITCTVSGFSLTNYGVHWVRQSPG | |
| | | KGLEWLGVIWSGGNTDYNTPFTSRLSINKD | |
| | | NSKSQVFFKMNSLQSQDTAIYYCARALTY | |
| | | YDYEFAYWGQGTLVTVSAASTKGPSVFPL | |
| | | APSSKSTSGGTAALGCLVKDYFPEPVTVSW | |
| | | NSGALTSGVHTFPAVLQSSGLYSLSSVVTV | |
| | | PSSSLGTQTYICNVNHKPSNTKVDKKVEPK | |
| | | SCGGHHHHHHHHGGGLNDIFEAQKIEWHE | |
| PC-10 | Light Chain Sequence: | QILLTQSPVILSVSPGERVSFSCRASQSIGTNI | 57 |
| | anti-EGFR Fab light | HWYQQRTNGSPRLLIKYASESISGIPSRFSG | |
| | chain | SGSGTDFTLSINSVESEDIADYYCQQNNNW | |
| | | PTTFGAGTKLELKRTVAAPSVFIFPPSDEQL | |
| | | KSGTASVVCLLNNFYPREAKVQWKVDNAL | |
| | | QSGNSQESVTEQDSKDSTYSLSSTLTLSKA | |
| | | DYEKHKVYACEVTHQGLSSPVTKSFNRGE | |
| | | C | |
| PC-10 | Heavy Chain Sequence: | EVQLVESGGGLVQPGNSLRLSCAASGFTFS | 73 |
| | N-[10G SDA]- | KFGMSWVRQAPGKGLEWVSSISGSGRDTL | |
| | [Peptide-18]-[linker]- | YADSVKGRFTISRDNAKTTLYLQMNSLRPE | |
| | [anti-CD3 scFv (light | DTAVYYCTIGGSLSVSSQGTLVTVSSGGGG | |
| | chain-heavy chain)]- | SGGGSQGQSGQGYLWGCEWNCGGITTGSS | |
| | [anti-EGFR Fab heavy | GGSGGSGGISSGLLSGRSDNHGGGSQTVVT | |
| | chain]-C | QEPSLTVSPGGTVTLTCRSSTGAVTTSNYA | |
| | | NWVQQKPGQAPRGLIGGTNKRAPGTPARF | |
| | | SGSLLGGKAALTLSGVQPEDEAEYYCALW | |
| | | YSNLWVFGGGTKLTVLGGGGSGGGGSGG | |
| | | GGSEVQLVESGGGLVQPGGSLKLSCAASGF | |
| | | TFNTYAMNWVRQAPGKGLEWVARIRSKY | |
| | | NNYATYYADSVKDRFTISRDDSKNTAYLQ | |
| | | MNNLKTEDTAVYYCVRHGNFGNSYVSWF | |
| | | AYWGQGTLVTVSSGGGGSQVQLKQSGPGL | |
| | | VQPSQSLSITCTVSGFSLTNYGVHWVRQSP | |
| | | GKGLEWLGVIWSGGNTDYNTPFTSRLSINK | |
| | | DNSKSQVFFKMNSLQSNDTAIYYCARALT | |
| | | YYDYEFAYWGQGTLVTVSAASTKGPSVFP | |
| | | LAPSSKSTSGGTAALGCLVKDYFPEPVTVS | |
| | | WNSGALTSGVHTFPAVLQSSGLYSLSSVVT | |
| | | VPSSSLGTQTYICNVNHKPSNTKVDKKVEP | |
| | | KSCGGHHHHHHHHGGGLNDIFEAQKIEWH | |
| | | E | |
| PC-11 | Light Chain Sequence: | DIQMTQSPSSLSASVGDRVTITCRASQDVN | 61 |
| | anti-HER2 Fab light | TAVAWYQQKPGKAPKLLIYSASFLYSGVPS | |
| | chain | RFSGSRSGTDFTLTISSLQPEDFATYYCQQH | |
| | | YTTPPTFGQGTKVEIKRTVAAPSVFIFPPSDE | |
| | | QLKSGTASVVCLLNNFYPREAKVQWKVDN | |
| | | ALQSGNSQESVTEQDSKDSTYSLSSTLTLSK | |
| | | ADYEKHKVYACEVTHQGLSSPVTKSFNRG | |
| | | EC | |
| PC-11 | Heavy Chain Sequence: | QTVVTQEPSLTVSPGGTVTLTCRSSTGAVT | 79 |
| | N-[anti-CD3 scFv | TSNYANWVQQKPGQAPRGLIGGTNKRAPG | |
| | (light chain-heavy | TPARFSGSLLGGKAALTLSGVQPEDEAEYY | |
| | chain)]-[anti-HER2 Fab | CALWYSNLWVFGGGTKLTVLGGGGSGGG | |
| | heavy chain]-C | GSGGGGSEVQLVESGGGLVQPGGSLKLSC | |
| | | AASGFTFNTYAMNWVRQAPGKGLEWVAR | |
| | | IRSKYNNYATYYADSVKDRFTISRDDSKNT | |
| | | AYLQMNNLKTEDTAVYYCVRHGNFGNSY | |
| | | VSWFAYWGQGTLVTVSSGGGGSEVQLVES | |
| | | GGGLVQPGGSLRLSCAASGFNIKDTYIHWV | |
| | | RQAPGKGLEWVARIYPTNGYTRYADSVKG | |
| | | RFTISADTSKNTAYLQMNSLRAEDTAVYYC | |
| | | SRWGGDGFYAMDYWGQGTLVTVSSASTK | |
| | | GPSVFPLAPSSKSTSGGTAALGCLVKDYFPE | |
| | | PVTVSWNSGALTSGVHTFPAVLQSSGLYSL | |
| | | SSVVTVPSSSLGTQTYICNVNHKPSNTKVD | |
| | | KKVEPKSCAAAHHHHHHHH | |
| PC-12 | Light Chain Sequence: | GGTGCTEGHWHWGTCSGGGGSGGGGSGG | 80 |
| | N-[ Peptide-14]- | ISSGLLSGRSDNHGSSGTDIQMTQSPSSLSA | |
| | [linker]-[anti-HER2 | SVGDRVTITCRASQDVNTAVAWYQQKPGK | |
| | Fab light chain]-C | APKLLIYSASFLYSGVPSRFSGSRSGTDFTLT | |
| | | ISSLQPEDFATYYCQQHYTTPPTFGQGTKV | |
| | | EIKRTVAAPSVFIFPPSDEQLKSGTASVVCL | |
| | | LNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| PC-12 | Heavy Chain Sequence: | EVQLVESGGGLVQPGNSLRLSCAASGFTFS | 81 |
| | N-[10G SDA]- | KFGMSWVRQAPGKGLEWVSSISGSGRDTL | |
| | [Peptide-18]-[linker]- | YADSVKGRFTISRDNAKTTLYLQMNSLRPE | |
| | [anti-CD3 scFv (light | DTAVYYCTIGGSLSVSSQGTLVTVSSGGGG | |
| | chain-heavy chain)]- | SGGGSQGQSGQGYLWGCEWNCGGITTGSS | |
| | [anti-EGFR Fab heavy | GGSGGSGGISSGLLSGRSDNHGGGSQTVVT | |
| | chain]-C | QEPSLTVSPGGTVTLTCRSSTGAVTTSNYA | |
| | | NWVQQKPGQAPRGLIGGTNKRAPGTPARF | |
| | | SGSLLGGKAALTLSGVQPEDEAEYYCALW | |
| | | YSNLWVFGGGTKLTVLGGGGSGGGGSGG | |
| | | GGSEVQLVESGGGLVQPGGSLKLSCAASGF | |
| | | TFNTYAMNWVRQAPGKGLEWVARIRSKY | |
| | | NNYATYYADSVKDRFTISRDDSKNTAYLQ | |
| | | MNNLKTEDTAVYYCVRHGNFGNSYVSWF | |
| | | AYWGQGTLVTVSSGGGGSEVQLVESGGGL | |
| | | VQPGGSLRLSCAASGFNIKDTYIHWVRQAP | |
| | | GKGLEWVARIYPTNGYTRYADSVKGRFTIS | |
| | | ADTSKNTAYLQMNSLRAEDTAVYYCSRW | |
| | | GGDGFYAMDYWGQGTLVTVSSASTKGPS | |
| | | VFPLAPSSKSTSGGTAALGCLVKDYFPEPV | |
| | | TVSWNSGALTSGVHTFPAVLQSSGLYSLSS | |
| | | VVTVPSSSLGTQTYICNVNHKPSNTKVDKK | |
| | | VEPKSCAAAHHHHHHHH | |

Polypeptides or polypeptide complexes, in some embodiments, comprise a sequence set forth in Table 1. In some embodiments, the sequence comprises at least or about 70%, 80%, 85%, 90%, 91%, 92%, 93%, 94%, 95%, 96%, 97%, 98%, 99%, or 100% sequence identity to SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81. In some instances, the sequence comprises at least or about 95% identity to SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81. In some instances, the sequence comprises at least or about 97% identity to SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81. In some instances, the sequence comprises at least or about 99% identity to SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81. In some instances, the sequence comprises at least or about 100% identity to SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81. In some instances, the sequence comprises at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, or more than 400 amino acids of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, or 81.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blast program. The program also allows use of an SEG filter to mask-off segments of the query sequences as determined by the SEG program of Wootton and Federhen, Computers and Chemistry 17: 149-163 (1993). High sequence identity generally includes ranges of sequence identity of approximately 80% to 100% and integer values there between.

### Embodiments

Embodiment 1 comprises a polypeptide or polypeptide complex according to Formula I:

A₂-A₁-L₁-P₁-H₁ (Formula I)

wherein: A₁ comprises a first antigen recognizing molecule that binds to a first target antigen; P₁ comprises a peptide that binds to A₁; L₁ comprises a linking moiety that connects A₁ to P₁ and is a substrate for a tumor specific protease; H₁ comprises a half-life extending molecule; and A₂ comprises a second antigen recognizing molecule that binds to a second target antigen.

Embodiment 2 comprises a polypeptide or polypeptide complex of embodiment 1, wherein the first target antigen comprises an effector cell antigen and the second target antigen comprises a tumor cell antigen.

Embodiment 3 comprises a polypeptide or polypeptide complex of any one of embodiments 1-2, wherein the effector cell antigen comprises CD3.

Embodiment 4 comprises a polypeptide or polypeptide complex of any one of embodiments 1-3, wherein the tumor cell antigen comprises EGFR, HER2, mesothelin, or CEACAM5.

Embodiment 5 comprises a polypeptide or polypeptide complex of any one of embodiments 1-4, wherein A₁ comprises an antibody or antibody fragment.

Embodiment 6 comprises a polypeptide or polypeptide complex of any one of embodiments 1-5, wherein A₁ comprises an antibody or antibody fragment that is human or humanized

Embodiment 7 comprises a polypeptide or polypeptide complex of any one of embodiments 1-6, wherein L₁ is bound to N-terminus of the antibody or antibody fragment.

Embodiment 8 comprises a polypeptide or polypeptide complex of any one of embodiments 1-7, wherein A₂ is bound to C-terminus of the antibody or antibody fragment.

Embodiment 9 comprises a polypeptide or polypeptide complex of any one of embodiments 1-8, wherein L₁ is bound to C-terminus of the antibody or antibody fragment.

Embodiment 10 comprises a polypeptide or polypeptide complex of any one of embodiments 1-9, wherein A₂ is bound to N-terminus of the antibody or antibody fragment.

Embodiment 11 comprises a polypeptide or polypeptide complex of any one of embodiments 1-10, wherein the antibody or antibody fragment comprises a single chain variable fragment, a single domain antibody, or a Fab fragment.

Embodiment 12 comprises a polypeptide or polypeptide complex of any one of embodiments 1-11, wherein A₁ is the single chain variable fragment (scFv).

Embodiment 13 comprises a polypeptide or polypeptide complex of any one of embodiments 1-12, wherein the scFv comprises a scFv heavy chain polypeptide and a scFv light chain polypeptide.

Embodiment 14 comprises a polypeptide or polypeptide complex of any one of embodiments 1-13, wherein A₁ is the single domain antibody.

Embodiment 15 comprises a polypeptide or polypeptide complex of any one of embodiments 1-14, A₁ is a single chain variable fragment (scFv), a heavy chain variable domain (VH domain), a light chain variable domain (VL domain), or a variable domain (VHH) of a camelid derived single domain antibody.

Embodiment 16 comprises a polypeptide or polypeptide complex of any one of embodiments 1-15, wherein A₁ comprises an anti-CD3e single chain variable fragment.

Embodiment 17 comprises a polypeptide or polypeptide complex of any one of embodiments 1-16, wherein A₁ comprises an anti-CD3e single chain variable fragment that has a K_{D} binding of 1 μM or less to CD3 on CD3 expressing cells.

Embodiment 18 comprises a polypeptide or polypeptide complex of any one of embodiments 1-17, wherein A₁ comprises a variable light chain and variable heavy chain each of which is capable of specifically binding to human CD3.

Embodiment 19 comprises a polypeptide or polypeptide complex of any one of embodiments 1-18, wherein A₁ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 20 comprises a polypeptide or polypeptide complex of any one of embodiments 1-19, wherein the polypeptide or polypeptide complex of formula I binds to an effector cell when L₁ is cleaved by the tumor specific protease.

Embodiment 21 comprises a polypeptide or polypeptide complex of any one of embodiments 1-20, wherein the polypeptide or polypeptide complex of formula I binds to an effector cell when L₁ is cleaved by the tumor specific protease and A₁ binds to the effector cell.

Embodiment 22 comprises a polypeptide or polypeptide complex of any one of embodiments 1-21, wherein the effector cell is a T cell.

Embodiment 23 comprises a polypeptide or polypeptide complex of any one of embodiments 1-22, wherein A₁ binds to a polypeptide that is part of a TCR-CD3 complex on the effector cell.

Embodiment 24 comprises a polypeptide or polypeptide complex of any one of embodiments 1-23, wherein the polypeptide that is part of the TCR-CD3 complex is human CD3_{ε}.

Embodiment 25 comprises a polypeptide or polypeptide complex of any one of embodiments 1-24, wherein the effector cell antigen comprises CD3, and the scFv comprises an amino acid sequence according to SEQ ID NOs: 66, 67, or 68.

Embodiment 26 comprises a polypeptide or polypeptide complex of any one of embodiments 1-25, wherein A₂ comprises an antibody or antibody fragment.

Embodiment 27 comprises a polypeptide or polypeptide complex of any one of embodiments 1-26, wherein the antibody or antibody fragment thereof comprises a single chain variable fragment, a single domain antibody, or a Fab.

Embodiment 28 comprises a polypeptide or polypeptide complex of any one of embodiments 1-27, wherein the antibody or antibody fragment thereof comprises a single chain variable fragment (scFv), a heavy chain variable domain (VH domain), a light chain variable domain (VL domain), a variable domain (VHH) of a camelid derived single domain antibody.

Embodiment 29 comprises a polypeptide or polypeptide complex of any one of embodiments 1-28, wherein the antibody or antibody fragment thereof is humanized or human.

Embodiment 30 comprises a polypeptide or polypeptide complex of any one of embodiments 1-29, wherein A₂ is the Fab.

Embodiment 31 comprises a polypeptide or polypeptide complex of any one of embodiments 1-30, wherein the Fab comprises (a) a Fab light chain polypeptide and (b) a Fab heavy chain polypeptide.

Embodiment 32 comprises a polypeptide or polypeptide complex of any one of embodiments 1-31, wherein the antibody or antibody fragment thereof comprises an epidermal growth factor receptor (EGFR) binding domain.

Embodiment 33 comprises a polypeptide or polypeptide complex of any one of embodiments 1-32, wherein the antibody or antibody fragment thereof comprises a mesothelin binding domain.

Embodiment 34 comprises a polypeptide or polypeptide complex of any one of embodiments 1-33, wherein the antibody or antibody fragment thereof comprises a carcinoembryonic antigen-related cell adhesion molecule CEACAM5 binding domain.

Embodiment 35 comprises a polypeptide or polypeptide complex of any one of embodiments 1-34, wherein the tumor cell antigen comprises EGFR, and the Fab light chain polypeptide comprises an amino acid sequence according to SEQ ID NOs: 56 or 57.

Embodiment 36 comprises a polypeptide or polypeptide complex of any one of embodiments 1-35, wherein the tumor cell antigen comprises EGFR, and the Fab heavy chain polypeptide comprises an amino acid sequence according to SEQ ID NOs: 59 or 60.

Embodiment 37 comprises a polypeptide or polypeptide complex of any one of embodiments 1-36, wherein the tumor cell antigen comprises HER2, and the Fab light chain polypeptide comprises an amino acid sequence according to SEQ ID NO: 61.

Embodiment 38 comprises a polypeptide or polypeptide complex of any one of embodiments 1-37, wherein the tumor cell antigen comprises HER2 and the Fab heavy chain polypeptide comprises an amino acid sequence according to SEQ ID NOs: 62 or 63.

Embodiment 39 comprises a polypeptide or polypeptide complex of any one of embodiments 1-38, wherein the Fab light chain polypeptide of A₂ is bound to a C-terminus of the single chain variable fragment (scFv) of A₁.

Embodiment 40 comprises a polypeptide or polypeptide complex of any one of embodiments 1-39, wherein the Fab heavy chain polypeptide of A₂ is bound to a C-terminus of the single chain variable fragment (scFv) A₁.

Embodiment 41 comprises a polypeptide or polypeptide complex of any one of embodiments 1-40, wherein the Fab light chain polypeptide of A₂ is bound to a N-terminus of the single chain variable fragment (scFv) of A₁.

Embodiment 42 comprises a polypeptide or polypeptide complex of any one of embodiments 1-41, wherein the Fab heavy chain polypeptide of A₂ is bound to a N-terminus of the single chain variable fragment (scFv) A₁.

Embodiment 43 comprises a polypeptide or polypeptide complex of any one of embodiments 1-42, wherein the Fab heavy chain polypeptide of A₂ is bound to the scFv heavy chain polypeptide of A₁.

Embodiment 44 comprises a polypeptide or polypeptide complex of any one of embodiments 1-43, wherein the Fab heavy chain polypeptide of A₂ is bound to the scFv heavy chain polypeptide of A₁, and the polypeptide complex comprises amino acid sequences of SEQ ID NO: 57 and SEQ ID NO: 76.

Embodiment 45 comprises a polypeptide or polypeptide complex of any one of embodiments 1-44, wherein the Fab heavy chain polypeptide of A₂ is bound to the scFv heavy chain polypeptide of A₁, and the polypeptide complex comprises amino acid sequences of SEQ ID NO: 57 and SEQ ID NO: 78.

Embodiment 46 comprises a polypeptide or polypeptide complex of any one of embodiments 1-45, wherein the Fab heavy chain polypeptide of A₂ is bound to the scFv heavy chain polypeptide of A₁, and the polypeptide complex comprises amino acid sequences of SEQ ID NO: 57 and SEQ ID NO: 73.

Embodiment 47 comprises a polypeptide or polypeptide complex of any one of embodiments 1-46, wherein the Fab light chain polypeptide of A₂ is bound to the scFv heavy chain polypeptide of A₁.

Embodiment 48 comprises a polypeptide or polypeptide complex of any one of embodiments 1-47, wherein the Fab heavy chain polypeptide of A₂ is bound to the scFv light chain polypeptide of A₁.

Embodiment 49 comprises a polypeptide or polypeptide complex of any one of embodiments 1-48, wherein the Fab heavy chain polypeptide of A₂ is bound to the scFv light chain polypeptide of A₁, and the polypeptide complex comprises amino acid sequences of SEQ ID NO: 57 and SEQ ID NO: 74.

Embodiment 50 comprises a polypeptide or polypeptide complex of any one of embodiments 1-49, wherein the Fab light chain polypeptide of A₂ is bound to the scFv light chain polypeptide of A₁.

Embodiment 51 comprises a polypeptide or polypeptide complex of any one of embodiments 1-50, wherein A₂ further comprises P₂ and L₂, wherein P₂ comprises a peptide that binds to A₂; and L₂ comprises a linking moiety that connects A₂to P₂ and is a substrate for a tumor specific protease.

Embodiment 52 comprises the polypeptide or polypeptide complex of any one of embodiments 1-51, wherein the polypeptide or polypeptide complex is according to Formula Ia

P₂-L₂-A₂-A₁-L₁-P₁-H₁ (Formula Ia)

Embodiment 53 comprises a polypeptide or polypeptide complex of any one of embodiments 1-52, wherein the Fab heavy chain polypeptide of A₂ is bound to the scFv heavy chain polypeptide of A₁and L₂ is bound to the Fab light chain polypeptide of A₂.

Embodiment 54 comprises a polypeptide or polypeptide complex of any one of embodiments 1-53, wherein the Fab heavy chain polypeptide of A₂ is bound to the scFv heavy chain polypeptide of A₁and L₂ is bound to the Fab light chain polypeptide of A₂ and the polypeptide complex comprises amino acid sequences of SEQ ID NO: 70 and SEQ ID NO: 73.

Embodiment 55 comprises a polypeptide or polypeptide complex of any one of embodiments 1-54, wherein the Fab heavy chain polypeptide of A₂ is bound to the scFv heavy chain polypeptide of A₁ and L₂ is bound to the Fab light chain polypeptide of A₂ and the polypeptide complex comprises amino acid sequences of SEQ ID NO: 80 and SEQ ID NO: 81.

Embodiment 56 comprises a polypeptide or polypeptide complex of any one of embodiments 1-55, wherein the Fab light chain polypeptide of A₂ is bound to the scFv heavy chain polypeptide of A₁ and L₂ is bound to the Fab heavy chain polypeptide of A₂.

Embodiment 57 comprises a polypeptide or polypeptide complex of any one of embodiments 1-56, wherein the Fab heavy chain polypeptide of A₂ is bound to the scFv light chain polypeptide of A₁ and L₂ is bound to the Fab light chain polypeptide of A₂.

Embodiment 58 comprises a polypeptide or polypeptide complex of any one of embodiments 1-57, wherein the Fab light chain polypeptide of A₂ is bound to the scFv light chain polypeptide of A₁ and L₂ is bound to the Fab heavy chain polypeptide of A₂.

Embodiment 59 comprises a polypeptide or polypeptide complex of any one of embodiments 1-58, wherein the first target antigen comprises a tumor cell antigen and the second target antigen comprises an effector cell antigen

Embodiment 60 comprises a polypeptide or polypeptide complex of any one of embodiments 1-59, wherein the tumor cell antigen comprises EGFR, HER2, mesothelin, or CEACAM5.

Embodiment 61 comprises a polypeptide or polypeptide complex of any one of embodiments 1-60, wherein the effector cell antigen comprises CD3.

Embodiment 62 comprises a polypeptide or polypeptide complex of any one of embodiments 1-61, wherein A₁ comprises an antibody or antibody fragment.

Embodiment 63 comprises a polypeptide or polypeptide complex of any one of embodiments 1-62, wherein A₁ comprises an antibody or antibody fragment that is human or humanized.

Embodiment 64 comprises a polypeptide or polypeptide complex of any one of embodiments 1-63, wherein L₁ is bound to N-terminus of the antibody or antibody fragment.

Embodiment 65 comprises a polypeptide or polypeptide complex of any one of embodiments 1-64, wherein A₂ is bound to C-terminus of the antibody or antibody fragment.

Embodiment 66 comprises a polypeptide or polypeptide complex of any one of embodiments 1-65, wherein L₁ is bound to C-terminus of the antibody or antibody fragment.

Embodiment 67 comprises a polypeptide or polypeptide complex of any one of embodiments 1-66, wherein A₂ is bound to N-terminus of the antibody or antibody fragment.

Embodiment 68 comprises a polypeptide or polypeptide complex of any one of embodiments 1-67, wherein the antibody or antibody fragment thereof comprises a single chain variable fragment, a single domain antibody, or a Fab.

Embodiment 69 comprises a polypeptide or polypeptide complex of any one of embodiments 1-68, wherein the antibody or antibody fragment thereof comprises a single chain variable fragment (scFv), a heavy chain variable domain (VH domain), a light chain variable domain (VL domain), a variable domain (VHH) of a camelid derived single domain antibody.

Embodiment 70 comprises a polypeptide or polypeptide complex of any one of embodiments 1-69, wherein the antibody or antibody fragment thereof is humanized or human.

Embodiment 71 comprises a polypeptide or polypeptide complex of any one of embodiments 1-70, wherein A₁ is the Fab.

Embodiment 72 comprises a polypeptide or polypeptide complex of any one of embodiments 1-71, wherein the Fab comprises (a) a Fab light chain polypeptide and (b) a Fab heavy chain polypeptide.

Embodiment 73 comprises a polypeptide or polypeptide complex of any one of embodiments 1-72, wherein the antibody or antibody fragment thereof comprises an epidermal growth factor receptor (EGFR) binding domain.

Embodiment 74 comprises a polypeptide or polypeptide complex of any one of embodiments 1-73, wherein the antibody or antibody fragment thereof comprises a mesothelin binding domain.

Embodiment 75 comprises a polypeptide or polypeptide complex of any one of embodiments 1-74, wherein the antibody or antibody fragment thereof comprises a carcinoembryonic antigen-related cell adhesion molecule CEACAM5 binding domain.

Embodiment 76 comprises a polypeptide or polypeptide complex of any one of embodiments 1-75, wherein the tumor cell antigen comprises EGFR, and the Fab light chain polypeptide comprises an amino acid sequence according to SEQ ID NOs: 56 or 57.

Embodiment 77 comprises a polypeptide or polypeptide complex of any one of embodiments 1-76, wherein the tumor cell antigen comprises EGFR, and the Fab heavy chain polypeptide comprises an amino acid sequence according to SEQ ID NOs: 59 or 60.

Embodiment 78 comprises a polypeptide or polypeptide complex of any one of embodiments 1-77, wherein the tumor cell antigen comprises HER2, and the Fab light chain polypeptide comprises an amino acid sequence according to SEQ ID NO: 61.

Embodiment 79 comprises a polypeptide or polypeptide complex of any one of embodiments 1-78, wherein the tumor cell antigen comprises HER2 and the Fab heavy chain polypeptide comprises an amino acid sequence according to SEQ ID NOs: 62 or 63.

Embodiment 80 comprises a polypeptide or polypeptide complex of any one of embodiments 1-79, wherein A₂ comprises an antibody or antibody fragment.

Embodiment 81 comprises a polypeptide or polypeptide complex of any one of embodiments 1-80, wherein A₂ comprises an antibody or antibody fragment that is human or humanized

Embodiment 82 comprises a polypeptide or polypeptide complex of any one of embodiments 1-81, wherein the antibody or antibody fragment comprises a single chain variable fragment, a single domain antibody, or a Fab fragment.

Embodiment 83 comprises a polypeptide or polypeptide complex of any one of embodiments 1-82, wherein A₂ is the single chain variable fragment (scFv).

Embodiment 84 comprises a polypeptide or polypeptide complex of any one of embodiments 1-83, wherein the scFv comprises a scFv heavy chain polypeptide and a scFv light chain polypeptide.

Embodiment 85 comprises a polypeptide or polypeptide complex of any one of embodiments 1-84, wherein A₂ is the single domain antibody.

Embodiment 86 comprises a polypeptide or polypeptide complex of any one of embodiments 1-85, wherein the single domain antibody comprises a single chain variable fragment (scFv), a heavy chain variable domain (VH domain), a light chain variable domain (VL domain), or a variable domain (VHH) of a camelid derived single domain antibody.

Embodiment 87 comprises a polypeptide or polypeptide complex of any one of embodiments 1-86, wherein A₂ comprises an anti-CD3e single chain variable fragment.

Embodiment 88 comprises a polypeptide or polypeptide complex of any one of embodiments 1-87, wherein A₂ comprises an anti-CD3e single chain variable fragment that has a K_{D} binding of 1 μM or less to CD3 on CD3 expressing cells.

Embodiment 89 comprises a polypeptide or polypeptide complex of any one of embodiments 1-88, wherein A₂ comprises a variable light chain and variable heavy chain each of which is capable of specifically binding to human CD3.

Embodiment 90 comprises a polypeptide or polypeptide complex of any one of embodiments 1-89, wherein A₂ comprises complementary determining regions (CDRs) selected from the group consisting of muromonab-CD3 (OKT3), otelixizumab (TRX4), teplizumab (MGA031), visilizumab (Nuvion), SP34, X35, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1, WT-31, 15865, 15865v12, 15865v16, and 15865v19.

Embodiment 91 comprises a polypeptide or polypeptide complex of any one of embodiments 1-90, wherein the polypeptide or polypeptide complex of formula I binds to an effector cell.

Embodiment 92 comprises a polypeptide or polypeptide complex of any one of embodiments 1-91, wherein the effector cell is a T cell.

Embodiment 93 comprises a polypeptide or polypeptide complex of any one of embodiments 1-92, wherein A₂ binds to a polypeptide that is part of a TCR-CD3 complex on the effector cell.

Embodiment 94 comprises a polypeptide or polypeptide complex of any one of embodiments 1-93, wherein the polypeptide that is part of the TCR-CD3 complex is human CD3_{ε}.

Embodiment 95 comprises a polypeptide or polypeptide complex of any one of embodiments 1-94, wherein the effector cell antigen comprises CD3, and the scFv comprises an amino acid sequence according to SEQ ID NOs: 66, 67, or 68.

Embodiment 96 comprises a polypeptide or polypeptide complex of any one of embodiments 1-95, wherein the Fab light chain polypeptide of A₁ is bound to a C-terminus of the single chain variable fragment (scFv) of A₂.

Embodiment 97 comprises a polypeptide or polypeptide complex of any one of embodiments 1-96, wherein the Fab heavy chain polypeptide of A₁ is bound to a C-terminus of the single chain variable fragment (scFv) A₂.

Embodiment 98 comprises a polypeptide or polypeptide complex of any one of embodiments 1-97, wherein the Fab light chain polypeptide of A₁ is bound to a N-terminus of the single chain variable fragment (scFv) of A₂.

Embodiment 99 comprises a polypeptide or polypeptide complex of any one of embodiments 1-98, wherein the Fab heavy chain polypeptide of A₁ is bound to a N-terminus of the single chain variable fragment (scFv) A₂.

Embodiment 100 comprises a polypeptide or polypeptide complex of any one of embodiments 1-99, wherein the Fab heavy chain polypeptide of A₁ is bound to the scFv heavy chain polypeptide of A₂ and L₁ is bound to the Fab light chain polypeptide of A₁.

Embodiment 101 comprises a polypeptide or polypeptide complex of any one of embodiments 1-100, wherein the Fab light chain polypeptide of A₁ is bound to the scFv heavy chain polypeptide of A₂ and L₁ is bound to the Fab heavy chain polypeptide of A₁.

Embodiment 102 comprises a polypeptide or polypeptide complex of any one of embodiments 1-101, wherein the Fab heavy chain polypeptide of A₁ is bound to the scFv light chain polypeptide of A₂ and L₁ is bound to the Fab light chain polypeptide of A₁.

Embodiment 103 comprises a polypeptide or polypeptide complex of any one of embodiments 1-102, wherein the Fab light chain polypeptide of A₁ is bound to the scFv light chain polypeptide of A₂ and L₁ is bound to the Fab heavy chain polypeptide of A₁.

Embodiment 104 comprises a polypeptide or polypeptide complex of any one of embodiments 1-103, wherein A₂ further comprises P₂ and L₂, wherein P₂ comprises a peptide that binds to A₂; and L₂ comprises a linking moiety that connects A₂ to P₂ and is a substrate for a tumor specific protease.

Embodiment 105 comprises the polypeptide or polypeptide complex of any one of embodiments 1-104, wherein the polypeptide or polypeptide complex is according to Formula Ia

P₂-L₂-A₂-A₁-L₁-P₁-H₁ (Formula Ia)

Embodiment 106 comprises a polypeptide or polypeptide complex of any one of embodiments 1-105, wherein the Fab heavy chain polypeptide of A₁ is bound to the scFv heavy chain polypeptide of A₂ and L₁ is bound to the Fab light chain polypeptide of A₁ and L₂ is bound to the scFv light chain polypeptide of A₂.

Embodiment 107 comprises a polypeptide or polypeptide complex of any one of embodiments 1-106, wherein the Fab heavy chain polypeptide of A₁ is bound to the scFv heavy chain polypeptide of A₂ and L₁ is bound to the Fab light chain polypeptide of A₁ and L₂ is bound to the scFv light chain polypeptide of A₂, and the polypeptide complex comprises amino acid sequences of SEQ ID NO: 72 and SEQ ID NO: 71.

Embodiment 108 comprises a polypeptide or polypeptide complex of any one of embodiments 1-107, wherein the Fab light chain polypeptide of A₁ is bound to the scFv heavy chain polypeptide of A₂ and L₁ is bound to the Fab heavy chain polypeptide of A₁ and L₂ is bound to the scFv light chain polypeptide of A₂.

Embodiment 109 comprises a polypeptide or polypeptide complex of any one of embodiments 1-108, wherein the Fab heavy chain polypeptide of A₁ is bound to the scFv light chain polypeptide of A₂ and L₁ is bound to the Fab light chain polypeptide of A₁ and L₂ is bound to the scFv heavy chain polypeptide of A₂.

Embodiment 110 comprises a polypeptide or polypeptide complex of any one of embodiments 1-109, wherein the Fab light chain polypeptide of A₁ is bound to the scFv light chain polypeptide of A₂ and L₁ is bound to the Fab heavy chain polypeptide of A₁ and L₂ is bound to the scFv heavy chain polypeptide of A₂.

Embodiment 111 comprises a polypeptide or polypeptide complex of any one of embodiments 1-110, wherein the polypeptide or polypeptide complex has weaker binding affinity for the tumor cell antigen as compared to the binding affinity for the tumor cell antigen of a polypeptide or polypeptide complex that does not have P₁ or L₁.

Embodiment 112 comprises a polypeptide or polypeptide complex of any one of embodiments 1-111, wherein the polypeptide or polypeptide complex has weaker binding affinity for the tumor cell antigen that is at least 10× higher than the binding affinity for the tumor cell antigen of a form of the polypeptide or polypeptide complex that does not have P₁ or L₁.

Embodiment 113 comprises a polypeptide or polypeptide complex of any one of embodiments 1-112, wherein the polypeptide or polypeptide complex has weaker binding affinity for the tumor cell antigen that is at least 100× higher than the binding affinity for the tumor cell antigen of a form of the polypeptide or polypeptide complex that does not have P₁ or L₁.

Embodiment 114 comprises a polypeptide or polypeptide complex of any one of embodiments 1-113, wherein the polypeptide or polypeptide complex has weaker binding affinity for the tumor cell antigen as compared to the binding affinity for the tumor cell antigen of the polypeptide or polypeptide complex in which L₁ has been cleaved by the tumor specific protease.

Embodiment 115 comprises a polypeptide or polypeptide complex of any one of embodiments 1-114, wherein the polypeptide or polypeptide complex has weaker binding affinity for the tumor cell antigen that is at least 10× higher than the binding affinity for the tumor cell antigen of the polypeptide or polypeptide complex in which L₁ has been cleaved by the tumor specific protease.

Embodiment 116 comprises a polypeptide or polypeptide complex of any one of embodiments 1-115, wherein the polypeptide or polypeptide complex has weaker binding affinity for the tumor cell antigen that is at least 100× higher than the binding affinity for the tumor cell antigen of the polypeptide or polypeptide complex in which L₁ has been cleaved by the tumor specific protease.

Embodiment 117 comprises a polypeptide or polypeptide complex of any one of embodiments 1-116, wherein P₁ impairs binding of A₁ to the first target antigen.

Embodiment 118 comprises a polypeptide or polypeptide complex of any one of embodiments 1-117, wherein P₁ is bound to A₁ through ionic interactions, electrostatic interactions, hydrophobic interactions, Pi-stacking interactions, and H-bonding interactions, or a combination thereof.

Embodiment 119 comprises a polypeptide or polypeptide complex of any one of embodiments 1-118, wherein P₁ has less than 70% sequence identity to the first target antigen.

Embodiment 120 comprises a polypeptide or polypeptide complex of any one of embodiments 1-119, wherein P₂ impairs binding of A₂ to the second target antigen.

Embodiment 121 comprises a polypeptide or polypeptide complex of any one of embodiments 1-120, wherein P₂ is bound to A₂ through ionic interactions, electrostatic interactions, hydrophobic interactions, Pi-stacking interactions, and H-bonding interactions, or a combination thereof.

Embodiment 122 comprises a polypeptide or polypeptide complex of any one of embodiments 1-121, wherein P₂ is bound to A₂ at or near an antigen binding site.

Embodiment 123 comprises a polypeptide or polypeptide complex of any one of embodiments 1-122, wherein P₂ has less than 70% sequence identity to the second target antigen.

Embodiment 124 comprises a polypeptide or polypeptide complex of any one of embodiments 1-123, wherein P₁ or P₂ comprises a peptide sequence of at least 10 amino acids in length.

Embodiment 125 comprises a polypeptide or polypeptide complex of any one of embodiments 1-124, wherein P₁ or P₂ comprises a peptide sequence of at least 10 amino acids in length and no more than 20 amino acids in length.

Embodiment 126 comprises a polypeptide or polypeptide complex of any one of embodiments 1-125, wherein P₁ or P₂ comprises a peptide sequence of at least 16 amino acids in length.

Embodiment 127 comprises a polypeptide or polypeptide complex of any one of embodiments 1-126, wherein P₁ or P₂ comprises a peptide sequence of no more than 40 amino acids in length.

Embodiment 128 comprises a polypeptide or polypeptide complex of any one of embodiments 1-127, wherein P₁ or P₂ comprises at least two cysteine amino acid residues.

Embodiment 129 comprises a polypeptide or polypeptide complex of any one of embodiments 1-128, wherein P₁ or P₂ comprises a cyclic peptide or a linear peptide.

Embodiment 130 comprises a polypeptide or polypeptide complex of any one of embodiments 1-129, wherein P₁ or P₂ comprises a cyclic peptide.

Embodiment 131 comprises a polypeptide or polypeptide complex of any one of embodiments 1-130, wherein P₁ or P₂ comprises a linear peptide.

Embodiment 132 comprises a polypeptide or polypeptide complex of any one of embodiments 1-131, wherein P₁ comprises at least two cysteine amino acid residues.

Embodiment 133 comprises a polypeptide or polypeptide complex of any one of embodiments 1-132, wherein the tumor cell antigen comprises EGFR, and the P₁ or P₂ comprises an amino acid sequence selected from the group consisting of GGDWCRSLMSYTDLCP (SEQ ID NO: 1), GGTSCADAHLIAPSCS (SEQ ID NO: 2), GGNCQWDRVEHTYACS (SEQ ID NO: 3), GGWVSCHDGSHMTCFH (SEQ ID NO: 4), GGMNCLNRLWVEYCLV (SEQ ID NO: 5), GGYCGQDNTWVREGCF (SEQ ID NO: 6) and QGQSGQLSCEGWAMNREQCRA (SEQ ID NO: 7).

Embodiment 134 comprises a polypeptide or polypeptide complex of any one of embodiments 1-133, wherein the tumor cell antigen comprises HER2, and the P₁ or P₂ comprises an amino acid sequence selected from the group consisting of GGPLCSDLDHITRLCD (SEQ ID NO: 8), GGIDCASLDHYTESCY (SEQ ID NO: 9), GGNPVCTLGDPYECSH (SEQ ID NO: 10), GGTFCQLNADPYECQS (SEQ ID NO: 11), GGGYCELIGDYVVCSP (SEQ ID NO: 12), GGLCDRWGWIDAPYCH (SEQ ID NO: 13), GGTGCTEGHWHWGTCS (SEQ ID NO: 14), GGNICMDYSWRSGCAV (SEQ ID NO: 15), GGHSCTFGDWSLGTCA (SEQ ID NO: 16), and GGFICTLGNWWDGSCE (SEQ ID NO: 17).

Embodiment 135 comprises a polypeptide or polypeptide complex of any one of embodiments 1-134, wherein the effector cell antigen comprises CD3, and the P₁ or P₂ comprises an amino acid sequence selected from the group consisting of QGQSGQGYLWGCEWNCGGITT (SEQ ID NO: 18), GGDSVCADPEVPICEI (SEQ ID NO: 19), GGMSDCGDPGVEICTH (SEQ ID NO: 20), GGIQCHDPDLPSPCYI (SEQ ID NO: 21), GGEWCLFDPDVPTCQD (SEQ ID NO: 22), GGLGCNDIDPGEQCIV (SEQ ID NO: 23), GGLECFDPEIPEAFCI (SEQ ID NO: 24), GGQGCGTIADPEPHCW (SEQ ID NO: 25), GGNCHDPDIPAYVLCS (SEQ ID NO: 26), GGLCPINDWEPQDICW (SEQ ID NO: 27), and GGLCMIGDWLPGDVCL (SEQ ID NO: 28).

Embodiment 136 comprises a polypeptide or polypeptide complex of any one of embodiments 1-135, wherein L₁ is bound to N-terminus of A₁.

Embodiment 137 comprises a polypeptide or polypeptide complex of any one of embodiments 1-136, wherein L₁ is bound to C-terminus of A₁.

Embodiment 138 comprises a polypeptide or polypeptide complex of any one of embodiments 1-137, wherein L₂ is bound to N-terminus of A₂.

Embodiment 139 comprises a polypeptide or polypeptide complex of any one of embodiments 1-138, wherein L₂ is bound to C-terminus of A₂.

Embodiment 140 comprises a polypeptide or polypeptide complex of any one of embodiments 1-139, wherein L₁ or L₂ is a peptide sequence having at least 5 to no more than 50 amino acids.

Embodiment 141 comprises a polypeptide or polypeptide complex of any one of embodiments 1-140, wherein L₁ or L₂ is a peptide sequence having at least 10 to no more than 30 amino acids.

Embodiment 142 comprises a polypeptide or polypeptide complex of any one of embodiments 1-141, wherein L₁ or L₂ is a peptide sequence having at least 10 amino acids.

Embodiment 143 comprises a polypeptide or polypeptide complex of any one of embodiments 1-142, wherein L₁ or L₂ is a peptide sequence having at least 18 amino acids.

Embodiment 144 comprises a polypeptide or polypeptide complex of any one of embodiments 1-143, wherein L₁ or L₂ is a peptide sequence having at least 26 amino acids.

Embodiment 145 comprises a polypeptide or polypeptide complex of any one of embodiments 1-144, wherein L₁ or L₂ has a formula comprising (G₂S)ₙ, wherein n is an integer from 1 to 3 (SEQ ID NO: 29).

Embodiment 146 comprises a polypeptide or polypeptide complex of any one of embodiments 1-145, wherein L₁ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1.

Embodiment 147 comprises a polypeptide or polypeptide complex of any one of embodiments 1-146, wherein P₁ becomes unbound from A₁ when L₁ is cleaved by the tumor specific protease thereby exposing A₁ to the first target antigen.

Embodiment 148 comprises a polypeptide or polypeptide complex of any one of embodiments 1-147, wherein P₂ becomes unbound from A₂ when L₂ is cleaved by the tumor specific protease thereby exposing A₂ to the second target antigen.

Embodiment 149 comprises a polypeptide or polypeptide complex of any one of embodiments 1-148, wherein the tumor specific protease is selected from the group consisting of metalloprotease, serine protease, cysteine protease, threonine protease, and aspartic protease.

Embodiment 150 comprises a polypeptide or polypeptide complex of any one of embodiments 1-149, wherein L₁ or L₂ comprises a urokinase cleavable amino acid sequence, a matriptase cleavable amino acid sequence, matrix metalloprotease cleavable amino acid sequence, or a legumain cleavable amino acid sequence.

Embodiment 151 comprises a polypeptide or polypeptide complex of any one of embodiments 1-150, wherein L₁ or L₂ comprises an amino acid sequence selected from the group consisting of

(SEQ ID NO: 34)
GGGGSLSGRSDNHGSSGT,
(SEQ ID NO: 35)
GGGGSSGGSGGSGLSGRSDNHGSSGT,
(SEQ ID NO: 36)
ASGRSDNH,
(SEQ ID NO: 37)
LAGRSDNH,
(SEQ ID NO: 38)
ISSGLASGRSDNH,
(SEQ ID NO: 39)
ISSGLLAGRSDNH,
(SEQ ID NO: 40)
LSGRSDNH,
(SEQ ID NO: 41)
ISSGLLSGRSDNP,
(SEQ ID NO: 42)
ISSGLLSGRSDNH,
(SEQ ID NO: 43)
LSGRSDNHSPLGLAGS,
(SEQ ID NO: 44)
SPLGLAGSLSGRSDNH,
(SEQ ID NO: 45)
SPLGLSGRSDNH,
(SEQ ID NO: 46)
LAGRSDNHSPLGLAGS,
(SEQ ID NO: 47)
LSGRSDNHVPLSLKMG,
(SEQ ID NO: 48)
LSGRSDNHVPLSLSMG,
(SEQ ID NO: 49)
GSSGGSGGSGGSGISSGLLSGRSDNHGSSGT,
and
(SEQ ID NO: 50)
GSSGGSGGSGGISSGLLSGRSDNHGGGS.

Embodiment 152 comprises a polypeptide or polypeptide complex of any one of embodiments 1-151, wherein L₁ or L₂ comprises an amino acid sequence ASGRSDNH (SEQ ID NO: 36), LAGRSDNH (SEQ ID NO: 37), ISSGLASGRSDNH (SEQ ID NO: 38), and ISSGLLAGRSDNH (SEQ ID NO: 39).

Embodiment 153 comprises a polypeptide or polypeptide complex of any one of embodiments 1-152, wherein H₁ comprises a polymer.

Embodiment 154 comprises a polypeptide or polypeptide complex of any one of embodiments 1-153, wherein the polymer is polyethylene glycol (PEG).

Embodiment 155 comprises a polypeptide or polypeptide complex of any one of embodiments 1-154, wherein H₁ comprises albumin.

Embodiment 156 comprises a polypeptide or polypeptide complex of any one of embodiments 1-155, wherein H₁ comprises an Fc domain.

Embodiment 157 comprises a polypeptide or polypeptide complex of any one of embodiments 1-156, wherein the albumin is serum albumin.

Embodiment 158 comprises a polypeptide or polypeptide complex of any one of embodiments 1-157, wherein the albumin is human serum albumin.

Embodiment 159 comprises a polypeptide or polypeptide complex of any one of embodiments 1-158, wherein H₁ comprises a polypeptide, a ligand, or a small molecule.

Embodiment 160 comprises a polypeptide or polypeptide complex of any one of embodiments 1-159, wherein the polypeptide, the ligand or the small molecule binds serum protein or a fragment thereof, a circulating immunoglobulin or a fragment thereof, or CD35/CR1.

Embodiment 161 comprises a polypeptide or polypeptide complex of any one of embodiments 1-160, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 162 comprises a polypeptide or polypeptide complex of any one of embodiments 1-161, wherein the circulating immunoglobulin molecule comprises IgG1, IgG2, IgG3, IgG4, slgA, IgM or IgD.

Embodiment 163 comprises a polypeptide or polypeptide complex of any one of embodiments 1-162, wherein the serum protein is albumin.

Embodiment 164 comprises a polypeptide or polypeptide complex of any one of embodiments 1-163, wherein the polypeptide is an antibody.

Embodiment 165 comprises a polypeptide or polypeptide complex of any one of embodiments 1-164, wherein the antibody comprises a single domain antibody, a single chain variable fragment, or a Fab.

Embodiment 166 comprises a polypeptide or polypeptide complex of any one of embodiments 1-165, wherein the single domain antibody comprises a single domain antibody that binds to albumin. wherein the single domain antibody is a human or humanized antibody.

Embodiment 166 comprises a polypeptide or polypeptide complex of any one of embodiments 1-165, wherein the single domain antibody is 645gH1gL1.

Embodiment 168 comprises a polypeptide or polypeptide complex of any one of embodiments 1-167, wherein the single domain antibody is 645dsgH5gL4.

Embodiment 169 comprises a polypeptide or polypeptide complex of any one of embodiments 1-168, wherein the single domain antibody is 23-13-A01 -sc02.

Embodiment 170 comprises a polypeptide or polypeptide complex of any one of embodiments 1-169, wherein the single domain antibody is A10m3 or a fragment thereof.

Embodiment 171 comprises a polypeptide or polypeptide complex of any one of embodiments 1-170, wherein the single domain antibody is DOM7r-31.

Embodiment 172 comprises a polypeptide or polypeptide complex of any one of embodiments 1-171, wherein the single domain antibody is DOM7h-11-15.

Embodiment 173 comprises a polypeptide or polypeptide complex of any one of embodiments 1-172, wherein the single domain antibody is Alb-1, Alb-8, or Alb-23.

Embodiment 174 comprises a polypeptide or polypeptide complex of any one of embodiments 1-173, wherein the single domain antibody is 10G or 10GE.

Embodiment 175 comprises a polypeptide or polypeptide complex of any one of embodiments 1-174, wherein the single domain antibody is 10G, and the single domain antibody comprises an amino acid sequence

(SEQ ID NO: 52)
EVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTI
GGSLSVSSQGTLVTVSS.

Embodiment 176 comprises a polypeptide or polypeptide complex of any one of embodiments 1-175, wherein the single domain antibody is SA21.

Embodiment 177 comprises a polypeptide or polypeptide complex of any one of embodiments 1-176, wherein the polypeptide or polypeptide complex comprises a modified amino acid, a non-natural amino acid, a modified non-natural amino acid, or a combination thereof.

Embodiment 178 comprises a polypeptide or polypeptide complex of any one of embodiments 1-177, wherein the modified amino acid or modified non-natural amino acid comprises a post-translational modification.

Embodiment 179 comprises a polypeptide or polypeptide complex of any one of embodiments 1-178, wherein H₁ comprises a linking moiety (L₃) that connects H1 to P₁.

Embodiment 180 comprises a polypeptide or polypeptide complex of any one of embodiments 1-179, wherein L₃ is a peptide sequence having at least 5 to no more than 50 amino acids.

Embodiment 181 comprises a polypeptide or polypeptide complex of any one of embodiments 1-180, wherein L₃ is a peptide sequence having at least 10 to no more than 30 amino acids.

Embodiment 182 comprises a polypeptide or polypeptide complex of any one of embodiments 1-181, wherein L₃ is a peptide sequence having at least 10 amino acids.

Embodiment 183 comprises a polypeptide or polypeptide complex of any one of embodiments 1-182, wherein L₃ is a peptide sequence having at least 18 amino acids.

Embodiment 184 comprises a polypeptide or polypeptide complex of any one of embodiments 1-183, wherein L₃ is a peptide sequence having at least 26 amino acids.

Embodiment 185 comprises a polypeptide or polypeptide complex of any one of embodiments 1-184, wherein L₃ has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1.

Embodiment 186 comprises a polypeptide or polypeptide complex of any one of embodiments 1-185, wherein L₃ comprises an amino acid sequence according to GGGGSGGGS (SEQ ID NO: 51).

Embodiment 187 comprises a pharmaceutical composition comprising: the polypeptide or polypeptide complex of any one of embodiments 1-186; and a pharmaceutically acceptable excipient.

Embodiment 188 comprises an isolated recombinant nucleic acid molecule encoding the polypeptide or polypeptide complex of any one of embodiments 1-187.

Embodiment 189 comprises a polypeptide or polypeptide complex according to Formula II:

L₁ₐ-P₁ₐ-H₁ₐ (Formula II)

wherein: L₁ₐ comprises a tumor specific protease-cleaved linking moiety that when uncleaved connects P₁ₐ to an antigen recognizing molecule that binds to a target antigen and; P₁ₐ comprises a peptide that binds to the antigen recognizing molecule when L₁ₐ is uncleaved; and H₁ₐ comprises a half-life extending molecule.

Embodiment 190 comprises a polypeptide or polypeptide complex of any one of embodiments 1-189, wherein P₁ₐ when L₁ is uncleaved impairs binding of the antigen recognizing molecule to the target antigen.

Embodiment 191 comprises a polypeptide or polypeptide complex of any one of embodiments 1-190, wherein the antigen recognizing molecule comprises an antibody or antibody fragment.

Embodiment 192 comprises a polypeptide or polypeptide complex of any one of embodiments 1-191, wherein the target antigen is an anti-CD3 effector cell antigen.

Embodiment 193 comprises a polypeptide or polypeptide complex of any one of embodiments 1-192, wherein the target antigen is a tumor cell antigen.

Embodiment 194 comprises a polypeptide or polypeptide complex of any one of embodiments 1-193, wherein the tumor cell antigen is EGFR, HER2, mesothelin, or CEACAM5.

Embodiment 195 comprises a polypeptide or polypeptide complex of any one of embodiments 1-194, wherein P₁ₐ has less than 70% sequence identity to the target antigen.

Embodiment 196 comprises a polypeptide or polypeptide complex of any one of embodiments 1-195, wherein P₁ₐ comprises a peptide sequence of at least 10 amino acids in length.

Embodiment 197 comprises a polypeptide or polypeptide complex of any one of embodiments 1-196, wherein P₁ₐ comprises a peptide sequence of at least 10 amino acids in length and no more than 20 amino acids in length.

Embodiment 198 comprises a polypeptide or polypeptide complex of any one of embodiments 1-197, wherein P₁ₐ comprises a peptide sequence of at least 16 amino acids in length.

Embodiment 199 comprises a polypeptide or polypeptide complex of any one of embodiments 1-198, wherein P₁ₐ comprises a peptide sequence of no more than 40 amino acids in length.

Embodiment 200 comprises a polypeptide or polypeptide complex of any one of embodiments 1-199, wherein P₁ₐ comprises at least two cysteine amino acid residues.

Embodiment 201 comprises a polypeptide or polypeptide complex of any one of embodiments 1-200, wherein P₁ₐ comprises a cyclic peptide or a linear peptide.

Embodiment 202 comprises a polypeptide or polypeptide complex of any one of embodiments 1-201, wherein P₁ₐ comprises a cyclic peptide.

Embodiment 203 comprises a polypeptide or polypeptide complex of any one of embodiments 1-202, wherein P₁ₐ comprises a linear peptide.

Embodiment 204 comprises a polypeptide or polypeptide complex of any one of embodiments 1-203, wherein the target antigen comprises EGFR, and the P₁ₐ comprises an amino acid sequence selected from the group consisting of GGDWCRSLMSYTDLCP (SEQ ID NO: 1), GGTSCADAHLIAPSCS (SEQ ID NO: 2), GGNCQWDRVEHTYACS (SEQ ID NO: 3), GGWVSCHDGSHMTCFH (SEQ ID NO: 4), GGMNCLNRLWVEYCLV (SEQ ID NO: 5), GGYCGQDNTWVREGCF (SEQ ID NO: 6) and QGQSGQLSCEGWAMNREQCRA (SEQ ID NO: 7).

Embodiment 205 comprises a polypeptide or polypeptide complex of any one of embodiments 1-204, wherein the target comprises HER2, and the P₁ₐ comprises an amino acid sequence selected from the group consisting of GGPLCSDLDHITRLCD (SEQ ID NO: 8), GGIDCASLDHYTESCY (SEQ ID NO: 9), GGNPVCTLGDPYECSH (SEQ ID NO: 10), GGTFCQLNADPYECQS (SEQ ID NO: 11), GGGYCELIGDYVVCSP (SEQ ID NO: 12), GGLCDRWGWIDAPYCH (SEQ ID NO: 13), GGTGCTEGHWHWGTCS (SEQ ID NO: 14), GGNICMDYSWRSGCAV (SEQ ID NO: 15), GGHSCTFGDWSLGTCA (SEQ ID NO: 16), and GGFICTLGNWWDGSCE (SEQ ID NO: 17).

Embodiment 206 comprises a polypeptide or polypeptide complex of any one of embodiments 1-205, wherein the target comprises CD3, and the P₁ₐ comprises an amino acid sequence selected from the group consisting of QGQSGQGYLWGCEWNCGGITT (SEQ ID NO: 18), GGDSVCADPEVPICEI (SEQ ID NO: 19), GGMSDCGDPGVEICTH (SEQ ID NO: 20), GGIQCHDPDLPSPCYI (SEQ ID NO: 21), GGEWCLFDPDVPTCQD (SEQ ID NO: 22), GGLGCNDIDPGEQCIV (SEQ ID NO: 23), GGLECFDPEIPEAFCI (SEQ ID NO: 24), GGQGCGTIADPEPHCW (SEQ ID NO: 25), GGNCHDPDIPAYVLCS (SEQ ID NO: 26), GGLCPINDWEPQDICW (SEQ ID NO: 27), and GGLCMIGDWLPGDVCL (SEQ ID NO: 28).

Embodiment 207 comprises a polypeptide or polypeptide complex of any one of embodiments 1-206, wherein H₁ₐ comprises a polymer.

Embodiment 208 comprises a polypeptide or polypeptide complex of any one of embodiments 1-207, wherein the polymer is polyethylene glycol (PEG).

Embodiment 209 comprises a polypeptide or polypeptide complex of any one of embodiments 1-208, wherein H₁ₐ comprises albumin.

Embodiment 210 comprises a polypeptide or polypeptide complex of any one of embodiments 1-209, wherein H₁ₐ comprises an Fc domain.

Embodiment 211 comprises a polypeptide or polypeptide complex of any one of embodiments 1-210, wherein the albumin is serum albumin.

Embodiment 212 comprises a polypeptide or polypeptide complex of any one of embodiments 1-211, wherein the albumin is human serum albumin.

Embodiment 213 comprises a polypeptide or polypeptide complex of any one of embodiments 1-212, wherein H₁ₐ comprises a polypeptide, a ligand, or a small molecule.

Embodiment 214 comprises a polypeptide or polypeptide complex of any one of embodiments 1-213, wherein the polypeptide, the ligand or the small molecule binds a serum protein or a fragment thereof, a circulating immunoglobulin or a fragment thereof, or CD35/CR1.

Embodiment 215 comprises a polypeptide or polypeptide complex of any one of embodiments 1-214, wherein the serum protein comprises a thyroxine-binding protein, a transthyretin, a 1-acid glycoprotein, a transferrin, transferrin receptor or a transferrin-binding portion thereof, a fibrinogen, or an albumin.

Embodiment 216 comprises a polypeptide or polypeptide complex of any one of embodiments 1-215, wherein the circulating immunoglobulin molecule comprises IgG1, IgG2, IgG3, IgG4, slgA, IgM or IgD.

Embodiment 217 comprises a polypeptide or polypeptide complex of any one of embodiments 1-216, wherein the serum protein is albumin.

Embodiment 218 comprises a polypeptide or polypeptide complex of any one of embodiments 1-217, wherein the polypeptide is an antibody.

Embodiment 219 comprises a polypeptide or polypeptide complex of any one of embodiments 1-218, wherein the antibody comprises a single domain antibody, a single chain variable fragment or a Fab.

Embodiment 220 comprises a polypeptide or polypeptide complex of any one of embodiments 1-219, wherein the antibody comprises a single domain antibody that binds to albumin.

Embodiment 221 comprises a polypeptide or polypeptide complex of any one of embodiments 1-220, wherein the antibody is a human or humanized antibody.

Embodiment 222 comprises a polypeptide or polypeptide complex of any one of embodiments 1-221, wherein the single domain antibody is 645gH1gL1.

Embodiment 223 comprises a polypeptide or polypeptide complex of any one of embodiments 1-222, wherein the single domain antibody is 645dsgH5gL4.

Embodiment 224 comprises a polypeptide or polypeptide complex of any one of embodiments 1-223, wherein the single domain antibody is 23-13-A01 -sc02.

Embodiment 225 comprises a polypeptide or polypeptide complex of any one of embodiments 1-224, wherein the single domain antibody is A10m3 or a fragment thereof.

Embodiment 226 comprises a polypeptide or polypeptide complex of any one of embodiments 1-225, wherein the single domain antibody is DOM7r-31.

Embodiment 227 comprises a polypeptide or polypeptide complex of any one of embodiments 1-226, wherein the single domain antibody is DOM7h-11-15.

Embodiment 228 comprises a polypeptide or polypeptide complex of any one of embodiments 1-227, wherein the single domain antibody is Alb-1, Alb-8, or Alb-23.

Embodiment 229 comprises a polypeptide or polypeptide complex of any one of embodiments 1-228, wherein the single domain antibody is 10G or 10GE.

Embodiment 230 comprises a polypeptide or polypeptide complex of any one of embodiments 1-229, wherein the single domain antibody is 10G, and the single domain antibody comprises an amino acid sequence

(SEQ ID NO: 52)
EVQLVESGGGLVQPGNSLRLSCAASGFTFSKFGMSWVRQAPGKGLEWVS
SISGSGRDTLYADSVKGRFTISRDNAKTTLYLQMNSLRPEDTAVYYCTI
GGSLSVSSQGTLVTVSS.

Embodiment 231 comprises a polypeptide or polypeptide complex of any one of embodiments 1-230, wherein the single domain antibody is SA21.

Embodiment 232 comprises a polypeptide or polypeptide complex of any one of embodiments 1-231, wherein H₁ₐ comprises a linking moiety (L₃ₐ) that connects H₁ₐ to P₁ₐ.

Embodiment 233 comprises a polypeptide or polypeptide complex of any one of embodiments 1-232, wherein L₃a is a peptide sequence having at least 5 to no more than 50 amino acids.

Embodiment 234 comprises a polypeptide or polypeptide complex of any one of embodiments 1-233, wherein L₃a is a peptide sequence having at least 10 to no more than 30 amino acids.

Embodiment 235 comprises a polypeptide or polypeptide complex of any one of embodiments 1-234, wherein L₃a is a peptide sequence having at least 10 amino acids.

Embodiment 236 comprises a polypeptide or polypeptide complex of any one of embodiments 1-235, wherein L₃a is a peptide sequence having at least 18 amino acids.

Embodiment 237 comprises a polypeptide or polypeptide complex of any one of embodiments 1-236, wherein L₃a is a peptide sequence having at least 26 amino acids.

Embodiment 238 comprises a polypeptide or polypeptide complex of any one of embodiments 1-237, wherein L₃a has a formula selected from the group consisting of (G₂S)ₙ, (GS)ₙ, (GSGGS)ₙ (SEQ ID NO: 30), (GGGS)ₙ (SEQ ID NO: 31), (GGGGS)ₙ (SEQ ID NO: 32), and (GSSGGS)ₙ (SEQ ID NO: 33), wherein n is an integer of at least 1.

Embodiment 239 comprises a polypeptide or polypeptide complex of any one of embodiments 1-238, wherein L₃a comprises an amino acid sequence GGGGSGGGS (SEQ ID NO: 51).

Embodiment 240 comprises a polypeptide or polypeptide complex a structural arrangement according to the configuration shown in FIG. 50A, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the heavy chain variable domain of the scFv, and wherein the Fab is linked to P₂ and L₂, wherein P₂ comprises a peptide that impairs binding of the Fab to the tumor cell antigen; and L₂ comprises a linking moiety that connects the Fab light chain polypeptide to P₂ and is a substrate for a tumor specific protease.

Embodiment 241 comprises a polypeptide or polypeptide complex comprising a structural arrangement according to the configuration shown in FIG. 50Q, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the heavy chain variable domain of the scFv.

Embodiment 242 comprises a polypeptide or polypeptide complex comprising a structural arrangement according to the configuration shown in FIG. 50R, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the light chain variable domain of the scFv with a linking moiety that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Embodiment 243 comprises a polypeptide or polypeptide complex comprising a structural arrangement according to the configuration in FIG. 50S, wherein the polypeptide or polypeptide complex comprises a single chain variable fragment (scFv) comprising a light chain variable domain and a heavy chain variable domain, wherein the scFv is further linked to a peptide that impairs binding of the scFv to an effector cell antigen and the peptide is linked to a N-terminus of the light chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the heavy chain variable domain of the scFv.

Embodiment 244 comprises a polypeptide or polypeptide complex comprising a structural arrangement according to the configuration shown in FIG. 50T, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab heavy chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab light chain polypeptide to P₂ and is a substrate for a tumor specific protease.

Embodiment 245 comprises a polypeptide or polypeptide complex comprising a structural arrangement according to the configuration shown in FIG. 50U, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide chain and a Fab heavy chain polypeptide chain, and wherein the Fab heavy chain polypeptide chain is linked to a C terminus of the light chain variable domain of the scFv.

Embodiment 246 comprises a polypeptide or polypeptide complex comprising a structural arrangement according to the configuration shown in FIG. 50V, wherein the polypeptide or polypeptide complex comprises a single chain variable fragment (scFv) comprising a light chain variable domain and a heavy chain variable domain, wherein the scFv is linked to a peptide (P₁) that impairs binding of the scFv to an effector cell antigen and P₁ is linked to a N-terminus of the heavy chain variable domain of the scFv with a linking moiety (L₁) that is a substrate for a tumor specific protease, and P₁ is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv, and wherein the Fab is linked to P₂ and L₂, wherein P₂ comprises a peptide that impairs binding to the tumor cell antigen; and L₂ comprises a linking moiety that connects the Fab heavy chain polypeptide to P₂ and is a substrate for a tumor specific protease.

Embodiment 247 comprises a polypeptide or polypeptide complex comprising a structural arrangement according to the configuration shown in FIG. 50W, wherein the polypeptide or polypeptide complex comprises a single chain variable fragment (scFv) comprising a light chain variable domain and a heavy chain variable domain, wherein the scFv is linked to a peptide that impairs binding of the scFv to an effector cell antigen and the peptide is linked to a N-terminus of the heavy chain variable domain of the scFv with a linking moiety that is a substrate for a tumor specific protease, and the peptide is further linked to a half-life extending molecule; and a Fab that binds to a tumor cell antigen, wherein the Fab comprises a Fab light chain polypeptide and a Fab heavy chain polypeptide, wherein the Fab light chain polypeptide is linked to a C terminus of the light chain variable domain of the scFv.

Embodiment 248 comprises a polypeptide or polypeptide complex comprising a structural arrangement according to the configuration shown in FIG. 50I, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and the P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Embodiment 249 comprises a polypeptide or polypeptide complex comprising a structural arrangement according to the configuration shown in FIG. 50J, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab heavy chain polypeptide.

Embodiment 250 comprises a polypeptide or polypeptide complex comprising a structural arrangement according to the configuration shown in FIG. 50K, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide, wherein the scFv further is linked to P₂ and L₂, wherein P₂ comprises a peptide that impairs binding of the scFv to the effector cell antigen, and L₂ comprises a linking moiety that connects the light chain variable domain of the scFv to P₂ and is a substrate for a tumor specific protease.

Embodiment 251 comprises a polypeptide or polypeptide complex comprising a structural arrangement according to the configuration shown in FIG. 50L, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the heavy chain variable domain of the scFv is linked to an N terminus of the Fab light chain polypeptide.

Embodiment 252 comprises a polypeptide or polypeptide complex comprising a structural arrangement according to the configuration shown in FIG. 50M, wherein the polypeptide or polypeptide complex comprises a Fab that binds to a tumor cell antigen, the Fab comprising a Fab light chain polypeptide and a Fab heavy chain polypeptide, wherein the Fab is linked to a peptide (P₁) that impairs binding of the Fab to the tumor cell antigen and P₁ is linked to a N terminus of the Fab light chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Embodiment 253 comprises a polypeptide or polypeptide complex comprising a structural arrangement according to the configuration shown in FIG. 50N, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab light chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab heavy chain polypeptide.

Embodiment 254 comprises a polypeptide or polypeptide complex comprising a structural arrangement according to the configuration shown in FIG. 50O, wherein the polypeptide or polypeptide complex comprises a Fab that binds to a tumor cell antigen, the Fab comprising a Fab light chain polypeptide and a Fab heavy chain polypeptide, wherein the Fab is linked to a (P₁) that impairs binding of the Fab to the tumor cell antigen and P₁ is linked to a N terminus of the Fab heavy chain polypeptide with a linking moiety (L₁) that is a substrate for a tumor specific protease, and P₁ is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide, wherein the scFv is linked to P₂ and L₂, wherein P₂ comprises a peptide that impairs binding of the scFv to the effector cell antigen, and L₂ comprises a linking moiety that connects the heavy chain variable domain of the scFv to P₂ and is a substrate for a tumor specific protease.

Embodiment 255 comprises a polypeptide or polypeptide complex comprising a structural arrangement according to the configuration shown in FIG. 50P, wherein the polypeptide or polypeptide complex comprises a Fab that binds to a tumor cell antigen, the Fab comprising a Fab light chain polypeptide and a Fab heavy chain polypeptide, wherein the Fab is linked to a peptide that impairs binding of the Fab to the tumor cell antigen and the peptide is linked to a N terminus of the Fab heavy chain polypeptide with a linking moiety that is a substrate for a tumor specific protease, and the peptide is further linked to a half-life extending molecule; and a single chain variable fragment (scFv) that binds to an effector cell antigen, the scFv comprising a light chain variable domain and a heavy chain variable domain, wherein the light chain variable domain of the scFv is linked to an N terminus of the Fab light chain polypeptide.

### EXAMPLES

### Example 1

### Preparation and Evaluation of Biotinylated Antibodies

Cetuximab (Ab-1, Ab-3) and Trastuzumab (Ab-6, Ab-7) were sourced from SelleckChem (A2000 and A2007, respectively) while SP34 (Ab-9, Ab-10) was sourced from BD Biosciences (551916). Identification and confirmation of phagemid-displayed peptides that bind directly to antibodies required antibodies be biotinylated and loaded onto streptavidin coated beads. Antibodies were chemically biotinylated using EZ-Link Sulfo NHS-LC-LC-Biotin reagent from Thermo Fisher Scientific (A35358) following the manufacturer's instructions. Briefly, antibody was buffer exchanged into PBS and concentrated to 1 mg/mL. EZ-link Sulfo NHS-LC-LC reagent was prepared according to manufacturer's instructions, added in 20× molar excess relative to antibody, and incubated at room temperature for 30 min. Reaction was cooled on ice for 30 min and then dialyzed into cold PBS to remove biotin reagent. The percent of biotinylated antibody was measured by streptavidin bead subtraction (TABLE 2). A sample of naked or biotinylated antibody was incubated with excess streptavidin beads for 1 hour at room temperature. Beads were pelleted with a magnet and the supernatant harvested. The concentration of protein in the supernatant was measured using the Pierce BCA assay and compared to that of the naked antibody control. The percent of bead bound antibody was calculated by mass balance and used to approximate the amount of biotinylated antibody relative to total protein. Antibodies, pre and post biotinylation, were then verified for their ability to bind their cognate antigen.

TABLE 2
Calculated percent biotinylation for each antibody.

| Sample | % biotinylation |
|---|---|
| Ab-1, Ab-3 (Cetuximab) | 66 |
| Ab-6, Ab-7 (Trastuzumab) | 95 |
| Ab-9, Ab-10 (SP34) | 93 |

Kinetic binding of antibodies pre and post biotinylation was measured using a ForteBio Octet RED96 instrument. For evaluation of naked antibodies biotinylated cognate antigen, EGFR-biotin, HER2-biotin, or CD3 biotin was first captured on streptavidin biosensors. Sensors were quenched using excess biocytin and then baselined in buffer. Naked antibodies were titrated in a 2-fold dilution series starting from 50 nM and was associated onto the antigen loaded biosensor. Association signal was monitored in real-time. Biosensors were then transferred to buffer and the dissociation of antibody was measured in real-time. Data was background corrected, fit to a classic 1:1 binding model, and used to calculate kinetic rate constants. Biotinylated antibodies were analyzed to ensure that the biotinylation did not interfere with the antibody ability to recognize its cognate antigen. Biotinylated antibodies were first captured on streptavidin biosensors. Sensors were quenched using excess biocytin and then baselined in buffer. Naked antigens, EGFR, HER2 or CD3, were titrated in a 2-fold dilution series starting from 50 nM and associated onto the antibody loaded biosensor. Association signal was monitored in real-time. Biosensors were then transferred to buffer and the dissociation of antibody was measured in real-time. Data was background corrected, fit to a classic 1:1 binding model, and used to calculate kinetic rate constants

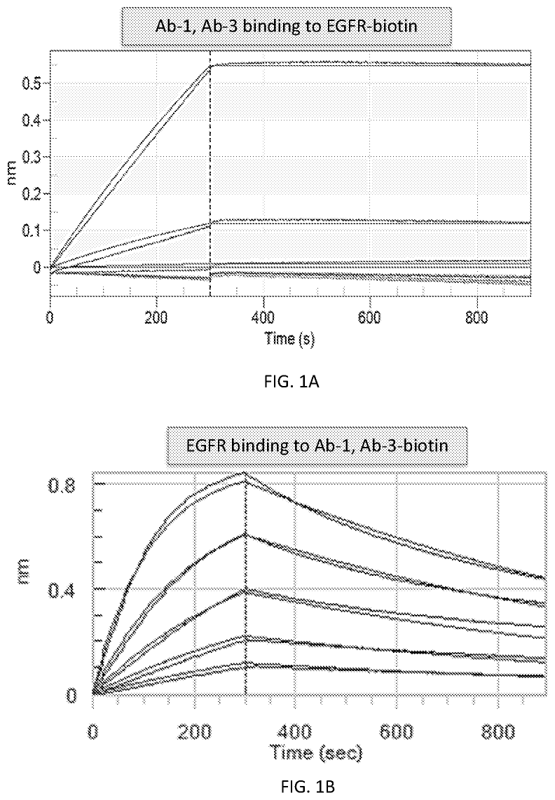

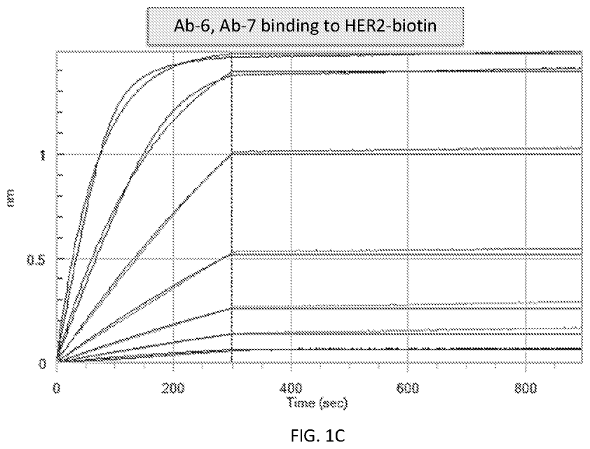

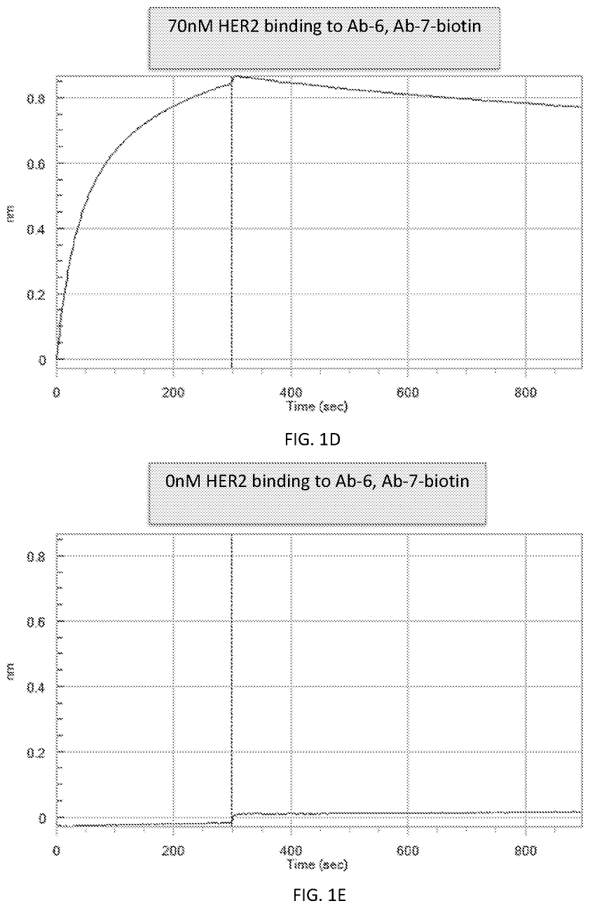

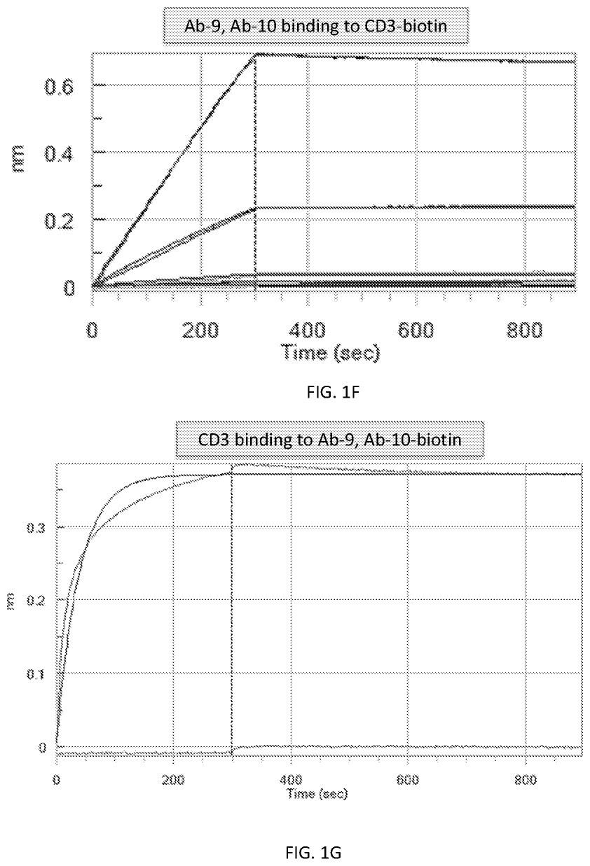

Representative examples of the binding interactions between Cetuximab and EGFR-biotin, Cetuximab-biotin and EGFR, Trastuzumab and HER2-biotin, Trastuzumab-biotin and HER2, SP34 and CD3-biotin, as well as SP34-biotin and CD3 are shown in FIGS. 1A-1G.

### Example 2

### Identification and Confirmation of Phagemid-Displayed Peptides that Bind Directly to the Antibody and Compete for Cognate Antigen Binding Sites

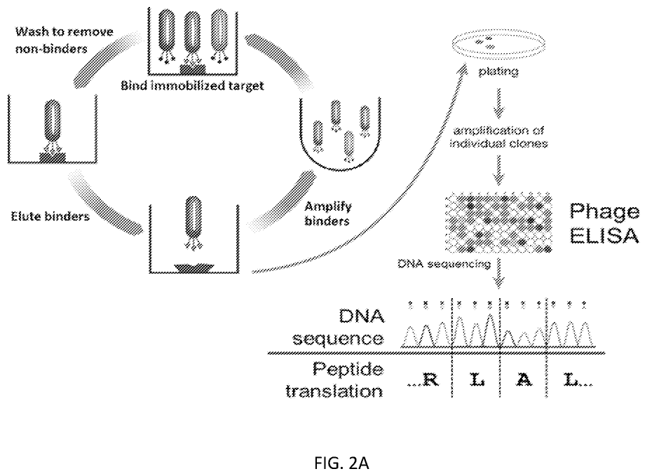

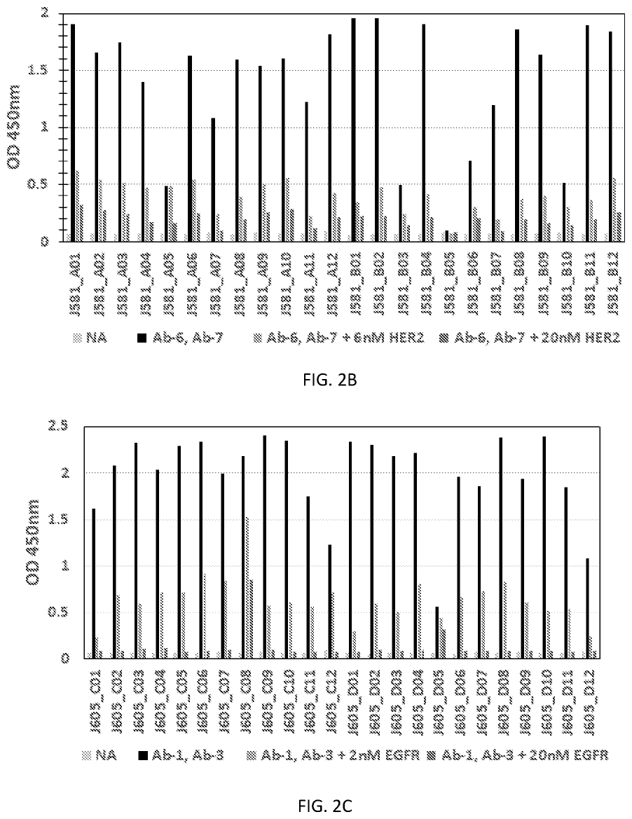

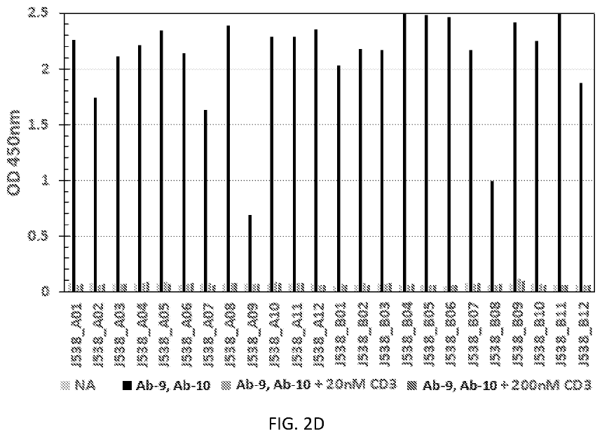

Peptides with the ability to bind to an antibody of interest were identified by biopanning phagemid-display libraries of candidate peptides (FIG. 2A). Libraries were created via the introduction of recombinant expression of peptides fused to the m13 bacteriophage coat protein III (p3) or VIII (p8), resulting in display of the candidate peptides on the surface of the secreted bacteriophage. The candidate peptide libraries had variable amino acid sequences and collectively variable amino acid lengths.

Biopanning of m13 phagemid p3 displayed peptide libraries was performed with biotin conjugated antibodies immobilized on streptavidin coated paramagnetic beads. Antibodies were chemically biotinylated using Sulfo-NHS-LC-LC biotin reagent as described in Example 1. Following binding to the target at pH 7.4 and subsequent washing steps, specifically bound phage were recovered by elution at pH 2.2, or at pH 11.0. Though individual clones could be sequenced or tested after a single round, enrichment of specific binding clones was typically accomplished by 2-4 rounds of successive biopanning and amplification. Following the enrichment of pools, phage biopanning phage pools were infected into TG1 cells and plated out on LB-ampicillin/agar plates for subsequent clonal isolation, DNA sequencing, and characterization (FIG. 2A).

Phagemid Hit Identification ELISA

For hit identification, individual colonies were grown in 96-deep well plates for 2-4 hours and infected with helper phage to produce peptide displayed phagemid following an overnight growth. The next day the deep well plates were centrifuged to separate the soluble phagemid from the E. coli cells. The phagemid containing supernatants were then combined with PBS-Tween 20 (0.05%)+BSA (1%) pH neutral blocking buffer and incubated in previously antibody coated and blocked wells. After binding at 4° C. the plates were washed, and specifically bound phage were detected by anti-m13 HRP conjugated antibodies using standard TMB-based chromogenic ELISA procedures. Daughter plates or individual wells were subjected to standard DNA sequencing for peptide identification.

A representative example of the phagemid binding ELISA is seen in FIG. 2B from a collection of enriched clones isolated after three rounds of biopanning against Trastuzumab (Ab-6, Ab-7). A representative example of the phagemid binding ELISA is seen in FIG. 2C from a collection of enriched clones isolated after three rounds of biopanning against Cetuximab (Ab-1, Ab-3). A representative example of the phagemid binding ELISA is seen in FIG. 2D from a collection of enriched clones isolated after three rounds of biopanning against SP34 (Ab-9, Ab-10).

Phagemid Competition ELISA Assay

Phagemid peptide clones were next tested to determine whether they bound within the cognate antigen binding space of the antibody using a target-based competition assay. We prepared biotin conjugated antibody immobilized and blocked 96-well ELISA plates similar to above. Next we added cognate antigen to the well to block the antigen binding site. After a brief incubation period, phagemid supernatants were next added to the wells. Following an incubation at 4° C. the plates were washed, and specifically bound phage were detected by anti-m13 HRP conjugated antibodies using standard TMB-based chromogenic ELISA procedures. Phagemid clones binding within the antigen binding pocket of the antibody were blocked and identified by a decreased ELISA signal relative to wells lacking antigen pre-incubation.

A representative example of the phagemid competition ELISA is seen in FIG. 2B from a collection of enriched clones isolated after three rounds of biopanning against Trastuzumab (Ab-6, Ab-7). A representative example of the phagemid competition ELISA is seen in FIG. 2B from a collection of enriched clones isolated after three rounds of biopanning against Cetuximab (Ab-1, Ab-3). A representative example of the phagemid competition ELISA is seen in FIG. 2B from a collection of enriched clones isolated after three rounds of biopanning against SP34 (Ab-9, Ab-10).

### Example 3

### Synthetic Peptide Evaluations for Antibody Binding and Inhibition

Peptides expressed on clonal phage that exhibit antibody specific binding and inhibition were chosen for further characterization. Exemplary phagemid peptides that bind to Trastuzumab (Ab-6, Ab-7), Cetuximab (Ab-1, Ab-3), or SP34 (Ab-9, Ab-10) are listed in TABLES 3A, 3B, and 4 and selected for peptide synthesis. Peptides selected for additional evaluation were first chemically synthesized and then evaluated for antibody binding and antigen competition.

TABLE 3A
Example phagemid peptide sequences that bind Ab-6, Ab-7 advanced into solid
phase synthesis. Table discloses SEQ ID NOS 8-17, respectively, in order of appearance.

| | | Phage ELISA | | | | | | | | | | | | | | | | | | | |
|---|---|---|---|---|---|---|---|---|---|---|---|---|---|---|---|---|---|---|---|---|---|
| | | | | 6 nM | 20 nM | | | | | | | | | | | | | | | | |
| | | | Ab-6, | Her2 | Her2 | | | | | | | | | | | | | | | | |
| Phage | Peptide | NAv | Ab-7 | % | % | Peptide amino acid sequence from clonal phage | | | | | | | | | | | | | | | |
| Clone | ID | bkgd | signal | inhib | inhib | 1 | 2 | 3 | 4 | 5 | 6 | 7 | 8 | 9 | 10 | 11 | 12 | 13 | 14 | 15 | 16 |
| J551A03 | Peptide- | 0.080 | 2.066 | 92% | 96% | G | G | P | L | C | S | D | L | D | H | I | T | R | L | C | D |
| | 8 | | | | | | | | | | | | | | | | | | | | |
| J551A10 | Peptide- | 0.084 | 2.004 | 90% | 96% | G | G | I | D | C | A | S | L | D | H | Y | T | E | S | C | Y |
| | 9 | | | | | | | | | | | | | | | | | | | | |
| J549G12 | Peptide- | 0.083 | 1.406 | 94% | 94% | G | G | N | P | V | C | T | L | G | D | P | Y | E | C | S | H |
| | 10 | | | | | | | | | | | | | | | | | | | | |
| J550G07 | Peptide- | 0.074 | 1.571 | 95% | 96% | G | G | T | F | C | Q | L | N | A | D | p | Y | E | C | Q | S |
| | 11 | | | | | | | | | | | | | | | | | | | | |
| J550B09 | Peptide- | 0.086 | 1.789 | 95% | 95% | G | G | G | Y | C | E | L | I | G | D | Y | V | V | C | S | P |
| | 12 | | | | | | | | | | | | | | | | | | | | |
| J579A01 | Peptide- | 0.089 | 2.205 | 82% | 96% | G | G | L | C | D | R | W | G | W | I | D | A | P | Y | C | H |
| | 13 | | | | | | | | | | | | | | | | | | | | |
| J578H04 | Peptide- | 0.057 | 2.070 | 80% | 91% | G | G | T | G | C | T | E | G | H | W | H | W | G | T | C | S |
| | 14 | | | | | | | | | | | | | | | | | | | | |
| J577A02 | Peptide- | 0.084 | 1.865 | 95% | 98% | G | G | N | I | C | M | D | Y | S | W | R | S | G | C | A | V |
| | 15 | | | | | | | | | | | | | | | | | | | | |
| J578A05 | Peptide- | 0.072 | 1.487 | 85% | 98% | G | G | H | S | C | T | F | G | D | W | S | L | G | T | C | A |
| | 16 | | | | | | | | | | | | | | | | | | | | |
| J578C08 | Peptide- | 0.057 | 1.025 | 97% | 97% | G | G | F | I | C | T | L | G | N | W | W | D | G | S | C | E |
| | 17 | | | | | | | | | | | | | | | | | | | | |

TABLE 3B
Example phagemid peptide sequences that bind Ab-1, Ab-3 advanced into solid
phase synthesis. Table discloses SEQ ID NOS 1-6, respectively, in order of appearance.

| | | Phage ELISA | | | | | | | | | | | | | | | | | | | |
|---|---|---|---|---|---|---|---|---|---|---|---|---|---|---|---|---|---|---|---|---|---|
| | | | | 2 nM | 20 nM | | | | | | | | | | | | | | | | |
| | | | Ab-1, | EGFR | EGFR | | | | | | | | | | | | | | | | |
| Phage | Peptide | NAv | Ab-3 | % | % | Peptide amino acid sequence from clonal phage | | | | | | | | | | | | | | | |
| Clone | ID | bkgd | signal | inhib | inhib | 1 | 2 | 3 | 4 | 5 | 6 | 7 | 8 | 9 | 10 | 11 | 12 | 13 | 14 | 15 | 16 |
| J455A12 | Peptide- | 0.080 | 1.221 | 94% | 94% | G | G | D | W | C | R | S | L | M | S | Y | T | D | L | C | P |
| | 1 | | | | | | | | | | | | | | | | | | | | |
| J455F12 | Peptide | 0.065 | 0.541 | 85% | 86% | G | G | T | S | C | A | D | A | H | L | I | A | P | S | C | S |
| | 2 | | | | | | | | | | | | | | | | | | | | |
| J456E05 | Peptide- | 0.077 | 1.400 | 95% | 95% | G | G | N | C | Q | W | D | R | V | E | H | T | Y | A | C | S |
| | 3 | | | | | | | | | | | | | | | | | | | | |
| J461A02 | Peptide- | 0.075 | 0.109 | 39% | 39% | G | G | W | V | S | C | H | D | G | S | H | M | T | C | F | H |
| | 4 | | | | | | | | | | | | | | | | | | | | |
| J462A05 | Peptide- | 0.089 | 0.107 | 39% | 20% | G | G | M | N | C | L | N | R | L | W | V | E | Y | C | L | V |
| | 5 | | | | | | | | | | | | | | | | | | | | |
| J464D03 | Peptide- | 0.060 | 1.300 | 95% | 95% | G | G | Y | C | G | Q | D | N | T | W | V | R | E | G | C | F |
| | 6 | | | | | | | | | | | | | | | | | | | | |

TABLE 4
Example phagemid peptide sequences that bind Ab-9, Ab-10 advanced into solid
phase synthesis. Table discloses SEQ ID NOS 19-28, respectively, in order of
appearance.

| | | Phage ELISA | | | | | | | | | | | | | | | | | | | |
|---|---|---|---|---|---|---|---|---|---|---|---|---|---|---|---|---|---|---|---|---|---|
| | | | | 20 nM | 200 nM | | | | | | | | | | | | | | | | |
| | | | Ab-9, | CD3 | CD3 | Peptide | | | | | | | | | | | | | | | |
| Phage | Peptide | NAv | Ab-10 | % | % | amino acid sequence from clonal phage | | | | | | | | | | | | | | | |
| Clone | ID | bkgd | signal | inhib | inhib | 1 | 2 | 3 | 4 | 5 | 6 | 7 | 8 | 9 | 10 | 11 | 12 | 13 | 14 | 15 | 16 |
| J476C07 | Peptide- | 0.051 | 2.652 | 92% | 94% | G | G | D | S | V | C | A | D | P | E | V | P | I | C | E | I |
| | 19 | | | | | | | | | | | | | | | | | | | | |
| J457A04 | Peptide- | 0.092 | 2.979 | 97% | 98% | G | G | M | S | D | C | G | D | P | G | V | E | I | C | T | H |
| | 20 | | | | | | | | | | | | | | | | | | | | |
| J465E03 | Peptide- | 0.052 | 2.107 | 89% | 89% | G | G | I | Q | C | H | D | P | D | L | P | S | P | C | Y | I |
| | 21 | | | | | | | | | | | | | | | | | | | | |
| J465F09 | Peptide- | 0.051 | 2.281 | 87% | 93% | G | G | E | W | C | L | F | D | P | D | V | P | T | C | Q | D |
| | 22 | | | | | | | | | | | | | | | | | | | | |
| J465D07 | Peptide- | 0.055 | 2.279 | 85% | 85% | G | G | L | G | C | N | D | I | D | P | G | E | Q | C | I | V |
| | 23 | | | | | | | | | | | | | | | | | | | | |
| J466C07 | Peptide- | 0.053 | 2.273 | 90% | 90% | G | G | L | E | C | F | D | P | E | I | P | E | A | F | C | I |
| | 24 | | | | | | | | | | | | | | | | | | | | |
| J466C02 | Peptide- | 0.061 | 2.473 | 84% | 84% | G | G | Q | G | C | G | T | I | A | D | P | E | P | H | C | W |
| | 25 | | | | | | | | | | | | | | | | | | | | |
| J479B08 | Peptide- | 0.084 | 0.540 | 81% | 85% | G | G | N | C | H | D | P | D | 1 | P | A | Y | V | L | C | s |
| | 26 | | | | | | | | | | | | | | | | | | | | |
| J467F08 | Peptide- | 0.062 | 2.576 | 94% | 95% | G | G | L | C | P | I | N | D | W | E | P | Q | D | I | C | W |
| | 27 | | | | | | | | | | | | | | | | | | | | |
| J467D04 | Peptide- | 0.058 | 2.587 | 93% | 94% | G | G | L | C | M | I | G | D | W | L | P | G | D | V | C | L |
| | 28 | | | | | | | | | | | | | | | | | | | | |

Peptides were synthesized via standard peptide chemistry. Peptides were synthesized as linear or cyclic as appropriate. A C-terminal linker consisting of Gly4Ser (SEQ ID NO: 82), PEG4, and Lys(Biotin) was added to the phagemid peptide sequence identified from panning and DNA sequencing. The C-terminal acids were also capped via amidation. Peptides were purified by HPLC to ≥95% purity and verified by liquid chromatography assisted mass spectrometry (LC-MS). Peptides were lyophilized prior to dissolution in DMSO.

Synthetic peptides were initially screened for binding to their panning target. As an example, peptides listed bind to Ab-6, Ab-7 (TABLE 2), Ab-1, Ab-3 (TABLE 3), or Ab-9, Ab-10 (TABLE 4). Peptide binding was evaluated using both kinetic measurements via Bio-layer Interferometry (BLI) or equilibrium measurements using enzyme linked immunosorbent assays (ELISAs).

Kinetic Binding of Antibody to Peptides

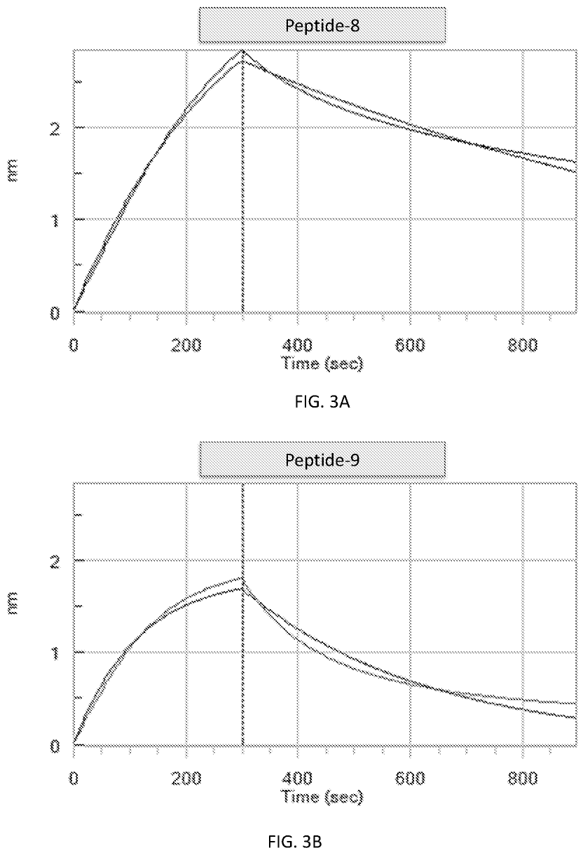

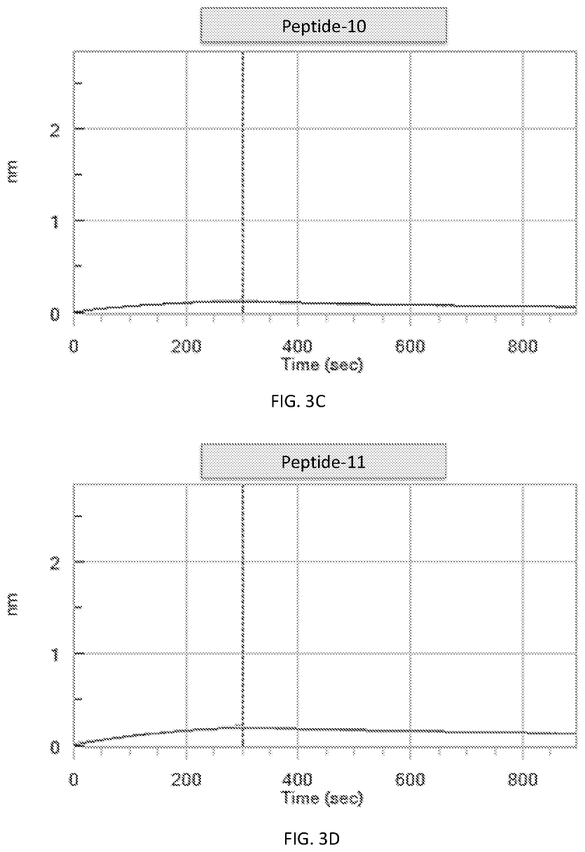

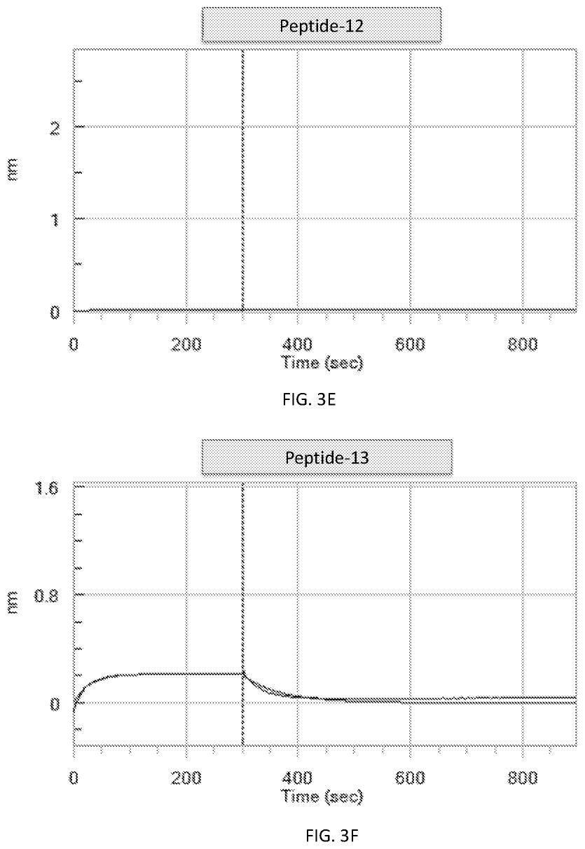

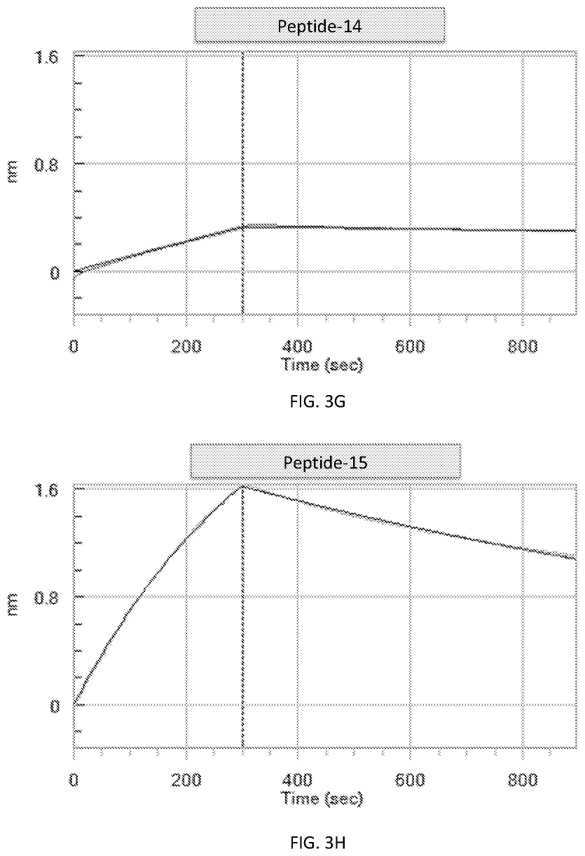

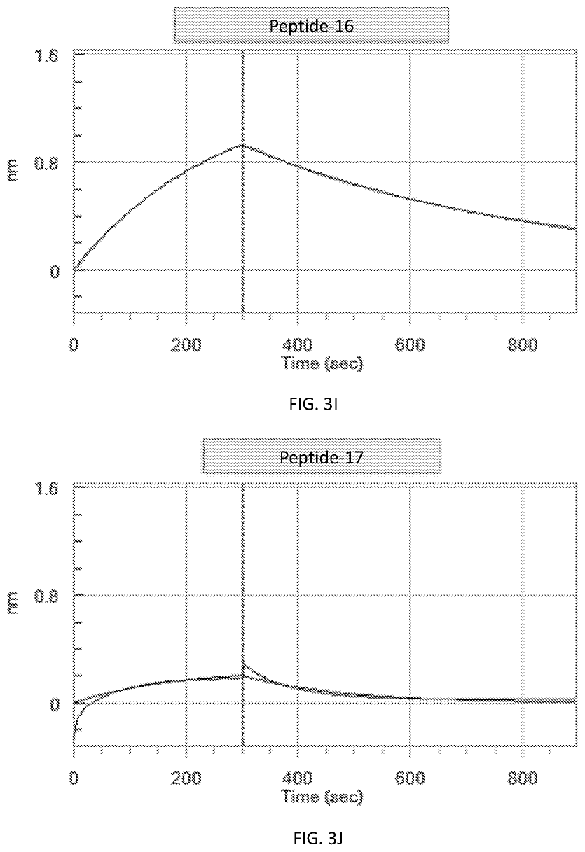

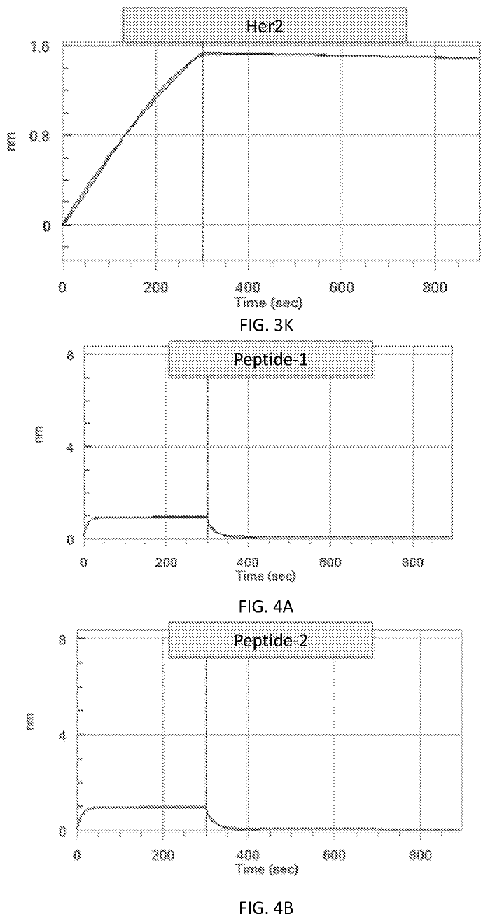

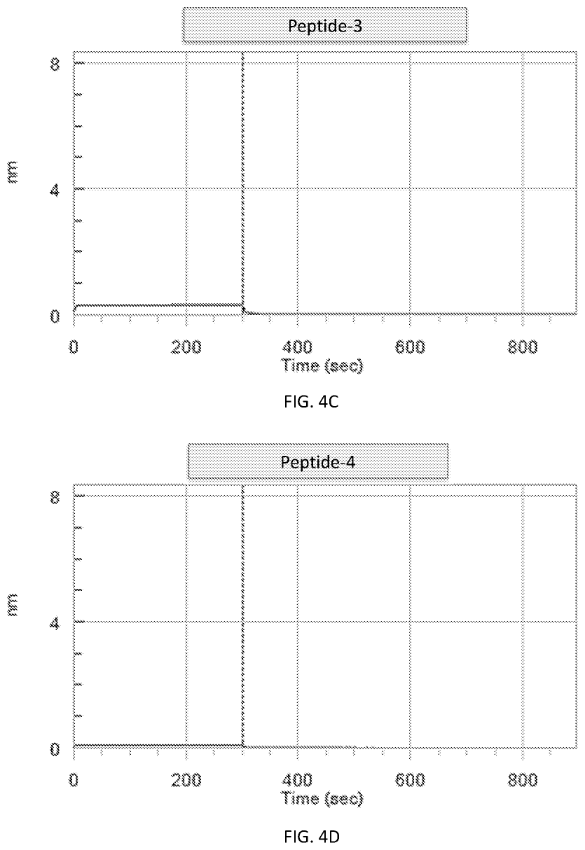

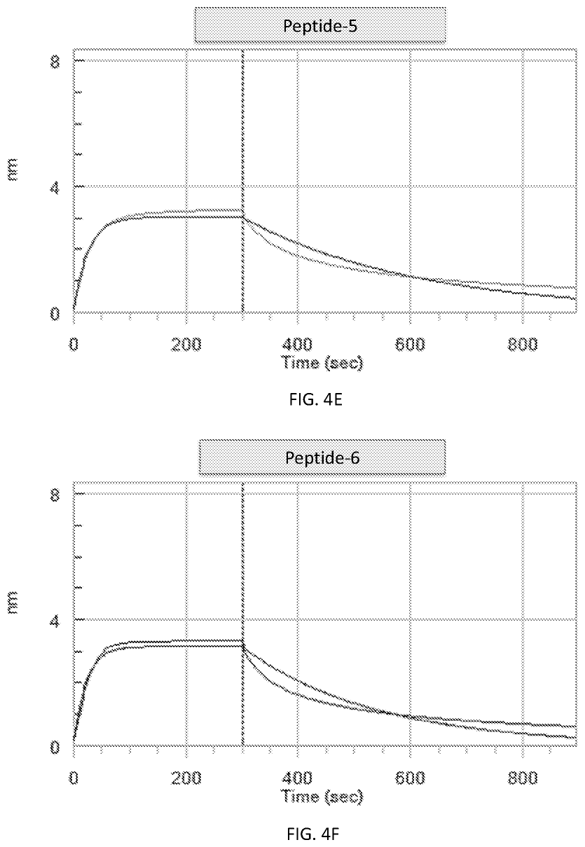

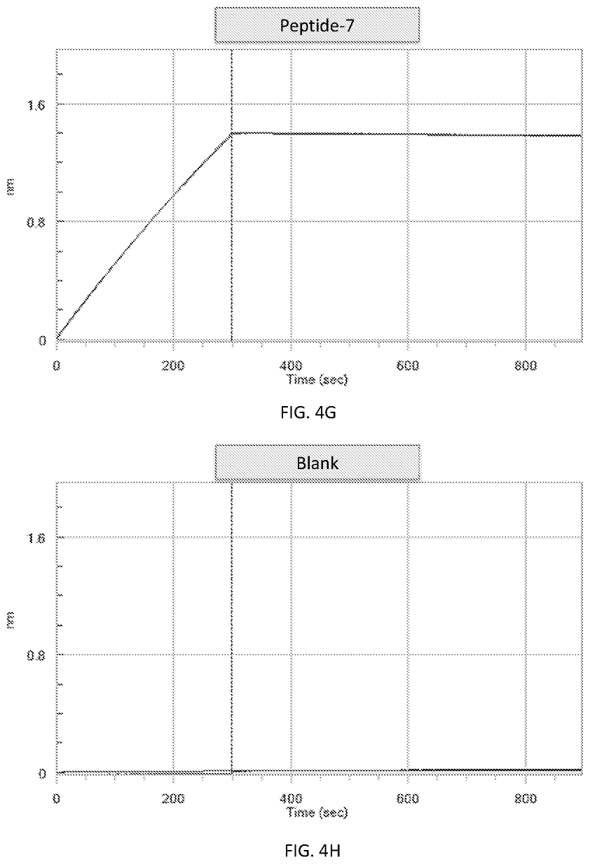

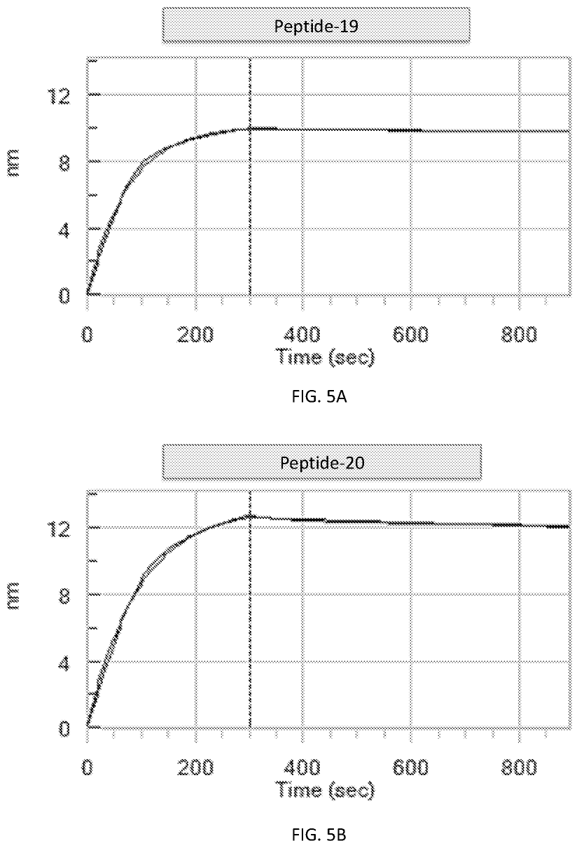

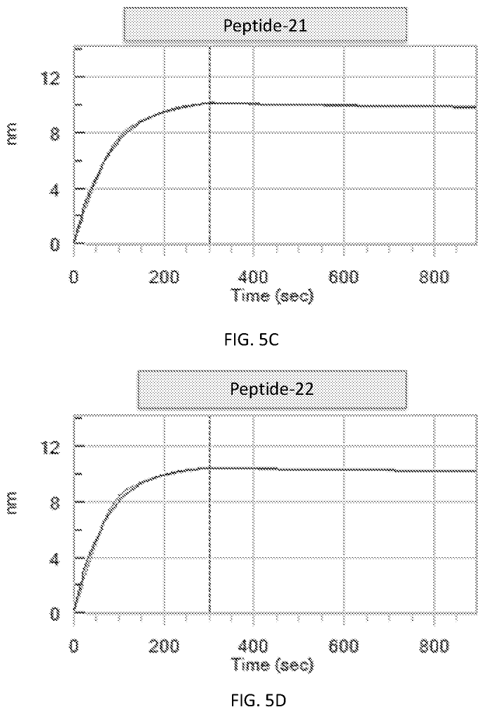

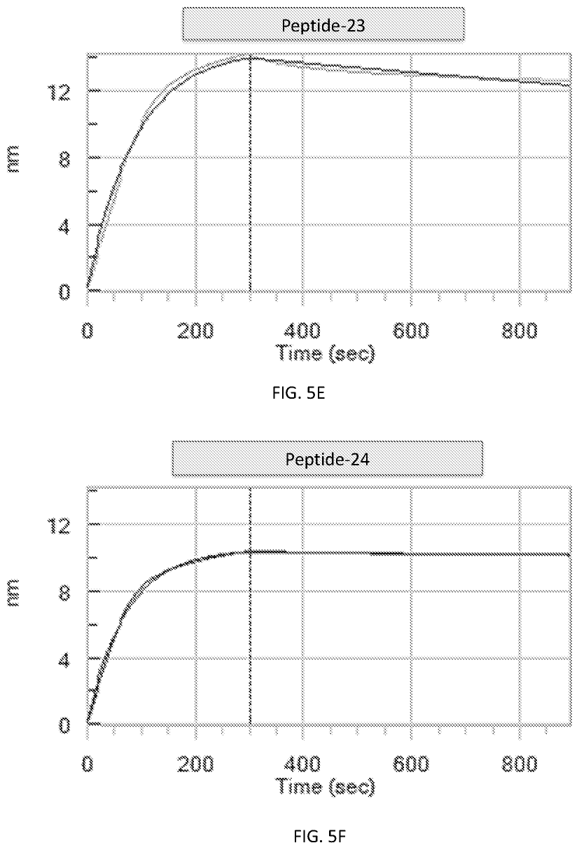

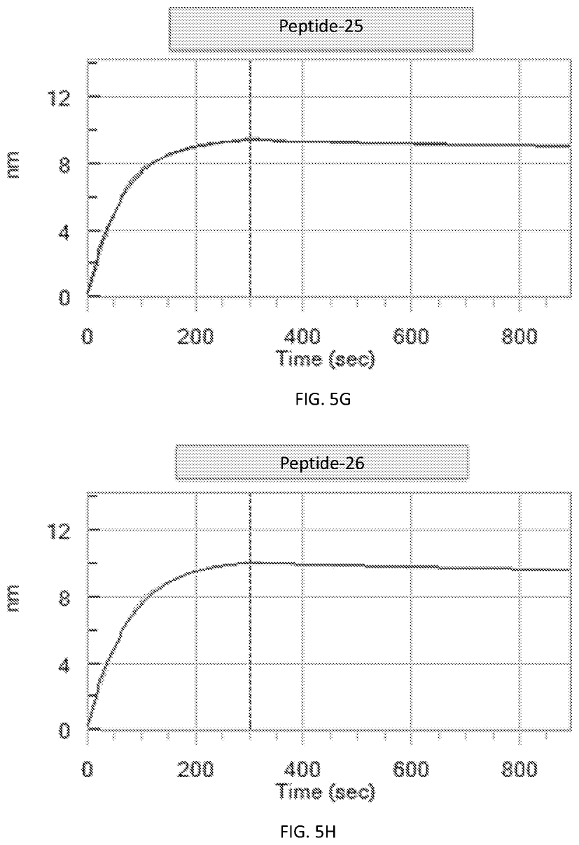

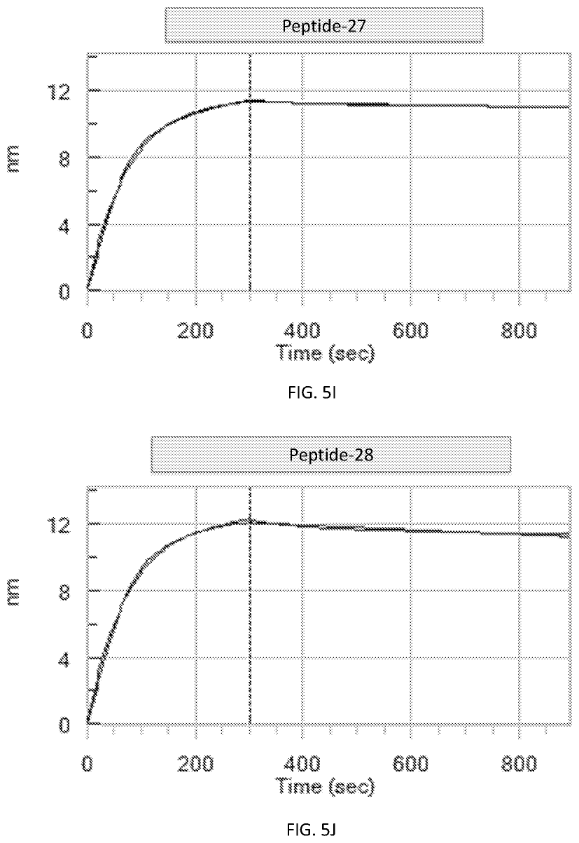

BLI based kinetic binding of antibody to peptides was measured using a ForteBio Octet RED96 instrument. Biotinylated peptides were first directly captured on streptavidin biosensors. Sensors were quenched using excess biocytin and then baselined in buffer. A dilution series of antibody or antibody fragments was made and associated onto the peptide loaded biosensor. Association and dissociation signals were monitored in real-time. Signals were fit to a 1:1 binding model in order to derive binding constants, kon and koff, as well as KD. Exemplary kinetic binding sensorgrams of Trastuzumab (Ab-6, Ab-7) recognition of peptides relative to HER2 antigen are shown in FIGS. 3A-3K. Exemplary kinetic binding sensorgrams of Cetuximab (Ab-1, Ab-3) recognition of peptides relative to Her2 antigen are shown in FIGS. 4A-4H. Exemplary kinetic binding sensorgrams of 5P34 (Ab-9, Ab-10) recognition of peptides relative to Her2 antigen are shown in FIGS. 5A-5L.

Equilibrium Binding of Antibody to Peptides

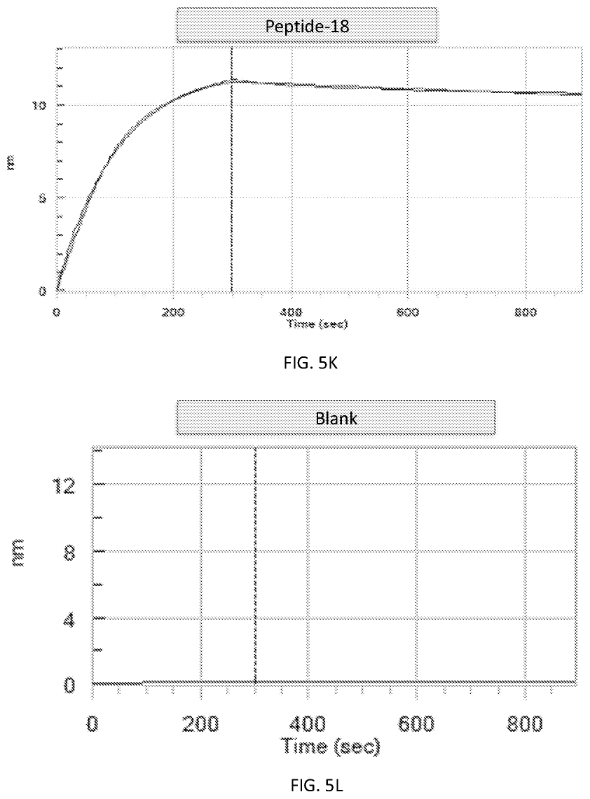

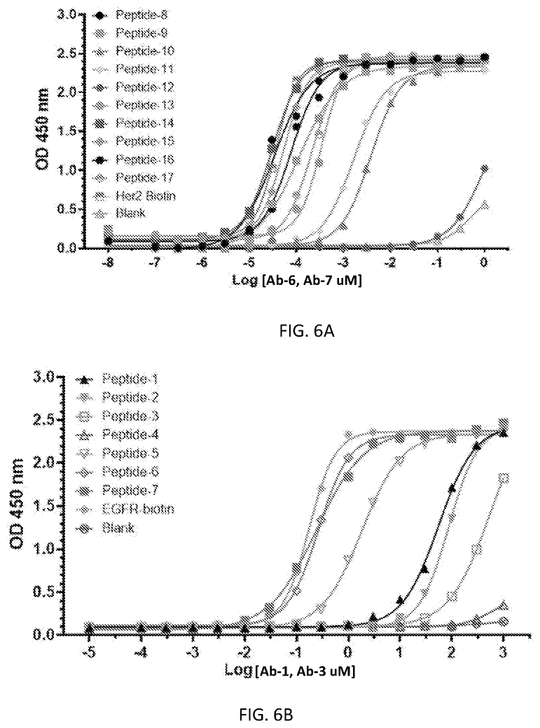

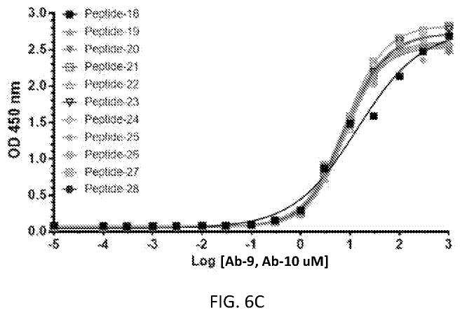

Peptide binding was also examined in an ELISA format. Biotinylated peptides were captured on neutravidin coated plates. Antibody was then prepared in a half-log dilution series starting from 10 uM and titrated onto the peptide captured plates. A secondary horse radish peroxidase (HRP) antibody conjugate that recognizes mouse or human antibody was used to detect bound antibody. The concentration of antibody required to observe half maximal binding signal (EC50) was then calculated using Graphpad Prism. Example binding of Trastuzumab (Ab-6, Ab-7), Cetuximab (Ab-1, Ab-3) or SP34 (Ab-9, Ab-10) to captured peptide or antigen is shown in FIGS. 6A-6C, respectively. A wide range of peptide EC50s was observed from 10 nM to greater than 10 uM whereas the cognate antigen EC50s were less than 1 nM. EC50 binding data for peptides is summarized in TABLE 5. Promising peptides that exhibited reasonable binding by both BLI and ELISA were progressed into competitive binding experiments.

TABLE 5
Summary of example peptide binding characteristics.

| | | | Octet BLI | | | |
|---|---|---|---|---|---|---|
| | | | | Competes | ELISA | |
| | Peptide | | | at | EC50 | IC50 |
| Target | ID | Peptide Sequence | Binds | 100 uM | nM | uM |
| Trastuzumab | Peptide- | GGPLCSDLDHITRLCDGGGGS[PEG4]Lys(biotin)-NH2 | YES | YES | 0.03 | 15.98 |
| | 8 | (SEQ ID NO: 83) | | | | |
| Trastuzumab | Peptide- | GGIDCASLDHYTESCYGGGGS[PEG4]Lys(biotin)-NH2 | YES | YES | 0.11 | 37.16 |
| | 9 | (SEQ ID NO: 84) | | | | |
| Trastuzumab | Peptide- | GGNPVCTLGDPYECSHGGGGS[PEG4]Lys(biotin)-NH2 | YES | NO | 3.84 | >300 |
| | 10 | (SEQ ID NO: 85) | | | | |
| Trastuzumab | Peptide- | GGTFCQLNADPYECQSGGGGS[PEG4]Lys(biotin)-NH2 | NO | NO | 1.67 | >300 |
| | 11 | (SEQ ID NO: 86) | | | | |
| Trastuzumab | Peptide- | GGGYCELIGDYWCSPGGGGS[PEG4]Lys(biotin)-NH2 | NO | NO | 972 | >300 |
| | 12 | (SEQ ID NO: 87) | | | | |
| Trastuzumab | Peptide- | GGLCDRWGWIDAPYCHGGGGS[PEG4]Lys(biotin)-NH2 | NO | | 0.34 | >300 |
| | 13 | (SEQ ID NO: 88) | | | | |
| Trastuzumab | Peptide- | GGTGCTEGHWHWGTCSGGGGS[PEG4]Lys(biotin)-NH2 | Weak | | 0.03 | 1.31 |
| | 14 | (SEQ ID NO: 89) | | | | |
| Trastuzumab | Peptide- | GGNICMDYSWRSGCAVGDGGS[PEG4]Lys(biotin)-NH2 | YES | | 0.05 | 4.09 |
| | 15 | (SEQ ID NO: 90) | | | | |
| Trastuzumab | Peptide- | GGHSCTFGDWSLGTCAGGGGS[PEG4]Lys(biotin)-NH2 | YES | | 0.08 | 3.06 |
| | 16 | (SEQ ID NO: 91) | | | | |
| Trastuzumab | Peptide- | GGFICTLGNWWDGSCEGGGGS[PEG4]Lys(biotin)-NH2 | YES | | 0.22 | 11.41 |
| | 17 | (SEQ ID NO: 92) | | | | |
| Cetuximab | Peptide- | GGDWCRSLMSYTDLCPGGGGS[PEG4]Lys(biotin)-NH2 | YES | NO | 52.03 | >300 |
| | 1 | (SEQ ID NO: 93) | | | | |
| Cetuximab | Peptide- | GGTSCADAHLIAPSCSGGGGS[PEG4]Lys(biotin)-NH2 | YES | NO | 84.47 | >300 |
| | 2 | (SEQ ID NO: 94) | | | | |
| Cetuximab | Peptide- | GGNCQWDRVEHTYACSGGGGS[PEG4]Lys(biotin)-NH2 | Weak | NO | 412.70 | >300 |
| | 3 | (SEQ ID NO: 95) | | | | |
| Cetuximab | Peptide- | GGWVSCHDGSHMTCFHGGGGS[PEG4]Lys(biotin)-NH2 | NO | NO | >1000 | >300 |
| | 4 | (SEQ ID NO: 96) | | | | |
| Cetuximab | Peptide- | GGMNCLNRLWVEYCLVGGGGS[PEG4]Lys(biotin)-NH2 | YES | NO | 1.96 | >300 |
| | 5 | (SEQ ID NO: 97) | | | | |
| Cetuximab | Peptide- | GGYCGQDNTWVREGCFGGGGS[PEG4]Lys(biotin)-NH2 | YES | YES | 0.27 | 28.03 |
| | 6 | (SEQ ID NO: 98) | | | | |
| Cetuximab | Peptide- | QGQSGQLSCEGWAMNREQCRAGGGGS[PEG4]Lys(biotin)- | YES | YES | 0.25 | 10.02 |
| | 7 | NH2 (SEQ ID NO: 99) | | | | |
| SP34 | Peptide- | QGQSGQGYLWGCEWNCGGITTGGGGS[PEG4]Lys(biotin)- | YES | YES | 2.75 | 4.76 |
| | 18 | NH2 (SEQ ID NO: 100) | | | | |
| SP34 | Peptide- | GGDSVCADPEVPICEIGGGGS[PEG4]Lys(biotin)-NH2 | YES | YES | 8.03 | >300 |
| | 19 | (SEQ ID NO: 101) | | | | |
| SP34 | Peptide- | GGMSDCGDPGVEICTHGGGGS[PEG4]Lys(biotin)-NH2 | YES | YES | 7.13 | >300 |
| | 20 | (SEQ ID NO: 102) | | | | |
| SP34 | Peptide- | GGIQCHDPDLPSPCYIGGGGS[PEG4]Lys(biotin)-NH2 | YES | YES | 8.25 | >300 |
| | 21 | (SEQ ID NO: 103) | | | | |
| SP34 | Peptide- | GGEWCLFDPDVPTCQDGGGGS[PEG4]Lys(biotin)-NH2 | YES | YES | 2.56 | 1.81 |
| | 22 | (SEQ ID NO: 104) | | | | |
| SP34 | Peptide- | GGLGCNDIDPGEQCIVGGGGS[PEG4]Lys(biotin)-NH2 | YES | YES | 8.20 | >300 |
| | 23 | (SEQ ID NO: 105) | | | | |
| SP34 | Peptide- | GGLECFDPEIPEAFCIGGGGS[PEG4]Lys(biotin)-NH2 | YES | YES | 2.66 | 2.22 |
| | 24 | (SEQ ID NO: 106) | | | | |
| SP34 | Peptide- | GGQGCGTIADPEPHCWGGGGS[PEG4]Lys(biotin)-NH2 | YES | YES | 1.95 | 2.49 |
| | 25 | (SEQ ID NO: 107) | | | | |
| SP34 | Peptide- | GGNCHDPDIPAYVLCSGGGGS[PEG4]Lys(biotin)-NH2 | YES | YES | 8.69 | >300 |
| | 26 | (SEQ ID NO: 108) | | | | |
| SP34 | Peptide- | GGLCPINDWEPQDICWGGGGS[PEG4]Lys(biotin)-NH2 | YES | YES | 7.62 | >300 |
| | 27 | (SEQ ID NO: 109) | | | | |
| SP34 | Peptide- | GGLCMIGDWLPGDVCLGGGGS[PEG4]Lys(biotin)-NH2 | YES | YES | 6.76 | 96.01 |
| | 28 | (SEQ ID NO: 110) | | | | |

Inhibition of Kinetic Binding for Antibody to its Cognate Antigen Using Inhibitory Peptides

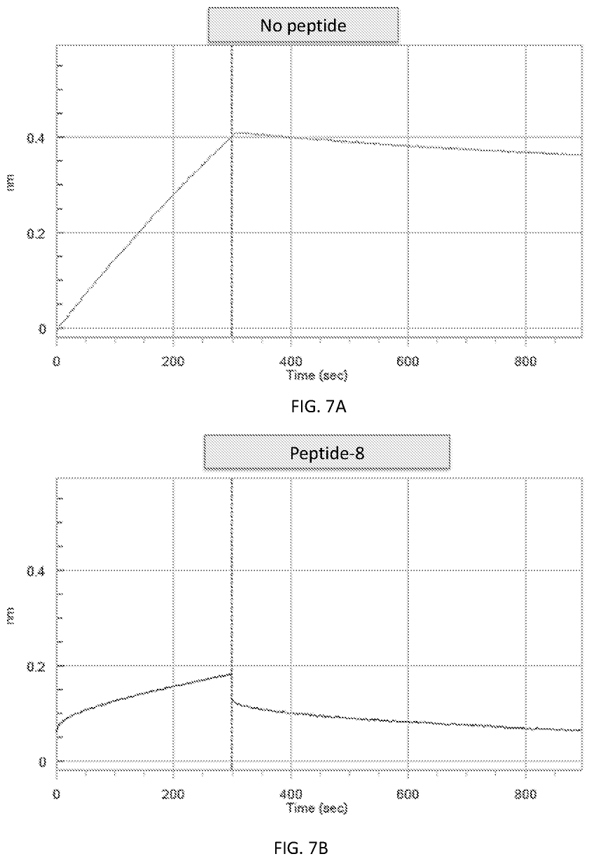

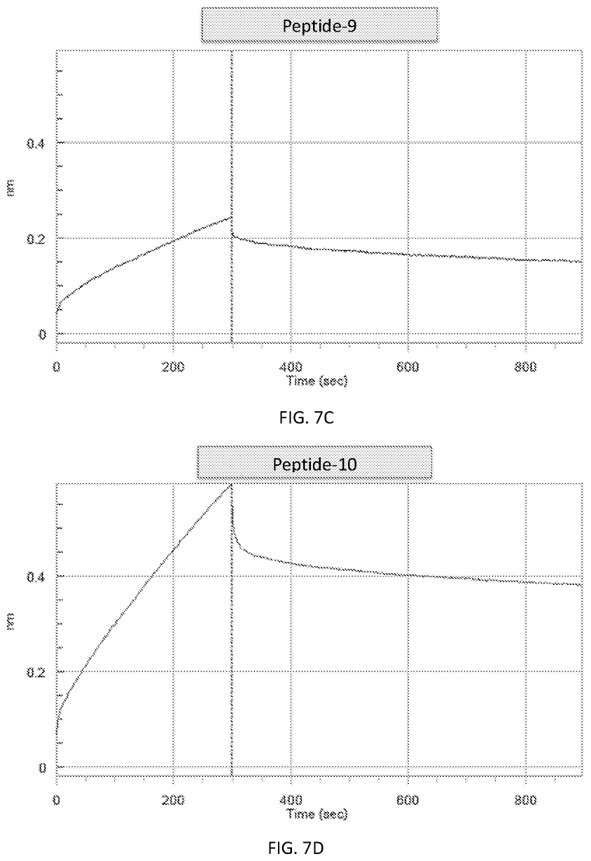

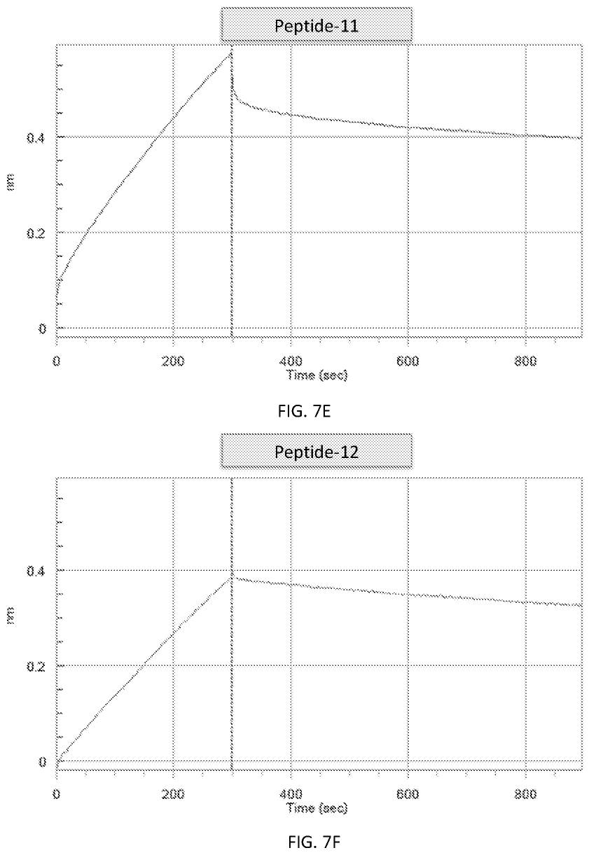

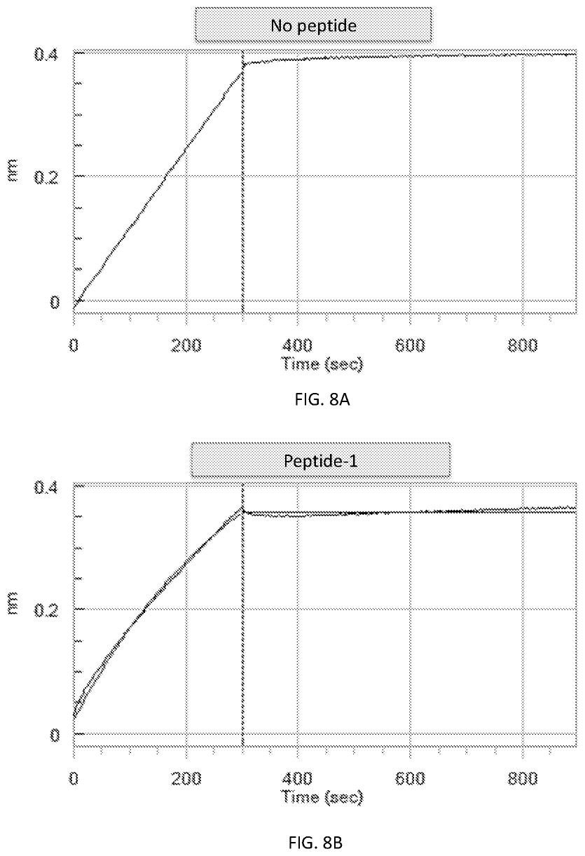

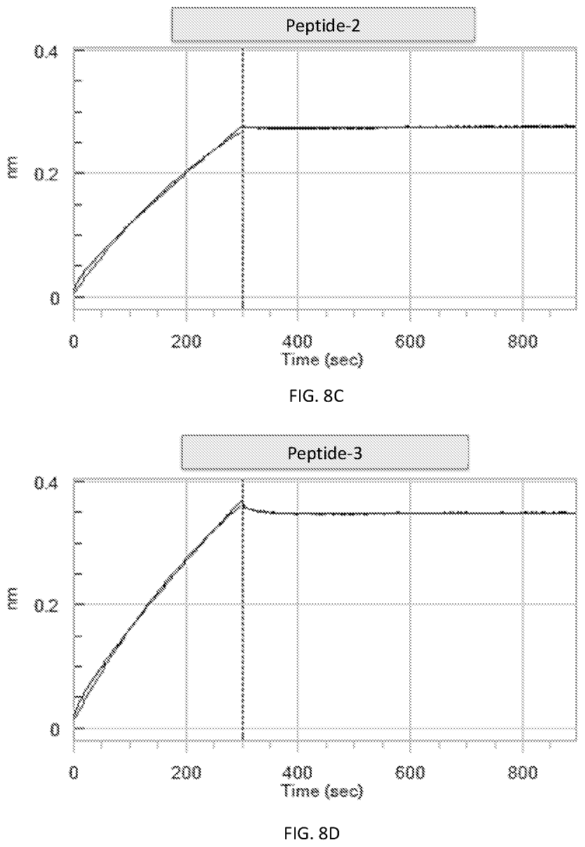

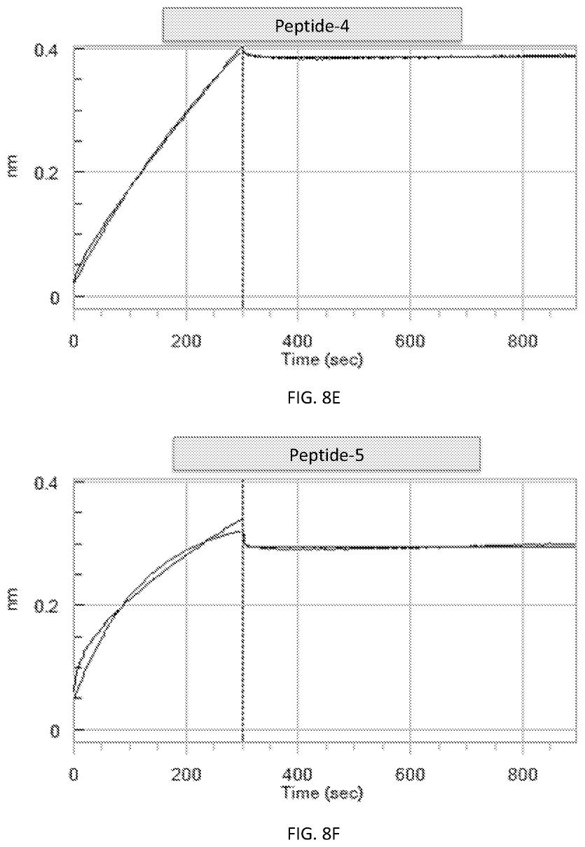

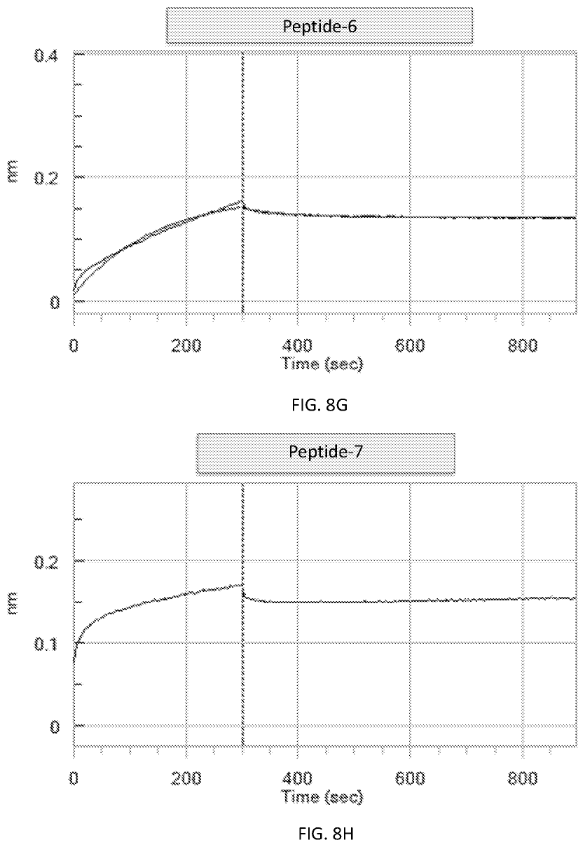

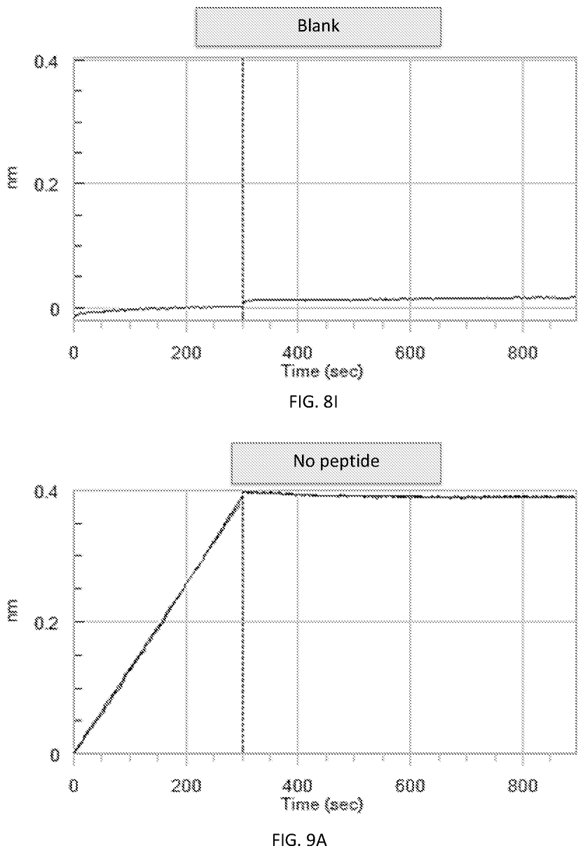

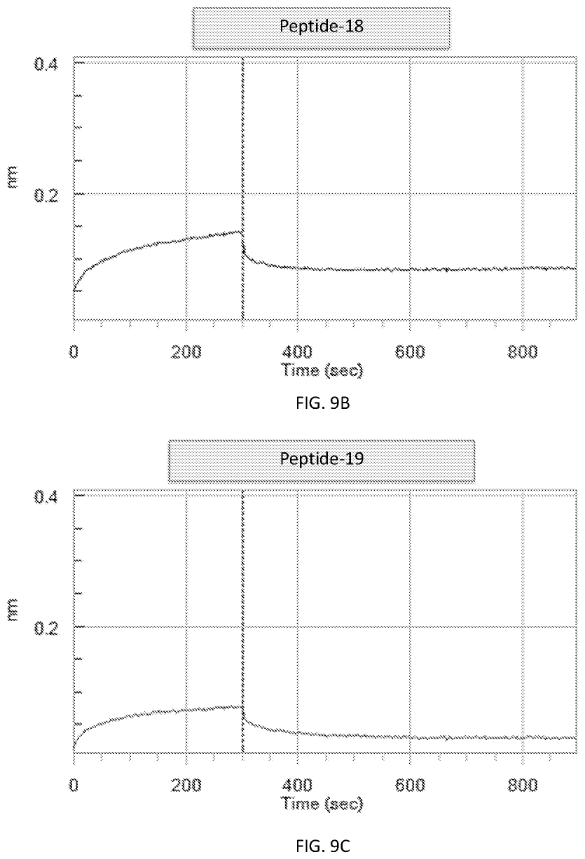

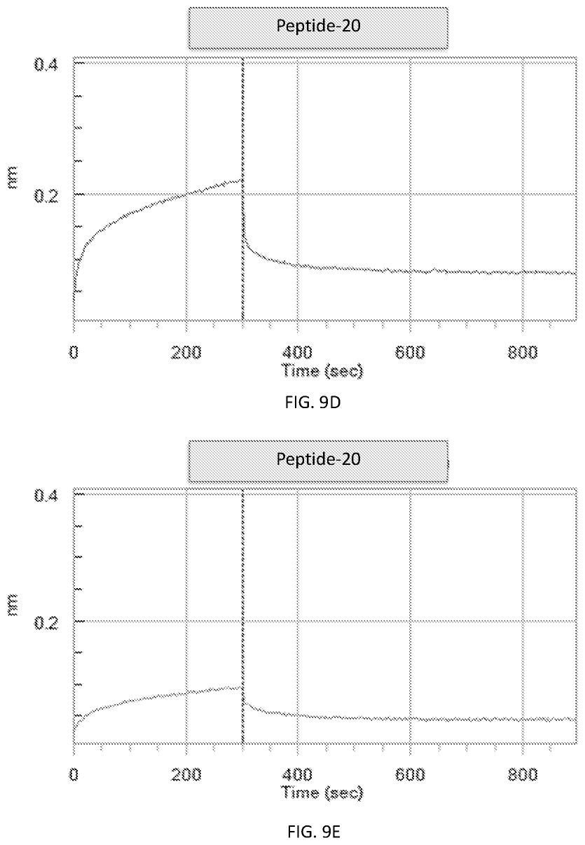

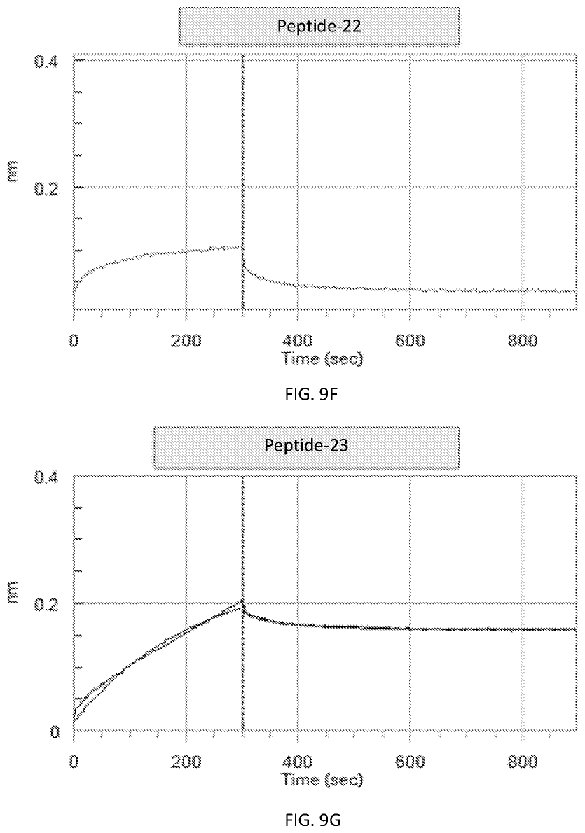

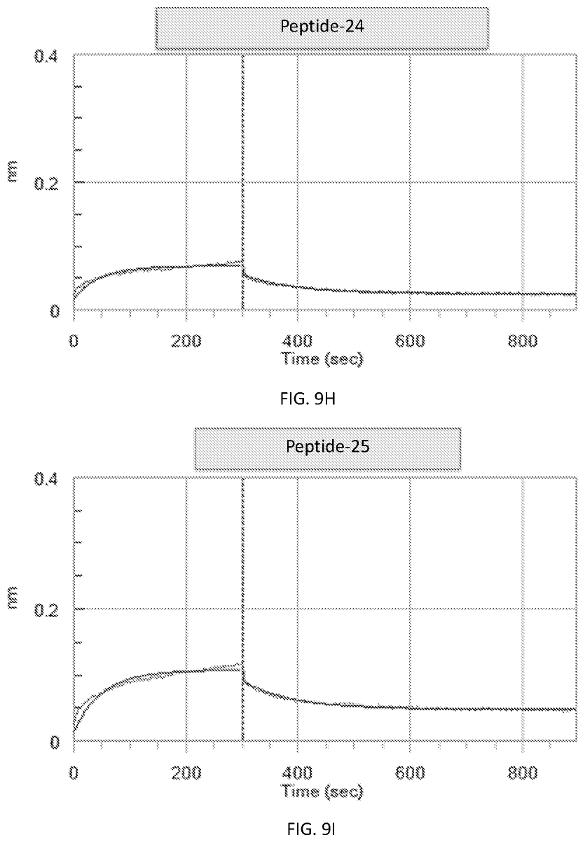

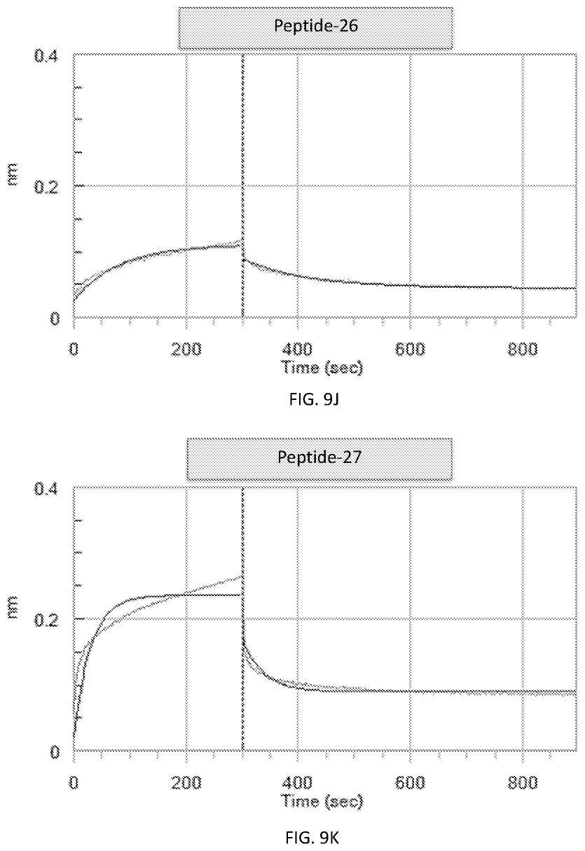

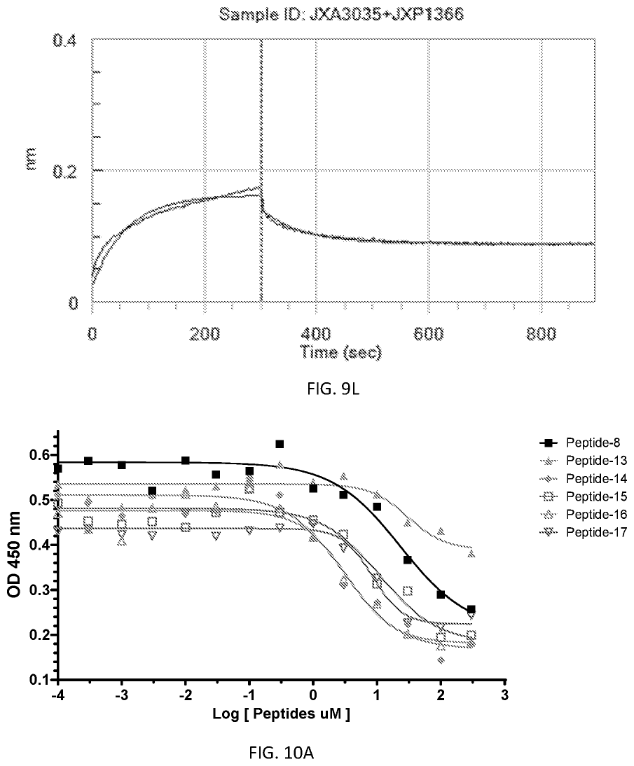

Peptides that bind do not necessarily exhibit desired function. For a peptide to function as a mask it must by definition inhibit the antibody of interest from binding its cognate antigen. Therefore, peptides that bind the example antibody targets were progressed into competitive inhibition studies designed to test the inhibitory function of each peptide. Multiple peptides were evaluated for masking function via BLI and ELISA. FIGS. 7A-7F, FIGS. 8A-8I, and FIGS. 9A-9L provide example peptide inhibition of indicated antibody binding to cognate antigen in a dose dependent manner IC50 data for all peptides is summarized in TABLE 5.

Dose dependent kinetic inhibition of antibody binding to its cognate using the identified peptide binders was measured via BLI using a ForteBio Octet RED96 instrument . First, biotinylated antigen was captured on streptavidin biosensors. Sensors were quenched using excess biocytin and then baselined in buffer. Inhibitory peptide was titrated in a twofold dilution series starting from 100 uM and pre-incubated with a constant concentration of antibody. Peptide and antibody mixtures were then associated onto the antigen loaded biosensor. Zero concentration of inhibitory peptide or zero concentration of antibody were used as controls. Association and dissociation signals were monitored in real-time. The maximal association signal was normalized from 100% (0 uM inhibitory peptide control) to 0% (0 nM antibody control) and plotted versus log-scale inhibitory peptide concentration. Graphpad Prism was used to calculate the inhibitory concentration of peptide required to achieve 50% maximal signal (IC50) summarized in TABLE 5.

Inhibition of Equilibrium Binding for Antibody to its Cognate Antigen Using Inhibitory Peptides

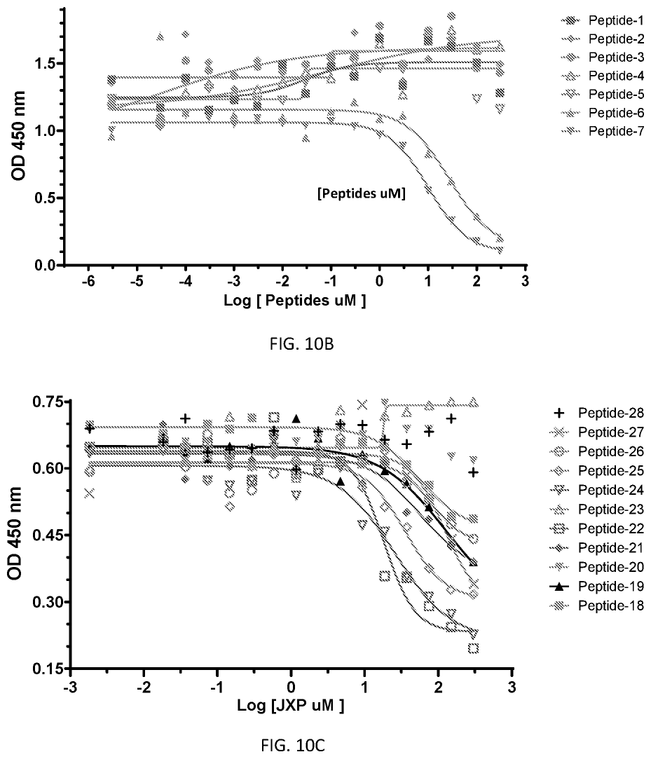

Inhibition of antibody binding to its cognate antigen was also measured in an ELISA format. Biotinylated antigen was captured on neutravidin coated plates, quenched using excess biocytin, and washed. Inhibitory peptide was titrated in a half-log dilution series starting from 100 uM and pre-incubated with a constant concentration of antibody. Inhibitory peptide and antibody mixtures were then incubated on the antigen captured plates. A secondary HRP antibody conjugate that recognized mouse or human antibody was then used to detect the plate bound antibody. The ELISA signal was normalized from 100% (0 nM inhibitory peptide control) to 0% (0 nM antibody control) and plotted versus log-scale inhibitory peptide concentration (FIGS. 10A-10C). Dose dependent decrease of signal was indicative of peptides that compete for antibody binding to its cognate antigen. Graphpad Prism was used to calculate the inhibitory concentration of peptide required to achieve 50% maximal signal (IC50) summarized in TABLE 5.

### Example 4

### Design, Synthesis, and In Vitro Efficacy of Tumor Activated T Cell Engagers

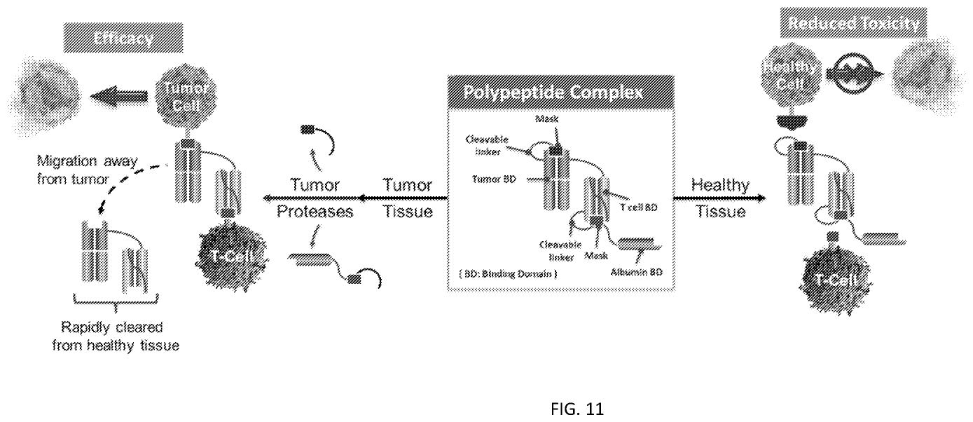

Bispecific T cell engagers typically have poor pharmacokinetics (PK) properties. We hypothesized that adding a half-life extension molecule in tandem with the proteolytically cleavable mask would exhibit crossover PK defined by a long half-life in systemic circulation but fast clearance after mask and PK extender cleavage at the tumor site due to specific proteolytic activity. Thus these cross over PK molecules would have an additional safety switch preventing accumulation in healthy tissue once activated at the tumor site. FIG. 11 illustrates the tumor specific activity and cross over PK for tumor activated T cell engager molecules.

Various polypeptide complex constructs were made by fusing an anti-albumin single domain antibody (SDA) in tandem to the inhibitory peptide masks separated by a short GlySer linker. The tandem SDA peptide mask was genetically fused to the polypeptide complex using cleavable or non-cleavable linkers recognized by tumor specific proteases. Functional binding, tumor cell killing, and T cell activation of the polypeptide complexes were then evaluated. TrastuzuFab SP34 scFv or CetuxiFab SP34 scFv polypeptide complexes were tested against HER2 or EGFR positive tumor cells lines, respectively. In addition, mouse PK and mouse efficacy were evaluated in Balb/c and HCT116 tumor bearing NCG mice, respectively, using polypeptide complex constructs. The crossover PK enhanced polypeptide complex constructs were also evaluated in Cynomolgus monkey PK and safety studies. Data suggested that polypeptide complex molecules maintained potent anti-tumor activity while improving PK and safety.

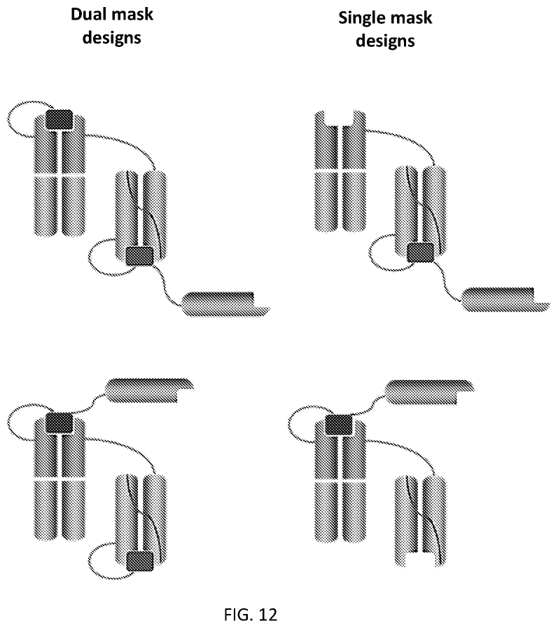

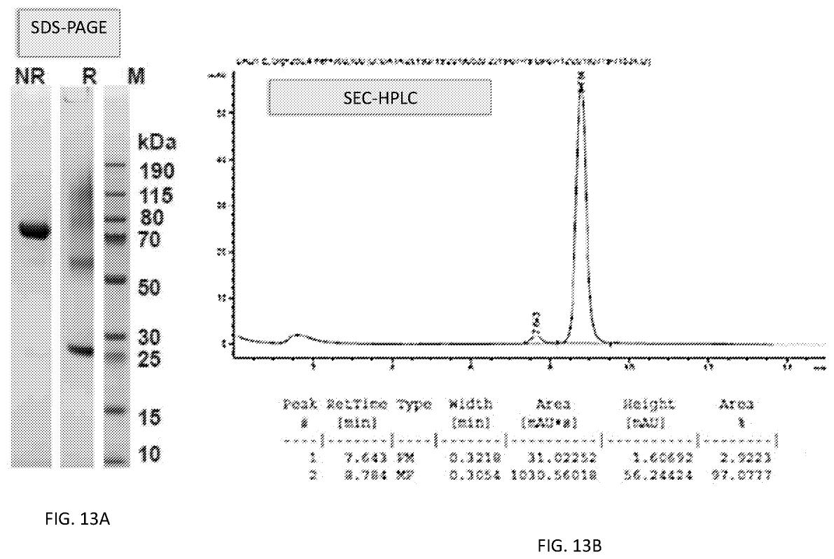

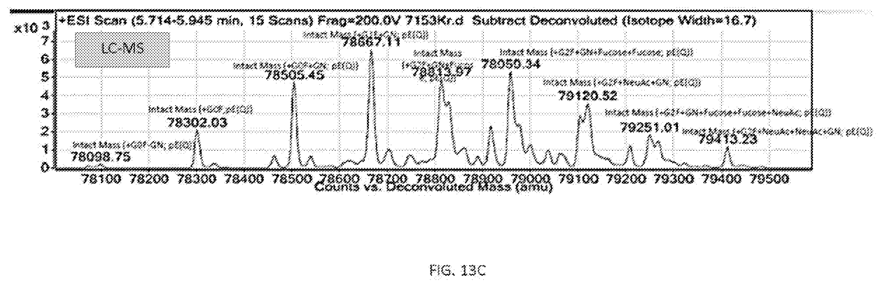

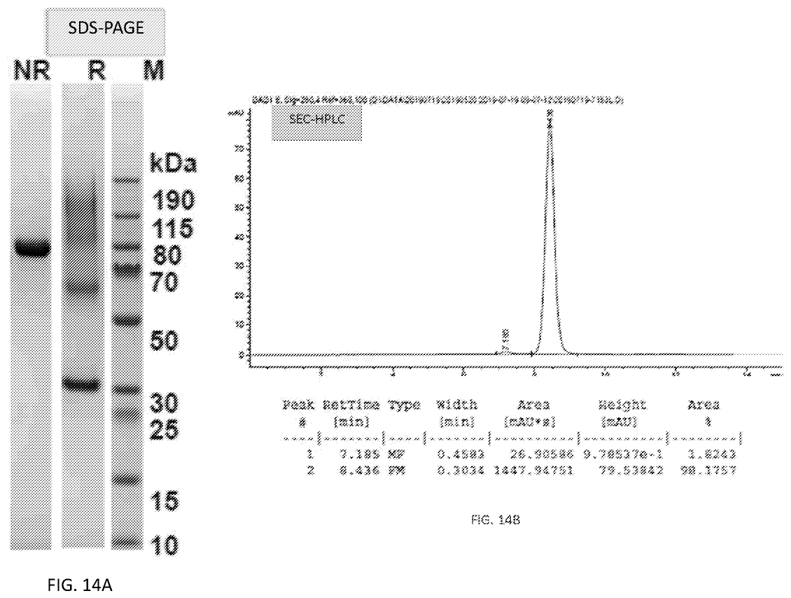

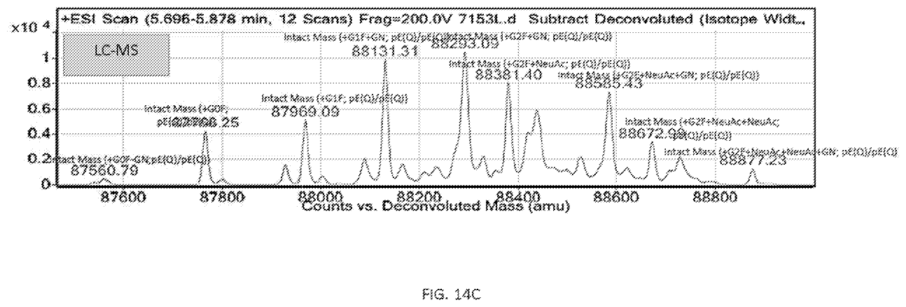

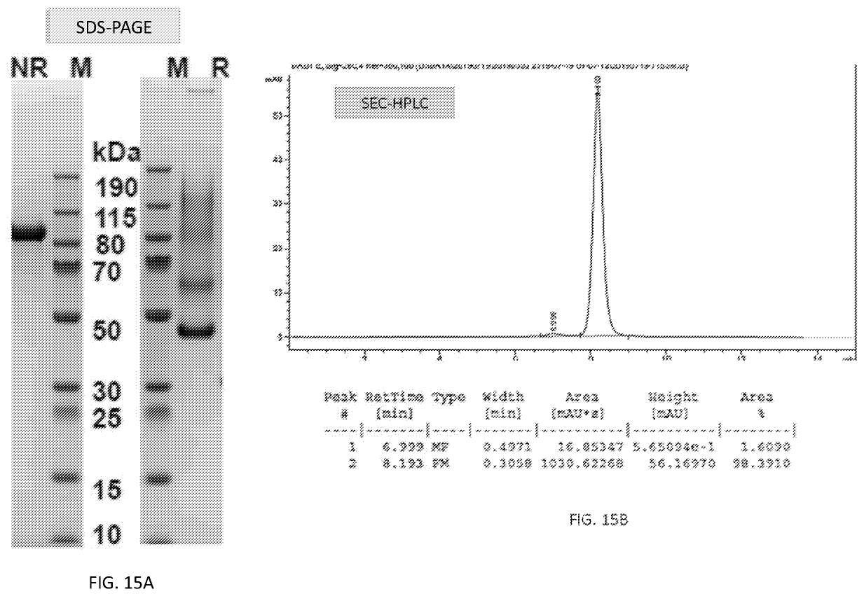

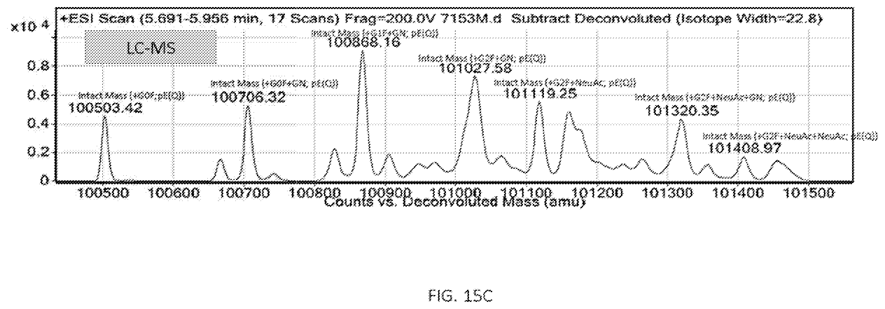

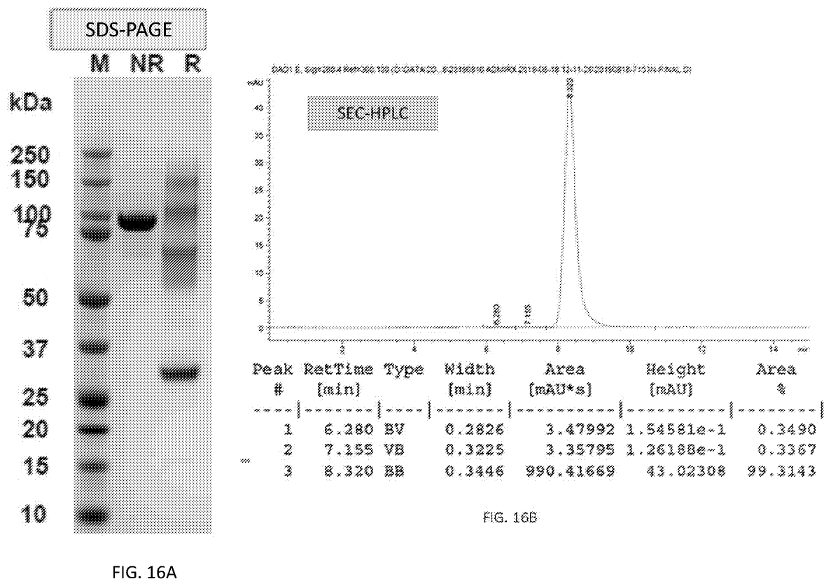

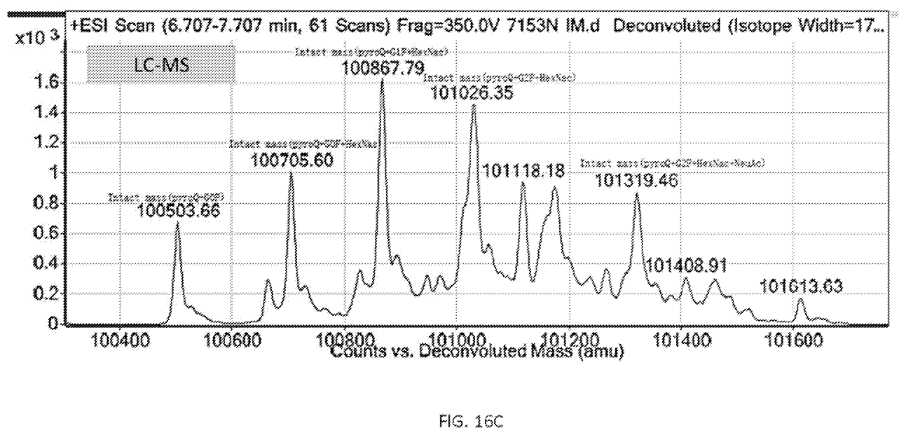

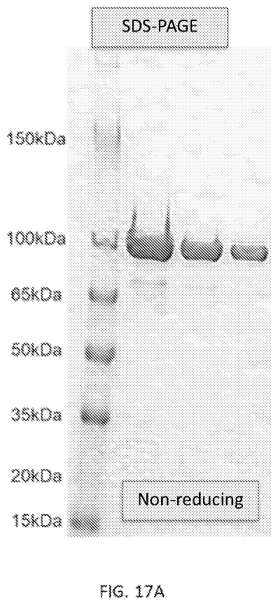

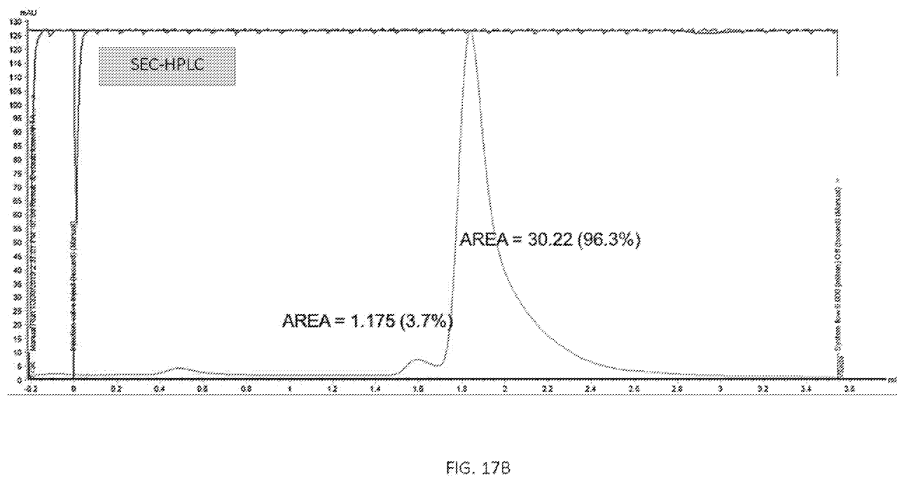

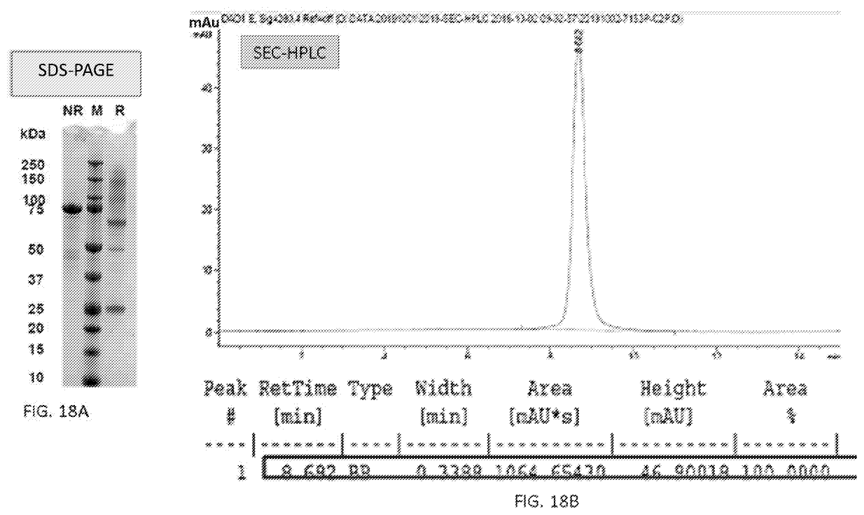

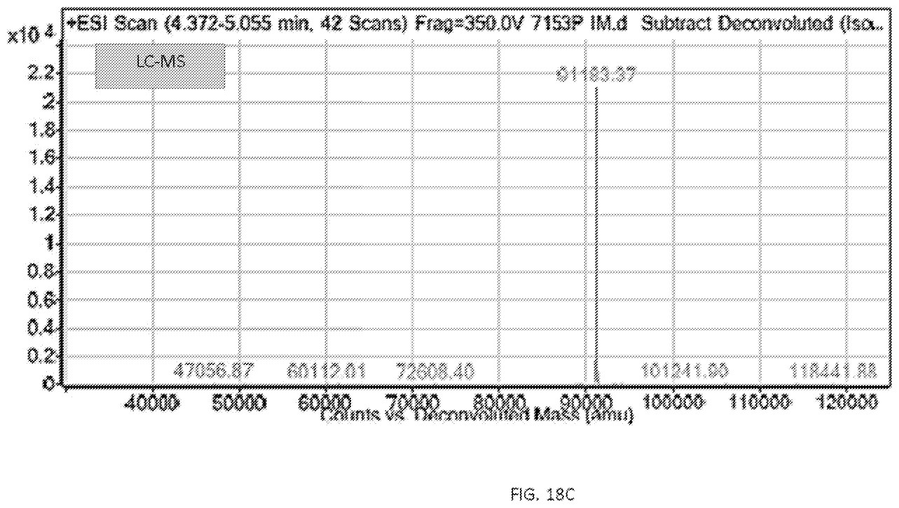

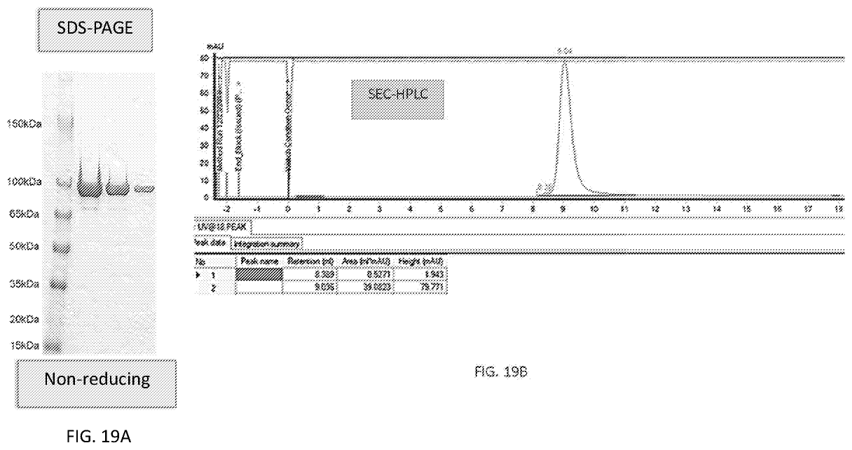

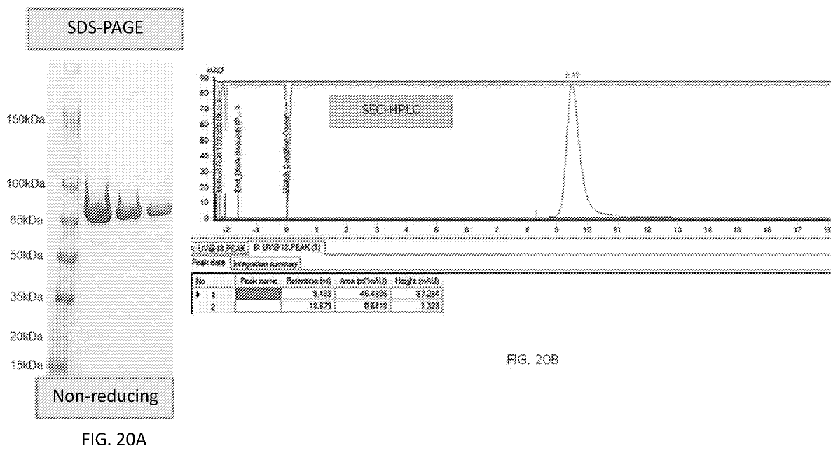

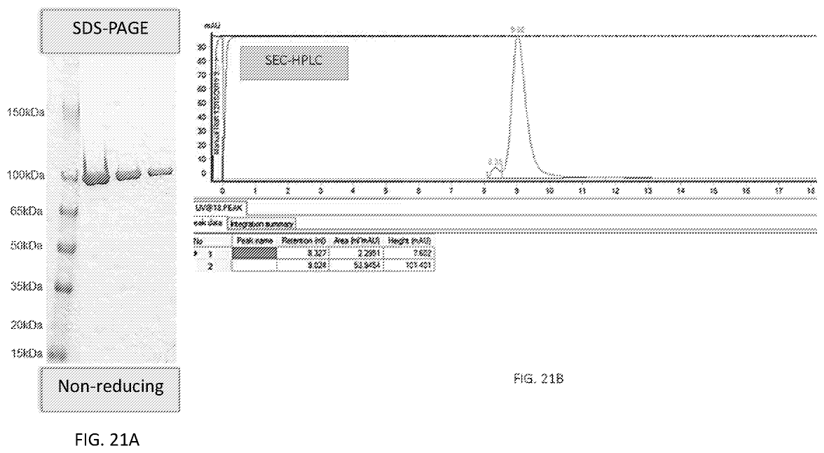

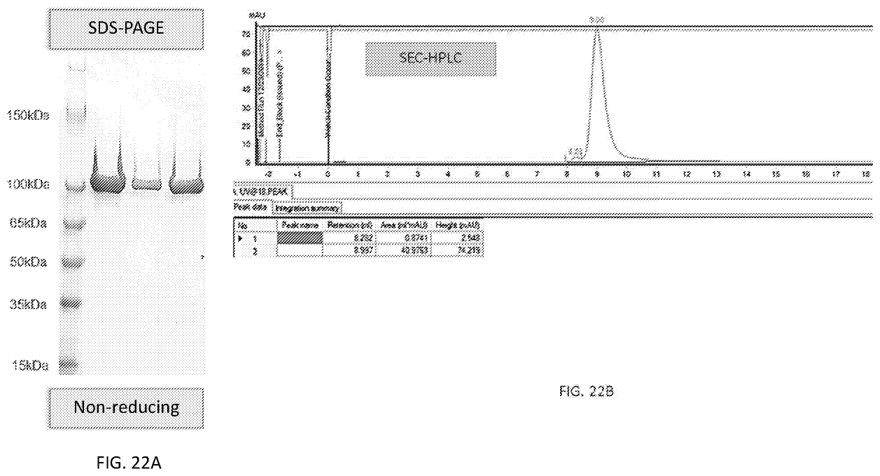

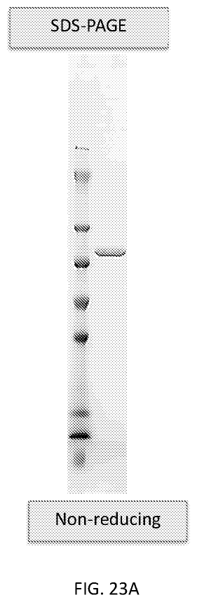

Generalized polypeptide complex molecule designs are shown in FIG. 12. Proteins were produced recombinantly in mammalian host cells and purified as described. FIGS. 13A-13C through FIGS. 24A-24B highlight the production quality of polypeptide complex constructs.

Polypeptide Complex Production and Purification

Expression plasmids encoding the polypeptide complex were produced using standard molecular biology techniques. Plasmids were transfected into CHOs or HEK293 cells and incubated for 10 days feeding every other day using standard mammalian host recombinant protein production techniques. Supernatants were harvested after 10 days, filtered, and purified using anti-CH1 affinity chromatography followed by ion exchange polishing step. Purified proteins were buffer exchanged into storage buffer and stored frozen. The resulting soluble proteins were tested for their biochemical integrity and quality by three analytical methods. First, portions of the purified polypeptide complex were tested by heating in loading buffer in the presence or absence of reducing agent. Total protein was then examined by SDS-PAGE which indicated relative protein purity and correct molecular weight and disulfide pairing. Second, a portion of the resulting polypeptide complex batches were tested by size exclusion chromatography to determine whether there were smaller or larger than expected molecular weight components, indicating degraded or aggregated protein, respectively. Lastly, the polypeptide complex batches were analyzed by LC-MS methods to further prove correct disulfide pairing as well as demonstrate a lack of mask cleavage in the final polypeptide complex batch.

Representative examples of protein production, purification, and bioanalytics of various polypeptide complex molecules are shown in FIGS. 13A-13C through FIGS. 24A-24B.

Polypeptide Complex Equilibrium Binding of Albumin Via ELISA

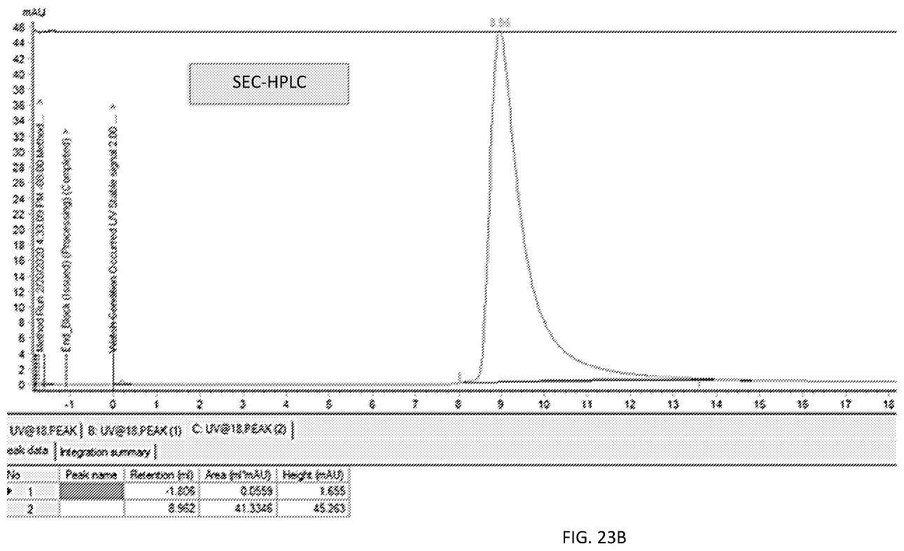

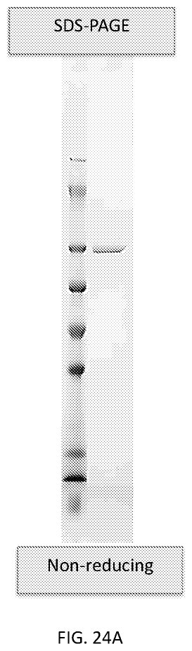

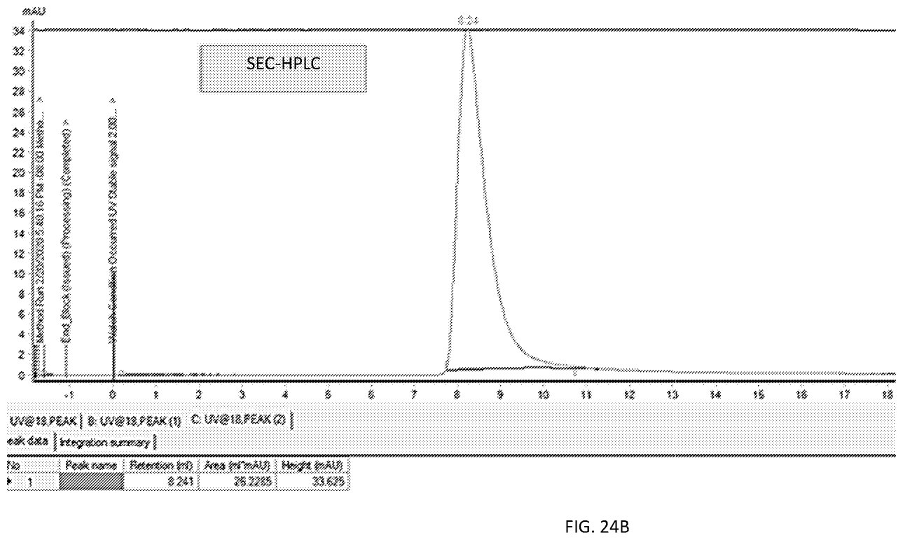

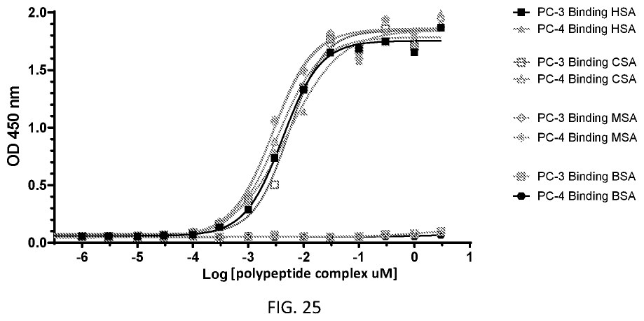

Anti-albumin single domain antibody (SDA) was tethered in tandem to the polypeptide complex mask attached to the core bispecific structure to form a complete crossover PK molecule of various formats (FIG. 12). In order to test the functional binding of the anti-albumin domain while tethered to the polypeptide complex molecule, polypeptide complex molecules were first tested for their ability to bind albumin of several species. FIG. 25 illustrates binding to bovine, mouse, cyno, and human albumin. Briefly, serum albumin from different species were coated directly on high binding ELISA plates, washed, blocked in non-fat dry milk, and washed again. polypeptide complex molecules were diluted in non-fat dry milk to the desired concentrations, added to the albumin coated plates, and washed. A secondary anti-histag HRP conjugate was used to detect bound polypeptide complex. After washing, plates were developed, stopped, and measured using standard ELISA techniques. OD450 nm signals were plotted against logarithmic polypeptide complex concentration. The concentration of polypeptide complex to achieve half maximal signal (EC50) was calculated in Graphpad Prism. Despite tethering the anti-albumin SDA to the TCR mask within the polypeptide complex molecule, potent albumin recognition was maintained.

Kinetic Binding of Polypeptide Complexes to Cognate Antigens Via BLI

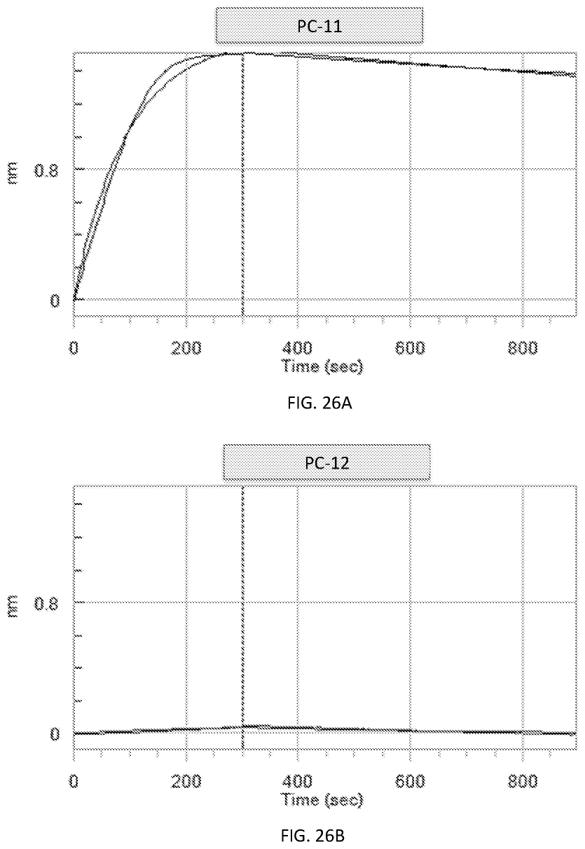

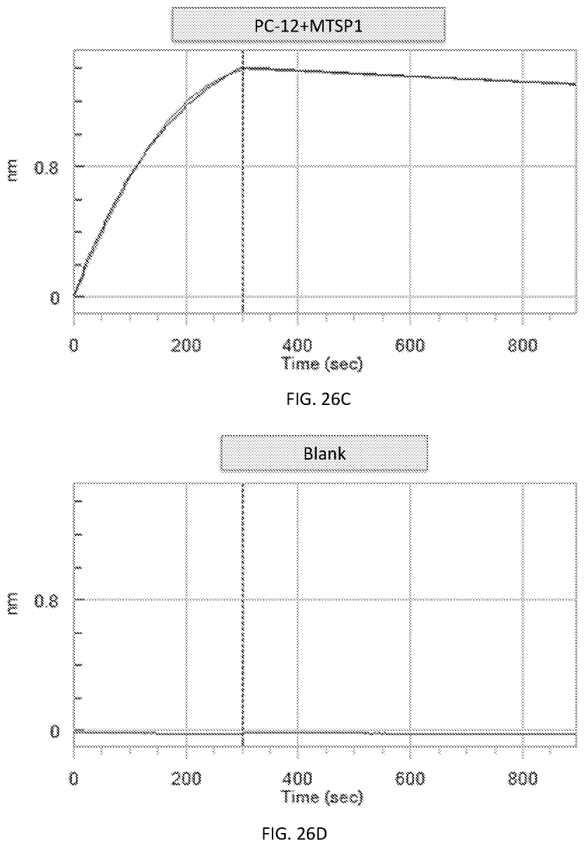

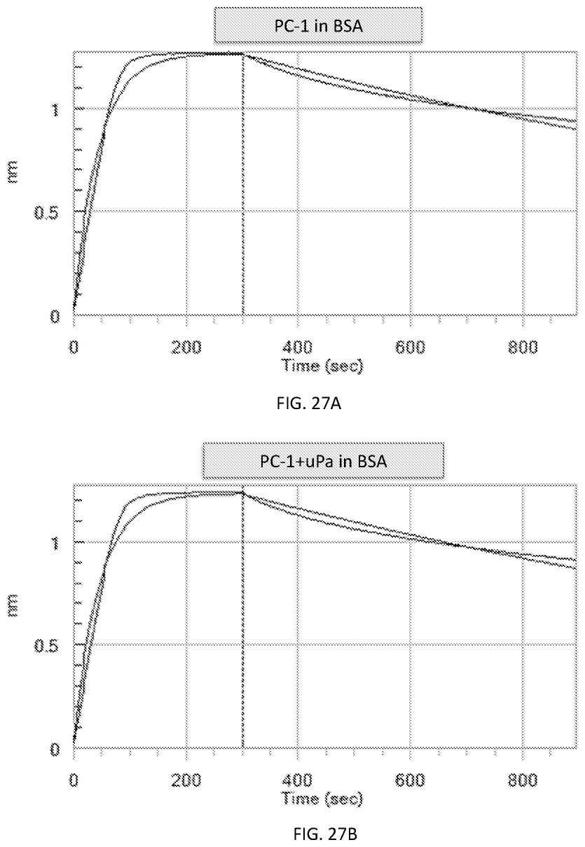

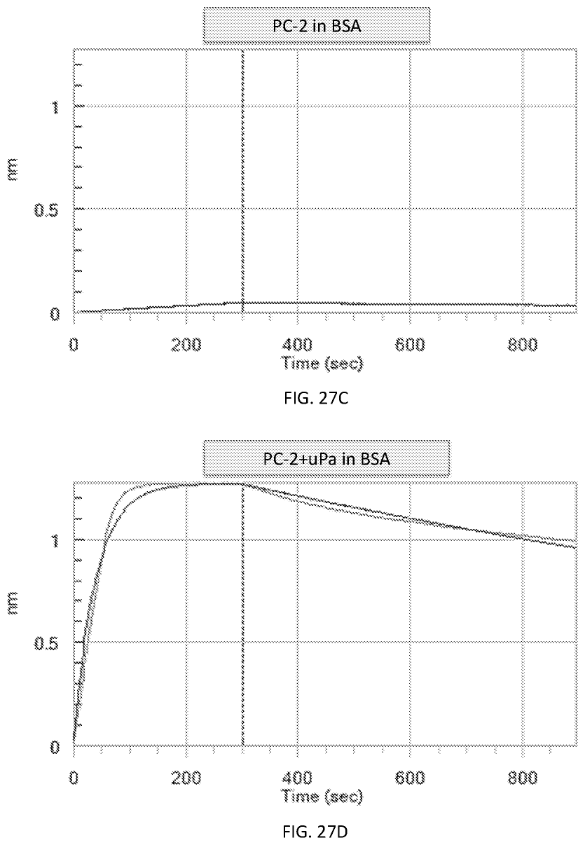

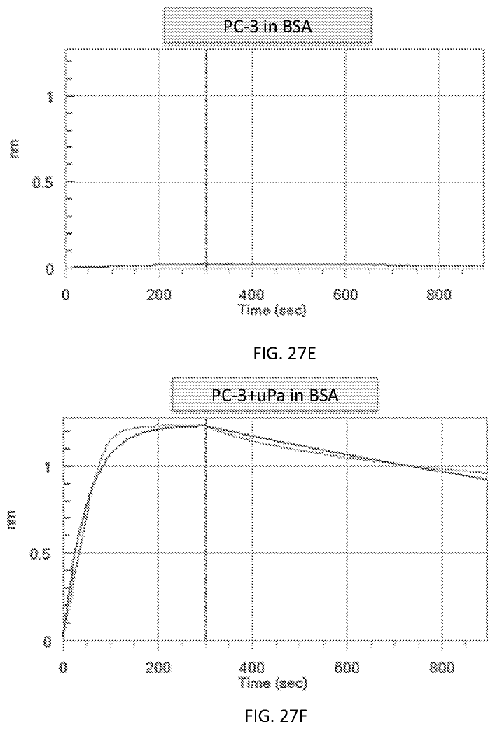

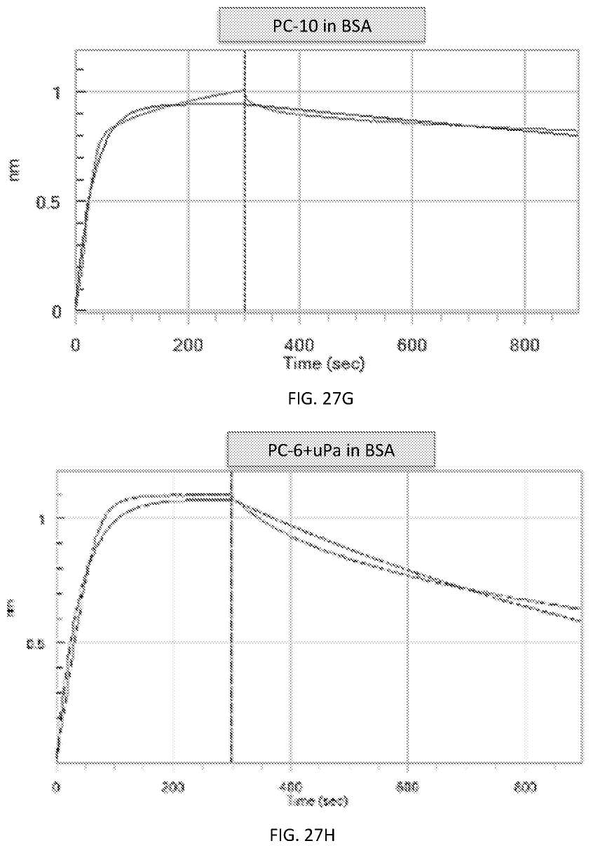

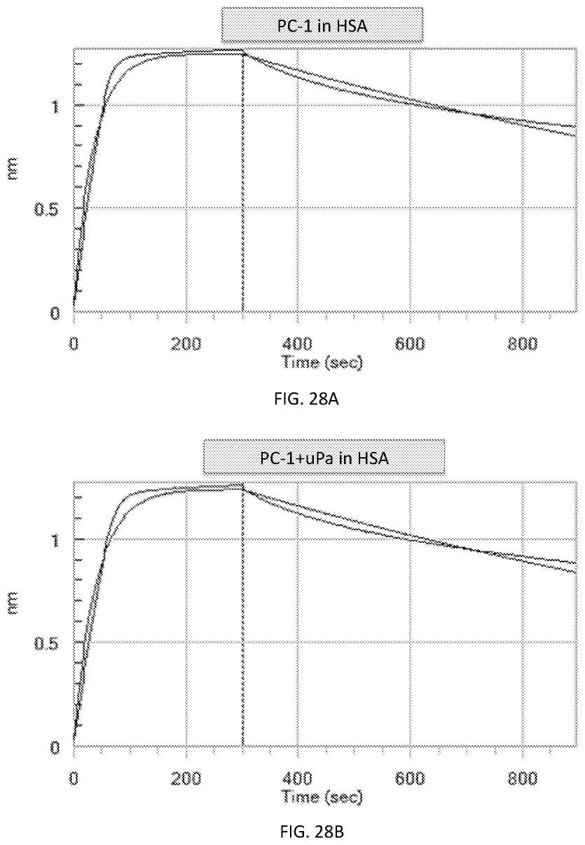

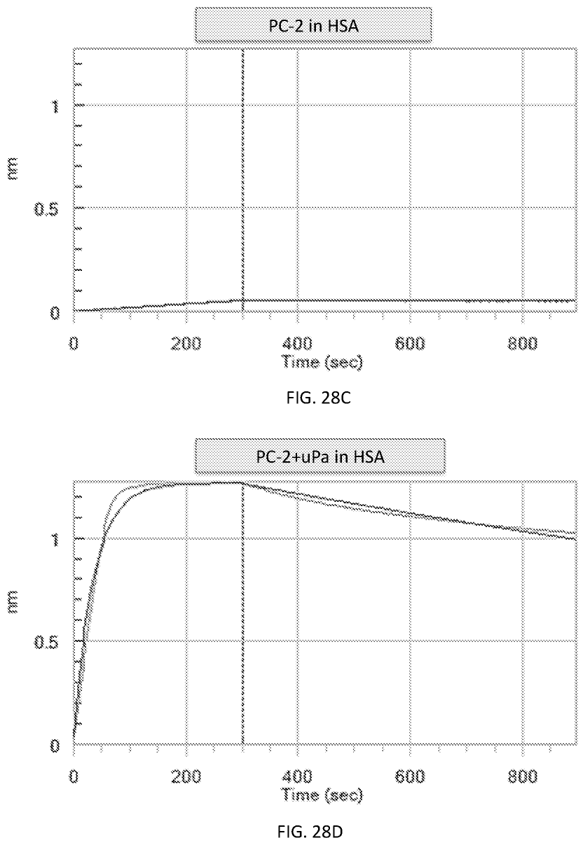

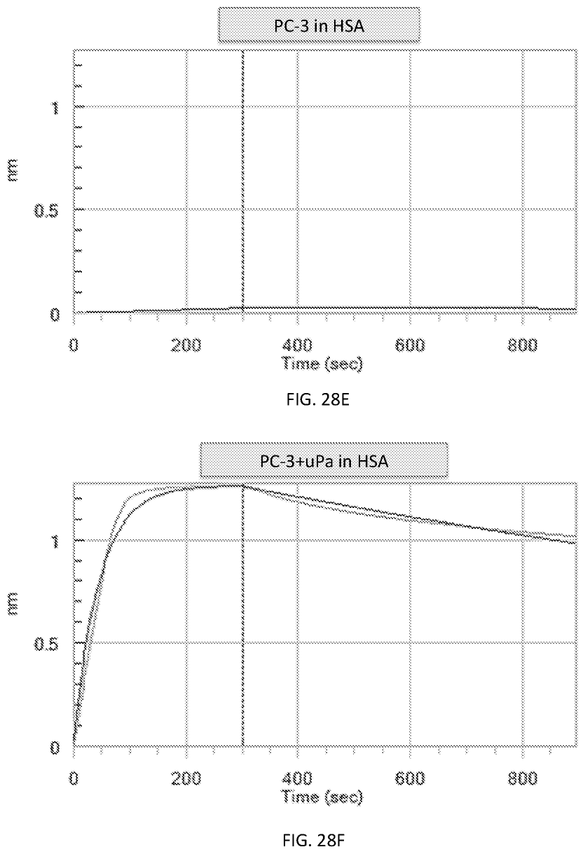

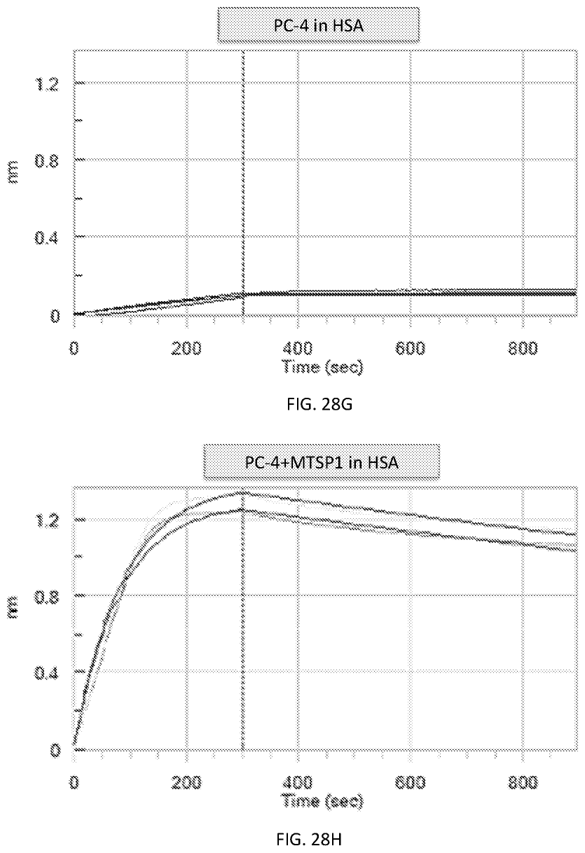

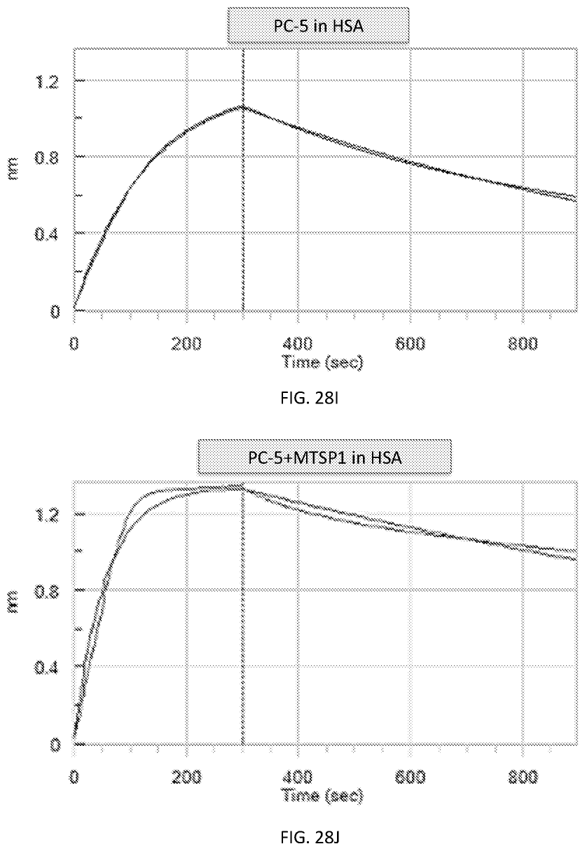

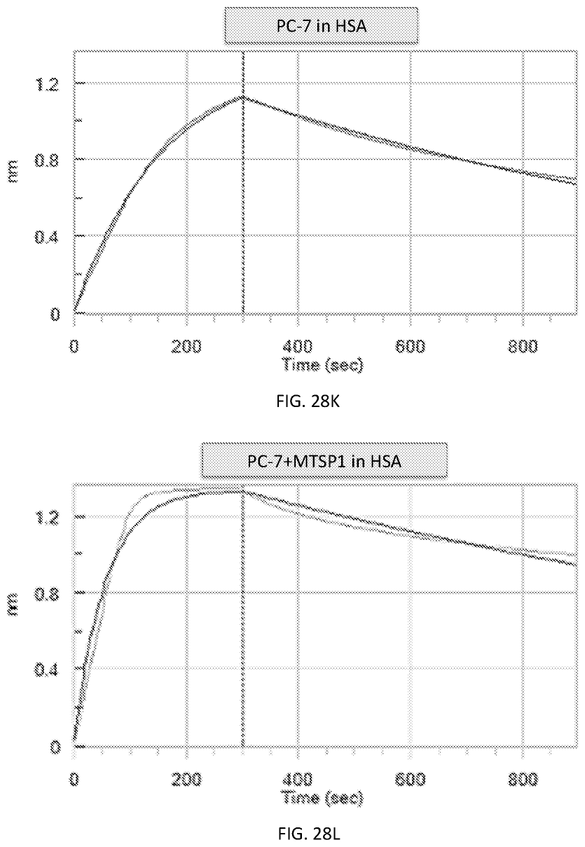

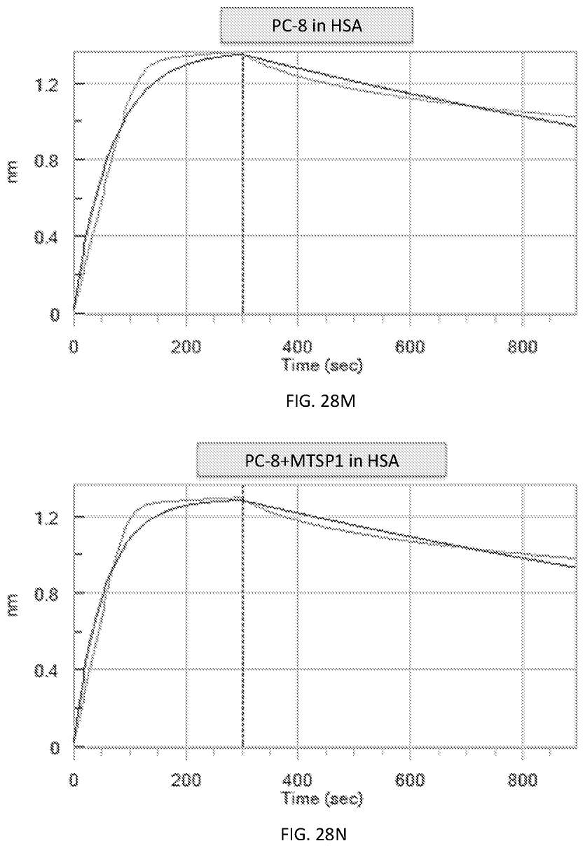

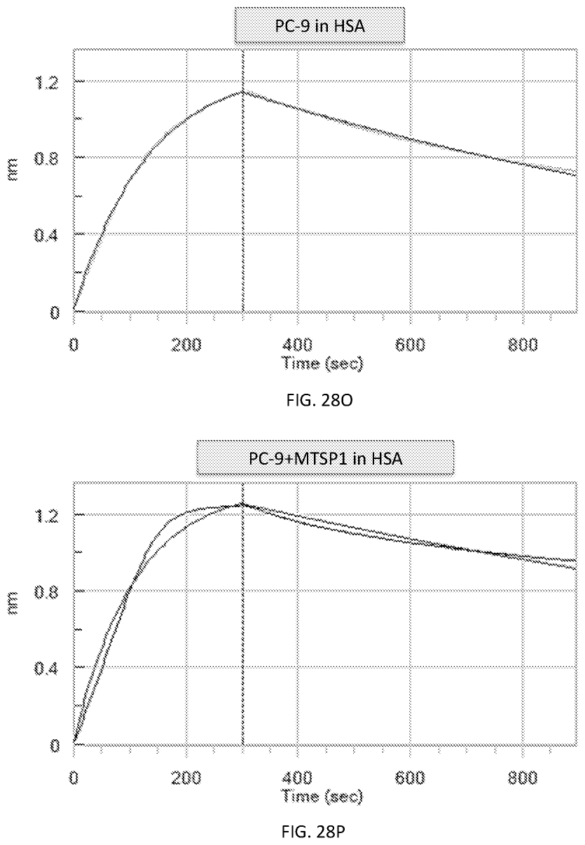

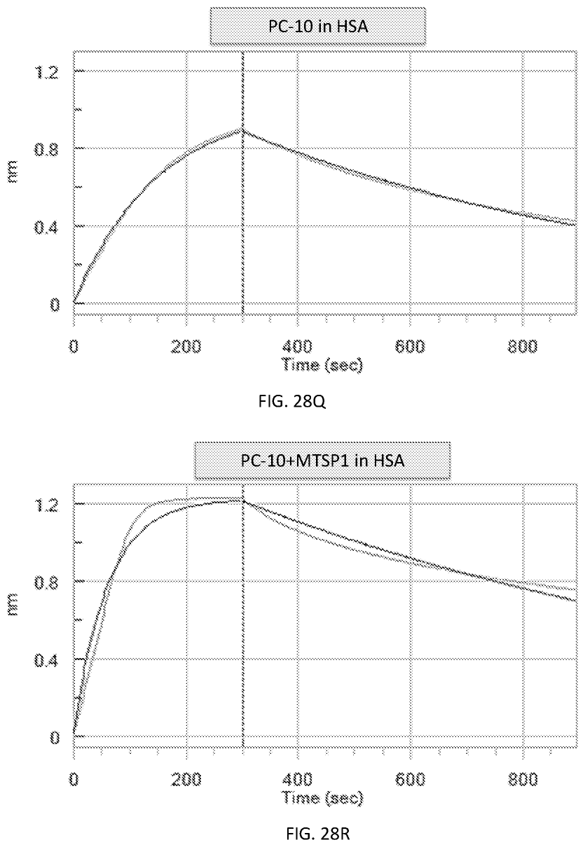

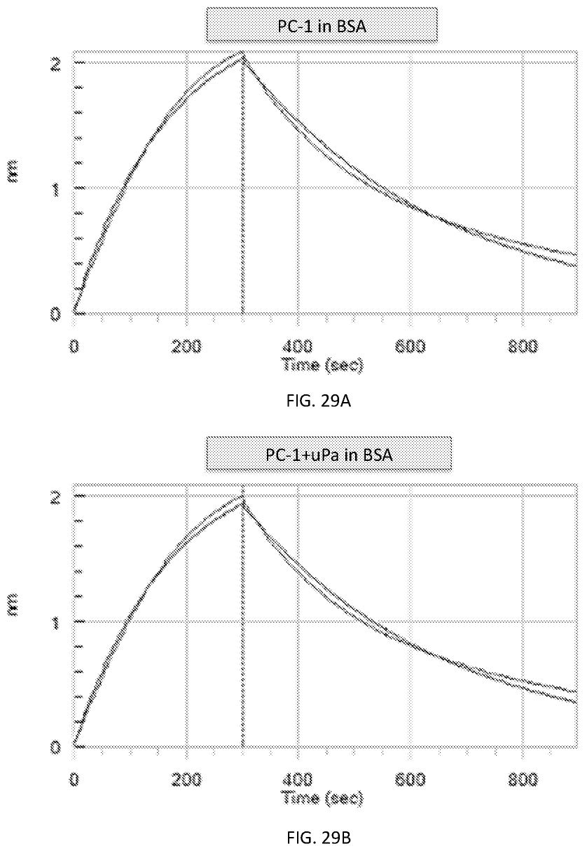

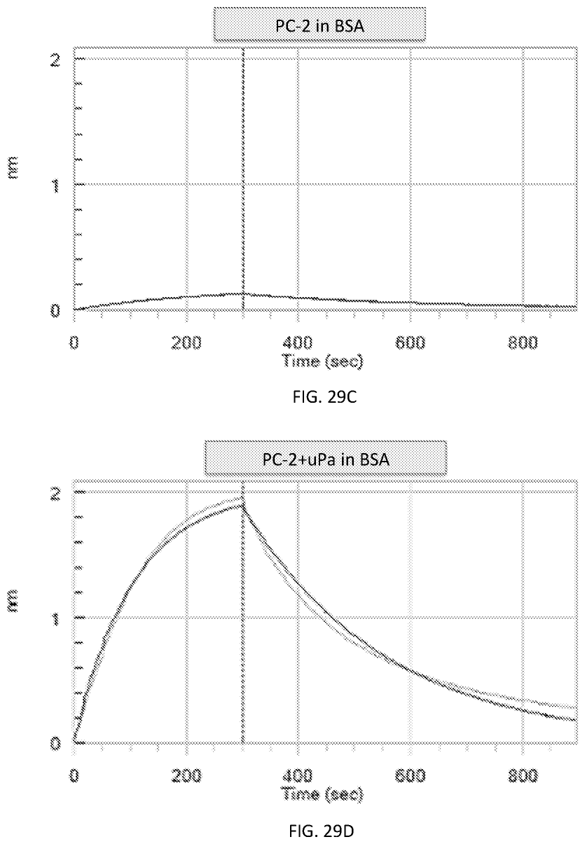

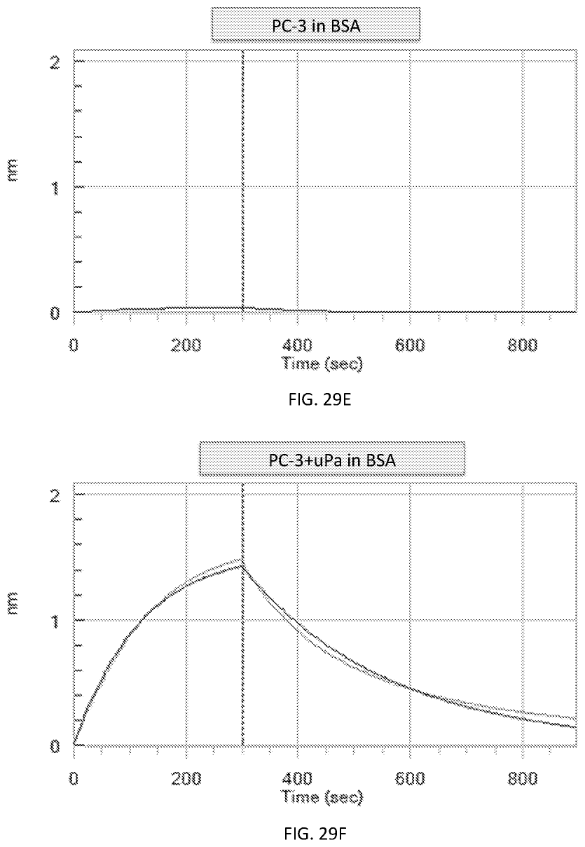

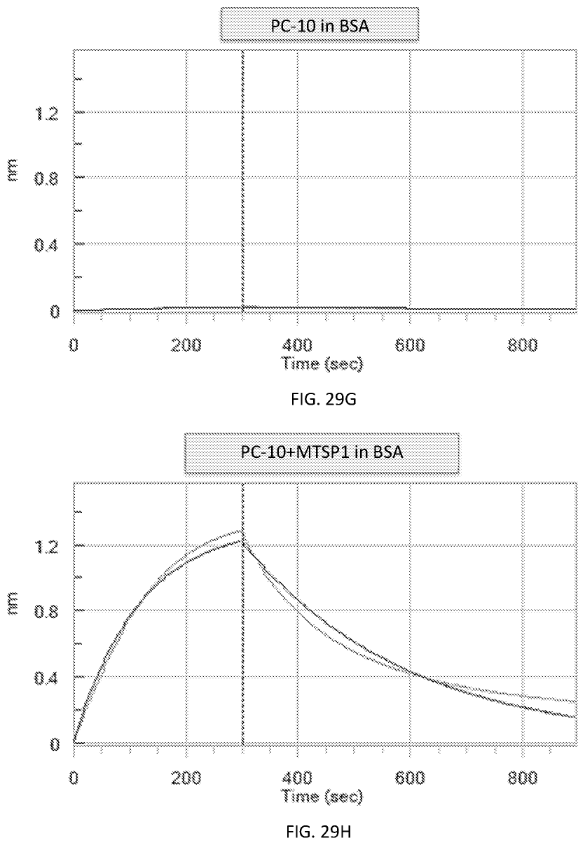

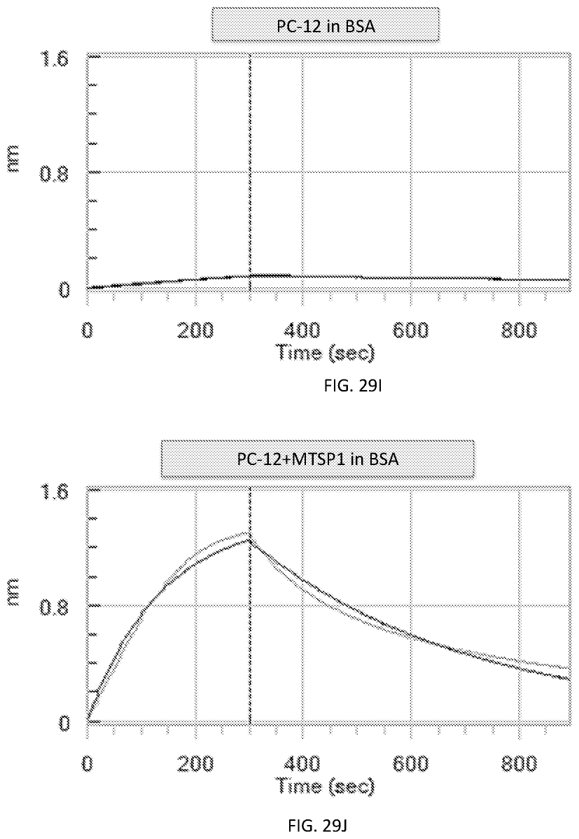

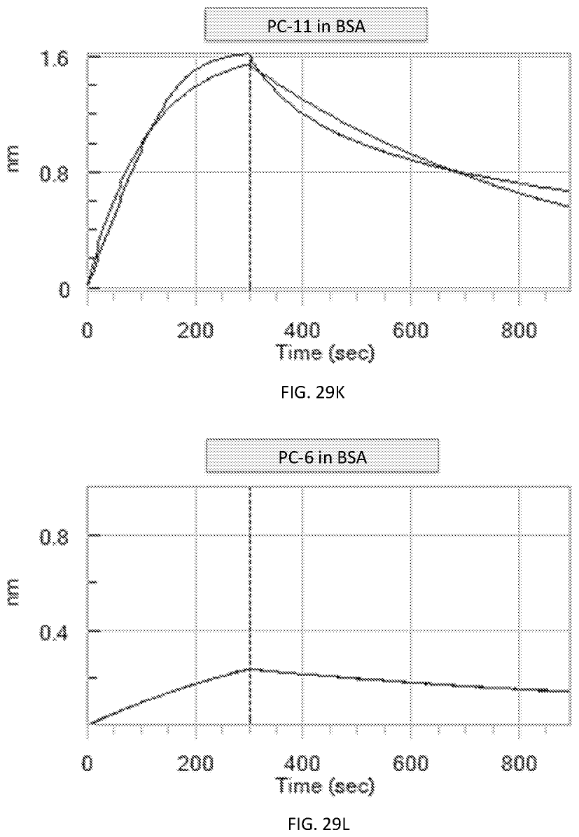

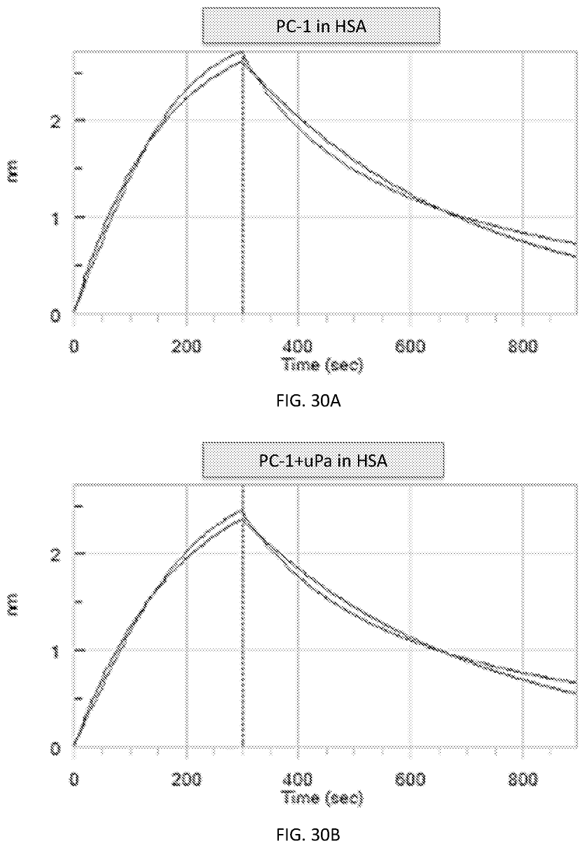

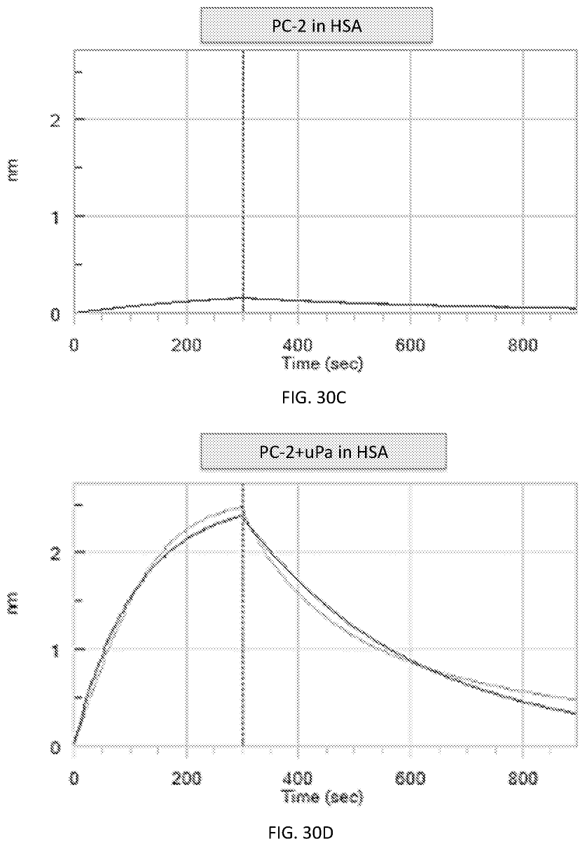

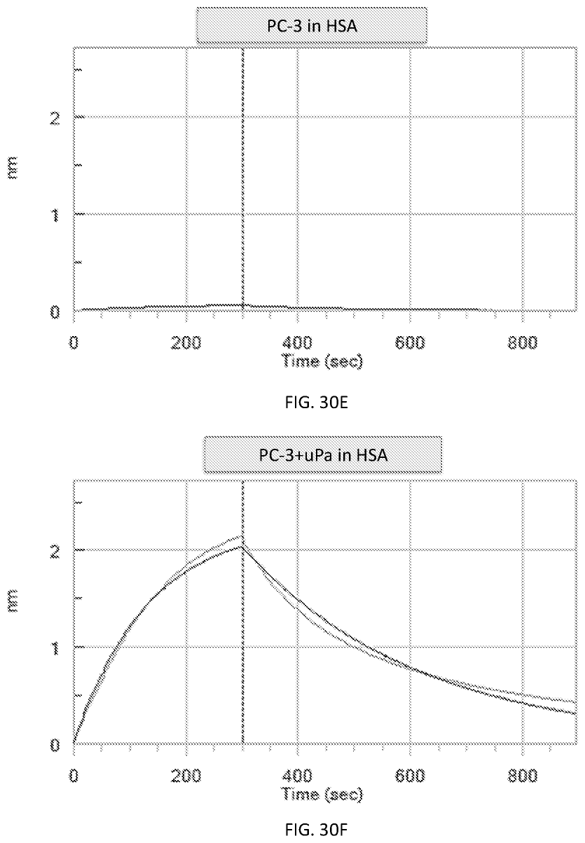

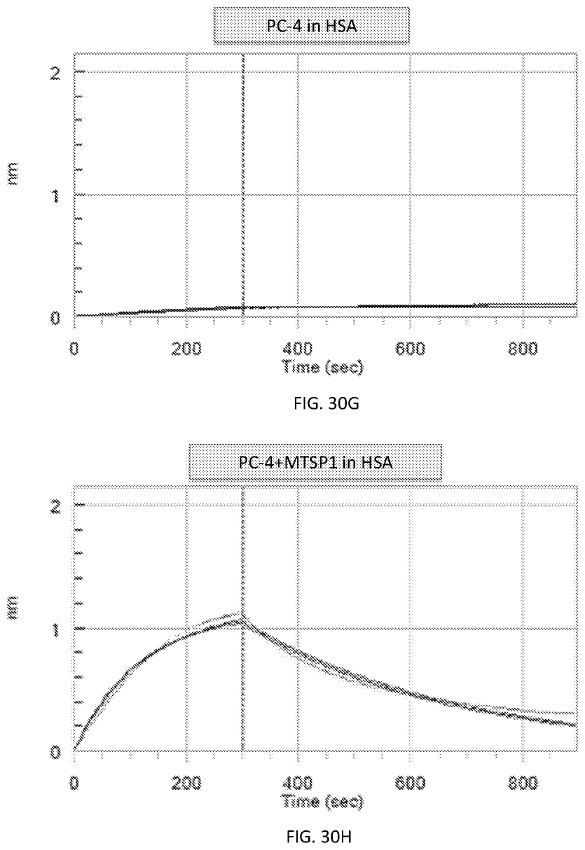

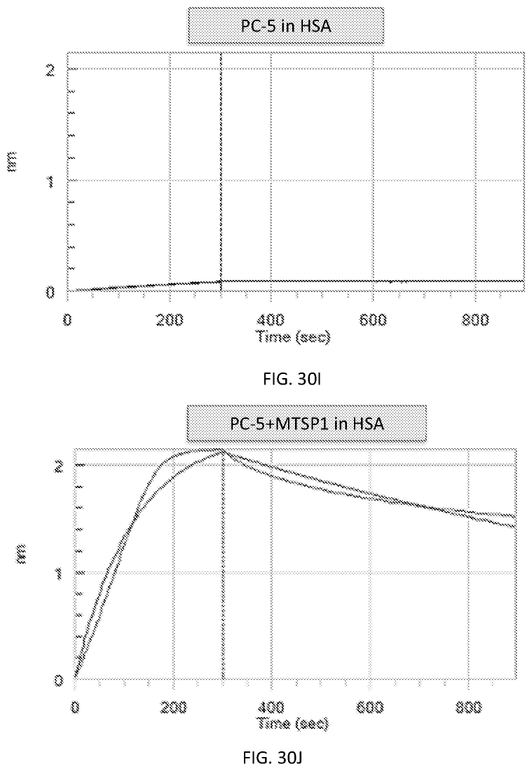

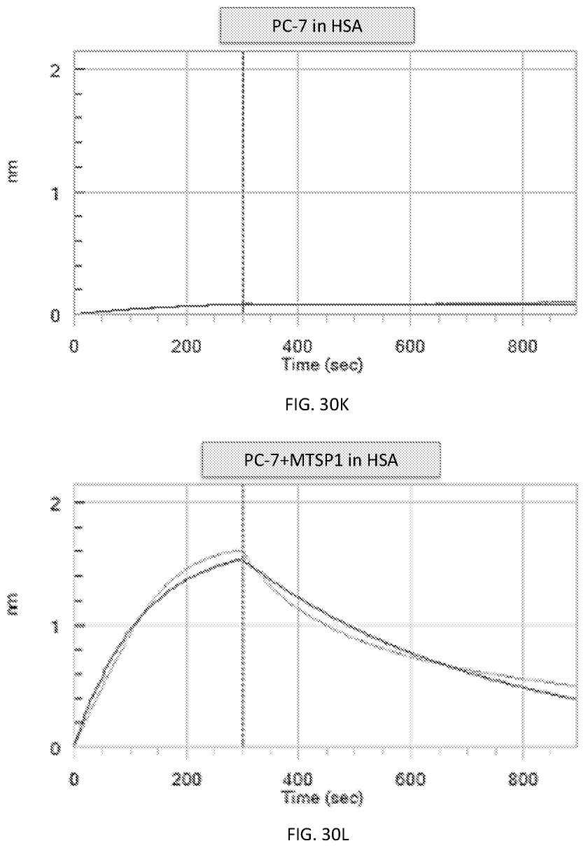

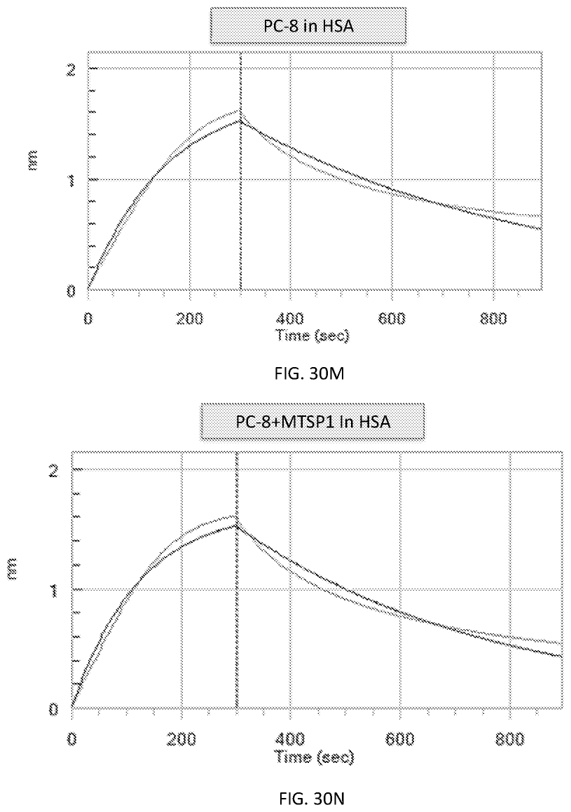

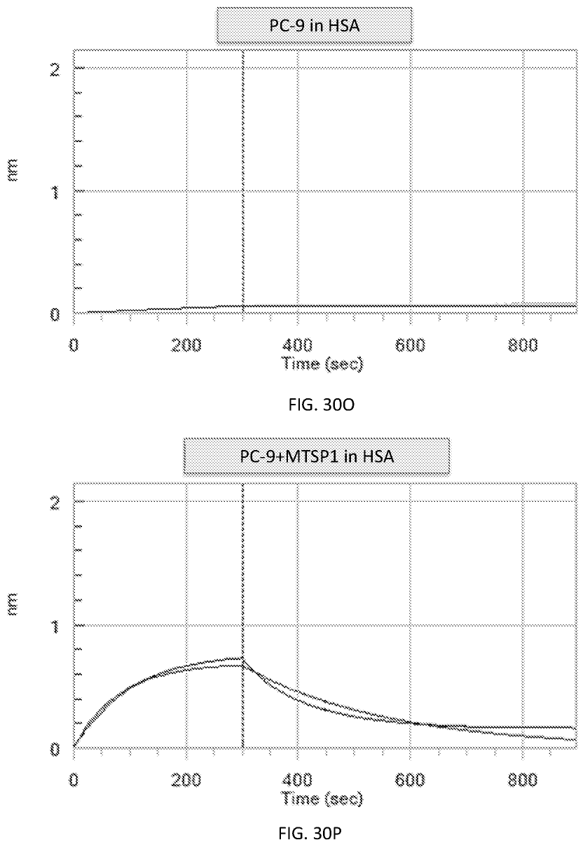

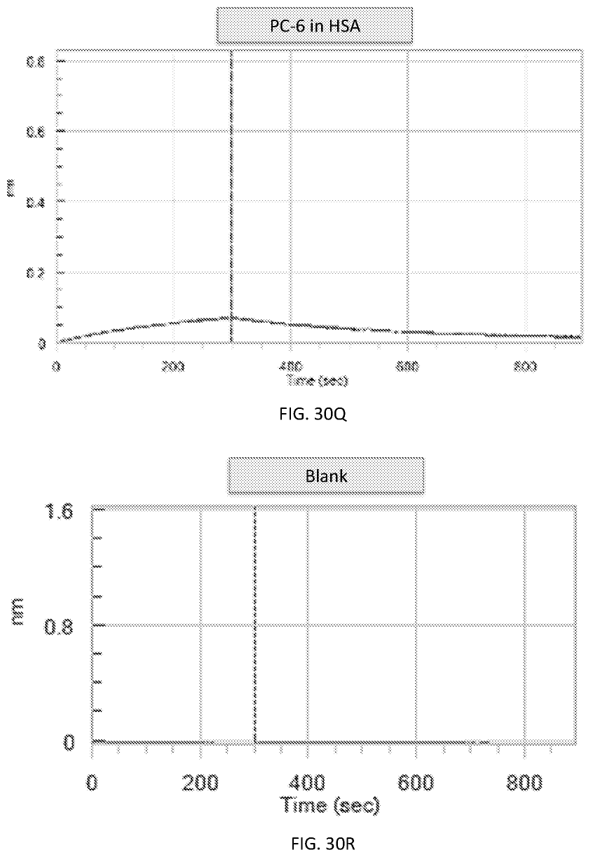

polypeptide complex molecules were evaluated for their ability to bind the cognate antigens, HER2 or EGFR, as well as CD3. polypeptide complex binding kinetics of HER2, EGFR, or CD3 were measured before and after protease treatment. Briefly, biotinylated antigen was loaded onto streptavidin coated biosensors, quenched in biocytin, and baselined in buffer. polypeptide complex molecules were treated with active matriptase (MTSP₁) or urokinase (uPa) where indicated. polypeptide complex molecules diluted in buffer were then associated onto the antigen loaded biosensors. Sensors were then transferred to buffer where polypeptide complex molecules then dissociated from the sensors. Association and dissociation rates were measured in real time using an OCTET RED96 instrument. Example sensorgrams are shown in FIGS. 26A-26D through FIGS. 30A-30R. polypeptide complexes contained the cleavable substrate, IGGLLSGRSDNH (SEQ ID NO: 111), between the peptide masks and the antigen binding domains. Kinetic binding data suggested that polypeptide complexes required treatment with protease in order to bind antigens. Furthermore, tethering the anti-albumin SDA to the peptide mask did not hinder the inhibition properties of peptide masks. Some polypeptide complexes were produced with non-cleavable linkers where the ISSGLLSGRSDNH sequence (SEQ ID NO: 42) was replaced with GlySer repeats, for example PC-6. Kinetic binding data suggested that these non-cleavable versions lacked the ability to bind antigens despite treatment with protease. The related data further supported the ability to use different linkers sequences between the peptide mask and the core antigen binding domains of the polypeptide complex without giving up masking efficiency. In some instances polypeptide complex binding was performed in the presence of human albumin to test if occupation of the albumin binding domain with human albumin influenced masking efficiency. The concentration of human albumin used was at a level expected to saturate the polypeptide complex albumin binding site. polypeptide complex masking efficiency was not significantly influenced by saturation with human albumin buffer indicated by the similar binding kinetics using either bovine or human albumin buffers.

Polypeptide Complex Equilibrium Binding To Cognate Antigens by ELISA

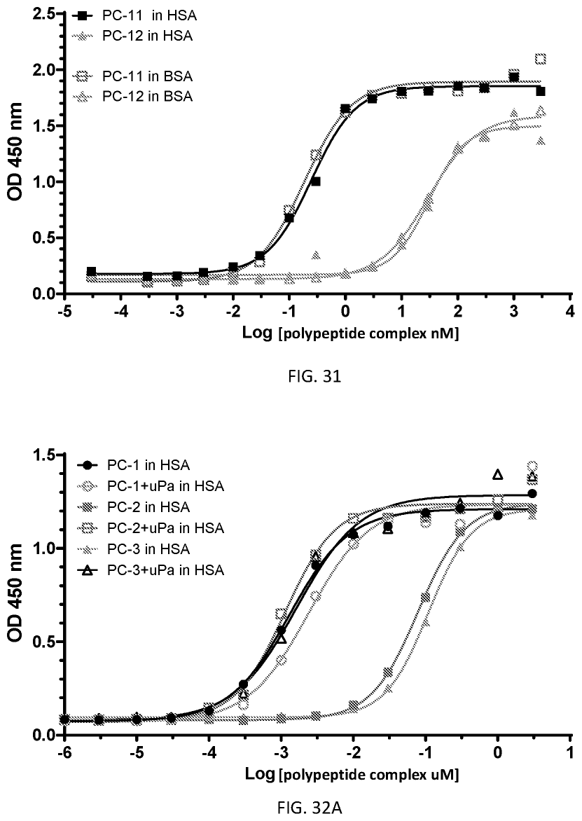

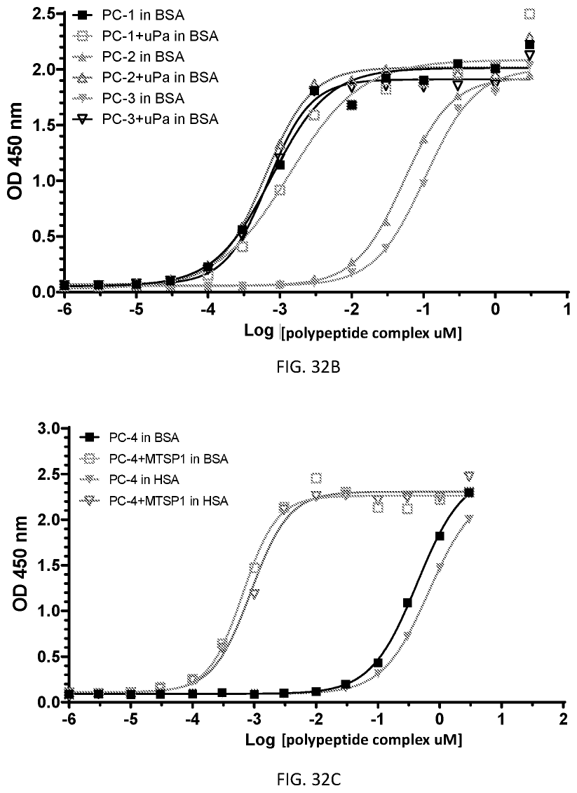

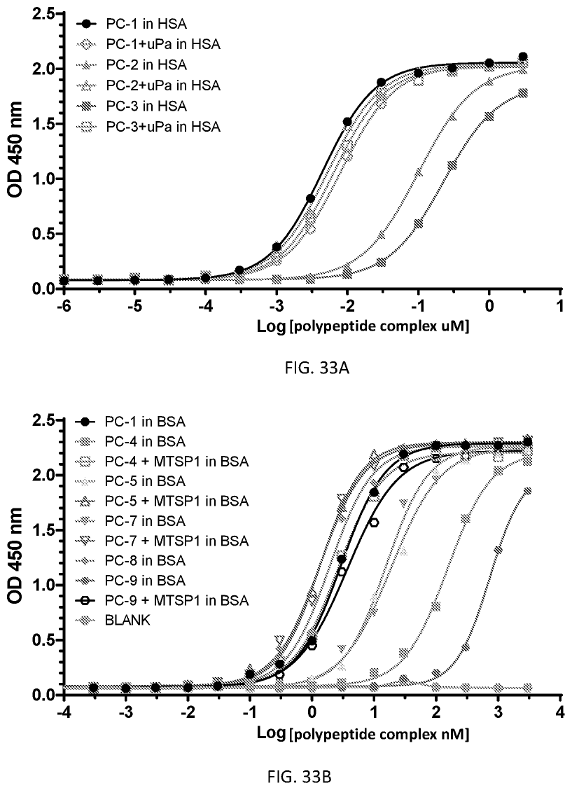

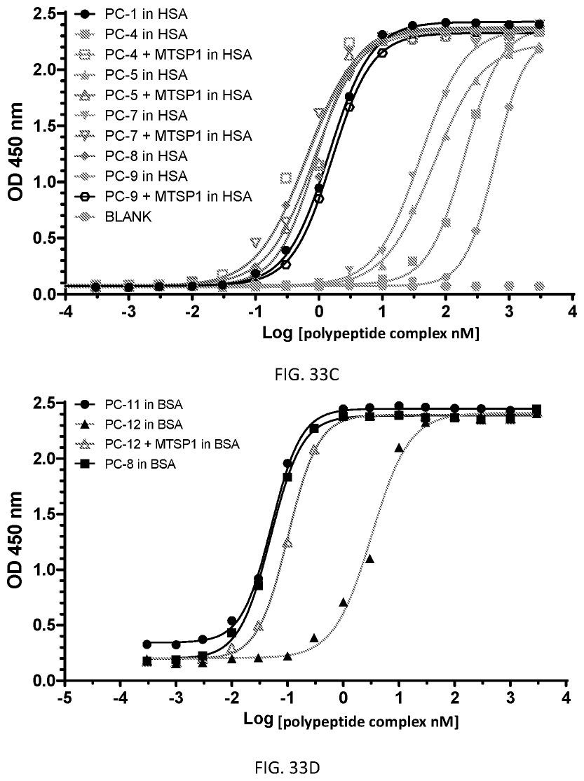

Polypeptide complexes were also characterized for their ability to recognize cognate antigens in ELISA based binding experiments. In some instances the polypeptide complexes were treated with protease where indicated. Briefly, biotinylated antigens were captured on neutravidin coated plates followed by the addition of titrated polypeptide complex in bovine or human albumin buffer. Plates were then incubated for a short time followed by a wash. A secondary anti-human HRP conjugate antibody was used to detect bound polypeptide complex to the plate. Concentrations of polypeptide complexes required to achieve half maximal ELISA signal (EC50) were calculated in Graphpad Prism. Example ELISA data shown in FIG. 31 through FIGS. 33A-33D demonstrated hindered ability of masked polypeptide complex to bind cognate antigens independent of albumin occupying the SDA binding site. polypeptide complex masking efficiency was not significantly influenced by use of bovine versus human albumin buffer, indicated by the similar EC50s with either of the two buffers. Human albumin buffer was expected to saturate the polypeptide complex albumin binding site with human albumin. In contrast, polypeptide complexes do not bind bovine albumin and are therefore expected to have unoccupied SDA sites during experiments using bovine albumin buffer. In addition, polypeptide complex binding signals are restored to low nanomolar levels after treatment with protease regardless of bovine or human albumin buffer.

Ternary Complex Formation of Polypeptide Complexes on the Surface of Human T Cells Via Flow Cytometry

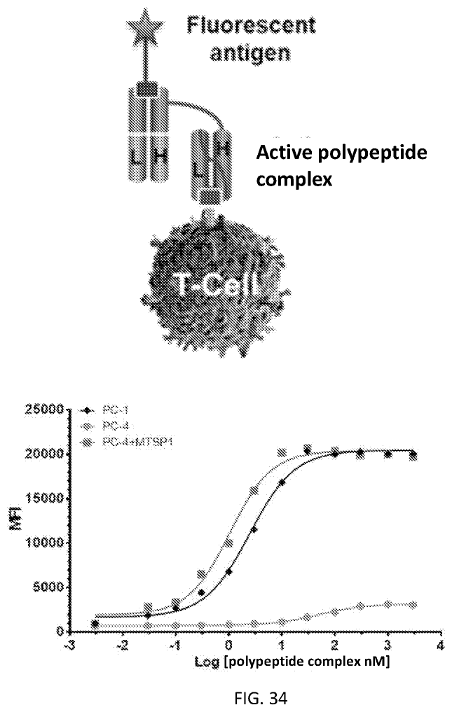

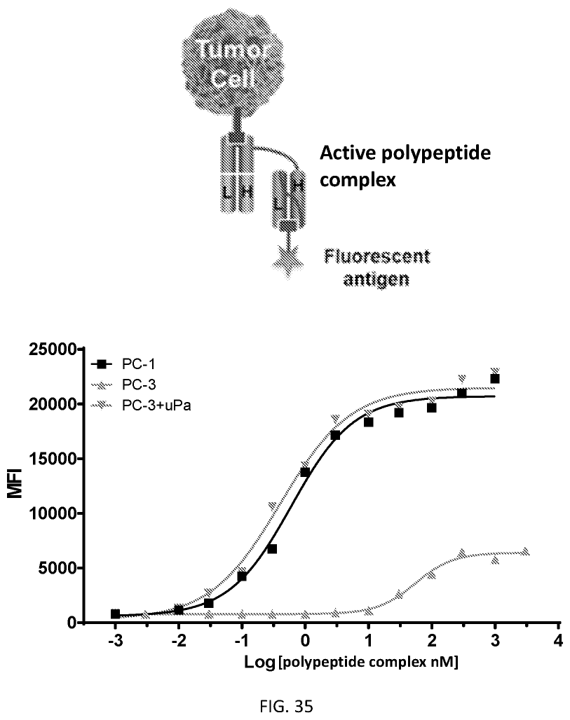

Polypeptide complexes were further characterized for their ability to form a ternary complex on the surface of human cells via binding of cellular CD3 or EGFR and subsequently stained using fluorescently labeled EGFR or CD3 tetramer (FIGS. 34-35). Cellular fluorescence measured by flow cytometry was indicative of complex formation between cell and antigen tetramer where the polypeptide complex acts as the bridging molecule.

Briefly, 100,000 T cells per well were distributed in a 96 well plate, washed cold, followed by incubation with the indicated concentration of non-masked polypeptide complex, polypeptide complexes, or protease treated polypeptide complexes in human albumin buffer. Cells were incubated cold for a few hours, then washed with cold buffer, followed by a short incubation with cold HER2 or EGFR tetramer formed using fluorescently labeled streptavidin. Cells were washed cold, resuspended in cold buffer, and run on a Novocyte flow cytometer. Scattering signals were gated in the typical fashion to exclude debris of incorrect cellular shape and size. Mean fluorescent intensity was normalized, plotted against polypeptide complex concentration, and the concentration of polypeptide complex required to achieve 50% maximal signal (EC50) was calculated in Graphpad Prism. In general, treatment of polypeptide complex molecules with protease enabled potent ternary complex formation equivalent to the non-masked TCR bispecific controls with low nanomolar EC50s. In contrast, minimal ternary complex formation could be detected using polypeptide complex molecules at the highest concentrations tested. Data suggests functional polypeptide complex ternary complex formation requires specific protease activity.

Polypeptide Complex Mediated Tumor Cytotoxicity and T Cell Activation

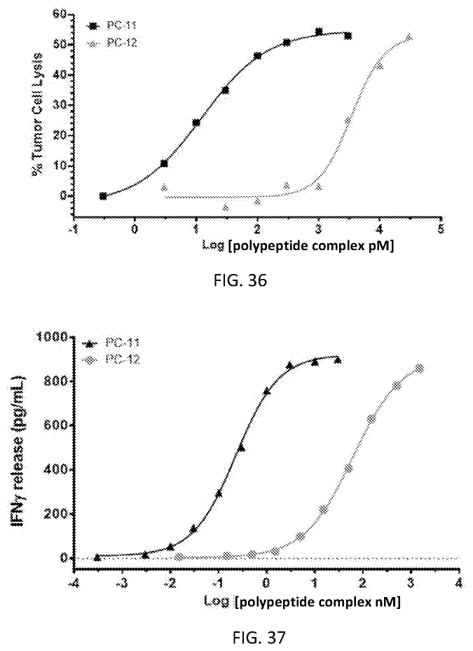

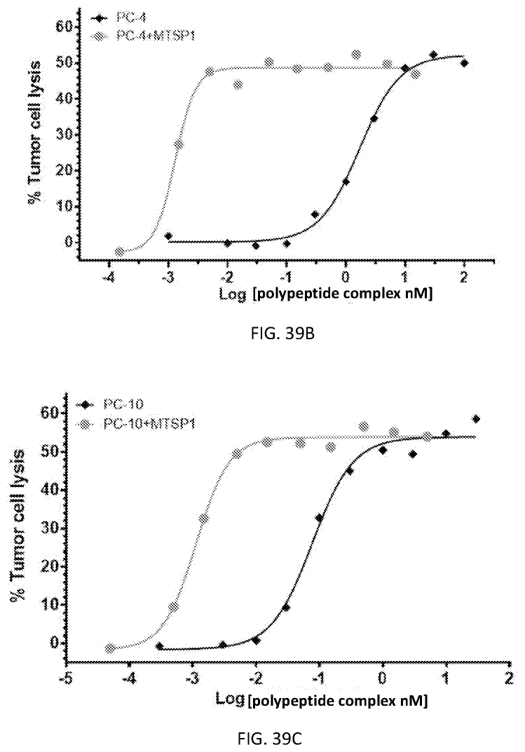

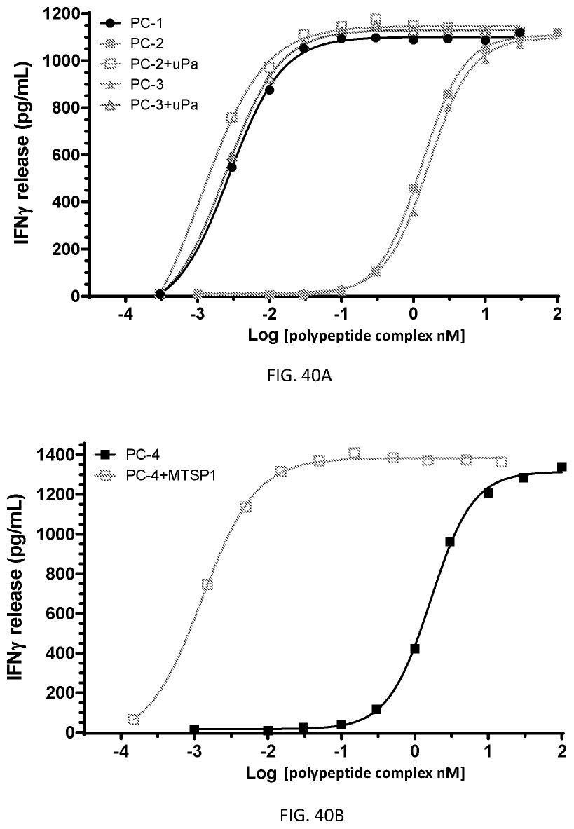

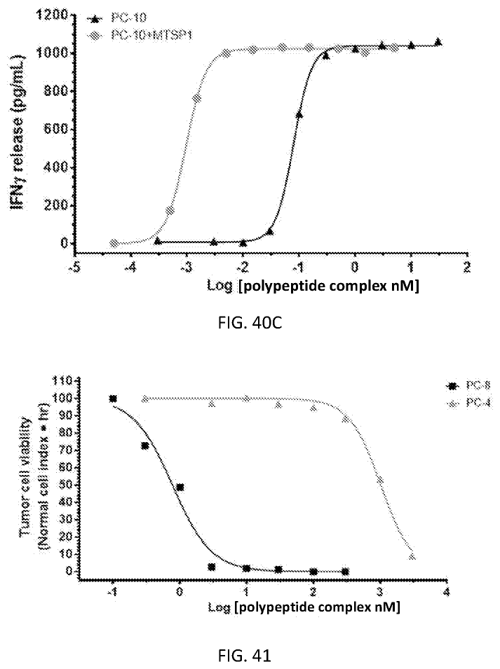

Polypeptide complexes were evaluated in functional in vitro tumor cell killing and related T cell activation studies using the HER2 positive HCC1569 (FIGS. 36-38) and EGFR positive HCT116 (FIGS. 39A-39C through FIG. 41) tumor cell lines using bovine or human serum supplemented medium. Briefly, antigen positive tumor cell lines were seeded onto 96 well tissue culture treated flat bottom plates and allowed to adhere overnight. The following day, culture medium and nonadherent cells were removed and replaced with fresh medium containing titrated polypeptide complex at concentrations indicated. In some instances, polypeptide complexes were treated with protease prior to addition to target cells. CD8+ T cells were then added in an effector cell:target cell ratio of 2:1 relative to tumor cell seeding density. Adherent tumor cells, CD8+ T cells, and polypeptide complexes were co-cultured for 48 hours. Plates were gently spun down to collect cells at the bottom of the plate and the clarified supernatants collected. Lactate dehydrogenase (LDH) dependent cytotoxicity was measured using the Promega LDH-Glo assay kit. Interferon-gamma (IFNγ) released by activated T cells was measured in the supernatants using an Invitrogen ELISA kit. LDH or IFNγ signals were plotted against concentration of polypeptide complexes in order to calculate the concentration of polypeptide complex required to achieve 50% maximal signal (EC50). EC50s were calculated using Graphpad Prism.

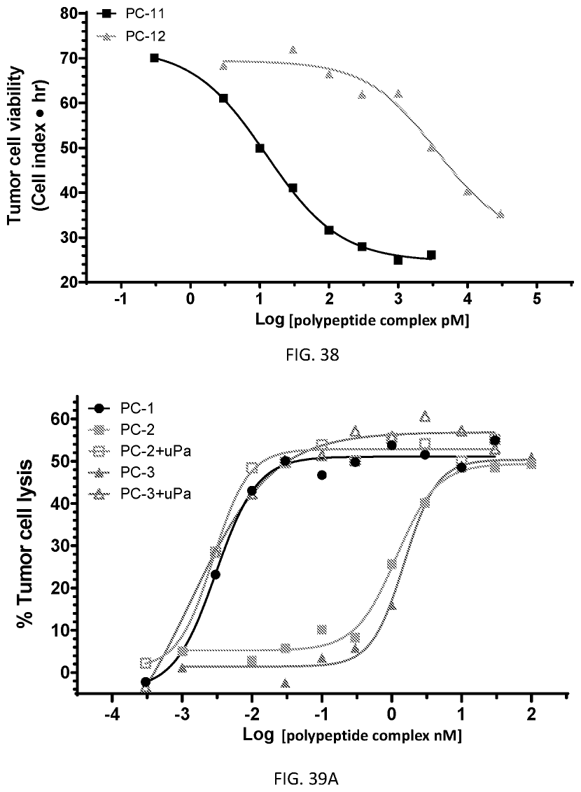

In parallel, tumor cell killing was measured using a real time cell analyzer from Acea Biosciences that relies on sensor impedance measurements (cell index) that increased as tumor cells adhere, spread, and expand on the surface of the sensor. Likewise, as the tumor cells were killed the impedance decreased. Int the example assays (FIG. 38 and FIG. 41), 25,000 tumor cells were added per well and allowed to adhere overnight. The following day polypeptide complexes titrated in human serum supplemented medium along with 75,000 CD8+ T cells were added to the wells. Cell index measurements were taken every 10 minutes for an additional 96 hours. The cell index times number of hours (tumor cell growth kinetics) was then plotted versus concentration of polypeptide complex where the concentration required to reduce the tumor growth 50% (IC50) was calculated using Graphpad Prism. Similar to the LDH Glo assay, masked polypeptide complex molecules had poor tumor killing efficiency while the non-masked and protease treated polypeptide complexes were potent with IC50s in the 10 pM range targeting HER2 and 1 pM range targeting EGFR.

### Example 5

### In Vivo Safety and Efficacy of Polypeptide Complex Constructs

Polypeptide Complex Pharmacokinetics in Balb/c Mice

Polypeptide complex pharmacokinetics were determined in male 6-8 week old Balb/c mice. Briefly, animals were assessed for their general health by a member of veterinary staff or other designated personnel upon arrival and allowed to acclimate for at least 3 days before study commencement. Animals were group housed during acclimation and throughout the study. The animal room environment was controlled according to facility operation with temperature between 20 to 26° C. and relative humidity between 30 and 70%. Lighting was controlled on a 12 hour light dark cycle. Animals were fed certified pellet diet (Certified Rodent Diet #5002, LabDiet). Purified water (reverse osmosis) was provided to the animals ad libitum. Periodic analysis of water quality was performed.

Concentrated test articles were diluted to appropriate dosing volume in sterile phosphate buffered saline and administered intravenously via tail vein at 10 mL/kg. Dose volume was determined individually by body weight obtained immediately prior to dosing for each animal

Blood samples were collected before and after dosing at the indicated time points. For each timepoint a subset of 3 mice were euthanized by carbon dioxide inhalation. Following confirmation of death, blood samples were collected through the inferior vena cava using a syringe. The blood samples were placed in pre-labeled EDTA tubes and processed to plasma. The blood samples were centrifuged cold at 3000×g for 10 min to separate cells from plasma. The plasma supernatant was harvested and stored frozen prior to analysis.

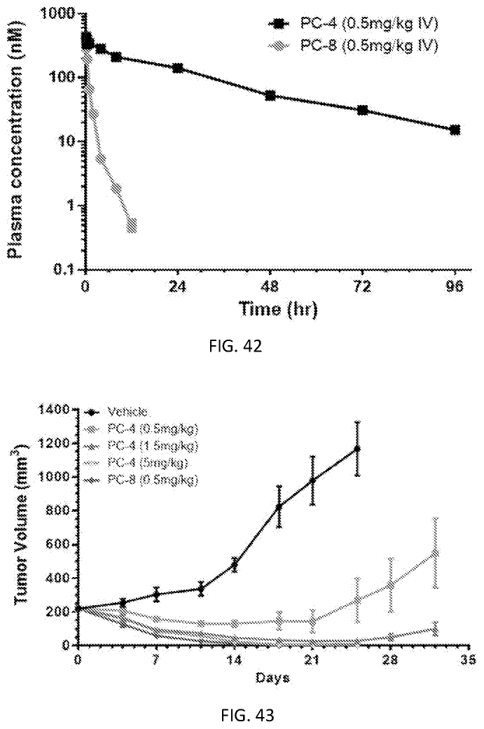

The concentration of polypeptide complex in mouse plasma samples was determined by ELISA. Briefly, anti-histag capture antibody was coated directly on ELISA plates. Standard dilutions of polypeptide complex in mouse serum were used to generate a calibration curve to which animal PK test samples could be compared. Standards and test samples were added to the plate and incubated cold overnight. Several different dilutions of test samples were used to make sure signals landed within appropriate dynamic range of the standard curve. Plates were washed and incubated with an anti-human HRP detection antibody for a brief time. Plates were washed, developed, and stopped using standard ELISA techniques. Standard curves plotting absorbance at 450 nm versus known polypeptide complex concentration were used to calculate the concentration of unknown test articles in each mouse PK plasma sample. Concentration of polypeptide complex were plotted versus time and fit to a standard two stage distribution and elimination pharmacokinetic model. The calculated pharmacokinetics and parameters for PC-8 and PC-4 from Balb/c mice are shown in FIG. 42 and TABLE 6.

TABLE 5
Pharmacokinetics and parameters for PC-8 and PC-4.

| PC-8 Mouse PK | | | PC-4 Mouse PK | | |
|---|---|---|---|---|---|
| (0.5 mg/kg IV bolus) | | | (0.5 mg/kg IV bolus) | | |
| T_{MAX} | 0.0 | hr | T_{MAX} | 0.0 | hr |
| C_{MAX} | 512.78 | nM | C_{MAX} | 361.78 | nM |
| t_{1/2} | 1.86 | hr | t_{1/2} | 26.73 | hr |
| Vd | 0.38 | mL | Vd | 0.42 | mL |
| VSS | 5.06 | mL | VSS | 0.80 | mL |
| CL | 4.72 | mL/hr/kg | CL | 0.36 | mL/hr/kg |
| BW | 0.03 | kg | BW | 0.03 | kg |

Polypeptide Complex In Vivo Efficacy in a NCG Mouse HCT116 Xenograft Model

Mice

Female NCG mice (NOD-Prkdc^{em26Cd52}Il2rg^{em26Cd22}/NjuCrl, Charles River) were nine weeks old with a body weight (BW) range of 20.8 to 28.3 grams on Day 1 of the study. The animals were fed ad libitum water (reverse osmosis, 1 ppm Cl), and NIH 31 Modified and Irradiated Lab Diet® consisting of 18.0% crude protein, 5.0% crude fat, and 5.0% crude fiber and were supplemented with Diet Gel during the in-life portion of the study. The mice were housed on irradiated Enrich-o'cobS™ Laboratory Animal Bedding in static microisolators on a 12-hour light cycle at 20-22° C. (68-72° F.) and 40-60% humidity. Charles River Discovery Services North Carolina (CR Discovery Services) specifically complies with the recommendations of the Guide for Care and Use of Laboratory Animals with respect to restraint, husbandry, surgical procedures, feed and fluid regulation, and veterinary care. The animal care and use program at CR Discovery Services is accredited by the Association for Assessment and Accreditation of Laboratory Animal Care International (AAALAC), which assures compliance with accepted standards for the care and use of laboratory animals

PBMCs

Human peripheral blood mononuclear cells (hPBMCs) were provided by Charles River Discovery Services NC. On the same day as and just prior to tumor cell implant, 2×10⁷ hPBMCs were engrafted intravenously via tail vein in a fixed volume of 0.2 mL/animal.

Tumor Cell Culture

HCT116 human colorectal carcinoma cells were maintained as exponentially growing cultures in RPMI-1640 medium containing 100 units/mL penicillin G sodium, 100 μg/mL streptomycin sulfate, 25 μg/mL gentamicin, 10% fetal bovine serum, and 2 mM glutamine. The tumor cells were grown in tissue culture flasks in a humidified incubator at 37° C., in an atmosphere of 5% CO₂ and 95% air.

In Vivo Implantation and Tumor Measurement

On the day of tumor implant, HCT116 cells were harvested during exponential growth and resuspended in 50% Matrigel™ (BD Biosciences) in phosphate buffered saline (PBS) to deliver 5×106 cells (in a 0.1 mL suspension) as subcutaneous xenograft implants in the right flank of each test animal Tumors were monitored as their volumes approached the target range of 100 to 300 mm3. Fourteen days later, designated as Day 1 of the study, animals were sorted by tumor volume into seven groups (n=10) with individual tumor volumes ranging from 172 to 288 mm3 and group mean tumor volumes between 220 and 221 mm3. Tumors were measured in two dimensions using calipers, and volume was calculated using the formula:

$$Tumor Volume ( {mm}^{3} ) = \frac{{w}^{2} \times l}{2}$$

where w=width and 1=length, in mm, of the tumor. Tumor weight was estimated based on the assumption that 1 mg is equivalent to 1 mm3 of tumor volume.

Therapeutic Agents

On each day of dosing vials containing stock solutions of PC-4 were gently mixed and diluted with sterile PBS to obtain dosing solutions at 0.5, 0.15, and 0.05 mg/mL, which delivered 5, 1.5, and 0.5 mg/kg, respectively, when administered in a volume of 10 mL/kg (0.2 mL per 20 g mouse), adjusted to the body weight (BW) of each animal On each day of dosing vials containing stock solutions of PC-8 were gently mixed and diluted with sterile PBS to obtain dosing solutions at 0.05 mg/mL, which delivered 0.5 mg/kg, respectively, when administered in a volume of 10 mL/kg (0.2 mL per 20 g mouse), adjusted to the body weight (BW) of each animal Dosing started when tumors reached >200mm³. Therapeutic agents were administered at the dose indicated daily for a total of 10 doses per group.

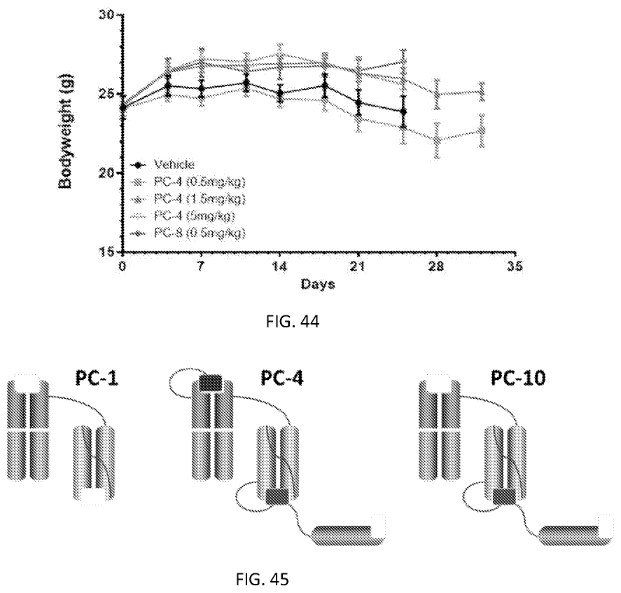

Tumor volume and body weight over time are shown in FIGS. 43-44.

Polypeptide Complex Pharmacokinetics and Safety in Cynomolgus Monkey

PC-1, PC-4, and PC-10 were dosed in Cynomolgus monkeys according to FIG. 45. PC-1 is a non-masked T cell engager used to establish a baseline reading for signs of toxicity and/or cytokine release similarly observed by others that have published data using bispecific T cell engagers (BiTEs). PC-4 is a fully human cyno cross reactive tumor activated T cell engager or polypeptide complex that is masked at both the tumor binding domain and the T cell binding domain. PC-10 is fully human cyno cross reactive polypeptide complex that is only masked at the T cell binding domain. FIG. 45 illustrates the three molecules. For all three molecules, the tumor binding domain recognized human and primate epidermal growth factor receptor (EGFR) and the T cell binding domain recognized human and primate cluster of differentiation 3 (CD3) . While CD3 is restricted to T cells, EGFR is expressed in many healthy tissues including liver, muscle, kidney, skin, intestine, and others. Typical adverse events directly related to EGFR targeted therapies include skin, liver, and intestine related toxicities, where EGFR is readily expressed.

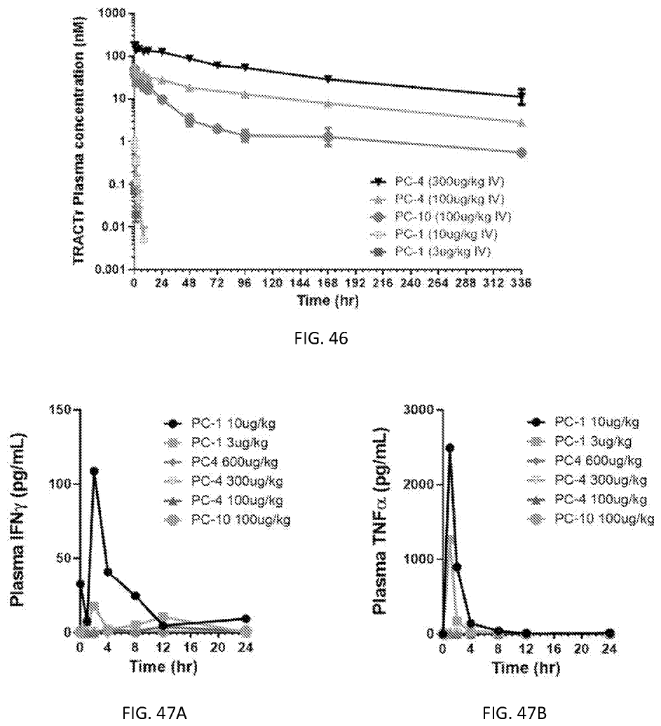

Cynomolgus monkeys were dosed according to TABLE 6. Blood samples were harvested at the timepoints listed in TABLE 7 and used for PK analysis as well as standard clinical chemistry, hematology, flow cytometry, and cytokine release panels as measures of safety. Summary of the safety data is shown in FIG. 46 and TABLE 8.

TABLE 6
Summary of dosages.

| Study/Report | Test article | Dose | Dose volume | Dose Route |
|---|---|---|---|---|
| JT1902 | PC-1 | 3 ug/kg | 1 mL/kg | IV bolus |
| JT1902 | PC-1 | 10 ug/kg | 1 mL/kg | IV bolus |
| JT1902 | PC-4 | 100 ug/kg | 1 mL/kg | IV bolus |
| JT1903 | PC-4 | 300 ug/kg | 1 mL/kg | IV bolus |
| JT1903 | PC-4 | 600 ug/kg | 1 mL/kg | IV bolus |
| JT2001 | PC-10 | 100 ug/kg | 1 mL/kg | IV bolus |

TABLE 7
Summary of time-points.

| Study Animals | Naive male cynomolgus primates (Macaca fascicularis) | |
|---|---|---|
| Test Article | PC-1, PC-4, and PC-10 | |
| Study Design | Group 1 | Time-Points |
| | PC-1 10 ug/kg @ | PK: Pre-dose, 15 min, 30 min, 1 hr, 2 hr, |
| | 0.01mg/mL IV Bolus (n = 2) | 4 hr, 8 hr, 12 hr, 24 hr, 48 hr. |
| | Dose Volume: 1 mL/kg | ALT/AST: Pre-dose, 24 hr. 48 hr |
| | Group 2 | Time-Points |
| | PC-1 3 ug/kg @ | PK: Pre-dose, 15 min, 30 min, 1 hr, 2 hr, |
| | 0.003 mg/mL IV Bolus | 4 hr, 8 hr, 12 hr, 24 hr, 48 hr. |
| | (n = 2) | ALT/AST: Pre-dose, 24 hr. 48 hr |
| | Dose Volume: 1 mL/kg | |
| | Group 3 | Time-Points |
| | PC-4 100 ug/kg @ | PK: Pre-dose, 15 min, 30 min, 1 hr, 2 hr, |
| | 0.1 mg/mL IV Bolus (n = 2) | 4 hr, 8 hr, 12 hr, 24 hr, 48 hr, 96 hr, 168 hr, |
| | Dose: Volume: 1 mL/kg | 336 hr. |
| | | AST/ALT: Pre-dose, 24 hr, 48 hr, 96 hr, |
| | | 168 hr |
| | Group 4 | Time-Points |
| | PC-4 300 ug/kg @ 0.3 | PK: Pre-dose, 15 min, 30 min, 1 hr, 2 hr, 4 hr, |
| | mg/mL IV Bolus (n = 2) | 8 hr, 12 hr, 24 hr, 48 hr, 72 hr, 96 hr, 168 hr, |
| | Dose Volume: 1 mL/kg | 336 hr. |
| | | Hematology/Clinical Chem: Pre-Dose, |
| | | 24 hr, 48 hr, 72 hr, 168 hr |
| | | Flow Cytometry: 2 Panels-Pre-Dose, 24 hr, |
| | | 48 hr, 72 hr, and 168 hr. |
| | Group 5 | Time-Points |
| | PC-4 600 ug/kg @ | PK: Pre-dose, 15 min, 30 min, 1 hr, 2 hr, 4 hr, |
| | 0.6 mg/mL IV Bolus (n = 2) | 8 hr, 12 hr, 24 hr, 48 hr, 72 hr, 96 hr, 168 hr, |
| | Dose Volume: 1 mL/kg | 336 hr. |
| | | Hematology/Clinical Chem: Pre-Dose, |
| | | 24 hr, 48 hr, 72 hr, 168 hr |
| | | Flow Cytometry: 2 Panels-Pre-Dose, 24 hr, |
| | | 48 hr, 72 hr, and 168 hr. |
| | Group 6 | Time-Points |
| | PC-10 100 ug/kg @ | PK: Pre-dose, 15 min, 30 min, 1 hr, 2 hr, 4 hr, |
| | 0.1 mg/mL IV Bolus (n = 2) | 8 hr, 12 hr, 24 hr, 48 hr, 72 hr, 96 hr, 168 hr, |
| | Dose Volume: 1 mL/kg | 336 hr. |
| | | Hematology/Clinical Chem: Pre-Dose, |
| | | 24 hr, 48 hr, 72 hr, 168 hr |
| | | Flow Cytometry: 2 Panels-Pre-Dose, 24 hr, |
| | | 48 hr, 72 hr, and 168 hr. |

| | |
|---|---|
| Endpoints | Observations twice a day |
| | Hematology/Clinical Chemistry |
| | Flow Cytometry: 2 Panels |
| | Blood processed to plasma using K2 EDTA |

TABLE 8
Summary of safety data.

| Cyno PK | PC-4 | PC-4 | PC-10 | PC-1 | PC-1 | |
|---|---|---|---|---|---|---|
| IV bolus | 300 ug/kg | 100 ug/kg | 100 ug/kg | 10 ug/kg | 3 ug/kg | Units |
| Tₘₐₓ | 0.0 | 0.0 | 0.0 | 0.0 | 0.0 | hr |
| Cₘₐₓ | 153.23 | 51.27 | 35.64 | 1.66 | 0.17 | nM |
| t_{1/2} | 108.90 | 110.86 | 132.77 | 1.30 | 0.32 | hr |
| Vd | 0.06 | 0.06 | 0.09 | 0.24 | 0.68 | L |
| VSS | 0.10 | 0.13 | 1.28 | 1.08 | 0.63 | L |
| CL | 0.13 | 0.12 | 0.16 | 41.72 | 500.62 | mL/hr/kg |
| BW | 3.00 | 3.00 | 3.00 | 3.00 | 3.00 | kg |

TABLE 9
Summary of cytokine release, clinical observations, clinical chemistry, hematology, and
flow cytometry analysis for each polypeptide complex dose group in cynomolgus monkey.

| Test | | IL-6 | Clinical | Clinical | Clinical | Flow |
|---|---|---|---|---|---|---|
| article | Dose | spike | observations | chemistry | pathology | cytometry |
| PC-1 | 3 ug/kg | 1933 | None | None | Not tested | Not tested |
| | | pg/mL | | | | |
| PC-1 | 10 ug/kg | 5902 | None | None | Not tested | Not tested |
| | | pg/mL | | | | |
| PC-4 | 100 ug/kg | 14 | None | None | Not tested | Not tested |
| | | pg/mL | | | | |
| PC-4 | 300 ug/kg | 587 | None | Transient or | None | None |
| | | pg/mL | | none | | |
| PC-4 | 600 ug/kg | 516 | Mild diarrhea | Transient or | None | None |
| | | pg/mL | 1 day | none | | |
| PC-10 | 100 ug/kg | 628 | Diarrhea | ALT persistence | Lymphocyte | CD3 decrease & |
| | | pg/mL | 4 days | & | decrease | Ki-67 persistence |
| | | | | Hypoalbuminemia | | |

Cynomolgus Monkeys

Young näive male Cynomolgus monkeys were paired housed by group and identified by unique body tattoo. All animals were acclimated to housing conditions for 3 days prior to the start of the study. Prior to initiation all animals had undergone a physical examination by the study veterinarian. Only animals that, in the opinion of the study veterinarian, were healthy and otherwise met the criteria were admitted to the study. Food was withheld overnight prior to dosing. Purina 5049 was provided daily in amounts appropriate for the size of the animal Tap water was provided ad libitum via automatic watering device.

Pharmacokinetics

Polypeptide complex pharmacokinetics were determined in näive male Cynomolgus monkeys weighing 2-3 kg. Briefly, two group housed monkeys were used per dosing group and allowed to acclimate to their surroundings prior to dosing Animals were sedated with Ketamine HCL 10-20 mg/kg IM prior to dosing and bleeding. Concentrated test articles were diluted in sterile phosphate buffered saline and administered to animals at a quantity relative to the animals' mass in kg. The dose for each test article was administered intravenously at 1 mL/kg dosing volume. For dosing, the left and right limbs were clipped and prepped with alcohol. The saphenous vein was identified, and a standard catheter was placed for IV bolus infusion (in either the left or right limb). The test article dosing solution was attached to the catheter via syringe and the bolus infusion occurred via manual compression of the syringe.

For blood collections, animals were sedated using ketamine, the femoral triangle was prepared, and blood was collected from the femoral vein using a 22G 1.5 inch needle, vacutainer sheath, and collection tube. Following venipuncture, manual compression of the vein was maintained until hemostasis was achieved. Blood collections were based on weight of the animals and did not exceed AGI maximum bleeds as set forth by IACUC. Blood was collected in EDTA tubes and processed to plasma. The blood samples were centrifuged cold at 3000×g for 10 min to separate cells from plasma. The plasma supernatant was harvested and stored frozen prior to analysis.

The concentration of polypeptide complex in cyno plasma samples was determined by ELISA. Briefly, anti-histag capture antibody was coated directly on ELISA plates. Standard dilutions of polypeptide complex in cyno serum were used to generate a calibration curve to which animal PK test samples could be compared. Standards and test samples were added to the plate and incubated cold overnight. Several different dilutions of test samples were used to make sure signals landed within appropriate dynamic range of the standard curve. Plates were washed and incubated with an anti-human HRP detection antibody for a brief time. Plates were washed, developed, and stopped using standard ELISA techniques. Standard curves plotting absorbance at 450 nm versus known polypeptide complex concentration were used to calculate the concentration of unknown test articles in each mouse PK plasma sample. Concentration of polypeptide complex were plotted versus time and fit to a standard two stage distribution and elimination pharmacokinetic model. The calculated pharmacokinetic and parameters for PC-1 and PC-4 from Cynomolgus monkey are shown in FIG. 46 and TABLE 8.

Cytokine Release

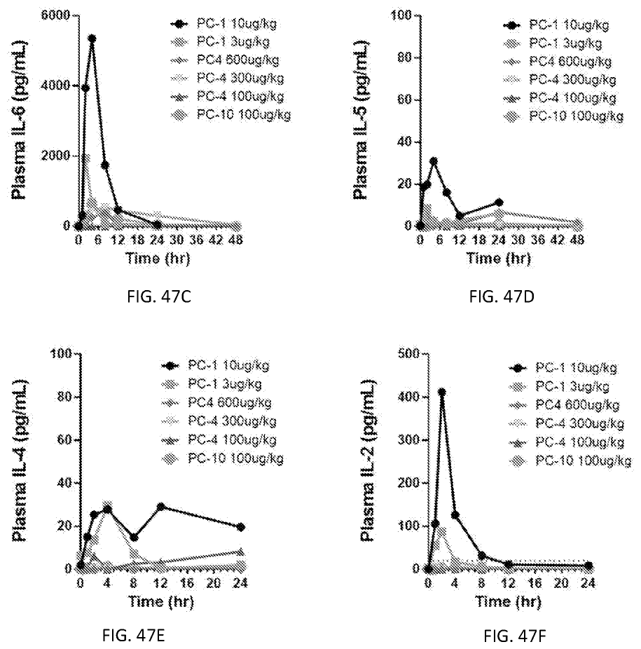

Cytokines present in plasma post treatment were measured using the non-human primate Th1/Th2 cytometric bead array assay kit from BD Biosciences (Cat no. 557800) according to the manufacturer's instructions. Data is shown in FIGS. 47A-47F.

Flow Cytometry

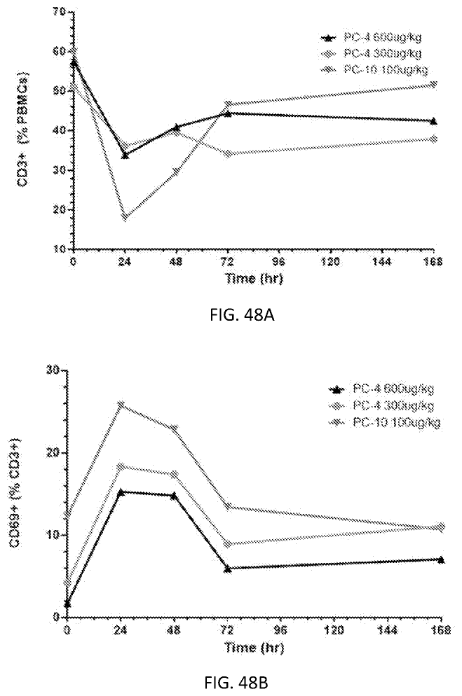

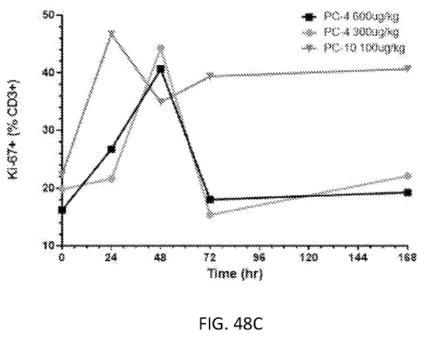

Blood samples were processed to PBMCs, stained and analyzed by flow cytometry. Cells were stained for CD45, CD3, CD4, CD8, CD69, and KI-67. Data is shown in FIGS. 48A-48C.

Clinical Chemistry and Hematology

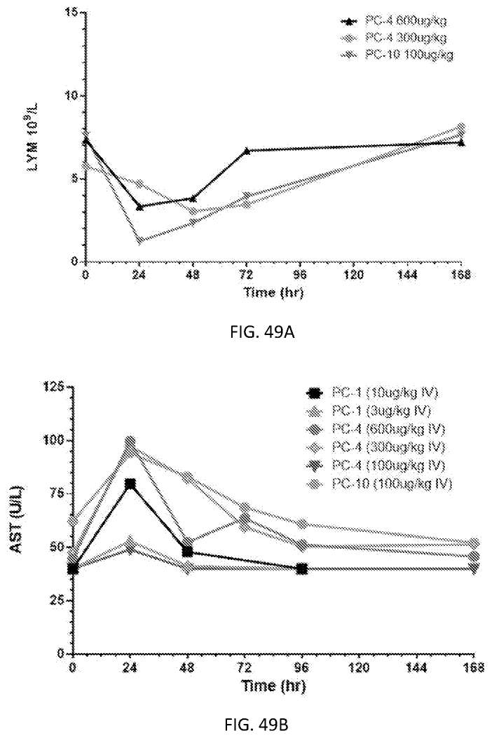

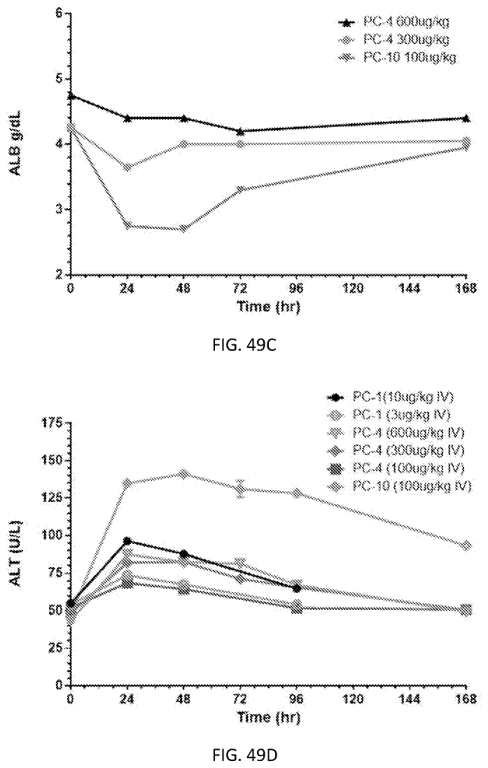

Blood samples were run in standard clinical chemistry and hematology panels. Example data is shown in FIGS. 49A-49D.

Clinical Observations

Animals were observed twice daily to qualify any potential clinical or behavioral observations as signs of toxicity.

PC-1 Pharmacokinetics and Safety Observations

PC-1 was cleared rapidly with a half-life of 1 hr after IV bolus administration in Cynomolgus monkey. Even with the rapid clearance, PC-1 at the low doses tested clearly caused a spike in the acute cytokine release measured by increases in IL-6, TNFα, IFNγ, and IL-2 analogous to several previously published BiTEs. Published data using BiTEs clearly argue that cytokine release is due to drug target engagement and activity in normal healthy tissue. No other signs of toxicity were measured nor observed with PC-1 presumably due to the fast clearance of the molecule. Continuous infusion of anti-EGFR BiTEs has been published to cause liver and gastrointestinal toxicity.

PC-4 Pharmacokinetics and Safety Observations

PC-4 exposure after IV bolus exhibited a long half-life around 110 hr in Cynomolgus monkey. The observed pharmacokinetic properties of PC-4 were in line with published anti-albumin single domain antibodies. Even with the vastly elevated C_{MAX}, AUC, and half-life of PC-4 relative to PC-1, PC-4 clearly did not cause acute cytokine release. Despite administering PC-4 at 200× higher doses relative to PC-1, minimal elevation in IL-6 was observed, a key driver of clinical complications related to cytokine release syndrome. Unlike PC-1, TNFα, IFNγ, and IL-2 levels remained equivalent to background after PC-4 administration. In addition, the clinical chemistry, hematology, and flow cytometry panels for all animals administered PC-4 were as expected and viewed as normal where none of the measurements met criteria to define a clear adverse event. Some early timepoint measurements in clinical chemistry and hematology drifted outside the historic normal ranges but were considered mild and transient that progressed to normal levels over a short time. The clinical observation of very mild lower eyelid swelling in the 100 ug/kg PC-4 group was not observed in the higher PC-4 dose groups. The eyelid swelling was attributed to rough cage play with pair housed male Cynomolgus monkeys. PC-4 at the highest dose group 600 ug/kg showed mild signs of diarrhea on Day 2 that cleared the following day. No other observations were noted for PC-4. Importantly, any changes in clinical chemistry, hematology, flow cytometry, as well as clinical and behavioral observations for PC-4 treated animals were mild and not dose dependent. Given these results it is clear that the maximum tolerated dose (MTD) of PC-4 was not reached despite testing up to 600 ug/kg IV bolus.

PC-10 Pharmacokinetics and Safety Observations

PC-10 exposure after IV bolus exhibited a long half-life around 130 hr in Cynomolgus monkey consistent with PC-4 and the published anti-albumin single domain antibodies. However, the PK alpha phase of PC-10 clearly demonstrated some target mediated drug disposition (TMDD) indicated by the steeper drop in early plasma concentrations relative to PC-4. TMDD implies PC-10 partitioned outside the blood compartment and accumulated in healthy tissues likely expressing EGFR. PC-10 also had higher cytokines release levels relative to PC-4 when comparing the 100 ug/kg dose groups. Furthermore, PC-10 caused a significant depletion and margination of lymphocytes and CD3+ T cells within 24 hours that slowly returned to normal over the course of the study. T cells appeared to continually proliferate after PC-10 dosing indicated by the elevated Ki-67 marker in CD3+ cells that persisted over the one week timeframe. Additionally, PC-10 appeared to elevate ALT levels outside the normal range for a sustained period of 72 hours. This is presumed to be due to accumulation of PC-10 in the liver or muscle where it causes some healthy tissue inflammation and the release of ALT. PC-10 also caused significant hypoalbuminemia through 72 hours. Significant decrease in serum albumin is often indicative of generalized inflammation as well as intestinal, hepatic, renal, or cardiac toxicity. Gut and renal losses of albumin can also cause hypoalbuminemia often times accompanied by proteinuria and diarrhea, respectively. PC-10 caused diarrhea in one animal for a sustained timeframe of 4 days. This animal with persistent diarrhea exhibited more severe hypoalbuminemia relative to the other animal Based on the clinical chemistry, hematology, flow cytometry, as well as clinical and behavioral observations it appears PC-10 started to show clear signs of toxicity whereas PC-4 at higher doses and exposures appeared to remain safe.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A polypeptide complex comprising:
a single chain variable fragment (scFv) comprising a light chain variable domain and a heavy chain variable domain, wherein the scFv is linked to a peptide (P₁) at an N-terminus of the scFv with a linking moiety (L₁) that is a substrate for a tumor specific protease, wherein P₁ impairs binding of the scFv to an effector cell antigen, and P₁ is further linked to a half-life extending molecule; and
an antigen recognizing molecule that binds to a tumor cell antigen, wherein the antigen recognizing molecule comprises a Fab light chain polypeptide and a Fab heavy chain polypeptide, wherein the antigen recognizing molecule is linked to the scFv, and the antigen recognizing molecule is further linked to P₂ and L₂, wherein P₂ comprises a peptide that impairs binding of the antigen recognizing molecule to the tumor cell antigen; and L₂ comprises a linking moiety that connects the antigen recognizing molecule to P₂ and is a substrate for a tumor specific protease.

2. The polypeptide complex of claim 1, wherein the antigen recognizing molecule is a Fab or a Fab′.

3. (canceled)

4. (canceled)

5. (canceled)

6. (canceled)

7. The polypeptide complex of claim 1, wherein the polypeptide complex has a molecular weight of less than about 110 kDa.

8. (canceled)

9. (canceled)

10. The polypeptide complex of claim 1, wherein the effector cell antigen comprises cluster of differentiation 3 (CD3).

11. (canceled)

12. The polypeptide complex of claim 1, wherein the scFv comprises complementary determining regions (CDR)s of SP34.

13. The polypeptide complex of claim 1, wherein the scFv comprises an amino acid sequence that has at least 85% sequence identity to SEQ ID NO: 66, SEQ ID NO: 67, or SEQ ID NO: 68.

14. (canceled)

15. (canceled)

16. The polypeptide complex of claim 1, wherein P₁ comprises an amino acid sequence of at least 10 amino acids in length and no more than 20 amino acids in length.

17. (canceled)

18. (canceled)

19. The polypeptide complex of claim 1, wherein P₁ comprises an amino acid sequence according to SEQ ID NOs: 19, 20, 21, 22, 23, 24, 25, 26, 27, or 28.

20. The polypeptide complex of claim 1, wherein L₁ comprises a urokinase cleavable amino acid sequence, a matriptase cleavable amino acid sequence, matrix metalloprotease cleavable amino acid sequence, or a legumain cleavable amino acid sequence.

21. (canceled)

22. The polypeptide complex of claim 1, wherein L₁ comprises an amino acid sequence according to SEQ ID NOs: 36, 37, 38, 39, or 46.

23. (canceled)

24. The polypeptide complex of claim 1, wherein the half-life extending molecule comprises a linking moiety (L₃) that connects the half-life extending molecule to P₁.

25. (canceled)

26. The polypeptide complex of claim 24, wherein L₃ comprises an amino acid sequence according to SEQ ID NO: 51.

27. The polypeptide complex of claim 1, wherein the half-life extending molecule comprises an antibody.

28. The polypeptide complex of claim 27, wherein the antibody comprises a single domain antibody, a single chain variable fragment, or a Fab.

29. The polypeptide complex of claim 28, wherein the single domain antibody binds to albumin.

30. The polypeptide complex of claim 28, wherein the single domain antibody comprises 10G or 10GE.

31. The polypeptide complex of claim 30, wherein the single domain antibody comprises 10G, and the single domain antibody comprises an amino acid sequence according to SEQ ID NO: 52.

32. (canceled)

33. (canceled)

34. (canceled)

35. (canceled)

36. (canceled)

37. The polypeptide complex of claim 3-61, wherein P₂ comprises an amino acid sequence according to SEQ ID NOs: 1, 2, 3, 4, 5, or 6.

38. (canceled)

39. (canceled)

40. (canceled)

41. (canceled)

42. (canceled)

43. (canceled)

44. The polypeptide complex of claim 1, wherein L₂ comprises a urokinase cleavable amino acid sequence, a matriptase cleavable amino acid sequence, matrix metalloprotease cleavable amino acid sequence, or a legumain cleavable amino acid sequence.

45. (canceled)

46. The polypeptide complex of claim 1, wherein L₂ comprises an amino acid sequence according to SEQ ID NOs: 36, 37, 38, 39, or 46.

47. (canceled)

48. (canceled)

49. (canceled)


SEQUENCE LISTING (113 sequences)

SEQ ID NO: 1 - protein, 16 aa, Artificial Sequence
  Description of Artificial Sequence Synthetic peptide
GGDWCRSLMSYTDLCP

SEQ ID NO: 2 - protein, 16 aa, Artificial Sequence
  Description of Artificial Sequence Synthetic peptide
GGTSCADAHLIAPSCS

SEQ ID NO: 3 - protein, 16 aa, Artificial Sequence
  Description of Artificial Sequence Synthetic peptide
GGNCQWDRVEHTYACS

SEQ ID NO: 4 - protein, 16 aa, Artificial Sequence
  Description of Artificial Sequence Synthetic peptide
GGWVSCHDGSHMTCFH

SEQ ID NO: 5 - protein, 16 aa, Artificial Sequence
  Description of Artificial Sequence Synthetic peptide
GGMNCLNRLWVEYCLV

SEQ ID NO: 6 - protein, 16 aa, Artificial Sequence
  Description of Artificial Sequence Synthetic peptide
GGYCGQDNTWVREGCF

SEQ ID NO: 7 - protein, 21 aa, Artificial Sequence
  Description of Artificial Sequence Synthetic peptide
QGQSGQLSCEGWAMNREQCRA

SEQ ID NO: 8 - protein, 16 aa, Artificial Sequence
  Description of Artificial Sequence Synthetic peptide
GGPLCSDLDHITRLCD

SEQ ID NO: 9 - protein, 16 aa, Artificial Sequence
  Description of Artificial Sequence Synthetic peptide
GGIDCASLDHYTESCY

SEQ ID NO: 10 - protein, 16 aa, Artificial Sequence
  Description of Artificial Sequence Synthetic peptide
GGNPVCTLGDPYECSH

SEQ ID NO: 11 - protein, 16 aa, Artificial Sequence
  Description of Artificial Sequence Synthetic peptide
GGTFCQLNADPYECQS

SEQ ID NO: 12 - protein, 16 aa, Artificial Sequence
  Description of Artificial Sequence Synthetic peptide
GGGYCELIGDYVVCSP

SEQ ID NO: 13 - protein, 16 aa, Artificial Sequence
  Description of Artificial Sequence Synthetic peptide
GGLCDRWGWIDAPYCH

SEQ ID NO: 14 - protein, 16 aa, Artificial Sequence
  Description of Artificial Sequence Synthetic peptide
GGTGCTEGHWHWGTCS

SEQ ID NO: 15 - protein, 16 aa, Artificial Sequence
  Description of Artificial Sequence Synthetic peptide
GGNICMDYSWRSGCAV

SEQ ID NO: 16 - protein, 16 aa, Artificial Sequence
  Description of Artificial Sequence Synthetic peptide
GGHSCTFGDWSLGTCA

SEQ ID NO: 17 - protein, 16 aa, Artificial Sequence
  Description of Artificial Sequence Synthetic peptide
GGFICTLGNWWDGSCE

SEQ ID NO: 18 - protein, 21 aa, Artificial Sequence
  Description of Artificial Sequence Synthetic peptide
QGQSGQGYLWGCEWNCGGITT

SEQ ID NO: 19 - protein, 16 aa, Artificial Sequence
  Description of Artificial Sequence Synthetic peptide
GGDSVCADPEVPICEI

SEQ ID NO: 20 - protein, 16 aa, Artificial Sequence
  Description of Artificial Sequence Synthetic peptide
GGMSDCGDPGVEICTH

SEQ ID NO: 21 - protein, 16 aa, Artificial Sequence
  Description of Artificial Sequence Synthetic peptide
GGIQCHDPDLPSPCYI

SEQ ID NO: 22 - protein, 16 aa, Artificial Sequence
  Description of Artificial Sequence Synthetic peptide
GGEWCLFDPDVPTCQD

SEQ ID NO: 23 - protein, 16 aa, Artificial Sequence
  Description of Artificial Sequence Synthetic peptide
GGLGCNDIDPGEQCIV

SEQ ID NO: 24 - protein, 16 aa, Artificial Sequence
  Description of Artificial Sequence Synthetic peptide
GGLECFDPEIPEAFCI

SEQ ID NO: 25 - protein, 16 aa, Artificial Sequence
  Description of Artificial Sequence Synthetic peptide
GGQGCGTIADPEPHCW

SEQ ID NO: 26 - protein, 16 aa, Artificial Sequence
  Description of Artificial Sequence Synthetic peptide
GGNCHDPDIPAYVLCS

SEQ ID NO: 27 - protein, 16 aa, Artificial Sequence
  Description of Artificial Sequence Synthetic peptide
GGLCPINDWEPQDICW

SEQ ID NO: 28 - protein, 16 aa, Artificial Sequence
  Description of Artificial Sequence Synthetic peptide
GGLCMIGDWLPGDVCL

SEQ ID NO: 29 - protein, 9 aa, Artificial Sequence
  Description of Artificial Sequence Synthetic peptide
  MISC_FEATURE (1)..9): This sequence may encompass 1-3 “Gly Gly Ser” repeating units
GGSGGSGGS

SEQ ID NO: 30 - protein, 5 aa, Artificial Sequence
  Description of Artificial Sequence Synthetic peptide
  See specification as filed for detailed description of substitutions and preferred embodiments
GSGGS

SEQ ID NO: 31 - protein, 4 aa, Artificial Sequence
  Description of Artificial Sequence Synthetic peptide
  See specification as filed for detailed description of substitutions and preferred embodiments
GGGS

SEQ ID NO: 32 - protein, 5 aa, Artificial Sequence
  Description of Artificial Sequence Synthetic peptide
  See specification as filed for detailed description of substitutions and preferred embodiments
GGGGS

SEQ ID NO: 33 - protein, 6 aa, Artificial Sequence
  Description of Artificial Sequence Synthetic peptide
  See specification as filed for detailed description of substitutions and preferred embodiments
GSSGGS

SEQ ID NO: 34 - protein, 18 aa, Artificial Sequence
  Description of Artificial Sequence Synthetic peptide
GGGGSLSGRSDNHGSSGT

SEQ ID NO: 35 - protein, 26 aa, Artificial Sequence
  Description of Artificial Sequence Synthetic peptide
GGGGSSGGSGGSGLSGRSDNHGSSGT

SEQ ID NO: 36 - protein, 8 aa, Artificial Sequence
  Description of Artificial Sequence Synthetic peptide
ASGRSDNH

SEQ ID NO: 37 - protein, 8 aa, Artificial Sequence
  Description of Artificial Sequence Synthetic peptide
LAGRSDNH

SEQ ID NO: 38 - protein, 13 aa, Artificial Sequence
  Description of Artificial Sequence Synthetic peptide
ISSGLASGRSDNH

SEQ ID NO: 39 - protein, 13 aa, Artificial Sequence
  Description of Artificial Sequence Synthetic peptide
ISSGLLAGRSDNH

SEQ ID NO: 40 - protein, 8 aa, Artificial Sequence
  Description of Artificial Sequence Synthetic peptide
LSGRSDNH

SEQ ID NO: 41 - protein, 13 aa, Artificial Sequence
  Description of Artificial Sequence Synthetic peptide
ISSGLLSGRSDNP

SEQ ID NO: 42 - protein, 13 aa, Artificial Sequence
  Description of Artificial Sequence Synthetic peptide
ISSGLLSGRSDNH

SEQ ID NO: 43 - protein, 16 aa, Artificial Sequence
  Description of Artificial Sequence Synthetic peptide
LSGRSDNHSPLGLAGS

SEQ ID NO: 44 - protein, 16 aa, Artificial Sequence
  Description of Artificial Sequence Synthetic peptide
SPLGLAGSLSGRSDNH

SEQ ID NO: 45 - protein, 12 aa, Artificial Sequence
  Description of Artificial Sequence Synthetic peptide
SPLGLSGRSDNH

SEQ ID NO: 46 - protein, 16 aa, Artificial Sequence
  Description of Artificial Sequence Synthetic peptide
LAGRSDNHSPLGLAGS

SEQ ID NO: 47 - protein, 16 aa, Artificial Sequence
  Description of Artificial Sequence Synthetic peptide
LSGRSDNHVPLSLKMG

SEQ ID NO: 48 - protein, 16 aa, Artificial Sequence
  Description of Artificial Sequence Synthetic peptide
LSGRSDNHVPLSLSMG

SEQ ID NO: 49 - protein, 31 aa, Artificial Sequence
  Description of Artificial Sequence Synthetic polypeptide
GSSGGSGGSGGSGISSGLLSGRSDNHGSSGT

SEQ ID NO: 50 - protein, 28 aa, Artificial Sequence
  Description of Artificial Sequence Synthetic peptide
GSSGGSGGSGGISSGLLSGRSDNHGGGS

SEQ ID NO: 51 - protein, 9 aa, Artificial Sequence
  Description of Artificial Sequence Synthetic peptide
GGGGSGGGS

SEQ ID NO: 52 - protein, 115 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSS

SEQ ID NO: 53 - protein, 21 aa, Artificial Sequence
  Description of Artificial Sequence Synthetic peptide
QGQSGSGYLWGCEWNCGGITT

SEQ ID NO: 54 - protein, 28 aa, Artificial Sequence
  Description of Artificial Sequence Synthetic peptide
GSSGGSGGSGGASSGAGGSGGGSGGGGS

SEQ ID NO: 55 - protein, 30 aa, Artificial Sequence
  Description of Artificial Sequence Synthetic polypeptide
GGGGSGGGGSGGISSGLLSGRSDNHGSSGT

SEQ ID NO: 56 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 214 aa, Artificial Sequence
  Description of Artificial Sequence Synthetic polypeptide
QILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 222 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 60 - protein, 222 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 61 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 223 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 64 - protein, 214 aa, Artificial Sequence
  Description of Artificial Sequence Synthetic polypeptide
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 65 - protein, 449 aa, Artificial Sequence
  Description of Artificial Sequence Synthetic polypeptide
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 66 - protein, 249 aa, Artificial Sequence
  Description of Artificial Sequence Synthetic polypeptide
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 67 - protein, 249 aa, Artificial Sequence
  Description of Artificial Sequence Synthetic polypeptide
QTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSS

SEQ ID NO: 68 - protein, 249 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 69 - protein, 503 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHHGGGLNDIFEAQKIEWHE

SEQ ID NO: 70 - protein, 266 aa, Artificial Sequence
  Description of Artificial Sequence Synthetic polypeptide
QGQSGQLSCEGWAMNREQCRAGSSGGSGGSGGSGISSGLLSGRSDNHGSSGTDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 552 aa, Artificial Sequence
  Description of Artificial Sequence Synthetic polypeptide
QGQSGQGYLWGCEWNCGGITTGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHHGGGLNDIFEAQKIEWHE

SEQ ID NO: 72 - protein, 390 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQGQSGQLSCEGWAMNREQCRAGSSGGSGGSGGSGISSGLLSGRSDNHGSSGTDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 676 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQGQSGQGYLWGCEWNCGGITTGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHHGGGLNDIFEAQKIEWHE

SEQ ID NO: 74 - protein, 655 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGQGCGTIADPEPHCWGSSGGSGGSGGISSGLLSGRSDNHGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGHHHHHHHH

SEQ ID NO: 75 - protein, 654 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGQGCGTIADPEPHCWGSSGGSGGSGGASSGAGGSGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHH

SEQ ID NO: 76 - protein, 655 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSGGQGCGTIADPEPHCWGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGHHHHHHHH

SEQ ID NO: 77 - protein, 503 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHHGGGLNDIFEAQKIEWHE

SEQ ID NO: 78 - protein, 676 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQGQSGSGYLWGCEWNCGGITTGSSGGSGGSGGISSGLLSGRSDNHGGGSQTVVTQEPSFSVSPGGTVTLTCRSSTGAVTTSNYANWVQQTPGQAPRGLIGGTNKRAPGVPDRFSGSILGNKAALTITGAQADDESDYYCALWYSNLWVFGGGTKLTVLGGGGSGGGGSGGGGSEVQLVESGGGLVQPGGSLKLSCAASGFTFSTYAMNWVRQASGKGLEWVGRIRSKYNNYATYYADSVKDRFTISRDDSKNTAYLQMNSLKTEDTAVYYCTRHGNFGNSYVSWFAYWGQGTLVTVSSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSQDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGHHHHHHHHGGGLNDIFEAQKIEWHE

SEQ ID NO: 79 - protein, 488 aa, Artificial Sequence
  Description of Artificial Sequence Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHHH

SEQ ID NO: 80 - protein, 260 aa, Artificial Sequence
  Description of Artificial Sequence Synthetic polypeptide
GGTGCTEGHWHWGTCSGGGGSGGGGSGGISSGLLSGRSDNHGSSGTDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 661 aa, Artificial Sequence
  Description of Artificial Sequence Synthetic polypeptide
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQGQSGQGYLWGCEWNCGGITTGSSGGSGGSGGISSGLLSGRSDNHGGGSQTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGGGGSGGGGSGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHHH

SEQ ID NO: 82 - protein, 5 aa, Artificial Sequence
  Description of Artificial Sequence Synthetic peptide
GGGGS

SEQ ID NO: 83 - protein, 21 aa, Artificial Sequence
  Description of Artificial Sequence Synthetic peptide
GGPLCSDLDHITRLCDGGGGS

SEQ ID NO: 84 - protein, 21 aa, Artificial Sequence
  Description of Artificial Sequence Synthetic peptide
GGIDCASLDHYTESCYGGGGS

SEQ ID NO: 85 - protein, 21 aa, Artificial Sequence
  Description of Artificial Sequence Synthetic peptide
GGNPVCTLGDPYECSHGGGGS

SEQ ID NO: 86 - protein, 21 aa, Artificial Sequence
  Description of Artificial Sequence Synthetic peptide
GGTFCQLNADPYECQSGGGGS

SEQ ID NO: 87 - protein, 21 aa, Artificial Sequence
  Description of Artificial Sequence Synthetic peptide
GGGYCELIGDYVVCSPGGGGS

SEQ ID NO: 88 - protein, 21 aa, Artificial Sequence
  Description of Artificial Sequence Synthetic peptide
GGLCDRWGWIDAPYCHGGGGS

SEQ ID NO: 89 - protein, 21 aa, Artificial Sequence
  Description of Artificial Sequence Synthetic peptide
GGTGCTEGHWHWGTCSGGGGS

SEQ ID NO: 90 - protein, 21 aa, Artificial Sequence
  Description of Artificial Sequence Synthetic peptide
GGNICMDYSWRSGCAVGDGGS

SEQ ID NO: 91 - protein, 21 aa, Artificial Sequence
  Description of Artificial Sequence Synthetic peptide
GGHSCTFGDWSLGTCAGGGGS

SEQ ID NO: 92 - protein, 21 aa, Artificial Sequence
  Description of Artificial Sequence Synthetic peptide
GGFICTLGNWWDGSCEGGGGS

SEQ ID NO: 93 - protein, 21 aa, Artificial Sequence
  Description of Artificial Sequence Synthetic peptide
GGDWCRSLMSYTDLCPGGGGS

SEQ ID NO: 94 - protein, 21 aa, Artificial Sequence
  Description of Artificial Sequence Synthetic peptide
GGTSCADAHLIAPSCSGGGGS

SEQ ID NO: 95 - protein, 21 aa, Artificial Sequence
  Description of Artificial Sequence Synthetic peptide
GGNCQWDRVEHTYACSGGGGS

SEQ ID NO: 96 - protein, 21 aa, Artificial Sequence
  Description of Artificial Sequence Synthetic peptide
GGWVSCHDGSHMTCFHGGGGS

SEQ ID NO: 97 - protein, 21 aa, Artificial Sequence
  Description of Artificial Sequence Synthetic peptide
GGMNCLNRLWVEYCLVGGGGS

SEQ ID NO: 98 - protein, 21 aa, Artificial Sequence
  Description of Artificial Sequence Synthetic peptide
GGYCGQDNTWVREGCFGGGGS

SEQ ID NO: 99 - protein, 26 aa, Artificial Sequence
  Description of Artificial Sequence Synthetic peptide
QGQSGQLSCEGWAMNREQCRAGGGGS

SEQ ID NO: 100 - protein, 26 aa, Artificial Sequence
  Description of Artificial Sequence Synthetic peptide
QGQSGQGYLWGCEWNCGGITTGGGGS

SEQ ID NO: 101 - protein, 21 aa, Artificial Sequence
  Description of Artificial Sequence Synthetic peptide
GGDSVCADPEVPICEIGGGGS

SEQ ID NO: 102 - protein, 21 aa, Artificial Sequence
  Description of Artificial Sequence Synthetic peptide
GGMSDCGDPGVEICTHGGGGS

SEQ ID NO: 103 - protein, 21 aa, Artificial Sequence
  Description of Artificial Sequence Synthetic peptide
GGIQCHDPDLPSPCYIGGGGS

SEQ ID NO: 104 - protein, 21 aa, Artificial Sequence
  Description of Artificial Sequence Synthetic peptide
GGEWCLFDPDVPTCQDGGGGS

SEQ ID NO: 105 - protein, 21 aa, Artificial Sequence
  Description of Artificial Sequence Synthetic peptide
GGLGCNDIDPGEQCIVGGGGS

SEQ ID NO: 106 - protein, 21 aa, Artificial Sequence
  Description of Artificial Sequence Synthetic peptide
GGLECFDPEIPEAFCIGGGGS

SEQ ID NO: 107 - protein, 21 aa, Artificial Sequence
  Description of Artificial Sequence Synthetic peptide
GGQGCGTIADPEPHCWGGGGS

SEQ ID NO: 108 - protein, 21 aa, Artificial Sequence
  Description of Artificial Sequence Synthetic peptide
GGNCHDPDIPAYVLCSGGGGS

SEQ ID NO: 109 - protein, 21 aa, Artificial Sequence
  Description of Artificial Sequence Synthetic peptide
GGLCPINDWEPQDICWGGGGS

SEQ ID NO: 110 - protein, 21 aa, Artificial Sequence
  Description of Artificial Sequence Synthetic peptide
GGLCMIGDWLPGDVCLGGGGS

SEQ ID NO: 111 - protein, 12 aa, Artificial Sequence
  Description of Artificial Sequence Synthetic peptide
IGGLLSGRSDNH

SEQ ID NO: 112 - DNA, 12 bp, Artificial Sequence
  Description of Artificial Sequence Synthetic oligonucleotide
cgcttggcatta

SEQ ID NO: 113 - protein, 4 aa, Artificial Sequence
  Description of Artificial Sequence Synthetic peptide
RLAL